(19) United States
(12) Patent Application Publication
(10) Pub. No.: US 2009/0306133 A1
(43) Pub. Date: Dec. 10, 2009

(54) New Acetyl Coenzyme A Carboxylase (ACC) Inhibitors And Uses In Treatments Of Obesity And Diabetes Mellitus - 087

(75) Inventors: David Blomberg, Molndal (SE); Anders Holmen, Molndal (SE); Petra Johannesson, Molndal (SE); Pernilla Sorme, Molndal (SE); Pernilla Stahlberg, Molndal (SE); Kay Brickmann, Molndal (SE); Volker Schnecke, Molndal (SE); Eric Wellner, Molndal (SE); Ragnar Hovland, Sodertalje (SE); Asa Mansson, Molndal (SE); Alleyn Plowright, Macclesfield (GB)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/339,600

(22) Filed: Dec. 19, 2008

Related U.S. Application Data
(60) Provisional application No. 61/085,059, filed on Jul. 31, 2008.
(60) Provisional application No. 61/015,787, filed on Dec. 21, 2007.

(51) Int. Cl. A61K 31/4709; C07D 215/38; C07D 401/14; A61P 9/10; A61P 3/10; A61K 31/47

(52) U.S. Cl. 514/314; 546/169; 546/167; 514/311

(57) ABSTRACT

The present invention relates to Acetyl Coenzyme A Carboxylase (ACC) inhibitors according to formula (I),

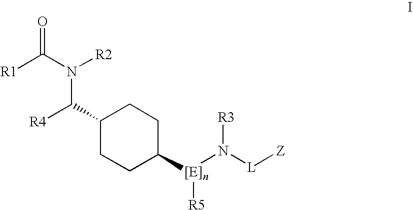

or an enantiomer thereof, or a pharmaceutically acceptable salt thereof, where R₁, R₂, R₃, R₄, R₅, E, L, Z and n are as defined herein, to processes for preparing such compounds, to pharmaceutical compositions containing them, to the use of such inhibitors and to methods for their therapeutic use, particularly in the treatments of obesity and diabetes mellitus.

### CROSS-REFERENCE TO RELATED APPLICATIONS

This application claims priority to U.S. Ser. No. 61/085,059 filed Jul. 31, 2008 and to U.S. Ser. No. 61/015,787 filed Dec. 21, 2007, each of which is incorporated herein by reference in its entirety.

### FIELD OF THE INVENTION

The present invention relates to Acetyl Coenzyme A Carboxylase (ACC) inhibitors, to processes for preparing such compounds, to pharmaceutical compositions containing them, to the use of such inhibitors and to methods for their therapeutic use, particularly in the treatments of obesity and diabetes mellitus.

### BACKGROUND OF THE INVENTION

Obesity and diabetes are reaching epidemic proportions in the USA, EU, Japan and developing countries. This epidemic is largely attributed to proliferation of key risk factors, which include a sedentary lifestyle, increased food intake and the demographic shift to a more aged population.

In mammals, ACC exists in two tissue specific isozymes, a liver, adipose and pancreas specific isozyme, ACC1, and a muscle specific isozyme, ACC2. The isozymes ACC1 and ACC2 have pivotal functions in fatty acid metabolism. They catalyse the production of malonyl-CoA from acetyl-CoA which is a component involved in fat oxidation. By inhibition of the ACC, the fat oxidation would be increased, which generates an improved insulin sensitivity in the body. Since the incidence of type 2 diabetes has increased dramatically during the past decade, there is a need for an effective ACC inhibitor to prevent or treat obesity and diabetes and quench their life threatening sequalae atherosclerosis, heart failure and stroke.

The compounds of the invention are ACC2 inhibitors. Also, some of the compounds have ACC1 inhibitory activity as well.

Compounds known as ACC inhibitors have been described, for example in J. Med. Chem. 2007, 50, 1078-1082, Yu Gui Gu et al., WO2007/011809, WO2003/072197 and WO2007/013691.

There are compounds related to formula (I) described in the literature by WO 97/20823 and Bioorganic & Medicinal Chemistry Letters 12 (2002) 1767-1769 (tert-butyl [(4-{[(1-naphthylsulfonyl)amino]methyl}cyclohexyl)methyl]carbamate and tert-butyl [(trans-4-{[(2-naphthylsulfonyl)amino]methyl}cyclohexyl)methyl]carbamate and tert-butyl({trans-4-[({[2-trifluoromethyl)phenyl]sulfonyl}amino)methyl]cyclohexyl) methyl) carbamate.) These compounds are disclosed as intermediates in a process for producing NPY Y5 receptor agonists. No pharmaceutical use of the prepared compounds is contemplated. The compounds disclosed in this document are disclaimed by proviso a).

Further, EP 1295 867 A1 describes a compound related to formula (I), which is an intermediate in the synthesis of the final compounds having obesity activity. This compound is disclaimed as no. 63 in proviso b) below.

Further, following compounds are known in Chemical Abstracts but no references are given (disclaimed by proviso b)):
1. tert-Butyl [(trans-4-{[(1-naphthylsulfonyl)amino]methyl}cyclohexyl)-methyl]carbamate;
2. N-{[4-({[(Cyclopropylmethyl)carbamoyl]amino}methyl)cyclohexyl]methyl}-4-fluorobenzamide;
3. 2-Fluoro-N-{[4-({[(3-methoxypropyl)carbamoyl]amino}methyl)cyclohexyl]-methyl}benzamide;
4. 2-Fluoro-N-{[4-({[(2-methoxypropyl)carbamoyl]amino}methyl)cyclohexyl]-methyl}benzamide;
5. 3,4-Dimethoxy-N-{[4-({[(4-methylbenzyl)carbamoyl]amino}methyl)cyclohexyl]-methyl}benzenesulfonamide;
6. 3-methyl-N-({4-[({[2-(trifluoromethyl)benzyl]carbamoyl}amino)methyl]cyclohexyl}methyl)benzamide;
7. 6-Chloro-N-{[4-({[(4-fluorobenzyl)carbamoyl]amino}methyl)cyclohexyl]-methyl}nicotinamide;
8. 6-Chloro-N-{[4-({[(2-methoxyethyl)carbamoyl]amino}methyl)cyclohexyl]-methyl}nicotinamide;
9. 2,3-Dichloro-N-{[4-({[(2-phenylethyl)carbamoyl]amino}methyl)cyclohexyl]-methyl}benzamide;
10. 4-Methyl-N-[(4-{[(methylcarbamoyl)amino]methyl}cyclohexyl)methyl]-benzamide;
11. 3-Chloro-4-fluoro-N-{[4-({[(2-phenylethyl)carbamoyl]amino}methyl)cyclohexyl]methyl}benzenesulfonamide;
12. N-[(4-{[(Benzylcarbamoyl)amino]methyl}cyclohexyl)methyl]-4-methylbenzenesulfonamide;
13. N-{[4-({[(2-Chlorobenzyl)carbamoyl]amino}methyl)cyclohexyl]methyl}-4-methylbenzenesulfonamide;
14. 4-Methyl-N-{[4-({[(2-phenylethyl)carbamoyl]amino}methyl)cyclohexyl]methyl}-benzenesulfonamide;
15. 4-Methyl-N-{[4-({[(4-methylbenzyl)carbamoyl]amino}methyl)cyclohexyl]-methyl}benzenesulfonamide;
16. N-{[4-({[(2-Chlorobenzyl)carbamoyl]amino}methyl)cyclohexyl]methyl}-3,4-dimethoxybenzenesulfonamide;
17. 3,4-Dimethoxy-N-{[4-({[(2-phenylethyl)carbamoyl]amino}methyl)cyclohexyl]-methyl}benzenesulfonamide;
18. 4-Methyl-N-{[4-({[(4-methylbenzyl)carbamoyl]amino}methyl)cyclohexyl]-methyl}benzamide;
19. N-{[4-({[(2-Chlorobenzyl)carbamoyl]amino}methyl)cyclohexyl]methyl}-4-fluorobenzamide;
20. 4-Chloro-N-{[4-({[(4-fluorobenzyl)carbamoyl]amino}methyl)cyclohexyl]-methyl}benzamide;
21. 2-Fluoro-N-({4-[({[2-(trifluoromethyl)benzyl]carbamoyl}amino)methyl]cyclohexyl}methyl)benzamide;
22. 4-Nitro-N-({4-[({[2-(trifluoromethyl)benzyl]carbamoyl}amino)methyl]cyclohexyl}methyl)benzamide;
23. N {[4-({[(4-Fluorobenzyl)carbamoyl]amino}methyl)cyclohexyl]methyl}-3-methylbenzamide;
24. N-[(4-{[(Benzylcarbamoyl)amino]methyl}cyclohexyl)methyl]-4-methylbenzamide;
25. 2,3-Dichloro-N-({4-[({[2-(trifluoromethyl)benzyl]carbamoyl}amino)methyl]-cyclohexyl}methyl)benzamide;
26. 4-Chloro-N-{[4-({[(3-chlorobenzyl)carbamoyl]amino}methyl)cyclohexyl]-methyl}benzamide;
27. 6-Chloro-N-{[4-({[(2-chlorobenzyl)carbamoyl]amino}methyl)cyclohexyl]-methyl}nicotinamide;
28. 2,3-Dichloro-N-{[4-({[(cyclopropylmethyl)carbamoyl]amino}methyl)cyclo-hexyl]methyl}benzamide;
29. 6-Chloro-N-[(4-{[(propylcarbamoyl)amino]methyl}cyclohexyl)methyl]-nicotinamide;
30. 6-Chloro-N-[(4-{[(isobutylcarbamoyl)amino]methyl}cyclohexyl)methyl]-nicotinamide;
31. 2-Fluoro-N-{[4-({[(4-fluorobenzyl)carbamoyl]amino}methyl)cyclohexyl]-methyl}benzamide;
32. 3-Cyano-N-({4-[({[2-(trifluoromethyl)benzyl]carbamoyl}amino)methyl]cyclo-hexyl}methyl)benzamide;
33. 4-Fluoro-N-{[4-({[(4-fluorobenzyl)carbamoyl]amino}methyl)cyclohexyl]-methyl}benzamide;
34. N {[4-({[(Cyclopropylmethyl)carbamoyl]amino}methyl)cyclohexyl]methyl}-2-fluorobenzamide;
35. N-[(4-{[(Isobutylcarbamoyl)amino]methyl}cyclohexyl)methyl]-4-methylbenzamide;
36. 2,3-Dichloro-N-{[4-({[(4-methylbenzyl)carbamoyl]amino}methyl)cyclohexyl]-methyl}benzamide;
37. N-({4-[({[2-(Trifluoromethyl)benzyl]carbamoyl}amino)methyl]cyclohexyl}-methyl)nicotinamide;
38. N {[4-({[(2-Methoxyethyl)carbamoyl]amino}methyl)cyclohexyl]methyl}-3-methylbenzamide;
39. 3-Methyl-N-{[4-({[(2-phenylethyl)carbamoyl]amino}methyl)cyclohexyl]-methyl}benzamide;
40. 2,3-Dichloro-N-{[4-({[(2-methoxyethyl)carbamoyl]amino}methyl)cyclohexyl]-methyl}benzamide;
41. 4-Fluoro-N-[(4-{[(propylcarbamoyl)amino]methyl}cyclohexyl)methyl]-benzamide;
42. N {[4-({[(3-Methoxypropyl)carbamoyl]amino}methyl)cyclohexyl]methyl}-4-methylbenzamide;
43. 6-Chloro-N-({4-[({[2-(trifluoromethyl)benzyl]carbamoyl}amino)methyl]cyclo-hexyl}methyl)nicotinamide;
44. 3-Cyano-N-{[4-({[(cyclopropylmethyl)carbamoyl]amino}methyl)cyclohexyl]-methyl}benzamide;
45. 3-Methyl-N-[(4-{[(propylcarbamoyl)amino]methyl}cyclohexyl)methyl]-benzamide;
46. 4-Methyl-N-{[4-({[(2-phenylethyl)carbamoyl]amino}methyl)cyclohexyl]methyl}-benzamide;
47. 3-Cyano-N-{[4-({[(2-methoxyethyl)carbamoyl]amino}methyl)cyclohexyl]-methyl}benzamide;
48. N {[4-({[(Cyclopropylmethyl)carbamoyl]amino}methyl)cyclohexyl]methyl}-4-fluorobenzamide;
49. 4-Fluoro-N-{[4-({[(2-methoxyethyl)carbamoyl]amino}methyl)cyclohexyl]-methyl}benzamide;
50. 4-Methoxy-N-[(4-{[(propylcarbamoyl)amino]methyl}cyclohexyl)methyl]-benzamide;
51. N-[(4-{[(Isobutylcarbamoyl)amino]methyl}cyclohexyl)methyl]-4-methoxybenzamide;
52. 2,3-Dichloro-N-[(4-{[(isobutylcarbamoyl)amino]methyl}cyclohexyl)methyl]-benzamide;
53. 2-Fluoro-N-[(4-{[(isobutylcarbamoyl)amino]methyl}cyclohexyl)methyl]-benzamide;
54. 6-Chloro-N-{[4-({[(2-phenylethyl)carbamoyl]amino}methyl)cyclohexyl]methyl}-nicotinamide;
55. N-[(4-{[(Benzylcarbamoyl)amino]methyl}cyclohexyl)methyl]-4-chlorobenzamide;
56. 2,3-Dichloro-N-{[4-({[(2-chlorobenzyl)carbamoyl]amino}methyl)cyclohexyl]-methyl}benzamide;
57. 4-Fluoro-N-({4-[({[2-(trifluoromethyl)benzyl]carbamoyl}amino)methyl]cyclo-hexyl}methyl)benzamide;
58. 6-Chloro-N-{[4-({[(cyclopropylmethyl)carbamoyl]amino}methyl)cyclohexyl]-methyl}nicotinamide;
59. N-{[4-({[(Cyclopropylmethyl)carbamoyl]amino}methyl)cyclohexyl]methyl}-4-methoxybenzamide;
60. 3-Cyano-N-[(4-{[(propylcarbamoyl)amino]methyl}cyclohexyl)methyl] benzamide;
61. N-{[4-({[(2-Chlorobenzyl)carbamoyl]amino}methyl)cyclohexyl]methyl}-3-methylbenzamide;
62. 2,3-Dichloro-N-{[4-({[(3-chlorobenzyl)carbamoyl]amino}methyl)cyclohexyl]-methyl}benzamide;
63. tert-Butyl ({4-[({[3′-({[2-(4-hydroxyphenyl)ethyl]amino}methyl)biphenyl-3-yl]carbonyl}amino)methyl]cyclohexyl}methyl)carbamate;
64. tert-Butyl [(trans-4-{[(1-naphthylsulfonyl)amino]methyl}cyclohexyl)methyl] carbamate;
65. 3-Chloro-N-{[4-({[(2-chlorobenzyl)carbamoyl]amino}methyl)cyclohexyl]methyl}-4-fluorobenzenesulfonamide;
66. 2,3-Dichloro-N-{[4-({[(2-chlorobenzyl)carbamoyl]amino}methyl)cyclohexyl]methyl}benzamide;
67. 2,3-Dichloro-N-[(4-{[(propylcarbamoyl)amino]methyl}cyclohexyl)methyl]-benzamide;
68. 4-Chloro-N-{[4-({[(3-methoxypropyl)carbamoyl]amino}methyl)cyclohexyl]-methyl}benzamide;
69. 3-Chloro-4-fluoro-N-{[4-({[(4-methylbenzyl)carbamoyl]amino}methyl)cyclo-hexyl]methyl}benzenesulfonamide;
70. N-[(trans-4-{[(Benzylcarbamoyl)(isopropyl)amino]methyl}cyclohexyl)methyl]benzenesulfonamide;
71. tert-Butyl [(4-{[(1-naphthylsulfonyl)amino]methyl}cyclohexyl)methyl] carbamate;
72. tert-Butyl [(trans-4-{[(2-naphthylsulfonyl)amino]methyl}cyclohexyl)methyl]-carbamate;
73. tert-Butyl [(trans-4-{[(1-naphthylsulfonyl)amino]methyl}cyclohexyl)methyl]-carbamate;
74. N-{[4-({[(Cyclopropylmethyl)carbamoyl]amino}methyl)cyclohexyl]methyl}-3-fluorobenzamide;
75. 2-Fluoro-N-{[4-({[(3-methoxypropyl)carbamoyl]amino}methyl)cyclohexyl]-methyl}benzamide;
76. 2-Fluoro-N-[[4-[[[[(2-methoxyethyl)amino]carbonyl]amino]methyl]cyclohexyl]methyl]benzamide;
77. 3,4-Dimethoxy-N-{[4-({[(4-methylbenzyl)carbamoyl]amino}methyl)cyclohexyl]methyl}benzenesulfonamide;
78. 3-Methyl-N-({4-[({[2-(trifluoromethyl)benzyl]carbamoyl}amino)methyl]cyclohexyl}methyl)benzamide;
79. 6-Chloro-N-{[4-({[(4-fluorobenzyl)carbamoyl]amino}methyl)cyclohexyl]-methyl}nicotinamide;
80. 6-Chloro-N-{[4-({[(2-methoxyethyl)carbamoyl]amino}methyl)cyclohexyl]-methyl}nicotinamide;
81. 2,3-Dichloro-N-{[4-({[(2-phenylethyl)carbamoyl]amino}methyl)cyclohexyl]-methyl}benzamide;
82. 4-Methyl-N-[(4-{[(methylcarbamoyl)amino]methyl}cyclohexyl)-methyl]benzamide;
83. 3-Chloro-4-fluoro-N-{[4-({[(2-phenylethyl)carbamoyl]amino}methyl)cyclo-hexyl]methyl}benzenesulfonamide;
84. N-[(4-{[(Benzylcarbamoyl)amino]methyl}cyclohexyl)methyl]-4-methylbenzenesulfonamide;
85. N {[4-({[(2-Chlorobenzyl)carbamoyl]amino}methyl)cyclohexyl]methyl}-4-methylbenzenesulfonamide;
86. 4-Methyl-N-{[4-({[(2-phenylethyl)carbamoyl]amino}methyl)cyclohexyl]-methyl}benzenesulfonamide;
87. 4-Methyl-N-{[4-({[(4-methylbenzyl)carbamoyl]amino}methyl)cyclohexyl]-methyl}benzenesulfonamide;
88. N {[4-({[(2-Chlorobenzyl)carbamoyl]amino}methyl)cyclohexyl]methyl}-3,4-dimethoxybenzenesulfonamide; and
89. 3,4-Dimethoxy-N-{[4-({[(2-phenylethyl)carbamoyl]amino}methyl)cyclohexyl]methyl}benzenesulfonamide;
   or salts thereof;
   or enantiomers thereof.

### SUMMARY OF THE INVENTION

The present invention provides compounds of formula (I)

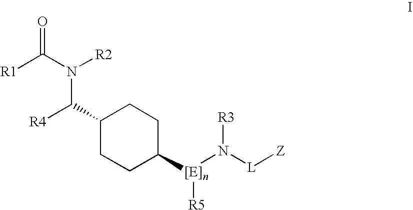

or a pharmaceutically acceptable salt thereof, in which R₁ represents —OR⁶ or —NR^{7a}R^{7b}; R₂ and R₃ independently represent hydrogen, C₁-C₆ alkyl, C₂-C₆ alkenyl, C₂-C₆ alkynyl, C₁-C₆ alkoxy, C₃-C₈ cycloalkyl, C₃-C₈ cycloalkenyl (which alkyl, alkenyl, alkynyl, alkoxy, cycloalkyl, cycloalkenyl groups are optionally substituted by one or more halo, OH, cyano, —C(O)NR^{a}R^{8b}, —C(O)OR⁹, aryl, heteroaryl, heterocyclyl); R⁴ represents hydrogen or C₁-C₆ alkyl which alkyl group is optionally substituted by one or more halo, OH, —C(O)NR^{10a}R^{10b}, C₁-C₄ alkoxy optionally substituted by one or more halo; R₅ represents hydrogen or C₁-C₆ alkyl which alkyl group is optionally substituted by one or more halo, OH, —C(O)NR^{10a}R^{10b}, C₁-C₄ alkoxy optionally substituted by one or more halo; E represents C₁-alkylene; n is an integer of 0 or 1; L represents C(O) or SO₂; Z represents aryl, heteroaryl, heterocyclyl, which groups are optionally substituted by one or more groups independently selected from any of A), B) and C) as defined; A) halo, —NR¹²C(O)R¹³, —NR¹⁴C(O)N(R^{15a})(R^{15b}), —N(R^{15a})(R^{15b}); B) C₁-C₆ alkyl, C₁-C₆ alkoxy, (which alkyl and alkoxy groups are optionally substituted by halo, OH, —N(R^{16a})(R^{16b}), heterocyclyl, heteroaryl (which heterocyclyl, heteroaryl groups are optionally substituted by OH, oxo, C₁-C₆ alkyl, C(O)R¹⁷)); C) C₃-C₈ cycloalkyl, C₃-C₈ cycloalkenyl, heterocyclyl, —(O)_{0 or 1} aryl, —(O)_{0 or 1}-heteroaryl, which cycloalkyl, cycloalkenyl, heterocyclyl, aryl, heteroaryl groups (group C) are optionally substituted by one or more groups independently selected from any of a), b) and c) as defined: a) halo, —OH, oxo, cyano, —C(O)N(R^{18a})(R^{18b}), —N(R^{19a})(R^{19b}), —SR²⁰, —OR^{20a}, —N(R²¹)C(O)R²², —SO₂(R²³), —N(R²⁴)(CHR²⁵)ₘN(R^{26a})(R^{26b}) or C(O)R³⁸; b) C₁-C₆ alkyl, C₁-C₆ alkoxy, C₃-C₈ cycloalkyl, C₃-C₈ cycloalkenyl (which alkyl, alkoxy, cycloalkyl, cycloalkenyl groups are optionally substituted by one or more halo, —OH, cyano, N(R^{27a})(R^{27b}), —N(R²⁸)SO₍₁₋₂₎(R²⁹), —N(R³⁰)C(O)R³¹, —N(R³²)C(O)N(R^{33a})(R^{33b}), —N(R³⁴)C(O)OR³⁵, —SO₍₁₋₂₎(R³⁶), —C(O)OR³⁷, —C(O)R³⁸, —C(O)N(R^{39a})(R^{39b}), heterocyclyl (optionally substituted by C₁-C₄ alkyl, C₁-C₄ alkoxy, oxo, hydroxy, carboxy, amino, C₁-C₆alkylamino, di(C₁-C₆alkyl)amino in which the alkyl groups may be the same or different)); c) aryl, heteroaryl, heterocyclyl (which aryl, heteroaryl, heterocyclyl are optionally substituted by —OH, —C(O)R⁴⁰, —C(O)N(R^{41a})(R^{41b}), oxo, —N(R^{42a})(R^{42b}), —O(R⁴³), C₁-C₆ alkyl, C₁-C₆ alkoxy (which alkyl and alkoxy groups are optionally substituted by halo, —OH)); R⁶ represents C₁-C₆ alkyl, C₃-C₈ cycloalkyl, C₃-C₈ cycloalkenyl, aryl, heteroaryl, heterocyclyl (which groups are optionally substituted by one or more of —OH, halo, cyano, C₁-C₆ alkyl, C₁-C₆ alkoxy, oxo, —NR⁴⁴C(O)N(R^{45a})(R^{45b}), —NR⁴⁴C(O)(R⁴⁰), heterocyclyl or heteroaryl (which heterocyclyl or heteroaryl groups are optionally substituted by C₁-C₄ alkyl, oxo)); R^{7a}, R^{7b}, R^{8a}, R^{8b}, R^{10a}, R^{10b}, R^{11a}, R^{11b}, R^{15a}, R^{15b}, R^{16a}, R^{16b}, R^{18a}, R^{18b}, R^{19a}, R^{19b}, R^{26a}, R^{26b}, R^{27a}, R^{27b}, R^{33a}, R^{33b}, R^{39a}, R^{39b}, R^{41a}, R^{41b}, R^{42a}, R^{42b}, R^{45a}, R^{45b}, R^{46a}, R^{46b} independently represent hydrogen, heterocyclyl or a C₁-C₆ alkyl optionally substituted by OH, halo, C₁-C₄ alkoxy, cyano, amino, C₁-C₆alkylamino, di(C₁-C₆alkyl)amino in which the alkyl groups may be the same or different, heterocyclyl or heteroaryl (which heterocyclyl and heteroaryl groups are optionally substituted by OH, halo, C₁-C₄ alkyl, C₁-C₄ alkoxy), R⁹, R¹³, R¹⁷, R²⁰, R²¹, R²², R²⁴, R²⁵, R³¹, R³⁴, R³⁵, R³⁷, R³⁸, R⁴⁰ independently represent hydrogen, C₁-C₆ alkyl, or C₁-C₆ alkoxy (which alkyl and alkyloxy are optionally substituted by halo, OH, cyano, C(O)NH₂); R¹², R¹⁴, R²⁸, R³⁰, R³², R³⁴, R⁴⁴ represents hydrogen, C₁-C₆ alkyl (which alkyl is optionally substituted by halo, OH, cyano, C(O)NH₂); R^{20a} represents phenyl(CH₂)₀₋₄—O— in which the phenyl is optionally substituted by halo, C₁-C₆ alkyl or C₁-C₆ alkoxy (which alkyl and alkoxy are optionally substituted by halo); R²³, R²⁹, R³⁶ represent N(R^{46a})(R^{46b}), C₁-C₆ alkyl, C₁-C₆ alkoxy (which alkyl and alkoxy are optionally substituted by halo, OH, cyano, C(O)NH₂); R⁴³ represents heteroaryl, heterocyclyl; m is an integer of 0, 1, 2, 3, 4, 5 or 6; with the proviso that the compound is not: a) tert-Butyl [(4-{[(1-naphthylsulfonyl)amino]methyl}cyclohexyl)methyl]-carbamate; tert-Butyl [(trans-4-{[(2-naphthylsulfonyl)amino]methyl}cyclohexyl)methyl]carbamate; tert-Butyl({trans-4-[({[2-trifluoromethyl)phenyl]sulfonyl}amino)methyl]cyclohexyl)methyl) carbamate; or b) tert-Butyl [(trans-4-{[(1-naphthylsulfonyl)amino]methyl}cyclohexyl)methyl]carbamate; N-{[4-({[(Cyclopropylmethyl)carbamoyl]amino}methyl)cyclohexyl]methyl}-4-fluorobenzamide; 2-Fluoro-N-{[4-({[(3-methoxypropyl)carbamoyl]amino}methyl)cyclohexyl]methyl}benzamide; 2-Fluoro-N-{[4-({[(2-methoxypropyl)carbamoyl]amino}methyl)cyclohexyl]methyl}benzamide; 3,4-Dimethoxy-N-{[4-({[(4-methylbenzyl)carbamoyl]amino}methyl)cyclohexyl]methyl}benzenesulfonamide; 3-Methyl-N-({4-[({[2-(trifluoromethyl)benzyl]carbamoyl}amino)methyl]cyclohexyl} methyl)benzamide; 6-Chloro-N-{[4-({[(4-fluorobenzyl)carbamoyl]amino}methyl)cyclohexyl]methyl}nicotinamide; 6-Chloro-N-{[4-({[(2-methoxyethyl)carbamoyl]amino}methyl)cyclohexyl]methyl}nicotinamide; 2,3-Dichloro-N-{[4-({[(2-phenylethyl)carbamoyl]amino}methyl)cyclohexyl]methyl}benzamide; 4-Methyl-N-[(4-{[(methylcarbamoyl)amino]methyl}cyclohexyl)methyl] benzamide; 3-Chloro-4-fluoro-N-{[4-({[(2-phenylethyl) carbamoyl]amino}methyl)cyclohexyl] methyl}benzenesulfonamide; N-[(4-{[(Benzylcarbamoyl)amino]methyl}cyclohexyl)methyl]-4-methylbenzenesulfonamide; N-{[4-({[(2-Chlorobenzyl)carbamoyl]amino}methyl)cyclohexyl]methyl}-4-methylbenzene sulfonamide; 4-Methyl-N-{[4-({[(2-phenylethyl)carbamoyl]amino}methyl)cyclohexyl]methyl}benzenesulfonamide; 4-Methyl-N-{[4-({[(4-methylbenzyl)carbamoyl]amino}methyl)cyclohexyl]methyl}benzenesulfonamide; N-{[4-({[(2-Chlorobenzyl)carbamoyl]amino}methyl)cyclohexyl]methyl}-3,4-dimethoxybenzenesulfonamide; 3,4-Dimethoxy-N-{[4-({[(2-phenylethyl)carbamoyl]amino}methyl)cyclohexyl]methyl}benzenesulfonamide; 4-Methyl-N-{[4-({[(4-methylbenzyl)carbamoyl]amino}methyl)cyclohexyl]methyl}benzamide; N-{[4-({[(2-Chlorobenzyl)carbamoyl]amino}methyl)cyclohexyl]methyl}-4-fluorobenzamide; 4-Chloro-N-{[4-({[(4-fluorobenzyl)carbamoyl]amino}methyl)cyclohexyl]methyl}benzamide; 2-Fluoro-N-({4-[({[2-(trifluoromethyl)benzyl]carbamoyl}amino)methyl]cyclohexyl}methyl)benzamide; 4-Nitro-N-({4-[({[2-(trifluoromethyl)benzyl]carbamoyl}amino)methyl]cyclohexyl}methyl)benzamide; N-{[4-({[(4-Fluorobenzyl)carbamoyl]amino}methyl)cyclohexyl]methyl}-3-methylbenzamide; N-[(4-{[(Benzylcarbamoyl)amino]methyl}cyclohexyl)methyl]-4-methylbenzamide; 2,3-Dichloro-N-({4-[({[2-(trifluoromethyl)benzyl]carbamoyl}amino)methyl]cyclohexyl}methyl)benzamide; 4-Chloro-N-{[4-({[(3-chlorobenzyl)carbamoyl]amino}methyl)cyclohexyl]methyl}benzamide; 6-Chloro-N-{[4-({[(2-chlorobenzyl)carbamoyl]amino}methyl)cyclohexyl]methyl}nicotinamide; 2,3-Dichloro-N-{[4-({[(cyclopropylmethyl)carbamoyl]amino}methyl)cyclohexyl]methyl} benzamide; 6-Chloro-N-[(4-{[(propylcarbamoyl)amino]methyl}cyclohexyl)methyl]nicotinamide; 6-Chloro-N-[(4-{[(isobutylcarbamoyl)amino]methyl}cyclohexyl)methyl]nicotinamide; 2-Fluoro-N-{[4-({[(4-fluorobenzyl)carbamoyl]amino}methyl)cyclohexyl]methyl}benzamide; 3-Cyano-N-({4-[({[2(trifluoromethyl)benzyl]carbamoyl}amino)methyl]cyclohexyl}methyl)benzamide; 4-Fluoro-N-{[4-({[(4-fluorobenzyl)carbamoyl]amino}methyl)cyclohexyl]methyl}benzamide; N-{[4-({[(Cyclopropylmethyl)carbamoyl]amino}methyl)cyclohexyl]methyl}-2-fluorobenzamide; N-[(4-{[(Isobutylcarbamoyl)amino]methyl}cyclohexyl)methyl]-4-methylbenzamide; 2,3-Dichloro-N-{[4-({[(4-methylbenzyl)carbamoyl] amino}methyl)cyclohexyl]methyl}benzamide; N-({4-[({[2-(Trifluoromethyl)benzyl]carbamoyl}amino)methyl]cyclohexyl}methyl)nicotinamide; N-{[4-({[(2-Methoxyethyl) carbamoyl]amino}methyl)cyclohexyl]methyl}-3-methylbenzamide; 3-Methyl-N-{[4-({[(2-phenylethyl)carbamoyl]amino}methyl)cyclohexyl]methyl}benzamide; 2,3-Dichloro-N-{[4-({[(2-methoxyethyl)carbamoyl]amino}methyl)cyclohexyl]methyl}benzamide; 4-Fluoro-N-[(4-{[(propylcarbamoyl)amino]methyl}cyclohexyl)methyl]benzamide; N-{[4-({[(3-Methoxypropyl)carbamoyl]amino}methyl)cyclohexyl]methyl}-4-methylbenzamide; 6-Chloro-N-({4-[({[2-(trifluoromethyl)benzyl]carbamoyl}amino)methyl]cyclohexyl} methyl)nicotinamide; 3-Cyano-N-{[4-({[(cyclopropylmethyl)carbamoyl]amino}methyl)cyclohexyl]methyl}benzamide; 3-Methyl-N-[(4-{[(propylcarbamoyl)amino]methyl}cyclohexyl)methyl] benzamide; 4-Methyl-N-{[4-({[(2-phenylethyl)carbamoyl]amino}methyl)cyclohexyl] methyl}benzamide; 3-Cyano-N-{[4-({[(2-methoxyethyl)carbamoyl]amino}methyl)cyclohexyl]methyl}benzamide; N-{[4-({[(Cyclopropylmethyl)carbamoyl]amino}methyl)cyclohexyl]methyl}-4-fluorobenzamide; 4-Fluoro-N-{[4-({[(2-methoxyethyl)carbamoyl]amino}methyl)cyclohexyl]methyl}benzamide; 4-Methoxy-N-[(4-{[(propylcarbamoyl)amino]methyl}cyclohexyl)methyl] benzamide; N-[(4-{[(Isobutylcarbamoyl)amino]methyl}cyclohexyl)methyl]-4-methoxybenzamide; 2,3-Dichloro-N-[(4-{[(isobutylcarbamoyl)amino]methyl}cyclohexyl)methyl] benzamide; 2-Fluoro-N-[(4-{[(isobutylcarbamoyl)amino]methyl}cyclohexyl)methyl] benzamide; 6-Chloro-N-{[4-({[(2-phenylethyl)carbamoyl]amino}methyl)cyclohexyl]methyl}nicotinamide; N-[(4-{[(Benzylcarbamoyl)amino]methyl}cyclohexyl)methyl]-4-chlorobenzamide; 2,3-Dichloro-N-{[4-({[(2-chlorobenzyl)carbamoyl]amino}methyl)cyclohexyl]methyl}benzamide; 4-Fluoro-N-({4-[({[2-(trifluoromethyl)benzyl]carbamoyl}amino)methyl]cyclohexyl}methyl)benzamide; 6-Chloro-N-{[4-({[(cyclopropylmethyl)carbamoyl]amino}methyl)cyclohexyl]methyl}nicotinamide; N-{[4-({[(Cyclopropylmethyl)carbamoyl]amino}methyl)cyclohexyl]methyl}-4-methoxybenzamide; 3-Cyano-N-[(4-{[(propylcarbamoyl)amino]methyl}cyclohexyl)methyl]benzamide; N-{[4-({[(2-Chlorobenzyl)carbamoyl]amino}methyl)cyclohexyl]methyl}-3-methylbenzamide; 2,3-Dichloro-N-{[4-({[(3-chlorobenzyl)carbamoyl]amino}methyl)cyclohexyl]methyl}benzamide; tert-Butyl ({4-[({[3′-({[2-(4-hydroxyphenyl)ethyl]amino}methyl)biphenyl-3-yl]carbonyl}amino)methyl]cyclohexyl}methyl)carbamate; tert-Butyl [(trans-4-{[(1-naphthylsulfonyl)amino]methyl}cyclohexyl)methyl] carbamate; 3-Chloro-N-{[4-({[(2-chlorobenzyl)carbamoyl]amino}methyl)cyclohexyl]methyl}-4-fluorobenzenesulfonamide; 2,3-Dichloro-N-{[4-({[(2-chlorobenzyl)carbamoyl]amino} methyl)cyclohexyl]methyl}benzamide; 2,3-Dichloro-N-[(4-{[(propylcarbamoyl)amino]methyl}cyclohexyl)methyl] benzamide; 4-Chloro-N-{[4-({[(3-methoxypropyl)carbamoyl]amino}methyl)cyclohexyl]methyl}benzamide; 3-Chloro-4-fluoro-N-{[4-({[(4-methylbenzyl)carbamoyl]amino}methyl)cyclohexyl]methyl}benzenesulfonamide; N-[(trans-4-{[(Benzylcarbamoyl)(isopropyl)amino]methyl}cyclohexyl)methyl]benzenesulfonamide; tert-Butyl [(4-{[(1-Naphthylsulfonyl)amino]methyl}cyclohexyl)methyl]carbamate; tert-Butyl [(trans-4-{[(2-naphthylsulfonyl)amino]methyl}cyclohexyl)methyl]carbamate; tert-Butyl [(trans-4-{[(1-naphthylsulfonyl)amino]methyl}cyclohexyl)methyl]carbamate; N-{[4-({[(Cyclopropylmethyl)carbamoyl]amino}methyl)cyclohexyl]methyl}-3-fluorobenzamide; 2-Fluoro-N-{[4-({[(3-methoxypropyl)carbamoyl]amino}methyl)cyclohexyl]methyl} benzamide; 2-Fluoro-N-[[4-[[[[(2-methoxyethyl)amino]carbonyl]amino]methyl]cyclohexyl]methyl] benzamide; 3,4-Dimethoxy-N-{[4-({[(4-methylbenzyl)carbamoyl]amino}methyl)cyclohexyl]methyl}benzenesulfonamide; 3-Methyl-N-({4-[({[2-(trifluoromethyl)benzyl]carbamoyl}amino)methyl]cyclohexyl}methyl)benzamide; 6-Chloro-N-{[4-({[(4-fluorobenzyl)carbamoyl]amino}methyl)cyclohexyl]methyl}nicotinamide; 6-Chloro-N-{[4-({[(2-methoxyethyl)carbamoyl]amino}methyl)cyclohexyl]methyl}nicotinamide; 2,3-Dichloro-N-{[4-({[(2-phenylethyl)carbamoyl]amino}methyl)cyclohexyl]methyl}benzamide; 4-Methyl-N-[(4-{[(methylcarbamoyl)amino]methyl}cyclohexyl)methyl]benzamide; 3-Chloro-4-fluoro-N-{[4-({[(2-phenylethyl)carbamoyl]amino}methyl)cyclohexyl]methyl} benzenesulfonamide; N-[(4-{[(Benzylcarbamoyl)amino]methyl}cyclohexyl)methyl]-4-methylbenzenesulfonamide; N-{[4-({[(2-Chlorobenzyl)carbamoyl]amino}methyl)cyclohexyl]methyl}-4-methylbenzenesulfonamide; 4-Methyl-N-{[4-({[(2-phenylethyl) carbamoyl]amino}methyl)cyclohexyl]methyl}benzenesulfonamide; 4-Methyl-N-{[4-({[(4-methylbenzyl)carbamoyl]amino}methyl)cyclohexyl]methyl}benzenesulfonamide; N-{[4-({[(2-Chlorobenzyl)carbamoyl]amino}methyl)cyclohexyl]methyl}-3,4-dimethoxybenzene sulfonamide; and 3,4-Dimethoxy-N-{[4-({[(2-phenylethyl)carbamoyl]amino}methyl)cyclo-hexyl]methyl}benzenesulfonamide.

In some embodiments, L represents C(O). In some embodiments, n is 1. In some embodiments, Z represents

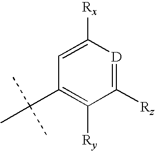

wherein: D represents N or C, optionally substituted by hydrogen, halo, C₁-C₄ alkyl, aryl, NR⁴⁷C(O)R⁴⁸; Rₓ and R_{z} independently represent hydrogen, halo, —OH, —OR⁴⁹, —SR⁵⁰, —N(R^{51a})(R^{51b}), aryl, C₃-C₈ cycloalkyl, C₃-C₈ cycloalkenyl, heterocyclyl, heteroaryl, (which aryl, cycloalkyl, cycloalkenyl, heterocyclyl and heteroaryl groups are optionally substituted by one or more groups of the following halo, OH, oxo, —C(O)N(R^{52a})(R^{52b}), —OR⁵³, —SR⁵⁴, —C(O)OR⁵⁵, cyano, —N(R⁵⁶)C(O)OR⁵⁷, —N(R⁵⁶)C(O)N(R^{52a})(R^{52b}), —N(R⁵⁶)S(O)₂R⁶⁰, —S(O)₂ N(R^{52a})(R^{52b}), —N(R⁵⁶)(CHR⁵⁸)ₘN(R^{52a})(R^{52b}), —N(R^{52a})(R^{52b}), —C(O)R⁵⁹, —N(R⁵⁶)C(O)R⁵⁹, —N(R⁵⁶)(CHR⁵⁸)ₘOR⁵⁵, —N(R⁵⁶)(CHR⁵⁸)ₘC(O)N(R^{52a})(R^{52b}), —N(R⁵⁶)(CHR⁵⁸)ₘC(O)O(R⁵⁷), —S(O)₍₁₋₂₎R⁶⁰, heteroaryl, heterocyclyl (which heteroaryl, heterocyclyl groups are optionally substituted by one or more of the following —OH, oxo, —C(O)R⁶¹, —C(O)N(R^{62a})(R^{62b}), C₁-C₄ alkyl (which alkyl is optionally substituted by OH)), C₁-C₆ alkyl, C₁-C₆ alkoxy (which alkyl and alkoxy groups are optionally substituted by one or more of the following —OH, halo, —N(R^{63a})(R^{63b}), —C(O)OR⁶⁴, N(R⁶⁵)C(O)OR⁶⁴, —N(R⁶⁵)C(O)R⁶⁶, —N(R⁶⁵)C(O)N(R^{63a})(R^{63b}), —N(R⁶⁵)S(O)₍₁₋₂₎(R⁶⁶), —C(O)N(R^{63a})(R^{63b}), heterocyclyl, heteroaryl (which heteroaryl, heterocyclyl groups are optionally substituted by one or more C₁-C₄ alkyl, oxo, —C(O)R⁶⁷)); R_{y} represents hydrogen, halo, C₁-C₆ alkyl, C₁-C₆ alkoxy; R_{y} and R_{z} may together with the carbon atoms to which they are attached represent a benzene ring; wherein R^{51a}, R^{51b}, R^{52a}, R^{52b}, R^{62a}, R^{62b}, R^{63a}, R^{63b} independently represent hydrogen, C₁-C₆ alkyl (optionally substituted by one or more OH, halo, heteroaryl (which heteroaryl optionally is substituted by one or more C₁-C₄ alkyl or C₁-C₄ alkoxy)); R⁴⁶, R⁴⁸, R⁵⁰, R⁵⁴, R⁵⁹, R⁶¹, R⁶⁶ independently represent C₁-C₆ alkyl, optionally substituted by one or more OH, halo, heteroaryl, heterocyclyl, which groups optionally are substituted by one or more C₁-C₆ alkyl, OH; R⁴⁵, R⁴⁷, R⁵⁵, R⁵⁶, R⁵⁸, R⁶⁰, R⁶⁴, R⁶⁵ independently represent hydrogen, C₁-C₆ alkyl, optionally substituted by one or more OH, halo, heteroaryl, heterocyclyl (which heteroaryl, heterocyclyl groups optionally are substituted by one or more C₁-C₆ alkyl, OH; R⁵⁰, R⁵⁷, R⁶⁷ independently represent hydrogen, C₁-C₆ alkyl (optionally substituted by one or more OH, halo); R⁴⁹, R⁵³ independently represent C₃-C₈ cycloalkyl, C₃-C₈ cycloalkenyl, heteroaryl, heterocyclic, C₁-C₆ alkyl (which alkyl is optionally substituted by one or more OH, halo, N(R^{63a})(R^{63b}), heteroaryl, heterocyclyl (which heteroaryl and heterocyclyl groups optionally are substituted by one or more OH, oxo, C₁-C₆ alkyl, C₁-C₆ alkoxy, —C(O)R⁶⁶)); and m is an integer of 0, 1, 2, 3, 4, 5, 6.

In any of the embodiments described above, R_{z} represents hydrogen, halo, C₁-C₆ alkyl, C₁-C₆ alkoxy, aryl or heteroaryl (which aryl and heteroaryl groups are optionally substituted by one or more of —OH, halo, cyano, amino, C₁-C₆ alkyl, C₁-C₆ alkoxy, oxo, —NR⁵⁶C(O)R⁵⁹). In any of the embodiments described above, R₁ represents —OR⁶. In any of the embodiments described above, Z represents

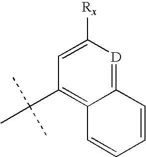

wherein: D represents N or C, optionally substituted by hydrogen, halo, C₁-C₄ alkyl, aryl; R^{x} represents hydrogen, halo, —OR⁶⁸, —SR⁶⁹, —N(R⁷⁰)C(O)R⁷¹, —N(R^{72a})(R^{72b})aryl, C₃-C₈ cycloalkyl, C₃-C₈ cycloalkenyl, heterocyclyl, heteroaryl, (which aryl, cycloalkyl, cycloalkenyl, heterocyclyl, heteroaryl groups are optionally substituted by one or more groups selected from halo, OH, oxo, —C(O)N(R^{73a})(R^{73b}), OR⁷⁴, —SR⁷⁵, —C(O)OR⁷⁶, cyano, —N(R⁷⁷)C(O)OR⁷⁹, —N(R⁷⁷)C(O)N(R^{73a})(R^{73b}), —N(R⁷⁷)S(O)₍₁₋₂₎R⁷⁸, —N(R⁷⁷)(CHR⁷⁹)ₘN(R^{73a})(R^{73b}), —N(R^{73a})(R^{73b}), —C(O)R⁸⁰, —N(R⁷⁷)C(O)R⁸⁰, —N(R⁷⁷)(CHR⁷⁹)ₘOR⁷⁹, —N(R⁷⁷)(CHR⁷⁹)ₘOH, —N(R⁷⁷)(CHR⁷⁹)ₘC(O)N(R^{73a})(R^{73b}), —N(R⁷⁷)(CHR⁷⁹)ₘC(O)O(R⁷⁶), —N(R⁷⁷)C(O)R⁸⁰, —S(O)₍₁₋₂₎R⁷⁸, —S(O)₍₁₋₂₎N(R^{73a})(R^{73b}), heteroaryl, heterocyclyl (which heteroaryl, heterocyclyl groups are optionally substituted by one or more of the following OH, oxo, C(O)R⁸¹, C(O)N(R^{82a})(R^{82b}), —N(R^{82a})(R^{82b}), C₁-C₄ alkyl, C₁-C₄ alkoxy (which alkyl or alkoxy is optionally substituted by one or more OH, —N(R^{82a})(R^{82b}))), C₁-C₆ alkyl, C₁-C₆ alkoxy (which alkyl and alkoxy groups are optionally substituted by one or more of the following —OH, halo, —C(O)OR⁸³, —N(R^{84a})(R^{84b}), —N(R⁸⁵)C(O)OR⁸³, —N(R⁸⁵)C(O)R⁸⁶, —N(R⁸⁵)C(O)N(R^{84a})(R^{84b}), —N(R⁸⁵)S(O)₍₁₋₂₎R⁸⁷, —C(O)N(R^{84a})(R^{84b}), heteroaryl, heterocyclyl (which heteroaryl, heterocyclyl groups are optionally substituted by one or more C₁-C₄ alkyl, oxo, —C(O)R⁸⁸)); R^{72a}, R^{72b}, R^{73a}, R^{73b}, R^{82a}, R^{82b}, R^{84a}, R^{84b} independently represent hydrogen, C₁-C₆ alkyl (optionally substituted by one or more OH, halo, heteroaryl (which heteroaryl optionally is substituted by one or more C₁-C₄ alkyl)); R⁶⁸, R⁶⁹, R⁷⁴, R⁷⁵, R⁷⁸, R⁸⁷ independently represent C₃-C₈ cycloalkyl, C₃-C₈ cycloalkenyl, heteroaryl, heterocyclyl, C₁-C₆ alkyl (which groups are optionally substituted by one or more OH, halo, —N(R^{84a})(R^{84b}), heteroaryl, heterocyclyl (which heteroaryl, heterocyclyl groups optionally are substituted by one or more OH, oxo, C₁-C₆ alkyl, C₁-C₆ alkoxy, C(O)R⁸⁹)); R⁷¹, R⁷⁶, R⁷⁸, R⁷⁹, R⁸⁰, R⁸¹, R⁸³, R⁸⁶, R⁸⁷, R⁸⁸, R⁸⁹ independently represent hydrogen, C₁-C₆ alkyl, optionally substituted by one or more OH, halo, heteroaryl, heterocyclyl (which groups optionally are substituted by one or more OH, C₁-C₆ alkyl, C(O)R⁸⁹); R⁷⁰, R⁷⁷, R⁸⁵ independently represent hydrogen, C₁-C₆ alkyl (optionally substituted by one or more OH, halo); and m is an integer of 0, 1, 2, 3, 4, 5 or 6.

In any of the embodiments described above, D represents N.

In some embodiments, the compound is represented by formula II

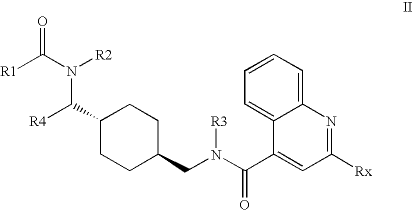

or a pharmaceutically acceptable salt thereof in which R₁, R₂, R₃ and R₄ are as defined above
and Rx represents a group a), b) or c): a)

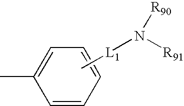

in which L₁ represents a bond or a C₁-C₄ alkylene chain and R⁹⁰ and R⁹¹ independently represent H or C₁-C₆ alkyl optionally substituted by hydroxy or C₁-C₄ alkoxy; b)

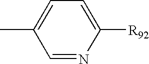

in which R₉₂ represents a group -L₂-L₃-NR⁹³R⁹⁴ in which L₂ is O or N, L₃ is a C₂-C₄ alkylene chain and R⁹³ and R⁹⁴ independently represent H or C₁-C₆ alkyl optionally substituted by hydroxy or C₁-C₄ alkoxy or R₉₂ represents azetidino, pyrrolidino, piperidino, morpholino or piperazino each of which is optionally substituted by one or more of the following: hydroxy, a C₁-C₄ alkyl, C₁-C₄ alkoxy or a C₁-C₄ alkanoyl; c)

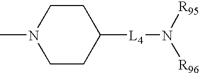

in which L₄ is a C₁-C₄ alkylene chain optionally substituted by hydroxy or fluoro provided that no carbon atom in the chain is attached to two hetero atoms (that is O or N) and R⁹⁵ and R⁹⁶ independently represent H or C₁-C₆ alkyl optionally substituted by hydroxy or C₁-C₄ alkoxy or R⁹⁵ and R⁹⁶ together with the nitrogen atom to which they are attached represent azetidino, pyrrolidino, piperidino, morpholino or piperazino each of which is optionally substituted by one or more of the following: hydroxy, a C₁-C₄ alkyl or C₁-C₄ alkoxy.

In some embodiments, R^{x} represents group a) in which L₁ represents a C₁ alkylene chain, and both R⁹⁰ and R⁹¹ represent H, or wherein R^{x} represents group b), in which R₉₂ represents a group -L₂-L₃-NR⁹³R⁹⁴ in which L₂ is O or N, L₃ is a C₂-C₃ alkylene chain, and R⁹³ and R⁹⁴ both represent H, or wherein or R₉₂ represents piperidino, morpholino or piperazino each of which is optionally substituted by one or more of the following: hydroxy, a C₁ alkyl, C₁, alkoxy or a C₁ alkanoyl, or wherein R^{x} represents group c) in which L₄ is a C₂-C₃ alkylene chain, which chain is optionally substituted by hydroxy provided that no carbon atom in the chain is attached to two hetero atom (that is O or N) and R⁹⁵ and R⁹⁶ independently C₁-C₂ alkyl optionally substituted by hydroxy, or R⁹⁵ and R⁹⁶ together with the nitrogen atom to which they are attached represent azetidino which is optionally substituted by a hydroxy.

In any of the embodiments described above, R₁ represents —OR⁶; R₂ and R₃ independently represent hydrogen or C₁-C₆ alkyl; R₄ represents hydrogen or C₁-C₆ alkyl; R₅ represents hydrogen or C₁-C₆ alkyl; and R⁶ represents C₃-C₅ alkyl.

The present invention also provides one or more of the following compounds:
tert-Butyl [(trans-4-{[({2-[6-(4-methylpiperazin-1-yl)pyridin-3-yl]quinolin-4-yl}carbonyl)amino]methyl}cyclohexyl)methyl]carbamate;
tert-Butyl ({trans-4-[({[2-(2-methyl-6-oxo-1,6-dihydropyridin-3-yl)quinolin-4-yl]carbonyl}amino)methyl]cyclohexyl}methyl)carbamate;
tert-Butyl {[trans-4-({[(2-pyrazin-2-ylquinolin-4-yl)carbonyl]amino}methyl)cyclohexyl]methyl}carbamate;
tert-Butyl ({trans-4-[({[2-(4-ethoxyphenyl)quinolin-4-yl]carbonyl}amino)methyl]-cyclohexyl}methyl)carbamate;
tert-Butyl ({trans-4-[({[2-(6-carbamoylpyridin-3-yl)quinolin-4-yl]carbonyl}amino)-methyl]cyclohexyl}methyl)carbamate;
tert-Butyl {[trans-4-({[(2-phenylquinolin-4-yl)carbonyl]amino}methyl)-cyclohexyl]-methyl}carbamate;
tert-Butyl {[trans-4-({[(2-pyridin-4-ylquinolin-4-yl)carbonyl]amino}methyl)-cyclohexyl]methyl}carbamate;
tert-Butyl ({trans-4-[({[2-(3-carbamoylphenyl)quinolin-4-yl]carbonyl}amino)methyl]-cyclohexyl}methyl)carbamate;
tert-Butyl ({trans-4-[({[2-(4-carbamoylphenyl)quinolin-4-yl]carbonyl}amino)methyl]-cyclohexyl}methyl)carbamate;
tert-Butyl {[trans-4-({[(2-{6-[3-(dimethylamino)propoxy]pyridin-3-yl}quinolin-4-yl)carbonyl]amino}methyl)cyclohexyl]methyl}carbamate;
tert-Butyl [(trans-4-{[({2-[6-(dimethylamino)pyridin-3-yl]quinolin-4-yl}carbonyl)amino]-methyl}cyclohexyl)methyl]carbamate;
tert-Butyl [(trans-4-{[({2-[4-(trifluoromethyl)phenyl]quinolin-4-yl}carbonyl)amino]-methyl}cyclohexyl)methyl]carbamate;
tert-Butyl ({trans-4-[({[2-(5-acetyl-2-thienyl)quinolin-4-yl]carbonyl}amino)methyl]-cyclohexyl}methyl)carbamate;
tert-Butyl ({trans-4-[({[2-(4-fluorophenyl)quinolin-4-yl]carbonyl}amino)methyl]-cyclohexyl}methyl)carbamate;
tert-Butyl ({trans-4-[({[2-(3,5-dimethylisoxazol-4-yl)quinolin-4-yl]carbonyl}amino)-methyl]cyclohexyl}methyl)carbamate;
tert-Butyl {[trans-4-({[(2-pyridin-3-ylquinolin-4-yl)carbonyl]amino}methyl)cyclo-hexyl]methyl}carbamate;
Ethyl ({trans-4-[({[2-(4-fluorophenyl)quinolin-4-yl]carbonyl}amino)methyl]cyclohexyl}methyl)carbamate;
Ethyl [(trans-4-{[({2-[4-(dimethylcarbamoyl)phenyl]quinolin-4-yl}carbonyl)amino]-methyl}cyclohexyl)methyl]carbamate;
Ethyl ({trans-4-[({[2-(3-carbamoylphenyl)quinolin-4-yl]carbonyl}amino)methyl]-cyclohexyl}methyl)carbamate;
tert-Butyl ({trans-4-[({[2-(4-methylphenyl)quinolin-4-yl]carbonyl}amino)methyl]-cyclohexyl}methyl)carbamate;
tert-Butyl ({trans-4-[({[2-(4-chlorophenyl)quinolin-4-yl]carbonyl}amino)methyl]-cyclohexyl}methyl)carbamate;
tert-Butyl ({trans-4-[({[2-(3-methoxyphenyl)quinolin-4-yl]carbonyl}amino)-methyl]cyclohexyl}methyl)carbamate;
tert-Butyl ({trans-4-[({[2-(2-methoxyphenyl)quinolin-4-yl]carbonyl}amino)methyl]-cyclohexyl}methyl)carbamate;
tert-Butyl [(trans-4-{[({2-[4-(methylthio)phenyl]quinolin-4-yl}carbonyl)amino]-methyl}cyclohexyl)methyl]carbamate;
tert-Butyl ({trans-4-[({[2-(2,4-dimethyl-1,3-thiazol-5-yl)quinolin-4-yl]carbonyl}amino)-methyl]cyclohexyl}methyl)carbamate;
Ethyl {[trans-4-({[(2-pyrazin-2-ylquinolin-4-yl)carbonyl]amino}methyl)cyclohexyl]-methyl}carbamate;
Ethyl ({trans-4-[({[2-(3-methoxyphenyl)quinolin-4-yl]carbonyl}amino)methyl]-cyclohexyl}methyl)carbamate;
Ethyl ({trans-4-[({[2-(4-methylphenyl)quinolin-4-yl]carbonyl}amino)methyl]cyclo-hexyl}methyl)carbamate;
Ethyl [(trans-4-{[({2-[4-(methylthio)phenyl]quinolin-4-yl}carbonyl)amino]methyl}-cyclohexyl)methyl]carbamate;
Ethyl ({trans-4-[({[2-(4-ethoxyphenyl)quinolin-4-yl]carbonyl}amino)methyl]-cyclohexyl}methyl)carbamate;
Ethyl ({trans-4-[({[2-(2-methoxyphenyl)quinolin-4-yl]carbonyl}amino)methyl]-cyclohexyl}methyl)carbamate;
Ethyl ({trans-4-[({[2-(4-chlorophenyl)quinolin-4-yl]carbonyl}amino)methyl]cyclo-hexyl}methyl)carbamate;
tert-Butyl {[trans-4-({[(2-pyrimidin-5-ylquinolin-4-yl)carbonyl]amino}methyl)-cyclohexyl]methyl}carbamate;
tert-Butyl ({trans-4-[({[2-(4-cyanophenyl)quinolin-4-yl]carbonyl}amino)methyl]-cyclohexyl}methyl)carbamate;
tert-Butyl {[trans-4-({[(2-chloroquinoline-4-yl)carbonyl]amino}methyl)cyclohexyl]-methyl}carbamate;
tert-Butyl ({trans-4-[({2-[4-(aminomethyl)phenyl]-6-methoxyisonicotinoyl}-amino)methyl]cyclohexyl}methyl)carbamate;
tert-Butyl ({trans-4-[({2-[4-(aminomethyl)phenyl]-5-chloroisonicotinoyl}amino)-methyl]cyclohexyl}methyl)carbamate;
tert-Butyl ({trans-4-[({2-[4-(aminomethyl)phenyl]-6-methylisonicotinoyl}amino)-methyl]cyclohexyl}methyl)carbamate;
tert-Butyl [(trans-4-{[({2-[3-(aminomethyl)phenyl]quinolin-4-yl}carbonyl)amino]-methyl}cyclohexyl)methyl]carbamate;
[3-(4-{[(trans-4-{[(tert-Butoxycarbonyl)amino]methyl}cyclohexyl)methyl]-carbamoyl}quinolin-2-yl)phenyl]acetic acid;
tert-Butyl [(trans-4-{[(2-phenylisonicotinoyl)amino]methyl}cyclohexyl)methyl]-carbamate;
tert-Butyl {[trans-4-({[2-(4-methoxyphenyl)isonicotinoyl]amino}methyl)cyclo-hexyl]methyl}carbamate;
tert-Butyl [(trans-4-{[(2-methyl-6-phenylisonicotinoyl)amino]methyl}cyclohexyl)-methyl]carbamate;
tert-Butyl {[trans-4-({[(5-phenylpyridin-3-yl)carbonyl]amino}methyl)cyclohexyl]-methyl}carbamate;
tert-Butyl {[trans-4-({[2-(1-benzothiophene-2-yl)isonicotinoyl]amino}methyl)cyclo-hexyl]methyl}carbamate;
tert-Butyl {[trans-4-({[(6′-ethoxy-2,3′-bipyridin-4-yl)carbonyl]amino}methyl)cyclo-hexyl]methyl}carbamate;
tert-Butyl {[trans-4-({[2-(2,4-dimethoxyphenyl)isonicotinoyl]amino}methyl)cyclo-hexyl]methyl}carbamate;
tert-Butyl {[trans-4-({[(2′,6′-dimethoxy-2,3′-bipyridin-4-yl)carbonyl]amino}methyl)-cyclohexyl]methyl}carbamate;
tert-Butyl {[trans-4-({[(6′-methoxy-2,3′-bipyridin-4-yl)carbonyl]amino}methyl)cyclo-hexyl]methyl}carbamate;
tert-Butyl {[trans-4-({[2-(4-carbamoylphenyl)isonicotinoyl]amino}methyl)cyclohexyl]methyl}carbamate;
tert-Butyl {[trans-4-({[2-(3-chlorophenyl)isonicotinoyl]amino}methyl)cyclohexyl]-methyl}carbamate;
tert-Butyl ({trans-4-[({2-[4-(aminomethyl)phenyl]isonicotinoyl}amino)methyl]cyclo-hexyl}methyl)carbamate;
tert-Butyl {[trans-4-({[2-(3,4-difluorophenyl)isonicotinoyl]amino}methyl)cyclohexyl]-methyl}carbamate;
Ethyl [(trans-4-{[(2-naphthylsulfonyl)amino]methyl}cyclohexyl)methyl]carbamate;
2,2-Dimethylpropyl {[trans-4-({[(2-pyridin-4-ylquinolin-4-yl)carbonyl]amino}methyl)-cyclohexyl]methyl}carbamate;
Cyclopentyl {[trans-4-({[(2-pyridin-4-ylquinolin-4-yl)carbonyl]amino}methyl)cyclo-hexyl]methyl}carbamate;
Isopropyl {[trans-4-({[(2-pyridin-4-ylquinolin-4-yl)carbonyl]amino}methyl)cyclo-hexyl]methyl}carbamate;
Propyl {[trans-4-({[(2-pyridin-4-ylquinolin-4-yl)carbonyl]amino}methyl)cyclohexyl]-methyl}carbamate;
2-Methoxyethyl {[trans-4-({[(2-pyridin-4-ylquinolin-4-yl)carbonyl]amino}-methyl)cyclohexyl]methyl}carbamate;
Ethyl {[trans-4-({[(2-pyridin-4-ylquinolin-4-yl)carbonyl]amino}methyl)cyclo-hexyl]methyl}carbamate;
Ethyl ({trans-4-[({[2-(4-carbamoylphenyl)quinolin-4-yl]carbonyl}amino)methyl]-cyclohexyl}methyl)carbamate;
Ethyl {[trans-4-({[(2-phenylquinolin-4-yl)carbonyl]amino}methyl)cyclohexyl]methyl}-carbamate;
tert-Butyl [(trans-4-{[(biphenyl-3-ylsulfonyl)amino]methyl}cyclohexyl)methyl]-carbamate;
tert-Butyl {[trans-4-({[(3,4-dibromophenyl)sulfonyl]amino}methyl)cyclohexyl]-methyl}carbamate;
tert-Butyl {[trans-4-({[(3,4-dichlorophenyl)sulfonyl]amino}methyl)cyclohexyl]methyl}-carbamate;
tert-Butyl [(trans-4-{[(quinolin-8-ylsulfonyl)amino]methyl}cyclohexyl)methyl]-carbamate;
tert-Butyl [(trans-4-{[(biphenyl-4-ylsulfonyl)amino]methyl}cyclohexyl)methyl]-carbamate;
tert-Butyl {[trans-4-({[(4-isopropylphenyl)sulfonyl]amino}methyl)cyclohexyl]-methyl}carbamate;
tert-Butyl [(trans-4-{[(2,3-dihydro-1,4-benzodioxin-6-ylsulfonyl)amino]methyl}cyclo-hexyl)methyl]carbamate;
tert-Butyl ({trans-4-[({[5-(2-methyl-1,3-thiazol-4-yl)-2-thienyl]sulfonyl}amino)-methyl]cyclohexyl}methyl)carbamate;
tert-Butyl {[trans-4-({[(5-acetamido-1-naphthyl)sulfonyl]amino}methyl)cyclohexyl]-methyl}carbamate;
tert-Butyl {[trans-4-({[(5-isoxazol-5-yl-2-thienyl)sulfonyl]amino}methyl)cyclo-hexyl]methyl}carbamate;
tert-Butyl {[trans-4-({[(4-acetamidophenyl)sulfonyl]amino}methyl)cyclohexyl]-methyl}carbamate;
tert-Butyl [(trans-4-{[(3-thienylsulfonyl)amino]methyl}cyclohexyl)methyl]-carbamate;
tert-Butyl ({trans-4-[({[3-(5-methyl-1,2,4-oxadiazol-3-yl)phenyl]sulfonyl}amino)-methyl]cyclohexyl}methyl)carbamate;
tert-Butyl [(trans-4-{[(isoquinolin-5-ylsulfonyl)amino]methyl}cyclohexyl)-methyl]carbamate;
tert-Butyl {[trans-4-({[(4-acetamido-2-methyl-1,3-thiazol-5-yl)sulfonyl]amino}-methyl)cyclohexyl]methyl}carbamate;
tert-Butyl [(trans-4-{[(1,3-benzothiazol-6-ylsulfonyl)amino]methyl}cyclohexyl)-methyl]carbamate;
tert-Butyl [(trans-4-{[ethyl(2-naphthylsulfonyl)amino]methyl}cyclohexyl)methyl]-carbamate;
tert-Butyl [(trans-4-{[(2-naphthylsulfonyl)(2-phenylethyl)amino]methyl}cyclohexyl)-methyl]carbamate;
Ethyl N-[(trans-4-{[(tert-butoxycarbonyl)amino]methyl}cyclohexyl)methyl]-N-(2-naphthylsulfonyl)glycinate;
tert-Butyl [(trans-4-{[(2-naphthylsulfonyl)(tetrahydro-2H-pyran-4-ylmethyl)amino]-methyl}cyclohexyl)methyl]carbamate;
tert-Butyl [(trans-4-{[(cyanomethyl)(2-naphthylsulfonyl)amino]methyl}cyclohexyl)-methyl]carbamate;
tert-Butyl [(trans-4-{[allyl(2-naphthylsulfonyl)amino]methyl}cyclohexyl)methyl]-carbamate;
tert-Butyl [(trans-4-{[butyl(2-naphthylsulfonyl)amino]methyl}cyclohexyl)methyl]-carbamate;
tert-Butyl [(trans-4-{[(2-naphthylsulfonyl)(2,2,2-trifluoroethyl)amino]methyl}cyclo-hexyl)methyl]carbamate;
tert-Butyl [(trans-4-{[(2-naphthylsulfonyl)(propyl)amino]methyl}cyclohexyl)-methyl]carbamate;
tert-Butyl [(trans-4-{[methyl(2-naphthylsulfonyl)amino]methyl}cyclohexyl)-methyl]carbamate;
tert-Butyl ({trans-4-[(methyl {[5-(2-methyl-1,3-thiazol-4-yl)-2-thienyl]sulfonyl}-amino)methyl]cyclohexyl}methyl)carbamate;
tert-Butyl ({trans-4-[(ethyl {[5-(2-methyl-1,3-thiazol-4-yl)-2-thienyl]sulfonyl} amino)-methyl]cyclohexyl}methyl)carbamate;
tert-Butyl {[trans-4-({methyl[(2-phenylquinolin-4-yl)carbonyl]amino}methyl)cyclo-hexyl]methyl}carbamate;
tert-Butyl [(trans-4-{[({2-[4-(aminomethyl)phenyl]quinolin-4-yl}carbonyl)amino]-methyl}cyclohexyl)methyl]carbamate acetate;
4-(4-{[(trans-4-{[(tert-Butoxycarbonyl)amino]methyl}cyclohexyl)methyl]carbamoyl}-quinolin-2-yl)benzoic acid;
tert-Butyl ({trans-4-[({[2-(1-oxidopyridin-4-yl)quinolin-4-yl]carbonyl}amino) methyl]-cyclohexyl}methyl)carbamate;
tert-Butyl {[trans-4-({[(2-phenylquinolin-4-yl)sulfonyl]amino}methyl)cyclohexyl]-methyl}carbamate;
tert-Butyl ({trans-4-[(1-naphthoylamino)methyl]cyclohexyl}methyl)carbamate;
N-[(trans-4-{[(tert-Butylcarbamoyl)amino]methyl}cyclohexyl)methyl]-2-pyridin-4-ylquinoline-4-carboxamide;
tert-Butyl ({trans-4-[({[2-(3-{[(methylsulfonyl)amino]methyl}phenyl)quinolin-4-yl]carbonyl}amino)methyl]cyclohexyl}methyl)carbamate;
tert-Butyl {[trans-4-({[(2-{3-[(carbamoylamino)methyl]phenyl}quinolin-4-yl)-carbonyl]amino}methyl)cyclohexyl]methyl}carbamate;
tert-Butyl [(trans-4-{[({2-[3-(acetamidomethyl)phenyl]quinolin-4-yl}carbonyl)amino]-methyl}cyclohexyl)methyl]carbamate;
Methyl [3-(4-{[(trans-4-{[(tert-butoxycarbonyl)amino]methyl}cyclohexyl)methyl]-carbamoyl}quinolin-2-yl)benzyl]carbamate;
tert-Butyl [(trans-4-{[({2-[4-(2-aminoethyl)phenyl]quinolin-4-yl}carbonyl)amino]-methyl}cyclohexyl)methyl]carbamate;
tert-Butyl [(trans-4-{[({2-[4-(acetamidomethyl)phenyl]quinolin-4-yl}carbonyl)-amino]methyl}cyclohexyl)methyl]carbamate;
Methyl [4-(4-{[(trans-4-{[(tert-butoxycarbonyl)amino]methyl}cyclohexyl)methyl]-carbamoyl}quinolin-2-yl)benzyl]carbamate;
tert-Butyl {[trans-4-({[(2-{4-[(carbamoylamino)methyl]phenyl}quinolin-4-yl)carbonyl]amino}methyl)cyclohexyl]methyl}carbamate;
tert-Butyl ({trans-4-[({[2-(4-{[(methylsulfonyl)amino]methyl}phenyl)quinolin-4-yl]carbonyl}amino)methyl]cyclohexyl}methyl)carbamate;
[4-(4-{[(trans-4-{[(tert-Butoxycarbonyl)amino]methyl}cyclohexyl)methyl]-carbamoyl}quinolin-2-yl)phenyl]acetic acid;
3-(4-{[(trans-4-{[(tert-Butoxycarbonyl)amino]methyl}cyclohexyl)methyl]-carbamoyl}quinolin-2-yl)benzoic acid;
Tetrahydro-2H-pyran-4-yl [(trans-4-{[({2-[6-(4-methylpiperazin-1-yl)pyridin-3-yl]quinolin-4-yl}carbonyl)amino]methyl}cyclohexyl)methyl]carbamate;
1-Methylpiperidin-4-yl [(trans-4-{[({2-[6-(4-methylpiperazin-1-yl)pyridin-3-yl]quinolin-4-yl}carbonyl)amino]methyl}cyclohexyl)methyl]carbamate;
Oxetan-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
(1S)-2-Methoxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
(1R)-2-Methoxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
Tetrahydrofuran-3-yl [(trans-4-{[({2-[6-(4-methylpiperazin-1-yl)pyridin-3-yl]quinolin-4-yl}carbonyl)amino]methyl}cyclohexyl)methyl]carbamate;
2-(2-Oxopyrrolidin-1-yl)ethyl [(trans-4-{[({2-[6-(4-methylpiperazin-1-yl)pyridin-3-yl]quinolin-4-yl}carbonyl)amino]methyl}cyclohexyl)methyl]carbamate;
(3S)-Tetrahydrofuran-3-yl [(trans-4-{[({2-[6-(4-methylpiperazin-1-yl)pyridin-3-yl]quinolin-4-yl}carbonyl)amino]methyl}cyclohexyl)methyl]carbamate;
2,2-Difluoroethyl [(trans-4-{[({2-[6-(4-methylpiperazin-1-yl)pyridin-3-yl]quinolin-4-yl}carbonyl)amino]methyl}cyclohexyl)methyl]carbamate;
2-Fluoroethyl [(trans-4-{[({2-[6-(4-methylpiperazin-1-yl)pyridin-3-yl]quinolin-4-yl}carbonyl)amino]methyl}cyclohexyl)methyl]carbamate;
Ethyl [(trans-4-{[({2-[6-(4-methylpiperazin-1-yl)pyridin-3-yl]quinolin-4-yl}carbonyl)amino]methyl}cyclohexyl)methyl]carbamate;
2-Methoxyethyl [(trans-4-{[({2-[6-(4-methylpiperazin-1-yl)pyridin-3-yl]quinolin-4-yl}carbonyl)amino]methyl}cyclohexyl)methyl]carbamate;
1-Cyanoethyl [(trans-4-{[({2-[6-(4-methylpiperazin-1-yl)pyridin-3-yl]quinolin-4-yl}carbonyl)amino]methyl}cyclohexyl)methyl]carbamate;
2-Acetamidoethyl [(trans-4-{[({2-[6-(4-methylpiperazin-1-yl)pyridin-3-yl]quinolin-4-yl}carbonyl)amino]methyl}cyclohexyl)methyl]carbamate;
(3-Methyloxetan-3-yl)methyl [(trans-4-{[({2-[6-(4-methylpiperazin-1-yl)pyridin-3-yl]quinolin-4-yl}carbonyl)amino]methyl}cyclohexyl)methyl]carbamate;
(3R)-5-Oxopyrrolidin-3-yl [(trans-4-{[({2-[6-(4-methylpiperazin-1-yl)pyridin-3-yl]quinolin-4-yl}carbonyl)amino]methyl}cyclohexyl)methyl]carbamate;
(3S)-5-Oxopyrrolidin-3-yl [(trans-4-{[({2-[6-(4-methylpiperazin-1-yl)pyridin-3-yl]quinolin-4-yl}carbonyl)amino]methyl}cyclohexyl)methyl]carbamate;
Tetrahydrofuran-3-ylmethyl [(trans-4-{[({2-[6-(4-methylpiperazin-1-yl)pyridin-3-yl]quinolin-4-yl}carbonyl)amino]methyl}cyclohexyl)methyl]carbamate;
Tetrahydrofuran-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
(5-Methylisoxazol-3-yl)methyl [(trans-4-{[({2-[6-(4-methylpiperazin-1-yl)pyridin-3-yl]quinolin-4-yl}carbonyl)amino]methyl}cyclohexyl)methyl]carbamate;
2-(1H-Pyrazol-1-yl)ethyl [(trans-4-{[({2-[6-(4-methylpiperazin-1-yl)pyridin-3-yl]quinolin-4-yl}carbonyl)amino]methyl}cyclohexyl)methyl]carbamate;
1,3-Thiazol-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
Pyrazin-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
2-Cyanoethyl [(trans-4-{[({2-[6-(4-methylpiperazin-1-yl)pyridin-3-yl]quinolin-4-yl}carbonyl)amino]methyl}cyclohexyl)methyl]carbamate;
(1S)-2-Hydroxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
tert-Butyl {[trans-4-({[(2-{4-[(methylamino)sulfonyl]-phenyl}quinolin-4-yl)carbonyl]amino}methyl)cyclohexyl]methyl}-carbamate;
tert-Butyl [(trans-4-{[({2-[4-(aminosulfonyl)phenyl]-quinolin-4-yl}carbonyl)amino]-methyl}-cyclohexyl)methyl]-carbamate;
Tetrahydro-2H-pyran-4-yl {[trans-4-({[(2-{4-[(methylamino)sulfonyl]phenyl}-quinolin-4-yl)carbonyl]amino}methyl)cyclohexyl]methyl}carbamate;
(3S)-Tetrahydrofuran-3-yl {[trans-4-({[(2-{4-[(methylamino)sulfonyl]phenyl}-quinolin-4-yl)carbonyl]amino}methyl)cyclohexyl]methyl}-carbamate;
Tetrahydro-2H-pyran-4-yl [(trans-4-{[({2-[4-(aminosulfonyl)phenyl]-quinolin-4-yl}carbonyl)amino]-methyl}cyclohexyl)-methyl]carbamate;
(3S)-Tetrahydrofuran-3-yl [(trans-4-{[({2-[4-(aminosulfonyl)phenyl]-quinolin-4-yl}carbonyl)amino]-methyl}cyclohexyl)-methyl]carbamate;
Tetrahydro-2H-pyran-4-yl {[trans-4-({[(2-{4-[(dimethylamino)sulfonyl]phenyl}-quinolin-4-yl)carbonyl]amino}methyl)cyclohexyl]-methyl}carbamate;
(3S)-Tetrahydrofuran-3-yl {[trans-4-({[(2-{4-[(dimethylamino)sulfonyl]phenyl}-quinolin-4-yl)carbonyl]amino}methyl)cyclohexyl]-methyl}carbamate;
tert-Butyl [(trans-4-{[({2-[6-(4-acetylpiperazin-1-yl)pyridin-3-yl]quinolin-4-yl}carbonyl)amino]methyl}cyclohexyl)methyl]carbamate;
tert-Butyl {[trans-4-({[(2-{6-[(2-hydroxyethyl)amino]pyridin-3-yl}quinolin-4-yl)carbonyl]amino}methyl)cyclohexyl]methyl}carbamate;
tert-Butyl ({trans-4-[({[2-(6-pyrrolidin-1-ylpyridin-3-yl)quinolin-4-yl]carbonyl}amino)methyl]cyclohexyl}methyl)carbamate;
tert-Butyl ({trans-4-[({[2-(6-morpholin-4-ylpyridin-3-yl)quinolin-4-yl]carbonyl}amino)methyl]cyclohexyl}methyl)carbamate;
tert-Butyl ({trans-4-[({[2-(6-{[2-(dimethylamino)ethyl]amino}pyridin-3-yl)quinolin-4-yl]carbonyl}amino)methyl]cyclohexyl}methyl)carbamate;
tert-Butyl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
tert-Butyl ({trans-4-[({[2-(6-{[(1S)-2-hydroxy-1-methylethyl]amino}pyridin-3-yl)quinolin-4-yl]carbonyl}amino)methyl]cyclohexyl}methyl)carbamate;
tert-Butyl ({trans-4-[({[2-(6-{[(1R)-2-hydroxy-1-methylethyl]amino}pyridin-3-yl)quinolin-4-yl]carbonyl}amino)methyl]cyclohexyl}methyl)carbamate;
tert-Butyl {[trans-4-({[(2-{6-[(3-hydroxypropyl)amino]pyridin-3-yl}quinolin-4-yl)carbonyl]amino}methyl)cyclohexyl]methyl}carbamate;
tert-Butyl [(trans-4-{[({2-[6-(4-hydroxypiperidin-1-yl)pyridin-3-yl]quinolin-4-yl}carbonyl)amino]methyl}cyclohexyl)methyl]carbamate;
tert-Butyl {[trans-4-({[(2-{6-[bis(2-hydroxyethyl)amino]pyridin-3-yl}quinolin-4-yl)carbonyl]amino}methyl)cyclohexyl]methyl}carbamate;
tert-Butyl [(trans-4-{[({2-[6-(4-carbamoylpiperidin-1-yl)pyridin-3-yl]quinolin-4-yl}carbonyl)amino]methyl}cyclohexyl)methyl]carbamate;
tert-Butyl ({trans-4-[({[2-(6-piperazin-1-ylpyridin-3-yl)quinolin-4-yl]carbonyl}amino)methyl]cyclohexyl}methyl)carbamate;
tert-Butyl ({trans-4-[({[2-(6-{[2-(2-hydroxyethoxy)ethyl]amino}pyridin-3-yl)quinolin-4-yl]carbonyl}amino)methyl]cyclohexyl}methyl)carbamate;
tert-Butyl {[trans-4-({[(2-{6-[(2-methoxyethyl)amino]pyridin-3-yl}quinolin-4-yl)carbonyl]amino}methyl)cyclohexyl]methyl}carbamate;
tert-Butyl [(trans-4-{[({2-[6-(3-hydroxypiperidin-1-yl)pyridin-3-yl]quinolin-4-yl}carbonyl)amino]methyl}cyclohexyl)methyl]carbamate;
3-{[5-(4-{[(trans-4-{[(tert-Butoxycarbonyl)amino]methyl}cyclohexyl)methyl]-carbamoyl}quinolin-2-yl)pyridin-2-yl]amino}-2-methylpropanoic acid;
tert-Butyl ({trans-4-[({[2-(6-{[(5-methylpyrazin-2-yl)methyl]amino}pyridin-3-yl)quinolin-4-yl]carbonyl}amino)methyl]cyclohexyl}methyl)carbamate;
tert-Butyl {[trans-4-({[(2-{6-[(3S)-3-(dimethylamino)pyrrolidin-1-yl]pyridin-3-yl}quinolin-4-yl)carbonyl]amino}methyl)cyclohexyl]methyl}carbamate;
tert-Butyl {[trans-4-({[(2-{6-[4-(hydroxymethyl)piperidin-1-yl]pyridin-3-yl}quinolin-4-yl)carbonyl]amino}methyl)cyclohexyl]methyl}carbamate;
tert-Butyl {[trans-4-({[(2-{6-[(2,3-dihydroxypropyl)amino]pyridin-3-yl}quinolin-4-yl)carbonyl]amino}methyl)cyclohexyl]methyl}carbamate;
tert-Butyl {[trans-4-({[(2-{6-[(2S)-2-carbamoylpyrrolidin-1-yl]pyridin-3-yl}quinolin-4-yl)carbonyl]amino}methyl)cyclohexyl]methyl}carbamate;
tert-Butyl ({trans-4-[({[2-(6-{[2-hydroxy-1-(hydroxymethyl)ethyl]amino}pyridin-3-yl)quinolin-4-yl]carbonyl}amino)methyl]cyclohexyl}methyl)carbamate;
tert-Butyl [(trans-4-{[({2-[6-(3-oxopiperazin-1-yl)pyridin-3-yl]quinolin-4-yl}carbonyl)amino]methyl}cyclohexyl)methyl]carbamate;
tert-Butyl ({trans-4-[({[2-(6-{[(1-methyl-1H-pyrazol-4-yl)methyl]amino}pyridin-3-yl)quinolin-4-yl]carbonyl}amino)methyl]cyclohexyl}methyl)carbamate;
tert-Butyl {[trans-4-({[(2-{6-[(3-amino-3-oxopropyl)amino]pyridin-3-yl}quinolin-4-yl)carbonyl]amino}methyl)cyclohexyl]methyl}carbamate;
tert-Butyl [(trans-4-{[({2-[6-(3-hydroxyazetidin-1-yl)pyridin-3-yl]quinolin-4-yl}carbonyl)amino]methyl}cyclohexyl)methyl]carbamate;
tert-Butyl ({trans-4-[({[2-(6-{[(1S)-1-carbamoylpropyl]amino}pyridin-3-yl)quinolin-4-yl]carbonyl}amino)methyl]cyclohexyl}methyl)carbamate;
tert-Butyl ({trans-4-[({[2-(6-{[(5-methyl-1,2,4-oxadiazol-3-yl)methyl]amino}pyridin-3-yl)quinolin-4-yl]carbonyl}amino)methyl]cyclohexyl}methyl)carbamate;
tert-Butyl {[trans-4-({[(2-{6-[2-(hydroxymethyl)morpholin-4-yl]pyridin-3-yl}quinolin-4-yl)carbonyl]amino}methyl)cyclohexyl]methyl}carbamate;
tert-Butyl [(trans-4-{[({2-[6-(4-oxoimidazolidin-1-yl)pyridin-3-yl]quinolin-4-yl}carbonyl)amino]methyl}cyclohexyl)methyl]carbamate;
tert-Butyl [(trans-4-{[({2-[6-(tetrahydrofuran-3-ylmethoxy)pyridin-3-yl]quinolin-4-yl}carbonyl)amino]methyl}cyclohexyl)methyl]carbamate;
tert-Butyl [(trans-4-{[({2-[6-(2-hydroxyethoxy)pyridin-3-yl]quinolin-4-yl}carbonyl)amino]methyl}cyclohexyl)methyl]carbamate;
tert-Butyl [(trans-4-{[({2-[6-(tetrahydro-2H-pyran-4-yloxy)pyridin-3-yl]quinolin-4-yl}carbonyl)amino]methyl}cyclohexyl)methyl]carbamate;
tert-Butyl [(trans-4-{[({2-[6-(2-hydroxy-1-methylpropoxy)pyridin-3-yl]quinolin-4-yl}carbonyl)amino]methyl}cyclohexyl)methyl]carbamate;
tert-Butyl {[trans-4-({[(2-{6-[(2-oxopyrrolidin-1-yl)methoxy]pyridin-3-yl}quinolin-4-yl)carbonyl]amino}methyl)cyclohexyl]methyl}carbamate;
tert-Butyl [(trans-4-{[({2-[6-(2-amino-2-oxoethoxy)pyridin-3-yl]quinolin-4-yl}carbonyl)amino]methyl}cyclohexyl)methyl]carbamate;
tert-Butyl {[trans-4-({[(2-{6-[2-(4-methylpiperazin-1-yl)ethoxy]pyridin-3-yl}quinolin-4-yl)carbonyl]amino}methyl)cyclohexyl]methyl}carbamate;
tert-Butyl [(trans-4-{[({2-[3-(4-methylpiperazin-1-yl)propoxy]quinolin-4-yl}carbonyl)amino]methyl}cyclohexyl)methyl]carbamate;
tert-Butyl [(trans-4-{[({2-[3-(dimethylamino)propoxy]quinolin-4-yl}carbonyl)amino]methyl}cyclohexyl)methyl]carbamate;
tert-Butyl [(trans-4-{[({2-[2-(4-methylpiperazin-1-yl)ethoxy]quinolin-4-yl}carbonyl)amino]methyl}cyclohexyl)methyl]carbamate;
tert-Butyl [(trans-4-{[({2-[3-(4-acetylpiperazin-1-yl)propoxy]quinolin-4-yl}carbonyl)amino]methyl}cyclohexyl)methyl]carbamate;
tert-Butyl {[trans-4-({[(2-{6-[2-(dimethylamino)ethoxy]pyridin-3-yl}quinolin-4-yl)carbonyl]amino}methyl)cyclohexyl]methyl}carbamate;
tert-Butyl [(trans-4-{[({2-[4-(2-hydroxyethyl)piperazin-1-yl]quinolin-4-yl}carbonyl)amino]methyl}cyclohexyl)methyl]carbamate;
tert-Butyl {[trans-4-({[(2-{4-[2-(dimethylamino)ethyl]-piperazin-1-yl}quinolin-4-yl)carbonyl]amino}-methyl)cyclohexyl]methyl}carbamate;
tert-Butyl ({trans-4-[({[2-(4-hydroxypiperidin-1-yl)quinolin-4-yl]carbonyl}amino)-methyl]cyclohexyl}methyl)carbamate;
tert-Butyl [(trans-4-{[({2-[4-(hydroxymethyl)-piperidin-1-yl]quinolin-4-yl}carbonyl)amino]-methyl}cyclohexyl)-methyl]carbamate;
tert-Butyl [(trans-4-{[({2-[4-(2-hydroxyethyl)piperidin-1-yl]quinolin-4-yl}carbonyl)amino]-methyl}cyclohexyl)-methyl]carbamate;
tert-Butyl {[trans-4-({[(2-piperazin-1-ylquinolin-4-yl)carbonyl]amino}methyl)cyclohexyl]methyl}carbamate;
tert-Butyl [(trans-4-{[({2-[4-(acetamidomethyl)piperidin-1-yl]quinolin-4-yl}carbonyl)amino]methyl}cyclohexyl)methyl]carbamate;
tert-Butyl [(trans-4-{[({2-[4-(2-acetamidoethyl)piperidin-1-yl]quinolin-4-yl}carbonyl)amino]methyl}cyclohexyl)methyl]carbamate;
tert-Butyl {[trans-4-({[(2-{4-[2-(dimethylamino)ethyl]piperidin-1-yl}quinolin-4-yl)carbonyl]amino}methyl)cyclohexyl]methyl}carbamate;
tert-Butyl [(trans-4-{[({2-[4-(2-aminoethyl)piperidin-1-yl]quinolin-4-yl}carbonyl)amino]methyl}cyclohexyl)methyl]carbamate;
Tetrahydro-2H-pyran-4-yl {[trans-4-({[(2-{6-[2-(dimethylamino)ethoxy]pyridin-3-yl}quinolin-4-yl)carbonyl]amino}methyl)cyclohexyl]methyl}carbamate;
(3S)-Tetrahydrofuran-3-yl {[trans-4-({[(2-{6-[2-(dimethylamino)ethoxy]pyridin-3-yl}quinolin-4-yl)carbonyl]amino}methyl)cyclohexyl]methyl}carbamate;
Tetrahydro-2H-pyran-4-yl {[trans-4-({[(2-{6-[(2-hydroxyethyl)amino]pyridin-3-yl}quinolin-4-yl)carbonyl]amino}methyl)cyclohexyl]methyl}carbamate;
Tetrahydro-2H-pyran-4-yl ({trans-4-[({[2-(6-{[2-(dimethylamino)ethyl]amino}-pyridin-3-yl)quinolin-4-yl]carbonyl}amino)methyl]cyclohexyl}methyl)carbamate;
(3S)-Tetrahydrofuran-3-yl ({trans-4-[({[2-(6-{[2-(dimethylamino)ethyl]amino}-pyridin-3-yl)quinolin-4-yl]carbonyl}amino)methyl]cyclohexyl}methyl)carbamate;
(3S)-Tetrahydrofuran-3-yl {[trans-4-({[(2-{6-[(2-hydroxyethyl)amino]pyridin-3-yl}quinolin-4-yl)carbonyl]amino}methyl)cyclohexyl]methyl}carbamate;
(3S)-Tetrahydrofuran-3-yl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
Tetrahydro-2H-pyran-4-yl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
tert-Butyl {[trans-4-({[(2-{4-[2-(dimethylamino)ethoxy]phenyl}quinolin-4-yl)carbonyl]amino}methyl)cyclohexyl]methyl}carbamate;
tert-Butyl ({trans-4-[({2-[6-(4-methylpiperazin-1-yl)pyridin-3-yl]quinolin-4-yl}carbonyl)amino]cyclohexyl}methyl)carbamate;
tert-Butyl {[trans-4-({[(2-{4-[3-(dimethylamino)-2-hydroxypropyl]piperidin-1-yl}quinolin-4-yl)carbonyl]amino}methyl)cyclohexyl]methyl}carbamate;
tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;
tert-Butyl [(trans-4-{[({2-[4-(2-hydroxy-3-morpholin-4-ylpropyl)piperidin-1-yl]quinolin-4-yl}-carbonyl)amino]methyl}cyclohexyl)methyl]-carbamate;
(R) or (S) tert-Butyl [(trans-4-{[({2-[4-(2-hydroxy-3-morpholin-4-ylpropyl)piperidin-1-yl]quinolin-4-yl}-carbonyl)amino]methyl}cyclohexyl)methyl]-carbamate;
(S) or (R) tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;
tert-Butyl {[trans-4-({[(2-{4-[2-(dimethylamino)-2-oxoethoxy]piperidin-1-yl}quinolin-4-yl)carbonyl]amino}methyl)cyclohexyl]methyl}carbamate;
tert-Butyl {[trans-4-({[(2-{4-[2-(dimethylamino)ethoxy]piperidin-1-yl}quinolin-4-yl)carbonyl]amino}methyl)cyclohexyl]methyl}carbamate;
2,2-Dimethylpropyl {[trans-4-({[(2-{4-[2-(dimethylamino)ethyl]piperidin-1-yl}quinolin-4-yl)carbonyl]amino}methyl)cyclohexyl]methyl}carbamate;
tert-Butyl [(trans-4-{[({2-[4-(morpholin-4-ylmethyl)piperidin-1-yl]quinolin-4-yl}carbonyl)amino]methyl}cyclohexyl)methyl]-carbamate;
tert-Butyl [(trans-4-{[({2-[4-(4-methylpiperazin-1-yl)piperidin-1-yl]quinolin-4-yl}carbonyl)amino]methyl}cyclohexyl)methyl]-carbamate;
tert-Butyl ({trans-4-[({[2-(pyridin-3-yloxy)quinolin-4-yl]carbonyl}amino)-methyl]cyclohexyl}methyl)carbamate;
tert-Butyl [(trans-4-{[({2-[(1-methylpiperidin-4-yl)methoxy]quinolin-4-yl}carbonyl)amino]methyl}cyclohexyl)methyl]-carbamate;
tert-Butyl ({trans-4-[({[2-(3-{[2-(dimethylamino)ethyl]carbamoyl}azetidin-1-yl)quinolin-4-yl]carbonyl}amino)methyl]cyclohexyl}methyl)carbamate;
tert-Butyl ({trans-4-[({[2-(4-aminophenyl)quinolin-4-yl]carbonyl}amino)-methyl]cyclohexyl}methyl)carbamate;
tert-Butyl {[trans-4-({[(2-{4-[2-(dimethylamino)ethyl]-3-oxopiperazin-1-yl}quinolin-4-yl)carbonyl]amino}methyl)cyclohexyl]methyl}carbamate;
tert-Butyl [(trans-4-{[({2-[4-(hydroxyacetyl)piperazin-1-yl]quinolin-4-yl}carbonyl)amino]methyl}cyclohexyl)methyl]carbamate;
tert-Butyl {[trans-4-({[(2-{4-[(4-fluorobenzyl)oxy]piperidin-1-yl}quinolin-4-yl)carbonyl]amino}methyl)cyclohexyl]methyl}carbamate;
tert-Butyl {[trans-4-({[(2-amino-1-benzothiophen-3-yl)carbonyl]amino}methyl)-cyclohexyl]methyl}carbamate;
tert-Butyl [(trans-4-{[({2-[(ethylcarbamoyl)amino]-1-benzothiophen-3-yl}carbonyl)amino]methyl}cyclohexyl)methyl]carbamate;
Tetrahydro-2H-pyran-4-yl {[trans-4-({[(2-{4-[2-(dimethylamino)ethyl]piperidin-1-yl}quinolin-4-yl)carbonyl]amino}methyl)cyclohexyl]methyl}carbamate;
tert-Butyl ({trans-4-[({[2-(1′-methyl-4,4′-bipiperidin-1-yl)quinolin-4-yl]carbonyl}amino)methyl]cyclohexyl}methyl)carbamate;
tert-Butyl ({trans-4-[({[2-(4-{2-[(2-methoxyethyl)amino]ethyl}piperidin-1-yl)quinolin-4-yl]carbonyl}amino)methyl]cyclohexyl}methyl)carbamate;
tert-Butyl ({trans-4-[({[2-(4-{2-[(2-methoxyethyl)(methyl)-amino]ethyl}piperidin-1-yl)quinolin-4-yl]carbonyl}amino)-methyl]cyclohexyl}-methyl)carbamate;
tert-Butyl {[trans-4-({[(2-{4-[2-(tetrahydro-2H-pyran-4-ylamino)ethyl]piperidin-1-yl}quinolin-4-yl)carbonyl]amino}-methyl)cyclohexyl]-methyl}carbamate;
tert-Butyl {[trans-4-({[(2-{4-[2-(3-methoxyazetidin-1-yl)ethyl]piperidin-1-yl}quinolin-4-yl)carbonyl]amino}-methyl)cyclohexyl]-methyl}carbamate;
tert-Butyl {[trans-4-({[(2-{4-[2-(3-hydroxypyrrolidin-1-yl)ethyl]piperidin-1-yl}quinolin-4-yl)carbonyl]amino}-methyl)cyclohexyl]-methyl}carbamate;
tert-Butyl [(trans-4-{[({2-[4-(2-pyrrolidin-1-ylethyl)piperidin-1-yl]quinolin-4-yl}carbonyl)amino]-methyl}cyclohexyl)-methyl]carbamate;
tert-Butyl ({trans-4-[({[2-(4-{2-[(2-hydroxyethyl)(methyl)-amino]ethyl}piperidin-1-yl)quinolin-4-yl]carbonyl}amino)-methyl]cyclohexyl}-methyl)carbamate;
tert-Butyl {[trans-4-({[(2-{4-[2-(3-hydroxyazetidin-1-yl)ethyl]piperidin-1-yl}quinolin-4-yl)carbonyl]amino}methyl)cyclohexyl]methyl}carbamate;
tert-Butyl [(trans-4-{[({2-[4-(2-azetidin-1-ylethyl)piperidin-1-yl]quinolin-4-yl}carbonyl)amino]methyl}cyclohexyl)methyl]carbamate;
1-{2-[1-(4-{[(trans-4-{[(tert-Butoxycarbonyl)amino]methyl}cyclohexyl)methyl]-carbamoyl}quinolin-2-yl)piperidin-4-yl]ethyl}azetidine-3-carboxylic acid;
tert-Butyl {[trans-4-({[(2-{4-[2-(3-aminoazetidin-1-yl)ethyl]piperidin-1-yl}quinolin-4-yl)carbonyl]amino}methyl)cyclohexyl]methyl}carbamate;
tert-Butyl {[trans-4-({[(2-{4-[3-(dimethylamino)-2-fluoropropyl]piperidin-1-yl}quinolin-4-yl)carbonyl]amino}methyl)cyclohexyl]methyl}carbamate;
tert-Butyl {[trans-4-({[(2-{3-[2-(dimethylamino)ethoxy]azetidin-1-yl}quinolin-4-yl)carbonyl]amino}methyl)cyclohexyl]methyl}carbamate;
tert-Butyl [(trans-4-{[({2-[3-(aminomethyl)phenyl]-6-(1H-pyrazol-4-yl)pyridin-4-yl}carbonyl)amino]methyl}cyclohexyl)methyl]carbamate;
tert-Butyl [(trans-4-{[({6-[3-(aminomethyl)phenyl]-2,3′-bipyridin-4-yl}carbonyl)amino]methyl}cyclohexyl)methyl]carbamate;
tert-Butyl [(trans-4-{[({6′-amino-6-[3-(aminomethyl)phenyl]-2,3′-bipyridin-4-yl}carbonyl)amino]methyl}cyclohexyl)methyl]carbamate;
tert-Butyl {[trans-4-({[(2-{1-[2-(dimethylamino)ethyl]-1H-pyrazol-4-yl}quinolin-4-yl)carbonyl]amino}methyl)cyclohexyl]methyl}carbamate;
tert-Butyl [(trans-4-{[({2-[1-(2-hydroxyethyl)-1H-pyrazol-4-yl]quinolin-4-yl}carbonyl)amino]methyl}cyclohexyl)methyl]carbamate;
tert-Butyl ({trans-4-[({[2-(4-{2-[(2-fluoroethylamino]ethyl}piperidin-1-yl)quinolin-4-yl]carbonyl}amino)methyl]cyclohexyl}methyl)carbamate;
tert-Butyl ({trans-4-[({[2-(4-(2-((2-fluoroethyl)(methyl)amino)ethyl}piperidin-1-yl)quinolin-4-yl]carbonyl}amino)methyl]cyclohexyl}methyl)carbamate;
   or a pharmaceutically acceptable salt thereof,
   or an enantiomer thereof.

The present invention also provides a compound according to any of the preceding compounds, including compounds of proviso b), for use in therapy.

The present invention also provides methods of treating obesity or being overweight, eating disorders, dyslipidemia, atherosclerosis, heart failure, stroke, type 2 diabetes mellitus and prevention of type 2 diabetes comprising administering a pharmacologically effective amount of a compound of formula (I) or formula (II) as defined in any of the preceeding embodiments, including proviso b) to a patient in need thereof.

The present invention also provides processes for preparing a compound of formula (I) or formula (II) as described herein.

### DESCRIPTION OF THE INVENTION

The present invention provides a compound of formula (I)

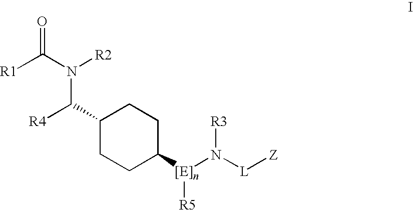

or a pharmaceutically acceptable salt thereof, in which

R₁ represents —OR⁶ or —NR^{7a}R^{7b};

R₂ and R₃ independently represent hydrogen, C₁-C₆ alkyl, C₂-C₆ alkenyl, C₂-C₆ alkynyl, C₁-C₆ alkoxy, C₃-C₈ cycloalkyl, C₃-C₈ cycloalkenyl (which alkyl, alkenyl, alkynyl, alkoxy, cycloalkyl, cycloalkenyl groups are optionally substituted by one or more halo, OH, cyano, —C(O)NR^{8a}R^{8b}, —C(O)OR⁹, aryl, heteroaryl, heterocyclyl);

R₄ represents hydrogen or C₁-C₆ alkyl, which alkyl group is optionally substituted by one or more halo, OH, —C(O)NR^{10a}R^{10b}, C₁-C₄ alkoxy, optionally substituted by one or more halo;

R₅ represents hydrogen or C₁-C₆ alkyl, which alkyl group is optionally substituted by one or more halo, OH, —C(O)NR^{11a}R^{11b}, C₁-C₄ alkoxy, optionally substituted by one or more halo;

E represents C₁-alkylene;

n is an integer of 0 or 1;

L represents C(O) or SO₂;

Z represents aryl, heteroaryl, heterocyclyl, which groups are optionally substituted by one or more groups independently selected from any of A), B) and C) as defined;

A) halo, —NR¹²C(O)R¹³, —NR¹⁴C(O)N(R^{15a})(R^{15b}), —N(R^{15a})(R^{15b})

B) C₁-C₆ alkyl, C₁-C₆ alkoxy, (which alkyl and alkoxy groups are optionally substituted by halo, OH, —N(R^{16a})(R^{16b}), heterocyclyl, heteroaryl (which heterocyclyl, heteroaryl groups are optionally substituted by OH, oxo, C₁-C₆ alkyl, C(O)R¹⁷));

C) C₃-C₈ cycloalkyl, C₃-C₈ cycloalkenyl, heterocyclyl, —(O)_{0 or 1} aryl, —(O)_{0 or 1}-heteroaryl, which cycloalkyl, cycloalkenyl, heterocyclyl, aryl, heteroaryl groups (group C) are optionally substituted by one or more groups independently selected from any of a), b) and c) as defined:
a) halo, —OH, oxo, cyano, —C(O)N(R^{18a})(R^{18b}), —N(R^{19a})(R^{19b}), SR²⁰, OR^{20a}, —N(R²¹)C(O)R²², —SO₂(R²³), —N(R²⁴)(CHR²⁵)ₘN(R^{26a})(R^{26b}) or C(O)R³⁸;
b) C₁-C₆ alkyl, C₁-C₆ alkoxy, C₃-C₈ cycloalkyl, C₃-C₈ cycloalkenyl (which alkyl, alkoxy, cycloalkyl, cycloalkenyl groups are optionally substituted by one or more halo, —OH, cyano, N(R^{27a})(R^{27b}), —N(R²⁸)SO₍₁₋₂₎(R²⁹), —N(R³⁰)C(O)R³¹, —N(R³²)C(O)N(R^{33a})(R^{33b}), —N(R³⁴)C(O)OR³⁵, —SO₍₁₋₂₎(R³⁶), —C(O)OR³⁷, —C(O)R³⁸, —C(O)N(R^{39a})(R^{39b}), heterocyclyl (optionally substituted by C₁-C₄ alkyl, C₁-C₄ alkoxy, oxo, hydroxy, carboxy, amino, C₁-C₆alkylamino, di(C₁-C₆alkyl)amino in which the alkyl groups may be the same or different));
c) aryl, heteroaryl, heterocyclyl (which aryl, heteroaryl, heterocyclyl are optionally substituted by —OH, —C(O)R⁴⁰, —C(O)N(R^{41a})(R^{41b}), oxo, —N(R^{42a})(R^{42b}), —O(R⁴³), C₁-C₆ alkyl, C₁-C₆ alkoxy (which alkyl and alkoxy groups are optionally substituted by halo, —OH));

R⁶ represents C₁-C₆ alkyl, C₃-C₈ cycloalkyl, C₃-C₈ cycloalkenyl, aryl, heteroaryl, heterocyclyl (which groups are optionally substituted by one or more of —OH, halo, cyano, C₁-C₆ alkyl, C₁-C₆ alkoxy, oxo, —NR⁴⁴C(O)N(R^{45a})(R^{45b}), —NR⁴⁴C(O)(R⁴⁰), heterocyclyl or heteroaryl (which heterocyclyl or heteroaryl groups are optionally substituted by C₁-C₄ alkyl, oxo));

R^{7a}, R^{7b}, R^{8a}, R^{8b}, R^{10a}, R^{10b}, R^{11a}, R^{11b}, R^{15a}, R^{15b}, R^{16a}, R^{16b}, R^{18a}, R^{18b}, R^{19a}, R^{19b}, R^{26a}, R^{26b}, R^{27a}, R^{27b}, R^{33a}, R^{33b}, R^{39a}, R^{39b}, R^{41a}, R^{41b}, R^{42a}, R^{42b}, R^{45a}, R^{45b}, R^{46a}, R^{46b} independently represent hydrogen, heterocyclyl or a C₁-C₆ alkyl optionally substituted by OH, halo, C₁-C₄ alkoxy, cyano, amino, C₁-C₆alkylamino, di(C₁-C₆alkyl)amino in which the alkyl groups may be the same or different, heterocyclyl or heteroaryl (which heterocyclyl and heteroaryl groups are optionally substituted by OH, halo, C₁-C₄ alkyl, C₁-C₄ alkoxy),

R⁹, R¹³, R¹⁷, R²⁰, R²¹, R²², R²⁴, R²⁵, R³¹, R³⁴, R³⁵, R³⁷, R³⁸, R⁴⁰ independently represent hydrogen, C₁-C₆ alkyl, or C₁-C₆ alkoxy (which alkyl and alkyloxy are optionally substituted by halo, OH, cyano, C(O)NH₂);

R¹², R¹⁴, R²⁸, R³⁰, R³², R³⁴, R⁴⁴ represents hydrogen, C₁-C₆ alkyl (which alkyl is optionally substituted by halo, OH, cyano, C(O)NH₂);

R^{20a} represents phenyl(CH₂)₀₋₄—O— in which the phenyl is optionally substituted by halo, C₁-C₆ alkyl or C₁-C₆ alkoxy (which alkyl and alkoxy are optionally substituted by halo);

R²³, R²⁹, R⁶ represent —N(R^{46a})(R^{46b}), C₁-C₆ alkyl, C₁-C₆ alkoxy (which alkyl and alkoxy are optionally substituted by halo, OH, cyano, C(O)NH₂);

R⁴³ represents heteroaryl, heterocyclyl;

m is an integer of 0, 1, 2, 3, 4, 5 or 6;

with the proviso that the compound is not:

a)
1. tert-Butyl [(4-{[(1-naphthylsulfonyl)amino]methyl}cyclohexyl)methyl]-carbamate;
2. tert-Butyl [(trans-4-{[(2-naphthylsulfonyl)amino]methyl}cyclohexyl)methyl] carbamate;
3. tert-Butyl({trans-4-[({[2-trifluoromethyl)phenyl]sulfonyl}amino)methyl]cyclohexyl)methyl)carbamate; or
   b)
1. tert-Butyl [(trans-4-{[(1-naphthylsulfonyl)amino]methyl}cyclohexyl)methyl] carbamate;
2. N-{[4-({[(Cyclopropylmethyl)carbamoyl]amino}methyl)cyclohexyl]methyl}-4-fluorobenzamide;
3. 2-Fluoro-N-{[4-({[(3-methoxypropyl)carbamoyl]amino}methyl)cyclohexyl]methyl}benzamide;
4. 2-Fluoro-N-{[4-({[(2-methoxypropyl)carbamoyl]amino}methyl)cyclohexyl]methyl}benzamide;
5. 3,4-Dimethoxy-N-{[4-({[(4-methylbenzyl)carbamoyl]amino}methyl)cyclohexyl]methyl}benzenesulfonamide;
6. 3-Methyl-N-({4-[({[2-(trifluoromethyl)benzyl]carbamoyl}amino)methyl]cyclohexyl}methyl)benzamide;
7. 6-Chloro-N-{[4-({[(4-fluorobenzyl)carbamoyl]amino}methyl)cyclohexyl]methyl}nicotinamide;
8. 6-Chloro-N-{[4-({[(2-methoxyethyl)carbamoyl]amino}methyl)cyclohexyl]methyl}nicotinamide;
9. 2,3-Dichloro-N-{[4-({[(2-phenylethyl)carbamoyl]amino}methyl)cyclohexyl]methyl}benzamide;
10. 4-Methyl-N-[(4-{[(methylcarbamoyl)amino]methyl}cyclohexyl)methyl] benzamide;
11. 3-Chloro-4-fluoro-N-{[4-({[(2-phenylethyl)carbamoyl]amino}methyl)cyclohexyl]methyl}benzenesulfonamide;
12. N-[(4-{[(Benzylcarbamoyl)amino]methyl}cyclohexyl)methyl]-4-methylbenzenesulfonamide;
13. N-{[4-({[(2-Chlorobenzyl)carbamoyl]amino}methyl)cyclohexyl]methyl}-4-methylbenzenesulfonamide;
14. 4-Methyl-N-{[4-({[(2-phenylethyl)carbamoyl]amino}methyl)cyclohexyl]methyl}benzenesulfonamide;
15. 4-Methyl-N-{[4-({[(4-methylbenzyl)carbamoyl]amino}methyl)cyclohexyl]methyl}benzenesulfonamide;
16. N-{[4-({[(2-Chlorobenzyl)carbamoyl]amino}methyl)cyclohexyl]methyl}-3,4-dimethoxybenzenesulfonamide;
17. 3,4-Dimethoxy-N-{[4-({[(2-phenylethyl)carbamoyl]amino}methyl)cyclohexyl]methyl}benzenesulfonamide;
18. 4-Methyl-N-{[4-({[(4-methylbenzyl)carbamoyl]amino}methyl)cyclohexyl]methyl}benzamide;
19. N-{[4-({[(2-Chlorobenzyl)carbamoyl]amino}methyl)cyclohexyl]methyl}-4-fluorobenzamide;
20. 4-Chloro-N-{[4-({[(4-fluorobenzyl)carbamoyl]amino}methyl)cyclohexyl]methyl}benzamide;
21. 2-Fluoro-N-({4-[({[2-(trifluoromethyl)benzyl]carbamoyl}amino)methyl]cyclohexyl}methyl)benzamide;
22. 4-Nitro-N-({4-[({[2-(trifluoromethyl)benzyl]carbamoyl}amino)methyl]cyclohexyl}methyl)benzamide;
23. N {[4-({[(4-Fluorobenzyl)carbamoyl]amino}methyl)cyclohexyl]methyl}-3-methylbenzamide;
24. N-[(4-{[(Benzylcarbamoyl)amino]methyl}cyclohexyl)methyl]-4-methylbenzamide;
25. 2,3-Dichloro-N-({4-[({[2-(trifluoromethyl)benzyl]carbamoyl}amino) methyl]cyclohexyl}methyl)benzamide;
26. 4-Chloro-N-{[4-({[(3-chlorobenzyl)carbamoyl]amino}methyl)cyclohexyl]methyl}benzamide;
27. 6-Chloro-N-{[4-({[(2-chlorobenzyl)carbamoyl]amino}methyl)cyclohexyl]methyl}nicotinamide;
28. 2,3-Dichloro-N-{[4-({[(cyclopropylmethyl)carbamoyl]amino}methyl)cyclohexyl]methyl}benzamide;
29. 6-Chloro-N-[(4-{[(propylcarbamoyl)amino]methyl}cyclohexyl)methyl]nicotinamide;
30. 6-Chloro-N-[(4-{[(isobutylcarbamoyl)amino]methyl}cyclohexyl)methyl]nicotinamide;
31. 2-Fluoro-N-{[4-({[(4-fluorobenzyl)carbamoyl]amino}methyl)cyclohexyl]methyl}benzamide;
32. 3-Cyano-N-({4-[({[2-(trifluoromethyl)benzyl]carbamoyl}amino)methyl]cyclohexyl}methyl)benzamide;
33. 4-Fluoro-N-{[4-({[(4-fluorobenzyl)carbamoyl]amino}methyl)cyclohexyl]methyl}benzamide;
34. N-{[4-({[(Cyclopropylmethyl)carbamoyl]amino}methyl)cyclohexyl]methyl}-2-fluorobenzamide;
35. N-[(4-{[(Isobutylcarbamoyl)amino]methyl}cyclohexyl)methyl]-4-methylbenzamide;
36. 2,3-Dichloro-N-{[4-({[(4-methylbenzyl)carbamoyl]amino}methyl)cyclohexyl]methyl}benzamide;
37. N-({4-[({[2-(Trifluoromethyl)benzyl]carbamoyl}amino)methyl]cyclohexyl}methyl)nicotinamide;
38. N-{[4-({[(2-Methoxyethyl)carbamoyl]amino}methyl)cyclohexyl]methyl}-3-methylbenzamide;
39. 3-Methyl-N-{[4-({[(2-phenylethyl)carbamoyl]amino}methyl)cyclohexyl]methyl}benzamide;
40. 2,3-Dichloro-N-{[4-({[(2-methoxyethyl)carbamoyl]amino}methyl)cyclohexyl]methyl}benzamide;
41. 4-Fluoro-N-[(4-{[(propylcarbamoyl)amino]methyl}cyclohexyl)methyl] benzamide;
42. N-{[4-({[(3-Methoxypropyl)carbamoyl]amino}methyl)cyclohexyl]methyl}-4-methylbenzamide;
43. 6-Chloro-N-({4-[({[2-(trifluoromethyl)benzyl]carbamoyl}amino)methyl]cyclohexyl}methyl)nicotinamide;
44. 3-Cyano-N-{[4-({[(cyclopropylmethyl)carbamoyl]amino}methyl)cyclohexyl]methyl}benzamide;
45. 3-Methyl-N-[(4-{[(propylcarbamoyl)amino]methyl}cyclohexyl)methyl] benzamide;
46. 4-Methyl-N-{[4-({[(2-phenylethyl)carbamoyl]amino}methyl)cyclohexyl]methyl}benzamide;
47. 3-Cyano-N-{[4-({[(2-methoxyethyl)carbamoyl]amino}methyl)cyclohexyl]methyl}benzamide;
48. N-{[4-({[(Cyclopropylmethyl)carbamoyl]amino}methyl)cyclohexyl]methyl}-4-fluorobenzamide;
49. 4-Fluoro-N-{[4-({[(2-methoxyethyl)carbamoyl]amino}methyl)cyclohexyl]methyl}benzamide;
50. 4-Methoxy-N-[(4-{[(propylcarbamoyl)amino]methyl}cyclohexyl)methyl] benzamide;
51. N-[(4-{[(Isobutylcarbamoyl)amino]methyl}cyclohexyl)methyl]-4-methoxybenzamide;
52. 2,3-Dichloro-N-[(4-{[(isobutylcarbamoyl)amino]methyl}cyclohexyl)methyl] benzamide;
53. 2-Fluoro-N-[(4-{[(isobutylcarbamoyl)amino]methyl}cyclohexyl)methyl] benzamide;
54. 6-Chloro-N-{[4-({[(2-phenylethyl)carbamoyl]amino}methyl)cyclohexyl]methyl}nicotinamide;
55. N-[(4-{[(Benzylcarbamoyl)amino]methyl}cyclohexyl)methyl]-4-chlorobenzamide;
56. 2,3-Dichloro-N-{[4-({[(2-chlorobenzyl)carbamoyl]amino}methyl)cyclohexyl]methyl}benzamide;
57. 4-Fluoro-N-({4-[({[2-(trifluoromethyl)benzyl]carbamoyl}amino)methyl]cyclohexyl}methyl)benzamide;
58. 6-Chloro-N-{[4-({[(cyclopropylmethyl)carbamoyl]amino}methyl)cyclohexyl]methyl}nicotinamide;
59. N-{[4-({[(Cyclopropylmethyl)carbamoyl]amino}methyl)cyclohexyl]methyl}-4-methoxybenzamide;
60. 3-Cyano-N-[(4-{[(propylcarbamoyl)amino]methyl}cyclohexyl)methyl] benzamide;
61. N-{[4-({[(2-Chlorobenzyl)carbamoyl]amino}methyl)cyclohexyl]methyl}-3-methylbenzamide;
62. 2,3-Dichloro-N-{[4-({[(3-chlorobenzyl)carbamoyl]amino}methyl)cyclohexyl]methyl}benzamide;
63. tert-Butyl ({4-[({[3′-({[2-(4-hydroxyphenyl)ethyl]amino}methyl)biphenyl-3-yl]carbonyl}amino)methyl]cyclohexyl}methyl)carbamate;
64. tert-Butyl [(trans-4-{[(1-naphthylsulfonyl)amino]methyl}cyclohexyl)methyl] carbamate;
65. 3-Chloro-N-{[4-({[(2-chlorobenzyl)carbamoyl]amino}methyl)cyclohexyl]methyl}-4-fluorobenzenesulfonamide;
66. 2,3-Dichloro-N-{[4-({[(2-chlorobenzyl)carbamoyl]amino}methyl)cyclohexyl]methyl}benzamide;
67. 2,3-Dichloro-N-[(4-{[(propylcarbamoyl)amino]methyl}cyclohexyl)methyl] benzamide;
68. 4-Chloro-N-{[4-({[(3-methoxypropyl)carbamoyl]amino}methyl)cyclohexyl]methyl}benzamide;
69. 3-Chloro-4-fluoro-N-{[4-({[(4-methylbenzyl)carbamoyl]amino}methyl)cyclohexyl]methyl}benzenesulfonamide;
70. N-[(trans-4-{[(Benzylcarbamoyl)(isopropyl)amino]methyl}cyclohexyl)methyl]benzenesulfonamide;
71. tert-Butyl [(4-{[(1-Naphthylsulfonyl)amino]methyl}cyclohexyl)methyl] carbamate;
72. tert-Butyl [(trans-4-{[(2-naphthylsulfonyl)amino]methyl}cyclohexyl)methyl]carbamate;
73. tert-Butyl [(trans-4-{[(1-naphthylsulfonyl)amino]methyl}cyclohexyl)methyl]carbamate;
74. N-{[4-({[(Cyclopropylmethyl)carbamoyl]amino}methyl)cyclohexyl]methyl}-3-fluorobenzamide;
75. 2-Fluoro-N-{[4-({[(3-methoxypropyl)carbamoyl]amino}methyl)cyclohexyl]methyl}benzamide;
76. 2-Fluoro-N-[[4-[[[[(2-methoxyethyl)amino]carbonyl]amino]methyl]cyclohexyl]methyl] benzamide;
77. 3,4-Dimethoxy-N-{[4-({[(4-methylbenzyl)carbamoyl]amino}methyl)cyclohexyl]methyl}benzenesulfonamide;
78. 3-Methyl-N-({4-[({[2-(trifluoromethyl)benzyl]carbamoyl}amino) methyl]cyclohexyl}methyl)benzamide;
79. 6-Chloro-N-{[4-({[(4-fluorobenzyl)carbamoyl]amino}methyl)cyclohexyl]methyl}nicotinamide;
80. 6-Chloro-N-{[4-({[(2-methoxyethyl)carbamoyl]amino}methyl)cyclohexyl]methyl}nicotinamide;
81. 2,3-Dichloro-N-{[4-({[(2-phenylethyl)carbamoyl]amino}methyl)cyclohexyl]methyl}benzamide;
82. 4-Methyl-N-[(4-{[(methylcarbamoyl)amino]methyl}cyclohexyl)methyl]benzamide;
83. 3-Chloro-4-fluoro-N-{[4-({[(2-phenylethyl)carbamoyl]amino}methyl)cyclohexyl]methyl}benzenesulfonamide;
84. N-[(4-{[(Benzylcarbamoyl)amino]methyl}cyclohexyl)methyl]-4-methylbenzenesulfonamide;
85. N-{[4-({[(2-Chlorobenzyl)carbamoyl]amino}methyl)cyclohexyl]methyl}-4-methylbenzenesulfonamide;
86. 4-Methyl-N-{[4-({[(2-phenylethyl)carbamoyl]amino}methyl)cyclohexyl]methyl}benzenesulfonamide;
87. 4-Methyl-N-{[4-({[(4-methylbenzyl)carbamoyl]amino}methyl)cyclohexyl]methyl}benzenesulfonamide;
88. N-{[4-({[(2-Chlorobenzyl)carbamoyl]amino}methyl)cyclohexyl]methyl}-3,4-dimethoxybenzenesulfonamide; and
89. 3,4-Dimethoxy-N-{[4-({[(2-phenylethyl)carbamoyl]amino}methyl)cyclo-hexyl]methyl}benzenesulfonamide.

In another aspect the present invention provides a compound of formula (I)

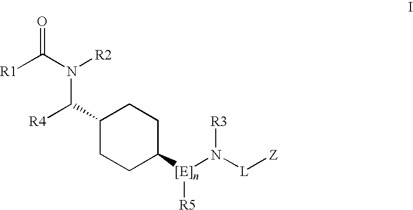

or an enantiomer thereof, or a pharmaceutically acceptable salt thereof,
in which

R₁ represents —OR⁶ or —NR^{7a}R^{7b};

R₂ and R₃ independently represent hydrogen, C₁-C₆ alkyl, C₂-C₆ alkenyl, C₂-C₆ alkynyl, C₁-C₆ alkoxy, C₃-C₈ cycloalkyl, C₃-C₈ cycloalkenyl (which alkyl, alkenyl, alkynyl, alkoxy, cycloalkyl, cycloalkenyl groups are optionally substituted by one or more halo, OH, cyano, —C(O)NR^{8a}R^{8b}, —C(O)OR⁹, aryl, heteroaryl, heterocyclyl);

R₄ represents hydrogen or C₁-C₆ alkyl, which alkyl group is optionally substituted by one or more halo, OH, —C(O)NR^{10a}R^{10b}, C₁-C₄ alkoxy, optionally substituted by one or more halo;

R₅ represents hydrogen or C₁-C₆ alkyl, which alkyl group is optionally substituted by one or more halo, OH, —C(O)NR^{11a}R^{11b}, C₁-C₄ alkoxy, optionally substituted by one or more halo;

E represents C₁-alkylene;

n is an integer of 0 or 1;

L represents C(O) or SO₂;

Z represents aryl, heteroaryl, heterocyclyl, which groups are optionally substituted by one or more groups independently selected from any of A), B) and C) as defined;

A) halo, —NR¹²C(O)R¹³, —NR¹⁴C(O)N(R^{15a})(R^{15b}), —N(R^{15a})(R^{15b})

B) C₁-C₆ alkyl, C₁-C₆ alkoxy, (which alkyl and alkoxy groups are optionally substituted by halo, OH, —N(R^{16a})(R^{16b}), heterocyclyl, heteroaryl (which heterocyclyl, heteroaryl groups are optionally substituted by OH, oxo, C₁-C₆ alkyl, C(O)R¹⁷));

C) C₃-C₈ cycloalkyl, C₃-C₈ cycloalkenyl, heterocyclyl, aryl, heteroaryl, which cycloalkyl, cycloalkenyl, heterocyclyl, aryl, heteroaryl groups (group C) are optionally substituted by one or more groups independently selected from any of a), b) and c) as defined:
a) halo, —OH, oxo, cyano, —C(O)N(R^{18a})(R^{18b}), —N(R^{19a})(R^{19b}), —SR²⁰, —N(R²¹)C(O)R²², —SO₂(R²³), —N(R²⁴)(CHR²⁵)ₘN(R^{26a})(R^{26b});
b) C₁-C₆ alkyl, C₁-C₆ alkoxy, C₃-C₈ cycloalkyl, C₃-C₈ cycloalkenyl (which alkyl, alkoxy, cycloalkyl, cycloalkenyl groups are optionally substituted by one or more halo, —OH, cyano, N(R^{27a})(R^{27b}), —N(R²⁸)SO₍₁₋₂₎(R²⁹), —N(R³⁰)C(O)R³¹, —N(R³²)C(O)N(R^{33a})(R^{33b}), —N(R³⁴)C(O)OR³⁵, —SO₍₁₋₂₎(R³⁶), —C(O)OR³⁷, —C(O)R³⁸, —C(O)N(R^{39a})(R^{39b}), heterocyclyl (optionally substituted by C₁-C₄ alkyl, oxo));
c) aryl, heteroaryl, heterocyclyl (which aryl, heteroaryl, heterocyclyl are optionally substituted by —OH, —C(O)R⁴⁰, —C(O)N(R^{41a})(R^{41b}), oxo, —N(R^{42a})(R^{42b}), —O(R⁴³), C₁-C₆ alkyl, C₁-C₆ alkoxy (which alkyl and alkoxy groups are optionally substituted by halo, —OH));

R⁶ represents C₁-C₆ alkyl, C₃-C₈ cycloalkyl, C₃-C₈ cycloalkenyl, aryl, heteroaryl, heterocyclyl (which groups are optionally substituted by one or more of —OH, halo, cyano, C₁-C₆ alkyl, C₁-C₆ alkoxy, oxo, —NR⁴⁴C(O)N(R^{45a})(R^{45b}), —NR⁴⁴C(O)(R⁴⁰), heterocyclyl or heteroaryl (which heterocyclyl or heteroaryl groups are optionally substituted by C₁-C₄ alkyl, oxo));

R^{7a}, R^{7b}, R^{8a}, R^{8b}, R^{10a}, R^{10b}, R^{11a}, R^{11b}, R^{15a}, R^{15b}, R^{16a}, R^{16b}, R^{18a}, R^{18b}, R^{19a}, R^{19b}, R^{26a}, R^{26b}, R^{27a}, R^{27b}, R^{33a}, R^{33b}, R^{39a}, R^{39b}, R^{41a}, R^{41b}, R^{42a}, R^{42b}, R^{45a}, R^{45b}, R^{46a}, R^{46b}, independently represent hydrogen, C₁-C₆ alkyl, optionally substituted by OH, halo, cyano, heterocyclyl or heteroaryl (which heterocyclyl and heteroaryl groups are optionally substituted by OH, halo, C₁-C₄ alkyl, C₁-C₄ alkoxy),

R⁹, R¹³, R¹⁷, R²⁰, R²¹, R²², R²⁴, R²⁵, R³¹, R³⁴, R³⁵, R³⁷, R³⁸, R⁴⁰ independently represent hydrogen, C₁-C₆ alkyl, or C₁-C₆ alkoxy (which alkyl and alkyloxy are optionally substituted by halo, OH, cyano, C(O)NH₂);

R¹², R¹⁴, R²⁸, R³⁰, R³², R³⁴, R⁴⁴ represents hydrogen, C₁-C₆ alkyl (which alkyl is optionally substituted by halo, OH, cyano, C(O)NH₂);

R²³, R²⁹, R³⁶ represent —N(R^{46a})(R^{46b}), C₁-C₆ alkyl, C₁-C₆ alkoxy (which alkyl and alkyloxy are optionally substituted by halo, OH, cyano, C(O)NH₂);

R⁴³ represents heteroaryl, heterocyclyl;

m is an integer of 0, 1, 2, 3, 4, 5 or 6;

with the proviso that the compound is not:

a)
1 tert-Butyl [(4-{[(1-naphthylsulfonyl)amino]methyl}cyclohexyl)methyl]-carbamate;
2. tert-Butyl [(trans-4-{[(2-naphthylsulfonyl)amino]methyl}cyclohexyl)methyl] carbamate;
3. tert-Butyl({trans-4-[({[2-trifluoromethyl)phenyl]sulfonyl}amino)methyl]cyclohexyl)methyl)carbamate; or
   b)
1. tert-Butyl [(trans-4-{[(1-naphthylsulfonyl)amino]methyl}cyclohexyl)methyl] carbamate;
2. N-{[4-({[(Cyclopropylmethyl)carbamoyl]amino}methyl)cyclohexyl]methyl}-4-fluorobenzamide;
3. 2-Fluoro-N-{[4-({[(3-methoxypropyl)carbamoyl]amino}methyl)cyclohexyl]methyl}benzamide;
4. 2-Fluoro-N-{[4-({[(2-methoxypropyl)carbamoyl]amino}methyl)cyclohexyl]methyl}benzamide;
5. 3,4-Dimethoxy-N-{[4-({[(4-methylbenzyl)carbamoyl]amino}methyl)cyclohexyl]methyl}benzenesulfonamide;
6. 3-Methyl-N-({4-[({[2-(trifluoromethyl)benzyl]carbamoyl}amino)methyl]cyclohexyl}methyl)benzamide;
7. 6-Chloro-N-{[4-({[(4-fluorobenzyl)carbamoyl]amino}methyl)cyclohexyl]methyl}nicotinamide;
8. 6-Chloro-N-{[4-({[(2-methoxyethyl)carbamoyl]amino}methyl)cyclohexyl]methyl}nicotinamide;
9. 2,3-Dichloro-N-{[4-({[(2-phenylethyl)carbamoyl]amino}methyl)cyclohexyl]methyl}benzamide;
10. 4-Methyl-N-[(4-{[(methylcarbamoyl)amino]methyl}cyclohexyl)methyl] benzamide;
11. 3-Chloro-4-fluoro-N-{[4-({[(2-phenylethyl)carbamoyl]amino}methyl)cyclohexyl]methyl}benzenesulfonamide;
12. N-[(4-{[(Benzylcarbamoyl)amino]methyl}cyclohexyl)methyl]-4-methylbenzenesulfonamide;
13. N-{[4-({[(2-Chlorobenzyl)carbamoyl]amino}methyl)cyclohexyl]methyl}-4-methylbenzenesulfonamide;
14. 4-Methyl-N-{[4-({[(2-phenylethyl)carbamoyl]amino}methyl)cyclohexyl]methyl}benzenesulfonamide;
15. 4-Methyl-N-{[4-({[(4-methylbenzyl)carbamoyl]amino}methyl)cyclohexyl]methyl}benzenesulfonamide;
16. N-{[4-({[(2-Chlorobenzyl)carbamoyl]amino}methyl)cyclohexyl]methyl}-3,4-dimethoxybenzenesulfonamide;
17. 3,4-Dimethoxy-N-{[4-({[(2-phenylethyl)carbamoyl]amino}methyl)cyclohexyl]methyl}benzenesulfonamide;
18. 4-Methyl-N-{[4-({[(4-methylbenzyl)carbamoyl]amino}methyl)cyclohexyl]methyl}benzamide;
19. N-{[4-({[(2-Chlorobenzyl)carbamoyl]amino}methyl)cyclohexyl]methyl}-4-fluorobenzamide;
20. 4-Chloro-N-{[4-({[(4-fluorobenzyl)carbamoyl]amino}methyl)cyclohexyl]methyl}benzamide;
21. 2-Fluoro-N-({4-[({[2-(trifluoromethyl)benzyl]carbamoyl}amino)methyl]cyclohexyl}methyl)benzamide;
22. 4-Nitro-N-({4-[({[2-(trifluoromethyl)benzyl]carbamoyl}amino)methyl]cyclohexyl}methyl)benzamide;
23. N {[4-({[(4-Fluorobenzyl)carbamoyl]amino}methyl)cyclohexyl]methyl}-3-methylbenzamide;
24. N-[(4-{[(Benzylcarbamoyl)amino]methyl}cyclohexyl)methyl]-4-methylbenzamide;
25. 2,3-Dichloro-N-({4-[({[2-(trifluoromethyl)benzyl]carbamoyl}amino) methyl]cyclohexyl}methyl)benzamide;
26. 4-Chloro-N-{[4-({[(3-chlorobenzyl)carbamoyl]amino}methyl)cyclohexyl]methyl}benzamide;
27. 6-Chloro-N-{[4-({[(2-chlorobenzyl)carbamoyl]amino}methyl)cyclohexyl]methyl}nicotinamide;
28. 2,3-Dichloro-N-{[4-({[(cyclopropylmethyl)carbamoyl]amino}methyl)cyclohexyl]methyl}benzamide;
29. 6-Chloro-N-[(4-{[(propylcarbamoyl)amino]methyl}cyclohexyl)methyl]nicotinamide;
30. 6-Chloro-N-[(4-{[(isobutylcarbamoyl)amino]methyl}cyclohexyl) methyl]nicotinamide;
31. 2-Fluoro-N-{[4-({[(4-fluorobenzyl)carbamoyl]amino}methyl)cyclohexyl]methyl}benzamide;
32. 3-Cyano-N-({4-[({[2-(trifluoromethyl)benzyl]carbamoyl}amino)methyl]cyclohexyl}methyl)benzamide;
33. 4-Fluoro-N-{[4-({[(4-fluorobenzyl)carbamoyl]amino}methyl)cyclohexyl]methyl}benzamide;
34. N {[4-({[(Cyclopropylmethyl)carbamoyl]amino}methyl)cyclohexyl]methyl}-2-fluorobenzamide;
35. N-[(4-{[(Isobutylcarbamoyl)amino]methyl}cyclohexyl)methyl]-4-methylbenzamide;
36. 2,3-Dichloro-N-{[4-({[(4-methylbenzyl)carbamoyl]amino}methyl)cyclohexyl]methyl}benzamide;
37. N-({4-[({[2-(Trifluoromethyl)benzyl]carbamoyl}amino)methyl]cyclohexyl}methyl)nicotinamide;
38. N {[4-({[(2-Methoxyethyl)carbamoyl]amino}methyl)cyclohexyl]methyl}-3-methylbenzamide;
39. 3-Methyl-N-{[4-({[(2-phenylethyl)carbamoyl]amino}methyl)cyclohexyl]methyl}benzamide;
40. 2,3-Dichloro-N-{[4-({[(2-methoxyethyl)carbamoyl]amino}methyl)cyclohexyl]methyl}benzamide;
41. 4-Fluoro-N-[(4-{[(propylcarbamoyl)amino]methyl}cyclohexyl)methyl] benzamide;
42. N {[4-({[(3-Methoxypropyl)carbamoyl]amino}methyl)cyclohexyl]methyl}-4-methylbenzamide;
43. 6-Chloro-N-({4-[({[2-(trifluoromethyl)benzyl]carbamoyl}amino)methyl]cyclohexyl}methyl)nicotinamide;
44. 3-Cyano-N-{[4-({[(cyclopropylmethyl)carbamoyl]amino}methyl)cyclohexyl]methyl}benzamide;
45. 3-Methyl-N-[(4-{[(propylcarbamoyl)amino]methyl}cyclohexyl)methyl] benzamide;
46. 4-Methyl-N-{[4-({[(2-phenylethyl)carbamoyl]amino}methyl)cyclohexyl]methyl}benzamide;
47. 3-Cyano-N-{[4-({[(2-methoxyethyl)carbamoyl]amino}methyl)cyclohexyl]methyl}benzamide;
48. N-{[4-({[(Cyclopropylmethyl)carbamoyl]amino}methyl)cyclohexyl]methyl}-4-fluorobenzamide;
49. 4-Fluoro-N-{[4-({[(2-methoxyethyl)carbamoyl]amino}methyl)cyclohexyl]methyl}benzamide;
50. 4-Methoxy-N-[(4-{[(propylcarbamoyl)amino]methyl}cyclohexyl)methyl] benzamide;
51. N-[(4-{[(Isobutylcarbamoyl)amino]methyl}cyclohexyl)methyl]-4-methoxybenzamide;
52. 2,3-Dichloro-N-[(4-{[(isobutylcarbamoyl)amino]methyl}cyclohexyl)methyl] benzamide;
53. 2-Fluoro-N-[(4-{[(isobutylcarbamoyl)amino]methyl}cyclohexyl)methyl] benzamide;
54. 6-Chloro-N-{[4-({[(2-phenylethyl)carbamoyl]amino}methyl)cyclohexyl]methyl}nicotinamide;
55. N-[(4-{[(Benzylcarbamoyl)amino]methyl}cyclohexyl)methyl]-4-chlorobenzamide;
56. 2,3-Dichloro-N-{[4-({[(2-chlorobenzyl)carbamoyl]amino}methyl)cyclohexyl]methyl}benzamide;
57. 4-Fluoro-N-({4-[({[2-(trifluoromethyl)benzyl]carbamoyl}amino)methyl]cyclohexyl}methyl)benzamide;
58. 6-Chloro-N-{[4-({[(cyclopropylmethyl)carbamoyl]amino}methyl)cyclohexyl]methyl}nicotinamide;
59. N-{[4-({[(Cyclopropylmethyl)carbamoyl]amino}methyl)cyclohexyl]methyl}-4-methoxybenzamide;
60. 3-Cyano-N-[(4-{[(propylcarbamoyl)amino]methyl}cyclohexyl)methyl] benzamide;
61. N-{[4-({[(2-Chlorobenzyl)carbamoyl]amino}methyl)cyclohexyl]methyl}-3-methylbenzamide;
62. 2,3-Dichloro-N-{[4-({[(3-chlorobenzyl)carbamoyl]amino}methyl)cyclohexyl]methyl}benzamide;
63. tert-Butyl ({4-[({[3′-({[2-(4-hydroxyphenyl)ethyl]amino}methyl)biphenyl-3-yl]carbonyl}amino)methyl]cyclohexyl}methyl)carbamate;
64. tert-Butyl [(trans-4-{[(1-naphthylsulfonyl)amino]methyl}cyclohexyl)methyl] carbamate;
65. 3-Chloro-N-{[4-({[(2-chlorobenzyl)carbamoyl]amino}methyl)cyclohexyl]methyl}-4-fluorobenzenesulfonamide;
66. 2,3-Dichloro-N-{[4-({[(2-chlorobenzyl)carbamoyl]amino}methyl)cyclohexyl]methyl}benzamide;
67. 2,3-Dichloro-N-[(4-{[(propylcarbamoyl)amino]methyl}cyclohexyl)methyl] benzamide;
68. 4-Chloro-N-{[4-({[(3-methoxypropyl)carbamoyl]amino}methyl)cyclohexyl]methyl}benzamide;
69. 3-Chloro-4-fluoro-N-{[4-({[(4-methylbenzyl)carbamoyl]amino}methyl)cyclohexyl]methyl}benzenesulfonamide;
70. N-[(trans-4-{[(Benzylcarbamoyl)(isopropyl)amino]methyl}cyclohexyl)methyl]benzenesulfonamide;
71. tert-Butyl [(4-{[(1-Naphthylsulfonyl)amino]methyl}cyclohexyl)methyl] carbamate;
72. tert-Butyl [(trans-4-{[(2-naphthylsulfonyl)amino]methyl}cyclohexyl)methyl]carbamate;
73. tert-Butyl [(trans-4-{[(1-naphthylsulfonyl)amino]methyl}cyclohexyl)methyl]carbamate;
74. N-{[4-({[(Cyclopropylmethyl)carbamoyl]amino}methyl)cyclohexyl]methyl}-3-fluorobenzamide;
75. 2-Fluoro-N-{[4-({[(3-methoxypropyl)carbamoyl]amino}methyl)cyclohexyl]methyl}benzamide;
76. 2-Fluoro-N-[[4-[[[[(2-methoxyethyl)amino]carbonyl]amino]methyl]cyclohexyl]methyl] benzamide;
77. 3,4-Dimethoxy-N-{[4-({[(4-methylbenzyl)carbamoyl]amino}methyl)cyclohexyl]methyl}benzenesulfonamide;
78. 3-Methyl-N-({4-[({[2-(trifluoromethyl)benzyl]carbamoyl}amino) methyl]cyclohexyl}methyl)benzamide;
79. 6-Chloro-N-{[4-({[(4-fluorobenzyl)carbamoyl]amino}methyl)cyclohexyl]methyl}nicotinamide;
80. 6-Chloro-N-{[4-({[(2-methoxyethyl)carbamoyl]amino}methyl)cyclohexyl]methyl}nicotinamide;
81. 2,3-Dichloro-N-{[4-({[(2-phenylethyl)carbamoyl]amino}methyl)cyclohexyl]methyl}benzamide;
82. 4-Methyl-N-[(4-{[(methylcarbamoyl)amino]methyl}cyclohexyl)methyl]benzamide;
83. 3-Chloro-4-fluoro-N-{[4-({[(2-phenylethyl)carbamoyl]amino}methyl)cyclohexyl]methyl}benzenesulfonamide;
84. N-[(4-{[(Benzylcarbamoyl)amino]methyl}cyclohexyl)methyl]-4-methylbenzenesulfonamide;
85. N-{[4-({[(2-Chlorobenzyl)carbamoyl]amino}methyl)cyclohexyl]methyl}-4-methylbenzenesulfonamide;
86. 4-Methyl-N-{[4-({[(2-phenylethyl)carbamoyl]amino}methyl)cyclohexyl]methyl}benzenesulfonamide;
87. 4-Methyl-N-{[4-({[(4-methylbenzyl)carbamoyl]amino}methyl)cyclohexyl]methyl}benzenesulfonamide;
88. N-{[4-({[(2-Chlorobenzyl)carbamoyl]amino}methyl)cyclohexyl]methyl}-3,4-dimethoxybenzenesulfonamide; and
89. 3,4-Dimethoxy-N-{[4-({[(2-phenylethyl)carbamoyl]amino}methyl)cyclo-hexyl]methyl}benzenesulfonamide.

In one embodiment L represents C(O).

In one embodiment of the invention n is 1.

In one embodiment Z represents

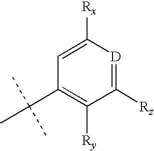

wherein

D represents N or C, optionally substituted by hydrogen, halo, C₁-C₄ alkyl, aryl, NR⁴⁷C(O)R⁴⁸;

Rₓ and R_{z} independently represent hydrogen, halo, —OH, —OR⁴⁹, —SR⁵⁰, —N(R^{51a})(R^{51b}), aryl, C₃-C₈ cycloalkyl, C₃-C₈ cycloalkenyl, heterocyclyl, heteroaryl, (which aryl, cycloalkyl, cycloalkenyl, heterocyclyl and heteroaryl groups are optionally substituted by one or more groups of the following halo, OH, oxo, —C(O)N(R^{52a})(R^{52b}), —OR⁵³, —SR⁵⁴, —C(O)OR⁵⁵, cyano, —N(R⁵⁶)C(O)OR⁵⁷, —N(R⁵⁶)C(O)N(R^{52a})(R^{52b}), —N(R⁵⁶)S(O)₂R⁶⁰, —S(O)₂ N(R^{52a})(R^{52b}), —N(R⁵⁶)(CHR⁵⁸)ₘN(R^{52a})(R^{52b}), —N(R^{52a})(R^{52b}), —C(O)R⁵⁹, —N(R⁵⁶)C(O)R⁵⁹, —N(R⁵⁶)(CHR⁵⁸)ₘOR⁵⁵, —N(R⁵⁶)(CHR⁵⁸)ₘC(O)N(R^{52a})(R^{52b}), —N(R⁵⁶)(CHR⁵⁸)ₘC(O)O(R⁵⁷), —S(O)₍₁₋₂₎R⁶⁰, heteroaryl, heterocyclyl (which heteroaryl, heterocyclyl groups are optionally substituted by one or more of the following —OH, oxo, —C(O)R⁶¹, —C(O)N(R^{62a})(R^{62b}), C₁-C₄ alkyl (which alkyl is optionally substituted by OH)), C₁-C₆ alkyl, C₁-C₆ alkoxy (which alkyl and alkoxy groups are optionally substituted by one or more of the following —OH, halo, —N(R^{63a})(R^{63b}), —C(O)OR⁶⁴, N(R⁶⁵)C(O)OR⁶⁴, —N(R⁶⁵)C(O)R⁶⁶, —N(R⁶⁵)C(O)N(R^{63a})(R^{63b}), —N(R⁶⁵)S(O)₍₁₋₂₎(R⁶⁶), —C(O)N(R^{63a})(R^{63b}), heterocyclyl, heteroaryl (which heteroaryl, heterocyclyl groups are optionally substituted by one or more C₁-C₄ alkyl, oxo, —C(O)R⁶⁷));

R_{y} represents hydrogen, halo, C₁-C₆ alkyl, C₁-C₆ alkoxy;

R_{y} and R_{z} may together with the carbon atoms to which they are attached represent a benzene ring;

wherein

R^{51a}, R^{51b}, R^{52a}, R^{52b}, R^{62a}, R^{62b}, R^{63a}, R^{63b} independently represent hydrogen, C₁-C₆ alkyl (optionally substituted by one or more OH, halo, heteroaryl (which heteroaryl optionally is substituted by one or more C₁-C₄ alkyl or C₁-C₄ alkoxy));

R⁴⁶, R⁴⁸, R⁵⁰, R⁵⁴, R⁵⁹, R⁶¹, R⁶⁶ independently represent C₁-C₆ alkyl, optionally substituted by one or more OH, halo, heteroaryl, heterocyclyl, which groups optionally are substituted by one or more C₁-C₆ alkyl, OH;

R⁴⁵, R⁴⁷, R⁵⁵, R⁵⁶, R⁵⁸, R⁶⁰, R⁶⁴, R⁶⁵ independently represent hydrogen, C₁-C₆ alkyl, optionally substituted by one or more OH, halo, heteroaryl, heterocyclyl (which heteroaryl, heterocyclyl groups optionally are substituted by one or more C₁-C₆ alkyl, OH;

R⁵⁰, R⁵⁷, R⁶⁷ independently represent hydrogen, C₁-C₆ alkyl (optionally substituted by one or more OH, halo);

R⁴⁹, R⁵³ independently represent C₃-C₈ cycloalkyl, C₃-C₈ cycloalkenyl, heteroaryl, heterocyclic, C₁-C₆ alkyl (which alkyl is optionally substituted by one or more OH, halo, N(R^{63a})(R^{63b}), heteroaryl, heterocyclyl (which heteroaryl and heterocyclyl groups optionally are substituted by one or more OH, oxo, C₁-C₆ alkyl, C₁-C₆ alkoxy, —C(O)R⁶⁶));

m is an integer of 0, 1, 2, 3, 4, 5, 6.

In one embodiment of the invention R_{z} represents hydrogen, halo, C₁-C₆ alkyl, C₁-C₆ alkoxy, aryl or heteroaryl (which aryl and heteroaryl groups are optionally substituted by one or more of —OH, halo, cyano, amino, C₁-C₆ alkyl, C₁-C₆ alkoxy, oxo, —NR⁵⁶C(O)R⁵⁹).

In one embodiment of the invention R₁ represents —OR⁶.

In one embodiment of the invention Z represents

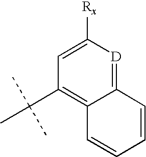

wherein

D represents N or C, optionally substituted by hydrogen, halo, C₁-C₄ alkyl, aryl;

R^{x} represents hydrogen, halo, —OR⁶⁸, —SR⁶⁹, —N(R⁷⁰)C(O)R⁷¹, —N(R^{72a})(R^{72b})aryl, C₃-C₈ cycloalkyl, C₃-C₈ cycloalkenyl, heterocyclyl, heteroaryl, (which aryl, cycloalkyl, cycloalkenyl, heterocyclyl, heteroaryl groups are optionally substituted by one or more groups selected from halo, OH, oxo, —C(O)N(R^{73a})(R^{73b}), OR⁷⁴, —SR⁷⁵, —C(O)OR⁷⁶, cyano, —N(R⁷⁷)C(O)OR⁷⁸, —N(R⁷⁷)C(O)N(R^{73a})(R^{73b}), —N(R⁷⁷)S(O)₍₁₋₂₎R⁷⁸, —N(R⁷⁷)(CHR⁷⁹)ₘN(R^{73a})(R^{73b}), —N(R^{73a})(R^{73b}), —C(O)R⁸⁰, —N(R⁷⁷)C(O)R⁸⁰, —N(R⁷⁷)(CHR⁷⁹)ₘOR⁷⁹, —N(R⁷⁷)(CHR⁷⁹)ₘOH, —N(R⁷⁷)(CHR⁷⁹)ₘC(O)N(R^{73a})(R^{73b}), —N(R⁷⁷)(CHR⁷⁹)ₘC(O)O(R⁷⁶), —N(R⁷⁷)C(O)R⁸⁰, —S(O)₍₁₋₂₎R⁷⁸, —S(O)₍₁₋₂₎N(R^{73a})(R^{73b}), heteroaryl, heterocyclyl (which heteroaryl, heterocyclyl groups are optionally substituted by one or more of the following OH, oxo, C(O)R⁸¹, C(O)N(R^{82a})(R^{82b}), —N(R^{82a})(R^{82b}), C₁-C₄ alkyl, C₁-C₄ alkoxy (which alkyl or alkoxy is optionally substituted by one or more OH, —N(R^{82a})(R^{82b}))), C₁-C₆ alkyl, C₁-C₆ alkoxy (which alkyl and alkoxy groups are optionally substituted by one or more of the following —OH, halo, —C(O)OR⁸³, —N(R^{84a})(R^{84b}), —N(R⁸⁵)C(O)OR⁸³, —N(R⁸⁵)C(O)R⁸⁶, —N(R⁸⁵)C(O)N(R^{84a})(R^{84b}), —N(R⁸⁵)S(O)₍₁₋₂₎R⁸⁷, —C(O)N(R^{84a})(R^{84b}), heteroaryl, heterocyclyl (which heteroaryl, heterocyclyl groups are optionally substituted by one or more C₁-C₄ alkyl, oxo, —C(O)R⁸⁸)),

R^{72a}, R^{72b}, R^{73a}, R^{73b}, R^{82a}, R^{82b}, R^{84a}, R^{84b} independently represent hydrogen, C₁-C₆ alkyl (optionally substituted by one or more OH, halo, heteroaryl (which heteroaryl optionally is substituted by one or more C₁-C₄ alkyl));

R⁶⁸, R⁶⁹, R⁷⁴, R⁷⁵, R⁷⁸, R⁸⁷ independently represent C₃-C₈ cycloalkyl, C₃-C₈ cycloalkenyl, heteroaryl, heterocyclyl, C₁-C₆ alkyl (which groups are optionally substituted by one or more OH, halo, —N(R^{84a})(R^{84b}), heteroaryl, heterocyclyl (which heteroaryl, heterocyclyl groups optionally are substituted by one or more OH, oxo, C₁-C₆ alkyl, C₁-C₆ alkoxy, C(O)R⁸⁹));

R⁷¹, R⁷⁶, R⁷⁸, R⁷⁹, R⁸⁰, R⁸¹, R⁸³, R⁸⁶, R⁸⁷, R⁸⁸, R⁸⁹ independently represent hydrogen, C₁-C₆ alkyl, optionally substituted by one or more OH, halo, heteroaryl, heterocyclyl (which groups optionally are substituted by one or more OH, C₁-C₆ alkyl, C(O)R⁸⁹);

R⁷⁰, R⁷⁷, R⁸⁵ independently represent hydrogen, C₁-C₆ alkyl (optionally substituted by one or more OH, halo);

m is an integer of 0, 1, 2, 3, 4, 5 or 6.

In one embodiment of the invention D represent N.

In one embodiment of the invention the compound is according to formula (I) wherein

Rₓ is selected from optionally substituted phenyl, thiazole, thienyl, pyridine, pyrazine, pyrimidine, piperidine, piperazine or imidazolidine.

A particular embodiment of the invention is provided by a compound of formula II

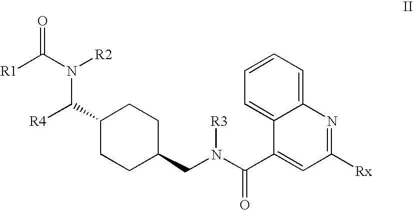

or a pharmaceutically acceptable salt thereof in which R₁, R₂, R₃ and R₄ are as previously defined and Rx represents a group a), b) or c):
a)

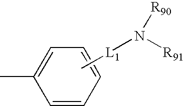

in which L₁ represents a bond or a C₁-C₄ alkylene chain and R⁹⁰ and R⁹¹ independently represent H or C₁-C₆ alkyl optionally substituted by hydroxy or C₁-C₄ alkoxy
b)

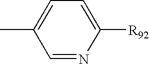

in which R₉₂ represents a group -L₂-L₃-NR⁹³R⁹⁴ in which L₂ is O or N, L₃ is a C₂-C₄ alkylene chain and R⁹³ and R⁹⁴ independently represent H or C₁-C₆ alkyl optionally substituted by hydroxy or C₁-C₄ alkoxy or R₉₂ represents azetidino, pyrrolidino, piperidino, morpholino or piperazino each of which is optionally substituted by one or more of the following: hydroxy, a C₁-C₄ alkyl, C₁-C₄ alkoxy or a C₁-C₄ alkanoyl
c)

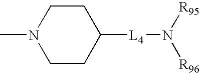

in which L₄ is a C₁-C₄ alkylene chain optionally substituted by hydroxy or fluoro provided that no carbon atom in the chain is attached to two hetero atom (that is O or N) and R⁹⁵ and R⁹⁶ independently represent H or C₁-C₆ alkyl optionally substituted by hydroxy or C₁-C₄ alkoxy or R⁹⁵ and R⁹⁶ together with the nitrogen atom to which they are attached represent azetidino, pyrrolidino, piperidino, morpholino or piperazino each of which is optionally substituted by one or more of the following: hydroxy, a C₁-C₄ alkyl or C₁-C₄ alkoxy.

In a particular group of compounds of formula II, R^{x} represents group a) as above.

In a second group of compounds of formula II, R^{x} represents group b) as above.

In a third group of compounds of formula II, R^{x} represents group c) as above.

In a further group of compounds of formula II, R^{x} represents group a) above,

wherein L₁ represents a bond or a C₁-C₂ alkylene chain, or L₁ represents a C₁ alkylene chain, and R⁹⁰ and R⁹¹ independently represent H, C₁-C₃ alkyl or C₁ alkyl optionally substituted by hydroxy or C₁ alkoxy, or both R⁹⁰ and R⁹¹ represent H.

In a even further group of compounds of formula II, R^{x} represents group b) above, wherein R₉₂ represents a group -L₂-L₃-NR⁹³R⁹⁴ in which L₂ is O or N, L₃ is a C₂-C₄ alkylene chain, or L₃ is a C₂-C₃ alkylene chain, and R⁹³ and R⁹⁴ independently represent H or C₁-C₃ alkyl optionally substituted by hydroxy or C₁ alkoxy or both R⁹³ and R⁹⁴ represent H, or wherein or R₉₂ represents piperidino, morpholino or piperazino each of which is optionally substituted by one or more of the following: hydroxy, a C₁ alkyl, C₁ alkoxy or a C₁ alkanoyl.

In a still further group of compounds of formula II, R^{x} represents group c) as above, wherein L₄ is a C₁-C₃ alkylene chain or a C₂-C₃ alkylene chain, which chain is optionally substituted by hydroxy provided that no carbon atom in the chain is attached to two hetero atom (that is O or N) and R⁹⁵ and R⁹⁶ independently represent H or C₁-C₂ alkyl optionally substituted by hydroxy or C₁ alkoxy, or R⁹⁵ and R⁹⁶ independently C₁-C₂ alkyl optionally substituted by hydroxy, or R⁹⁵ and R⁹⁶ together with the nitrogen atom to which they are attached represent azetidino or pyrrolidino each of which is optionally substituted by one or more of the following: hydroxy, a C₁ alkyl or C₁ alkoxy, or R⁹⁵ and R⁹⁶ together with the nitrogen atom to which they are attached represent azetidino which is optionally substituted by a hydroxy.

In a further embodiment of the invention a compound of formula II, or a pharmaceutically acceptable salt thereof, is provided, wherein R^{x} represents group a) in which L₁ represents a C₁ alkylene chain, and both R⁹⁰ and R⁹¹ represent H, or wherein R^{x} represents group b), in which R₉₂ represents a group -L₂-L₃-NR⁹³R⁹⁴ in which L₂ is O or N, L₃ is a C₂-C₃ alkylene chain, and R⁹³ and R⁹⁴ both represent H, or wherein or R₉₂ represents piperidino, morpholino or piperazino each of which is optionally substituted by one or more of the following: hydroxy, a C₁ alkyl, C₁, alkoxy or a C₁ alkanoyl, or wherein R^{x} represents group c) in which L₄ is a C₂-C₃ alkylene chain, which chain is optionally substituted by hydroxy provided that no carbon atom in the chain is attached to two hetero atom (that is O or N) and R⁹⁵ and R⁹⁶ independently C₁-C₂ alkyl optionally substituted by hydroxy, or R⁹⁵ and R⁹⁶ together with the nitrogen atom to which they are attached represent azetidino which is optionally substituted by a hydroxy.

Further embodiments relate to a compound of formula (I) or formula (II) as described herein, or a pharmaceutically acceptable salt thereof, in which

R₁ represents —OR⁶,

R₂ and R₃ independently represent hydrogen, C₁-C₆ alkyl, C₁-C₄ alkyl, C₁-C₃ alkyl, C₁-C₂ alkyl, C₂ alkyl or C₁ alkyl, or both R₂ and R₃ represent hydrogen,

R₄ represents hydrogen, C₁-C₆ alkyl, C₁-C₄ alkyl, C₁-C₃ alkyl, C₁-C₂ alkyl, C₂ alkyl or C₁ alkyl, or R₄ represents hydrogen,

R₅ represents hydrogen or C₁-C₆ alkyl, C₁-C₄ alkyl, C₁-C₃ alkyl, C₁-C₂ alkyl, C₂ alkyl or C₁ alkyl, or R₅ represents hydrogen, and

R⁶ represents C₃-C₅ alkyl, C₃-C₄ alkyl C₄-C₅ alkyl or C₄ alkyl, e.g. a t-butyl.

Still further embodiments relate to a compound of formula (I) or formula (II) as described herein, or a pharmaceutically acceptable salt thereof, in which

R₁ represents —OR⁶,

R₂ and R₃ independently represent hydrogen or C₁-C₆ alkyl,

R₄ represents hydrogen or C₁-C₆ alkyl,

R₅ represents hydrogen or C₁-C₆ alkyl, and

R⁶ represents C₃-C₅ alkyl.

In one embodiment there is provided a compound according to formula (I) or formula (II), including compounds of proviso b), for use in therapy.

In one embodiment a method for treating obesity or being overweight, eating disorders, dyslipidemia, atherosclerosis, heart failure, stroke, type 2 diabetes mellitus and prevention of type 2 diabetes is provided, comprising administering a pharmacologically effective amount of a compound of formula (I) or formula (II) as defined herein, including proviso b) to a patient in need thereof.

In one embodiment a process for preparing the compound of the invention as described herein.

The following definitions shall apply throughout the specification and the appended claims.

Unless otherwise specified, alkyl groups and alkoxy groups as defined herein may be linear-chain or, when there is a sufficient number (i.e. a minimum of three) of carbon atoms be branched-chain.

Alkylene groups as defined herein are divalent and may be linear-chain or, when there is a sufficient number (i.e. a minimum of three) of carbon atoms, be branched-chain.

The term “aryl”, when used herein, includes C₆-C₁₀ aryl groups such as phenyl, naphthyl, and the like. Unless otherwise specified, aryl and aryloxy groups may be substituted by one or more substituents including —OH, halo, cyano, nitro, C₁-C₆ alkyl, C₁-C₆ alkoxy, sulfamoyl, methylsulfonyl, aryl, amino and methylsulfinyl. When substituted, aryl and aryloxy groups are preferably substituted by between one and three substitutents.

The term “halo”, when used herein, includes fluoro, chloro, bromo and iodo.

The term “cycloalkyl” denotes a saturated monocarbocyclic ring composed of 3, 4, 5, 6, 7 or 8 carbon atoms or a saturated bicyclic ring system composed of 8, 9 or 10 carbon atoms. The cycloalkyl may be attached to an alkyl group in a spirocyclic manner.

The term “cycloalkenyl” denotes unsaturated non-aromatic monocarbocyclic ring composed of 3, 4, 5, 6, 7 or 8 carbon atoms or unsaturated non-aromatic or partly aromatic bicyclic ring system composed of 8, 9 or 10 carbon atoms. The cycloalkenyl may be attached to an alkyl group in a spirocyclic manner.

The term “heterocyclyl” denotes a saturated or unsaturated non-aromatic 3, 4, 5, 6, 7, 8, 9 or 10 membered monocyclic ring or a saturated or unsaturated non-aromatic or partly aromatic 9 or 10 membered bicyclic ring system in which one or more of the atoms in the monocyclic ring or bicyclic ring system is an element other than carbon independently selected from one or more of for example nitrogen, oxygen or sulfur. The term “sulfur” shall be understood to include sulfoxide (S(O)) and sulfone (SO₂). The term “nitrogen” shall be understood to include nitrogen oxide (NO). Examples of said “heterocyclyl” include, but are not limited to aziridinyl, azetidinyl, 2-pyrrolinyl, 3-pyrrolinyl, pyrrolidinyl, imidazolinyl, piperidinyl, piperazinyl, oxiranyl, oxetanyl, tetrahydrofuranyl, tetrahydropyranyl, dihydropyranyl, tetrahydroisoquinolinyl, tetrahydroquinolinyl, 1,4-dioxanyl, 1,3-dioxolanyl, 1,2-oxathiolanyl, morpholinyl, pyrazolidinyl, 1,4-dithianyl, 1,4-oxathianyl, thiomorpholinyl, indolinyl, 1,3-dihydro-2-benzofuranyl, 2,3-dihydro-1-benzofuranyl, 1,3-benzodioxolyl, 2,3-dihydro-1,4-benzodioxinyl, chromanyl, isochromanyl, benzomorpholinyl, benzoxazinonyl, oxopyrrolidinyl, dioxothiolanyl and pyrrolidinonyl.

The term “heteroaryl” denotes an aromatic 5 or 6 membered monocyclic ring or an aromatic 9 or 10 membered bicyclic ring in which one or more of the atoms in the monocyclic ring or bicyclic ring system is an element other than carbon independently selected from one or more of for example nitrogen, oxygen or sulfur. The term “sulfur” shall be understood to include sulfoxide (S(O)) and sulfone (SO₂). The term “nitrogen” shall be understood to include nitrogen oxide (NO). Examples of said “heteroaryl” include, but are not limited to, furanyl, pyrrolyl, pyrazinyl, 2-pyrazolinyl, 3-pyrazolinyl, pyrazolyl, imidazolyl, triazolyl, pyrimidinyl, pyridazinyl, pyridinyl, 1-oxido-pyridinyl, isoxazolyl, oxazolyl, isothiazolyl, thiazolyl, thienyl, 1,2,4-triazolyl, furazanyl, 1,2,3-oxadiazolyl, 1,2,4-oxadiazolyl, 1,2,5-oxadiazolyl, 1,3,4-oxadiazolyl, 1,2,5-thiadiazolyl, 1,2,4-thiadiazolyl, 1,2,3-thiadiazolyl, benzofuranyl, isobenzofuranyl, indolyl, isoindolyl, benzothienyl, benzo[c]thienyl, benzimidazolyl, purinyl, indazolyl, benzoxazolyl, benzisoxazolyl, benzthiazolyl, quinolinyl, quinoxalinyl, quinazolinyl, cinnolinyl, isoquionolinyl, aza-indolyl, pyrazinopyridinyl, pyrazolopyridinyl, pyrrolopyrazinyl, benzotriazolyl, imidazo[1,2-a]pyridinyl, phthalazinyl and tetrazolyl.

Substituents on heterocyclyl and heteroaryl groups may, where appropriate, be located on any atom in the ring system including a heteroatom. The point of attachment of heterocyclyl and heteroaryl groups may be via any atom in the ring system including (where appropriate) a heteroatom, or an atom on any fused carbocyclic ring that may be present as part of the ring system. Heterocyclyl and heteroaryl groups may also be in the N- or S-oxidised form.

Unless otherwise specified, the cycloalkyl, cycloalkenyl, heterocyclyl and heteroaryl may be substituted by one or more substituents including —OH, oxo, halo, cyano, amino, C₁-C₆ alkyl, C₁-C₆ alkoxy, C₂-C₆ alkenyl, C₂-C₆ alkynyl, amido, sulfamoyl, methylsulfonyl, methylsulfinyl, phenyl and C₁-C₆ alkanoyl.

The term “alkenyl” refers to a monovalent straight or branched chain alkyl group having at least one carbon-carbon double bond. The double bond of an alkenyl can be unconjugated or conjugated to another unsaturated group. Unless otherwise specified, alkenyl groups as defined herein may be straight-chain or, when there is a sufficient number (i.e. a minimum of three) of carbon atoms be branched-chain.

The term “alkynyl” refers to a monovalent straight or branched chain alkyl group having at least one carbon-carbon triple bond. The triple bond of an alkynyl can be unconjugated or conjugated to another unsaturated group. Unless otherwise specified, alkynyl groups as defined herein may be straight-chain or, when there is a sufficient number (i.e. a minimum of three) of carbon atoms be branched-chain.

Unless otherwise stated or indicated the term “alkoxy” denotes a group O-alkyl wherein alkyl is as defined above.

“Pharmaceutically acceptable salt”, where such salts are possible, includes both pharmaceutically acceptable acid and base addition salts. A suitable pharmaceutically acceptable salt of a compound of formula (I) or formula (II) is, for example, an acid-addition salt of a compound of formula (I) or formula (II) which is sufficiently basic, for example an acid-addition salt with an inorganic or organic acid such as hydrochloric, hydrobromic, sulphuric, trifluoroacetic, citric or maleic acid; or, for example a base-addition salt of a compound of formula (I) or formula (II) which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such stereoisomers and enantiomers exist, as well as pharmaceutically acceptable salts thereof and solvates thereof such as for instance hydrates including solvates of the free compounds or solvates of a salt of the compound. Stereoisomers may be separated using conventional techniques, e.g. chromatography or fractional crystallisation. The enantiomers may be isolated by separation of racemate for example by fractional crystallisation, resolution or chiral HPLC. The diastereomers may be isolated by separation of isomer mixtures for instance by fractional crystallisation, HPLC or flash chromatography. Alternatively the stereoisomers may be made by chiral synthesis from chiral starting materials under conditions that will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies. The present invention also encompasses prodrugs of a compound of formula (I) or formula (II) that is compounds which are converted into a compound of formula (I) or formula (II) in vivo.

The invention relates to any and all tautomeric forms of the compounds of the formula (I) or formula (II) that possess ACC inhibitory activity.

It is also to be understood that certain compounds of the formula (I) or formula (II) can exist in solvated as well as unsolvated forms such as, for example, hydrated forms. It is to be understood that the invention encompasses all such solvated forms, which possess ACC inhibitory activity.

Compounds of the present invention have been named with the aid of computer software (ACD Name (Product Version 9.04)).

The values of variable groups may be used where appropriate with any of the definitions, claims or embodiments defined hereinbefore or hereinafter, for compounds of formula (I) or formula (II).

Illustrative values and examples of any substituent, R group or any part of such groups include, but are not limited to:
C₁-C₄ alkyl: C₃-C₄ alkyl, C₄ alkyl, C₃ alkyl, C₁-C₃ alkyl, C₁-C₂ alkyl, C₂ alkyl, C₁ alkyl, methyl, ethyl, n-propyl, iso-propyl, 2-methyl-1-propyl, 2-methyl-2-propyl, butyl, iso-butyl and t-butyl (i.e. tert-butyl);
C₁-C₆ alkyl: C₄-C₆ alkyl, C₃-C₆ alkyl, C₆ alkyl, C₅ alkyl, C₄-C₅ alkyl, C₁-C₅ alkyl, C₁-C₄ alkyl, C₃-C₅ alkyl, C₃-C₄ alkyl, C₄ alkyl, C₃ alkyl, C₁-C₃ alkyl, C₁-C₂ alkyl, C₂ alkyl, C₁ alkyl, methyl, ethyl, n-propyl, iso-propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, iso-butyl, t-butyl (i.e. tert-butyl), pentyl, iso-pentyl, neo-pentyl and hexyl;
C₁-C₈ alkyl: C₁-C₆ alkyl (as above), C₁-C₄ alkyl (as above), methyl, ethyl, n-propyl, iso-propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, iso-butyl, t-butyl (i.e. tert-butyl), pentyl, iso-pentyl, neo-pentyl, hexyl, heptyl and octyl;
C₁-C₆ alkoxy: C₄-C₆ alkoxy, C₃-C₆ alkoxy, C₆ alkoxy, C₅ alkoxy, C₄-C₅ alkoxy, C₁-C₅ alkoxy, C₁-C₄ alkoxy, C₃-C₅ alkoxy, C₃-C₄ alkoxy, C₄ alkoxy, C₃ alkoxy, C₁-C₃ alkoxy, C₁-C₂ alkoxy, C₂ alkoxy, C₁ alkoxy, methoxy, ethoxy, n-propyloxy, iso-propyloxy, 2-methyl-1-propyloxy, 2-methyl-2-propyloxy, 2-methyl-1-butyloxy, 3-methyl-1-butyloxy, 2-methyl-3-butyloxy, 2,2-dimethyl-1-propyloxy, 2-methyl-1-pentyloxy, 3-methyl-1-pentyloxy, 4-methyl-1-pentyloxy, 2-methyl-2-pentyloxy, 3-methyl-2-pentyloxy, 4-methyl-2-pentyloxy, 2,2-dimethyl-1-butyloxy, 3,3-dimethyl-1-butyloxy, 2-ethyl-1-butyloxy, butyloxy, iso-butyloxy, t-butyloxy, pentyloxy, iso-pentyloxy, neo-pentyloxy, and hexyloxy;
C₂-C₆ alkenyl: C₄-C₆ alkenyl, C₃-C₆ alkenyl, C₆ alkenyl, C₅ alkenyl, C₄-C₅ alkenyl, C₂-C₅ alkenyl, C₂-C₄ alkenyl, C₃-C₅ alkenyl, C₃-C₄ alkenyl, C₄ alkenyl, C₃ alkenyl, C₂-C₃ alkenyl, C₂ alkenyl, vinyl, allyl, butenyl, pentenyl, hexenyl, cyclohexenyl, butadienyl, pentadienyl, and hexadienyl;
C₂-C₆ alkynyl: C₄-C₆ alkynyl, C₃-C₆ alkynyl, C₆ alkynyl, C₅ alkynyl, C₄-C₅ alkynyl, C₂-C₅ alkynyl, C₂-C₄ alkynyl, C₃-C₅ alkynyl, C₃-C₄ alkynyl, C₄ alkynyl, C₃ alkynyl, C₂-C₃ alkynyl, C₂ alkynyl, acetylene, propynyl, 1-butynyl, 2-butynyl, 2-pentynyl, 1-pentynyl;
C₃-C₈ cycloalkyl: C₃-C₇ cycloalkyl, C₃-C₅ cycloalkyl, C₃-C₄ cycloalkyl, C₄-C₈ cycloalkyl, C₅-C₈ cycloalkyl, C₆-C₈ cycloalkyl, C₇-C₈ cycloalkyl, C₄-C₇ cycloalkyl, C₅-C₇ cycloalkyl, C₆-C₇ cycloalkyl, C₄-C₆ cycloalkyl, C₄-C₅ cycloalkyl, cyclopropyl, cyclobutyl, cyclopentyl, cyclohexyl, cycloheptyl and cyclooctanyl;
C₃-C₈ cycloalkenyl: C₃-C₇ cycloalkenyl, C₃-C₅ cycloalkenyl, C₃-C₄ cycloalkenyl, C₄-C₈ cycloalkenyl, C₅-C₈ cycloalkenyl, C₆-C₈ cycloalkenyl, C₇-C₈ cycloalkenyl, C₄-C₇ cycloalkenyl, C₅-C₇ cycloalkenyl, C₆-C₇ cycloalkenyl, C₄-C₆ cycloalkenyl, C₄-C₅ cycloalkenyl, cyclobutenyl, cyclopentenyl, cyclohexenyl, cycloheptenyl, cyclooctenyl, cyclopentadienyl, cyclohexadienyl, cyclooctadienyl, decaline, hydrindane, indane, indene, and bicyclo[4.2.0]octa-1,3,5-triene.

Specific compounds of the invention include one or more of the following:
1. tert-Butyl [(trans-4-{[({2-[6-(4-methylpiperazin-1-yl)pyridin-3-yl]quinolin-4-yl}carbonyl)amino]methyl}cyclohexyl)methyl]carbamate;
2. tert-Butyl ({trans-4-[({[2-(2-methyl-6-oxo-1,6-dihydropyridin-3-yl)quinolin-4-yl]carbonyl}amino)methyl]cyclohexyl}methyl)carbamate;
3. tert-Butyl {[trans-4-({[(2-pyrazin-2-ylquinolin-4-yl)carbonyl]amino}methyl)cyclo-hexyl]methyl}carbamate;
4. tert-Butyl ({trans-4-[({[2-(4-ethoxyphenyl)quinolin-4-yl]carbonyl}amino) methyl]-cyclohexyl}methyl)carbamate;
5. tert-Butyl ({trans-4-[({[2-(6-carbamoylpyridin-3-yl)quinolin-4-yl]carbonyl}amino)-methyl]cyclohexyl}methyl)carbamate;
6. tert-Butyl {[trans-4-({[(2-phenylquinolin-4-yl)carbonyl]amino}methyl)-cyclohexyl]-methyl}carbamate;
7. tert-Butyl {[trans-4-({[(2-pyridin-4-ylquinolin-4-yl)carbonyl]amino}methyl)-cyclohexyl]methyl}carbamate;
8. tert-Butyl ({trans-4-[({[2-(3-carbamoylphenyl)quinolin-4-yl]carbonyl}amino)methyl]-cyclohexyl}methyl)carbamate;
9. tert-Butyl ({trans-4-[({[2-(4-carbamoylphenyl)quinolin-4-yl]carbonyl}amino)methyl]-cyclohexyl}methyl)carbamate;
10. tert-Butyl {[trans-4-({[(2-{6-[3-(dimethylamino)propoxy]pyridin-3-yl}quinolin-4-yl)carbonyl]amino}methyl)cyclohexyl]methyl}carbamate;
11. tert-Butyl [(trans-4-{[({2-[6-(dimethylamino)pyridin-3-yl]quinolin-4-yl}carbonyl)amino]-methyl}cyclohexyl)methyl]carbamate;
12. tert-Butyl [(trans-4-{[({2-[4-(trifluoromethyl)phenyl]quinolin-4-yl}carbonyl)amino]-methyl}cyclohexyl)methyl]carbamate;
13. tert-Butyl ({trans-4-[({[2-(5-acetyl-2-thienyl)quinolin-4-yl]carbonyl}amino)methyl]-cyclohexyl}methyl)carbamate;
14. tert-Butyl ({trans-4-[({[2-(4-fluorophenyl)quinolin-4-yl]carbonyl}amino)methyl]-cyclohexyl}methyl)carbamate;
15. tert-Butyl ({trans-4-[({[2-(3,5-dimethylisoxazol-4-yl)quinolin-4-yl]carbonyl}amino)-methyl]cyclohexyl}methyl)carbamate;
16. tert-Butyl {[trans-4-({[(2-pyridin-3-ylquinolin-4-yl)carbonyl]amino}methyl)cyclo-hexyl]methyl}carbamate;
17. Ethyl ({trans-4-[({[2-(4-fluorophenyl)quinolin-4-yl]carbonyl}amino)methyl]cyclo-hexyl}methyl)carbamate;
18. Ethyl [(trans-4-{[({2-[4-(dimethylcarbamoyl)phenyl]quinolin-4-yl}carbonyl)amino]-methyl}cyclohexyl)methyl]carbamate;
19. Ethyl ({trans-4-[({[2-(3-carbamoylphenyl)quinolin-4-yl]carbonyl}amino)methyl]-cyclohexyl}methyl)carbamate;
20. tert-Butyl ({trans-4-[({[2-(4-methylphenyl)quinolin-4-yl]carbonyl}amino)methyl]-cyclohexyl}methyl)carbamate;
21. tert-Butyl ({trans-4-[({[2-(4-chlorophenyl)quinolin-4-yl]carbonyl}amino)methyl]-cyclohexyl}methyl)carbamate;
22. tert-Butyl ({trans-4-[({[2-(3-methoxyphenyl)quinolin-4-yl]carbonyl}amino)-methyl]cyclohexyl}methyl)carbamate;
23. tert-Butyl ({trans-4-[({[2-(2-methoxyphenyl)quinolin-4-yl]carbonyl}amino)methyl]-cyclohexyl}methyl)carbamate;
24. tert-Butyl [(trans-4-{[({2-[4-(methylthio)phenyl]quinolin-4-yl}carbonyl)amino]-methyl}cyclohexyl)methyl]carbamate;
25. tert-Butyl ({trans-4-[({[2-(2,4-dimethyl-1,3-thiazol-5-yl)quinolin-4-yl]carbonyl}amino)-methyl]cyclohexyl}methyl)carbamate;
26. Ethyl {[trans-4-({[(2-pyrazin-2-ylquinolin-4-yl)carbonyl]amino}methyl)cyclohexyl]-methyl}carbamate;
27. Ethyl ({trans-4-[({[2-(3-methoxyphenyl)quinolin-4-yl]carbonyl}amino)methyl]-cyclohexyl}methyl)carbamate;
28. Ethyl ({trans-4-[({[2-(4-methylphenyl)quinolin-4-yl]carbonyl}amino) methyl]cyclo-hexyl}methyl)carbamate;
29. Ethyl [(trans-4-{[({2-[4-(methylthio)phenyl]quinolin-4-yl}carbonyl)amino]methyl}-cyclohexyl)methyl]carbamate;
30. Ethyl ({trans-4-[({[2-(4-ethoxyphenyl)quinolin-4-yl]carbonyl}amino)methyl]-cyclohexyl}methyl)carbamate;
31. Ethyl ({trans-4-[({[2-(2-methoxyphenyl)quinolin-4-yl]carbonyl}amino)methyl]-cyclohexyl}methyl)carbamate;
32. Ethyl ({trans-4-[({[2-(4-chlorophenyl)quinolin-4-yl]carbonyl}amino)methyl]cyclo-hexyl}methyl)carbamate;
33. tert-Butyl {[trans-4-({[(2-pyrimidin-5-ylquinolin-4-yl)carbonyl]amino}methyl)-cyclohexyl]methyl}carbamate;
34. tert-Butyl ({trans-4-[({[2-(4-cyanophenyl)quinolin-4-yl]carbonyl}amino) methyl]-cyclohexyl}methyl)carbamate;
35. tert-Butyl {[trans-4-({[(2-chloroquinolin-4-yl)carbonyl]amino}methyl)cyclohexyl]-methyl}carbamate;
36. tert-Butyl ({trans-4-[({2-[4-(aminomethyl)phenyl]-6-methoxyisonicotinoyl}-amino)methyl]cyclohexyl}methyl)carbamate;
37. tert-Butyl ({trans-4-[({2-[4-(aminomethyl)phenyl]-5-chloroisonicotinoyl} amino)-methyl]cyclohexyl}methyl)carbamate;
38. tert-Butyl ({trans-4-[({2-[4-(aminomethyl)phenyl]-6-methylisonicotinoyl} amino)-methyl]cyclohexyl}methyl)carbamate
39. tert-Butyl [(trans-4-{[({2-[3-(aminomethyl)phenyl]quinolin-4-yl}carbonyl)amino]-methyl}cyclohexyl)methyl]carbamate;
40. [3-(4-{[(trans-4-{[(tert-Butoxycarbonyl)amino]methyl}cyclohexyl)methyl]-carbamoyl}quinolin-2-yl)phenyl]acetic acid;
41. tert-Butyl [(trans-4-{[(2-phenylisonicotinoyl)amino]methyl}cyclohexyl) methyl]-carbamate;
42. tert-Butyl {[trans-4-({[2-(4-methoxyphenyl)isonicotinoyl]amino}methyl)cyclo-hexyl]methyl}carbamate;
43. tert-Butyl [(trans-4-{[(2-methyl-6-phenylisonicotinoyl)amino]methyl}cyclohexyl)-methyl]carbamate;
44. tert-Butyl {[trans-4-({[(5-phenylpyridin-3-yl)carbonyl]amino}methyl)cyclohexyl]-methyl}carbamate;
45. tert-Butyl {[trans-4-({[2-(1-benzothiophen-2-yl)isonicotinoyl]amino}methyl)cyclo-hexyl]methyl}carbamate;
46. tert-Butyl {[trans-4-({[(6′-ethoxy-2,3′-bipyridin-4-yl)carbonyl]amino}methyl)cyclo-hexyl]methyl}carbamate;
47. tert-Butyl {[trans-4-({[2-(2,4-dimethoxyphenyl)isonicotinoyl]amino}methyl)cyclo-hexyl]methyl}carbamate;
48. tert-Butyl {[trans-4-({[(2′,6′-dimethoxy-2,3′-bipyridin-4-yl)carbonyl]amino}methyl)-cyclohexyl]methyl}carbamate;
49. tert-Butyl {[trans-4-({[(6′-methoxy-2,3′-bipyridin-4-yl)carbonyl]amino}methyl)cyclo-hexyl]methyl}carbamate;
50. tert-Butyl {[trans-4-({[2-(4-carbamoylphenyl)isonicotinoyl]amino}methyl)cyclohexyl]methyl}carbamate;
51. tert-Butyl {[trans-4-({[2-(3-chlorophenyl)isonicotinoyl]amino}methyl)cyclohexyl]-methyl}carbamate;
52. tert-Butyl ({trans-4-[({2-[4-(aminomethyl)phenyl]isonicotinoyl}amino)methyl]cyclo-hexyl}methyl)carbamate;
53. tert-Butyl {[trans-4-({[2-(3,4-difluorophenyl)isonicotinoyl]amino}methyl)cyclohexyl]-methyl}carbamate;
54. Ethyl [(trans-4-{[(2-naphthylsulfonyl)amino]methyl}cyclohexyl)methyl] carbamate;
55. 2,2-Dimethylpropyl {[trans-4-({[(2-pyridin-4-ylquinolin-4-yl)carbonyl]amino}methyl)-cyclohexyl]methyl}carbamate;
56. Cyclopentyl {[trans-4-({[(2-pyridin-4-ylquinolin-4-yl)carbonyl]amino}methyl)cyclo-hexyl]methyl}carbamate;
57. Isopropyl {[trans-4-({[(2-pyridin-4-ylquinolin-4-yl)carbonyl]amino}methyl)cyclo-hexyl]methyl}carbamate;
58. Propyl {[trans-4-({[(2-pyridin-4-ylquinolin-4-yl)carbonyl]amino}methyl)cyclohexyl]-methyl}carbamate;
59. 2-Methoxyethyl {[trans-4-({[(2-pyridin-4-ylquinolin-4-yl)carbonyl]amino}-methyl)cyclohexyl]methyl}carbamate;
60. Ethyl {[trans-4-({[(2-pyridin-4-ylquinolin-4-yl)carbonyl]amino}methyl)cyclo-hexyl]methyl}carbamate;
61. Ethyl ({trans-4-[({[2-(4-carbamoylphenyl)quinolin-4-yl]carbonyl}amino) methyl]-cyclohexyl}methyl)carbamate;
62. Ethyl {[trans-4-({[(2-phenylquinolin-4-yl)carbonyl]amino}methyl)cyclohexyl]methyl}-carbamate;
63. tert-Butyl [(trans-4-{[(biphenyl-3-ylsulfonyl)amino]methyl}cyclohexyl)methyl]-carbamate;
64. tert-Butyl {[trans-4-({[(3,4-dibromophenyl)sulfonyl]amino}methyl)cyclohexyl]-methyl}carbamate;
65. tert-Butyl {[trans-4-({[(3,4-dichlorophenyl)sulfonyl]amino}methyl)cyclohexyl]methyl}-carbamate;
66. tert-Butyl [(trans-4-{[(quinolin-8-ylsulfonyl)amino]methyl}cyclohexyl)methyl]-carbamate;
67. tert-Butyl [(trans-4-{[(biphenyl-4-ylsulfonyl)amino]methyl}cyclohexyl)methyl]-carbamate;
68. tert-Butyl {[trans-4-({[(4-isopropylphenyl)sulfonyl]amino}methyl)cyclohexyl]-methyl}carbamate;
69. tert-Butyl [(trans-4-{[(2,3-dihydro-1,4-benzodioxin-6-ylsulfonyl)amino]methyl}cyclo-hexyl)methyl]carbamate;
70. tert-Butyl ({trans-4-[({[5-(2-methyl-1,3-thiazol-4-yl)-2-thienyl]sulfonyl} amino)-methyl]cyclohexyl}methyl)carbamate;
71. tert-Butyl {[trans-4-({[(5-acetamido-1-naphthyl)sulfonyl]amino}methyl)cyclohexyl]-methyl}carbamate;
72. tert-Butyl {[trans-4-({[(5-isoxazol-5-yl-2-thienyl)sulfonyl]amino}methyl)cyclo-hexyl]methyl}carbamate;
73. tert-Butyl {[trans-4-({[(4-acetamidophenyl)sulfonyl]amino}methyl)cyclohexyl]-methyl}carbamate;
74. tert-Butyl [(trans-4-{[(3-thienylsulfonyl)amino]methyl}cyclohexyl)methyl]-carbamate;
75. tert-Butyl ({trans-4-[({[3-(5-methyl-1,2,4-oxadiazol-3-yl)phenyl]sulfonyl} amino)-methyl]cyclohexyl}methyl)carbamate;
76. tert-Butyl [(trans-4-{[(isoquinolin-5-ylsulfonyl)amino]methyl}cyclohexyl)-methyl]carbamate;
77. tert-Butyl {[trans-4-({[(4-acetamido-2-methyl-1,3-thiazol-5-yl)sulfonyl]amino}-methyl)cyclohexyl]methyl}carbamate;
78. tert-Butyl [(trans-4-{[(1,3-benzothiazol-6-ylsulfonyl)amino]methyl}cyclohexyl)-methyl]carbamate;
79. tert-Butyl [(trans-4-{[ethyl(2-naphthylsulfonyl)amino]methyl}cyclohexyl)methyl]-carbamate;
80. tert-Butyl [(trans-4-{[(2-naphthylsulfonyl)(2-phenylethyl)amino]methyl}cyclohexyl)-methyl]carbamate;
81. Ethyl N-[(trans-4-{[(tert-butoxycarbonyl)amino]methyl}cyclohexyl)methyl]-N-(2-naphthylsulfonyl)glycinate;
82. tert-Butyl [(trans-4-{[(2-naphthylsulfonyl)(tetrahydro-2H-pyran-4-ylmethyl)amino]-methyl}cyclohexyl)methyl]carbamate;
83. tert-Butyl [(trans-4-{[(cyanomethyl)(2-naphthylsulfonyl)amino]methyl}cyclohexyl)-methyl]carbamate;
84. tert-Butyl [(trans-4-{[allyl(2-naphthylsulfonyl)amino]methyl}cyclohexyl)methyl]-carbamate;
85. tert-Butyl [(trans-4-{[butyl(2-naphthylsulfonyl)amino]methyl}cyclohexyl)methyl]-carbamate;
86. tert-Butyl [(trans-4-{[(2-naphthylsulfonyl)(2,2,2-trifluoroethyl)amino]methyl}cyclohexyl)methyl]carbamate;
87. tert-Butyl [(trans-4-{[(2-naphthylsulfonyl)(propyl)amino]methyl}cyclohexyl)-methyl]carbamate;
88. tert-Butyl [(trans-4-{[methyl(2-naphthylsulfonyl)amino]methyl}cyclohexyl)-methyl]carbamate;
89. tert-Butyl ({trans-4-[(methyl {[5-(2-methyl-1,3-thiazol-4-yl)-2-thienyl]sulfonyl}-amino)methyl]cyclohexyl}methyl)carbamate;
90. tert-Butyl ({trans-4-[(ethyl {[5-(2-methyl-1,3-thiazol-4-yl)-2-thienyl] sulfonyl}amino)-methyl]cyclohexyl}methyl)carbamate;
91. tert-Butyl {[trans-4-({methyl[(2-phenylquinolin-4-yl)carbonyl]amino}methyl)cyclo-hexyl]methyl}carbamate;
92. tert-Butyl [(trans-4-{[({2-[4-(aminomethyl)phenyl]quinolin-4-yl}carbonyl)amino]-methyl}cyclohexyl)methyl]carbamate acetate;
93. 4-(4-{[(trans-4-{[(tert-Butoxycarbonyl)amino]methyl}cyclohexyl)methyl]carbamoyl}-quinolin-2-yl)benzoic acid;
94. tert-Butyl ({trans-4-[({[2-(1-oxidopyridin-4-yl)quinolin-4-yl]carbonyl} amino)methyl]-cyclohexyl}methyl)carbamate;
95. tert-Butyl {[trans-4-({[(2-phenylquinolin-4-yl)sulfonyl]amino}methyl)cyclohexyl]-methyl}carbamate;
96. tert-Butyl ({trans-4-[(1-naphthoylamino)methyl]cyclohexyl}methyl)carbamate;
97. N-[(trans-4-{[(tert-Butylcarbamoyl)amino]methyl}cyclohexyl)methyl]-2-pyridin-4-ylquinoline-4-carboxamide;
98. tert-Butyl ({trans-4-[({[2-(3-{[(methylsulfonyl)amino]methyl}phenyl)quinolin-4-yl]carbonyl}amino)methyl]cyclohexyl}methyl)carbamate;
99. tert-Butyl {[trans-4-({[(2-{3-[(carbamoylamino)methyl]phenyl}quinolin-4-yl)-carbonyl]amino}methyl)cyclohexyl]methyl}carbamate;
100. tert-Butyl [(trans-4-{[({2-[3-(acetamidomethyl)phenyl]quinolin-4-yl}carbonyl)amino]-methyl}cyclohexyl)methyl]carbamate;
101. Methyl [3-(4-{[(trans-4-{[(tert-butoxycarbonyl)amino]methyl}cyclohexyl)methyl]-carbamoyl}quinolin-2-yl)benzyl]carbamate;
102. tert-Butyl [(trans-4-{[({2-[4-(2-aminoethyl)phenyl]quinolin-4-yl}carbonyl)amino]-methyl}cyclohexyl)methyl]carbamate;
103. tert-Butyl [(trans-4-{[({2-[4-(acetamidomethyl)phenyl]quinolin-4-yl}carbonyl)-amino]methyl}cyclohexyl)methyl]carbamate;
104. Methyl [4-(4-{[(trans-4-{[(tert-butoxycarbonyl)amino]methyl}cyclohexyl)methyl]-carbamoyl}quinolin-2-yl)benzyl]carbamate;
105. tert-Butyl {[trans-4-({[(2-{4-[(carbamoylamino)methyl]phenyl}quinolin-4-yl)carbonyl]amino}methyl)cyclohexyl]methyl}carbamate;
106. tert-Butyl ({trans-4-[({[2-(4-{[(methylsulfonyl)amino]methyl}phenyl) quinolin-4-yl]carbonyl}amino)methyl]cyclohexyl}methyl)carbamate;
107. [4-(4-{[(trans-4-{[(tert-Butoxycarbonyl)amino]methyl}cyclohexyl)methyl]-carbamoyl}quinolin-2-yl)phenyl]acetic acid;
108. 3-(4-{[(trans-4-{[(tert-Butoxycarbonyl)amino]methyl}cyclohexyl)methyl]-carbamoyl}quinolin-2-yl)benzoic acid;
109. Tetrahydro-2H-pyran-4-yl [(trans-4-{[({2-[6-(4-methylpiperazin-1-yl)pyridin-3-yl]quinolin-4-yl}carbonyl)amino]methyl}cyclohexyl)methyl]carbamate;
110. 1-Methylpiperidin-4-yl [(trans-4-{[({2-[6-(4-methylpiperazin-1-yl)pyridin-3-yl]quinolin-4-yl}carbonyl)amino]methyl}cyclohexyl)methyl]carbamate;
111. Oxetan-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
112. (1S)-2-Methoxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
113. (1R)-2-Methoxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
114. Tetrahydrofuran-3-yl [(trans-4-{[({2-[6-(4-methylpiperazin-1-yl)pyridin-3-yl]quinolin-4-yl}carbonyl)amino]methyl}cyclohexyl)methyl]carbamate;
115. 2-(2-Oxopyrrolidin-1-yl)ethyl [(trans-4-{[({2-[6-(4-methylpiperazin-1-yl)pyridin-3-yl]quinolin-4-yl}carbonyl)amino]methyl}cyclohexyl)methyl]carbamate;
116. (3S)-Tetrahydrofuran-3-yl [(trans-4-{[({2-[6-(4-methylpiperazin-1-yl)pyridin-3-yl]quinolin-4-yl}carbonyl)amino]methyl}cyclohexyl)methyl]carbamate;
117. 2,2-Difluoroethyl [(trans-4-{[({2-[6-(4-methylpiperazin-1-yl)pyridin-3-yl]quinolin-4-yl}carbonyl)amino]methyl}cyclohexyl)methyl]carbamate;
118. 2-Fluoroethyl [(trans-4-{[({2-[6-(4-methylpiperazin-1-yl)pyridin-3-yl]quinolin-4-yl}carbonyl)amino]methyl}cyclohexyl)methyl]carbamate;
119. Ethyl [(trans-4-{[({2-[6-(4-methylpiperazin-1-yl)pyridin-3-yl]quinolin-4-yl}carbonyl)amino]methyl}cyclohexyl)methyl]carbamate;
120. 2-Methoxyethyl [(trans-4-{[({2-[6-(4-methylpiperazin-1-yl)pyridin-3-yl]quinolin-4-yl}carbonyl)amino]methyl}cyclohexyl)methyl]carbamate;
121. 1-Cyanoethyl [(trans-4-{[({2-[6-(4-methylpiperazin-1-yl)pyridin-3-yl]quinolin-4-yl}carbonyl)amino]methyl}cyclohexyl)methyl]carbamate;
122. 2-Acetamidoethyl [(trans-4-{[({2-[6-(4-methylpiperazin-1-yl)pyridin-3-yl]quinolin-4-yl}carbonyl)amino]methyl}cyclohexyl)methyl]carbamate;
123. (3-Methyloxetan-3-yl)methyl [(trans-4-{[({2-[6-(4-methylpiperazin-1-yl)pyridin-3-yl]quinolin-4-yl}carbonyl)amino]methyl}cyclohexyl)methyl]carbamate;
124. (3R)-5-Oxopyrrolidin-3-yl [(trans-4-{[({2-[6-(4-methylpiperazin-1-yl)pyridin-3-yl]quinolin-4-yl}carbonyl)amino]methyl}cyclohexyl)methyl]carbamate;
125. (3S)-5-Oxopyrrolidin-3-yl [(trans-4-{[({2-[6-(4-methylpiperazin-1-yl)pyridin-3-yl]quinolin-4-yl}carbonyl)amino]methyl}cyclohexyl)methyl]carbamate;
126. Tetrahydrofuran-3-ylmethyl [(trans-4-{[({2-[6-(4-methylpiperazin-1-yl)pyridin-3-yl]quinolin-4-yl}carbonyl)amino]methyl}cyclohexyl)methyl]carbamate;
127. Tetrahydrofuran-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
128. (5-Methylisoxazol-3-yl)methyl [(trans-4-{[({2-[6-(4-methylpiperazin-1-yl)pyridin-3-yl]quinolin-4-yl}carbonyl)amino]methyl}cyclohexyl)methyl]carbamate;
129. 2-(1H-Pyrazol-1-yl)ethyl [(trans-4-{[({2-[6-(4-methylpiperazin-1-yl)pyridin-3-yl]quinolin-4-yl}carbonyl)amino]methyl}cyclohexyl)methyl]carbamate;
130. 1,3-Thiazol-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
131. Pyrazin-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
132. 2-Cyanoethyl [(trans-4-{[({2-[6-(4-methylpiperazin-1-yl)pyridin-3-yl]quinolin-4-yl}carbonyl)amino]methyl}cyclohexyl)methyl]carbamate;
133. (1S)-2-Hydroxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
134. tert-Butyl {[trans-4-({[(2-{4-[(methylamino)sulfonyl]-phenyl}quinolin-4-yl)carbonyl]amino}methyl)cyclohexyl]methyl}-carbamate;
135. tert-Butyl [(trans-4-{[({2-[4-(aminosulfonyl)phenyl]-quinolin-4-yl}carbonyl)amino]-methyl}-cyclohexyl)methyl]-carbamate;
136. Tetrahydro-2H-pyran-4-yl {[trans-4-({[(2-{4-[(methylamino)sulfonyl]phenyl}-quinolin-4-yl)carbonyl]amino}methyl)cyclohexyl]methyl}carbamate;
137. (3S)-Tetrahydrofuran-3-yl {[trans-4-({[(2-{4-[(methylamino)sulfonyl]phenyl}-quinolin-4-yl)carbonyl]amino}methyl)cyclohexyl]methyl}-carbamate;
138. Tetrahydro-2H-pyran-4-yl [(trans-4-{[({2-[4-(aminosulfonyl)phenyl]-quinolin-4-yl}carbonyl)amino]-methyl}cyclohexyl)-methyl]carbamate;
139. (3S)-Tetrahydrofuran-3-yl [(trans-4-{[({2-[4-(aminosulfonyl)phenyl]-quinolin-4-yl}carbonyl)amino]-methyl}cyclohexyl)-methyl]carbamate;
140. Tetrahydro-2H-pyran-4-yl {[trans-4-({[(2-{4-[(dimethylamino)sulfonyl]phenyl}-quinolin-4-yl)carbonyl]amino}methyl)cyclohexyl]-methyl}carbamate;
141. (3S)-Tetrahydrofuran-3-yl {[trans-4-({[(2-{4-[(dimethylamino)sulfonyl]phenyl}-quinolin-4-yl)carbonyl]amino}methyl)cyclohexyl]-methyl}carbamate;
142. tert-Butyl [(trans-4-{[({2-[6-(4-acetylpiperazin-1-yl)pyridin-3-yl]quinolin-4-yl}carbonyl)amino]methyl}cyclohexyl)methyl]carbamate;
143. tert-Butyl {[trans-4-({[(2-{6-[(2-hydroxyethyl)amino]pyridin-3-yl}quinolin-4-yl)carbonyl]amino}methyl)cyclohexyl]methyl}carbamate;
144. tert-Butyl ({trans-4-[({[2-(6-pyrrolidin-1-ylpyridin-3-yl)quinolin-4-yl]carbonyl}amino)methyl]cyclohexyl}methyl)carbamate;
145. tert-Butyl ({trans-4-[({[2-(6-morpholin-4-ylpyridin-3-yl)quinolin-4-yl]carbonyl}amino)methyl]cyclohexyl}methyl)carbamate;
146. tert-Butyl ({trans-4-[({[2-(6-{[2-(dimethylamino)ethyl]amino}pyridin-3-yl)quinolin-4-yl]carbonyl}amino)methyl]cyclohexyl}methyl)carbamate;
147. tert-Butyl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
148. tert-Butyl ({trans-4-[({[2-(6-{[(1S)-2-hydroxy-1-methylethyl]amino}pyridin-3-yl)quinolin-4-yl]carbonyl}amino)methyl]cyclohexyl}methyl)carbamate;
149. tert-Butyl ({trans-4-[({[2-(6-{[(1R)-2-hydroxy-1-methylethyl]amino}pyridin-3-yl)quinolin-4-yl]carbonyl}amino)methyl]cyclohexyl}methyl)carbamate;
150. tert-Butyl {[trans-4-({[(2-{6-[(3-hydroxypropyl)amino]pyridin-3-yl}quinolin-4-yl)carbonyl]amino}methyl)cyclohexyl]methyl}carbamate;
151. tert-Butyl [(trans-4-{[({2-[6-(4-hydroxypiperidin-1-yl)pyridin-3-yl]quinolin-4-yl}carbonyl)amino]methyl}cyclohexyl)methyl]carbamate;
152. tert-Butyl {[trans-4-({[(2-{6-[bis(2-hydroxyethyl)amino]pyridin-3-yl}quinolin-4-yl)carbonyl]amino}methyl)cyclohexyl]methyl}carbamate;
153. tert-Butyl [(trans-4-{[({2-[6-(4-carbamoylpiperidin-1-yl)pyridin-3-yl]quinolin-4-yl}carbonyl)amino]methyl}cyclohexyl)methyl]carbamate;
154. tert-Butyl ({trans-4-[({[2-(6-piperazin-1-ylpyridin-3-yl)quinolin-4-yl]carbonyl}amino)methyl]cyclohexyl}methyl)carbamate;
155. tert-Butyl ({trans-4-[({[2-(6-{[2-(2-hydroxyethoxy)ethyl]amino}pyridin-3-yl)quinolin-4-yl]carbonyl}amino)methyl]cyclohexyl}methyl)carbamate;
156. tert-Butyl {[trans-4-({[(2-{6-[(2-methoxyethyl)amino]pyridin-3-yl}quinolin-4-yl)carbonyl]amino}methyl)cyclohexyl]methyl}carbamate;
157. tert-Butyl [(trans-4-{[({2-[6-(3-hydroxypiperidin-1-yl)pyridin-3-yl]quinolin-4-yl}carbonyl)amino]methyl}cyclohexyl)methyl]carbamate;
158. 3-{[5-(4-{[(trans-4-{[(tert-Butoxycarbonyl)amino]methyl}cyclohexyl)methyl]-carbamoyl}quinolin-2-yl)pyridin-2-yl]amino}-2-methylpropanoic acid;
159. tert-Butyl ({trans-4-[({[2-(6-{[(5-methylpyrazin-2-yl)methyl]amino}pyridin-3-yl)quinolin-4-yl]carbonyl}amino)methyl]cyclohexyl}methyl)carbamate;
160. tert-Butyl {[trans-4-({[(2-{6-[(3S)-3-(dimethylamino)pyrrolidin-1-yl]pyridin-3-yl}quinolin-4-yl)carbonyl]amino}methyl)cyclohexyl]methyl}carbamate;
161. tert-Butyl {[trans-4-({[(2-{6-[4-(hydroxymethyl)piperidin-1-yl]pyridin-3-yl}quinolin-4-yl)carbonyl]amino}methyl)cyclohexyl]methyl}carbamate;
162. tert-Butyl {[trans-4-({[(2-{6-[(2,3-dihydroxypropyl)amino]pyridin-3-yl}quinolin-4-yl)carbonyl]amino}methyl)cyclohexyl]methyl}carbamate;
163. tert-Butyl {[trans-4-({[(2-{6-[(2S)-2-carbamoylpyrrolidin-1-yl]pyridin-3-yl}quinolin-4-yl)carbonyl]amino}methyl)cyclohexyl]methyl}carbamate;
164. tert-Butyl ({trans-4-[({[2-(6-{[2-hydroxy-1-(hydroxymethyl)ethyl]amino}pyridin-3-yl)quinolin-4-yl]carbonyl}amino)methyl]cyclohexyl}methyl)carbamate;
165. tert-Butyl [(trans-4-{[({2-[6-(3-oxopiperazin-1-yl)pyridin-3-yl]quinolin-4-yl}carbonyl)amino]methyl}cyclohexyl)methyl]carbamate;
166. tert-Butyl ({trans-4-[({[2-(6-{[(1-methyl-1H-pyrazol-4-yl)methyl]amino}pyridin-3-yl)quinolin-4-yl]carbonyl}amino)methyl]cyclohexyl}methyl)carbamate;
167. tert-Butyl {[trans-4-({[(2-{6-[(3-amino-3-oxopropyl)amino]pyridin-3-yl}quinolin-4-yl)carbonyl]amino}methyl)cyclohexyl]methyl}carbamate;
168. tert-Butyl [(trans-4-{[({2-[6-(3-hydroxyazetidin-1-yl)pyridin-3-yl]quinolin-4-yl}carbonyl)amino]methyl}cyclohexyl)methyl]carbamate;
169. tert-Butyl ({trans-4-[({[2-(6-{[(1S)-1-carbamoylpropyl]amino}pyridin-3-yl)quinolin-4-yl]carbonyl}amino)methyl]cyclohexyl}methyl)carbamate;
170. tert-Butyl ({trans-4-[({[2-(6-{[(5-methyl-1,2,4-oxadiazol-3-yl)methyl]amino}pyridin-3-yl)quinolin-4-yl]carbonyl}amino)methyl]cyclohexyl}methyl)carbamate;
171. tert-Butyl {[trans-4-({[(2-{6-[2-(hydroxymethyl)morpholin-4-yl]pyridin-3-yl}quinolin-4-yl)carbonyl]amino}methyl)cyclohexyl]methyl}carbamate;
172. tert-Butyl [(trans-4-{[({2-[6-(4-oxoimidazolidin-1-yl)pyridin-3-yl]quinolin-4-yl}carbonyl)amino]methyl}cyclohexyl)methyl]carbamate;
173. tert-Butyl [(trans-4-{[({2-[6-(tetrahydrofuran-3-ylmethoxy)pyridin-3-yl]quinolin-4-yl}carbonyl)amino]methyl}cyclohexyl)methyl]carbamate;
174. tert-Butyl [(trans-4-{[({2-[6-(2-hydroxyethoxy)pyridin-3-yl]quinolin-4-yl}carbonyl)amino]methyl}cyclohexyl)methyl]carbamate;
175. tert-Butyl [(trans-4-{[({2-[6-(tetrahydro-2H-pyran-4-yloxy)pyridin-3-yl]quinolin-4-yl}carbonyl)amino]methyl}cyclohexyl)methyl]carbamate;
176. tert-Butyl [(trans-4-{[({2-[6-(2-hydroxy-1-methylpropoxy)pyridin-3-yl]quinolin-4-yl}carbonyl)amino]methyl}cyclohexyl)methyl]carbamate;
177. tert-Butyl {[trans-4-({[(2-{6-[(2-oxopyrrolidin-1-yl)methoxy]pyridin-3-yl}quinolin-4-yl)carbonyl]amino}methyl)cyclohexyl]methyl}carbamate;
178. tert-Butyl [(trans-4-{[({2-[6-(2-amino-2-oxoethoxy)pyridin-3-yl]quinolin-4-yl}carbonyl)amino]methyl}cyclohexyl)methyl]carbamate;
179. tert-Butyl {[trans-4-({[(2-{6-[2-(4-methylpiperazin-1-yl)ethoxy]pyridin-3-yl}quinolin-4-yl)carbonyl]amino}methyl)cyclohexyl]methyl}carbamate;
180. tert-Butyl [(trans-4-{[({2-[3-(4-methylpiperazin-1-yl)propoxy]quinolin-4-yl}carbonyl)amino]methyl}cyclohexyl)methyl]carbamate;
181. tert-Butyl [(trans-4-{[({2-[3-(dimethylamino)propoxy]quinolin-4-yl}carbonyl)amino]methyl}cyclohexyl)methyl]carbamate;
182. tert-Butyl [(trans-4-{[({2-[2-(4-methylpiperazin-1-yl)ethoxy]quinolin-4-yl}carbonyl)amino]methyl}cyclohexyl)methyl]carbamate;
183. tert-Butyl [(trans-4-{[({2-[3-(4-acetylpiperazin-1-yl)propoxy]quinolin-4-yl}carbonyl)amino]methyl}cyclohexyl)methyl]carbamate;
184. tert-Butyl {[trans-4-({[(2-{6-[2-(dimethylamino)ethoxy]pyridin-3-yl}quinolin-4-yl)carbonyl]amino}methyl)cyclohexyl]methyl}carbamate;
185. tert-Butyl [(trans-4-{[({2-[4-(2-hydroxyethyl)piperazin-1-yl]quinolin-4-yl}carbonyl)amino]methyl}cyclohexyl)methyl]carbamate;
186. tert-Butyl {[trans-4-({[(2-{4-[2-(dimethylamino)ethyl]-piperazin-1-yl}quinolin-4-yl)carbonyl]amino}-methyl)cyclohexyl]methyl}carbamate;
187. tert-Butyl ({trans-4-[({[2-(4-hydroxypiperidin-1-yl)quinolin-4-yl]carbonyl}amino)-methyl]cyclohexyl}methyl)carbamate;
188. tert-Butyl [(trans-4-{[({2-[4-(hydroxymethyl)-piperidin-1-yl]quinolin-4-yl}carbonyl)amino]-methyl}cyclohexyl)-methyl]carbamate;
189. tert-Butyl [(trans-4-{[({2-[4-(2-hydroxyethyl)piperidin-1-yl]quinolin-4-yl}carbonyl)amino]-methyl}cyclohexyl)-methyl]carbamate;
190. tert-Butyl {[trans-4-({[(2-piperazin-1-ylquinolin-4-yl)carbonyl]amino}methyl)cyclohexyl]methyl}carbamate;
191. tert-Butyl [(trans-4-{[({2-[4-(acetamidomethyl)piperidin-1-yl]quinolin-4-yl}carbonyl)amino]methyl}cyclohexyl)methyl]carbamate;
192. tert-Butyl [(trans-4-{[({2-[4-(2-acetamidoethyl)piperidin-1-yl]quinolin-4-yl}carbonyl)amino]methyl}cyclohexyl)methyl]carbamate;
193. tert-Butyl {[trans-4-({[(2-{4-[2-(dimethylamino)ethyl]piperidin-1-yl}quinolin-4-yl)carbonyl]amino}methyl)cyclohexyl]methyl}carbamate;
194. tert-Butyl [(trans-4-{[({2-[4-(2-aminoethyl)piperidin-1-yl]quinolin-4-yl}carbonyl)amino]methyl}cyclohexyl)methyl]carbamate;
195. Tetrahydro-2H-pyran-4-yl {[trans-4-({[(2-{6-[2-(dimethylamino)ethoxy]pyridin-3-yl}quinolin-4-yl)carbonyl]amino}methyl)cyclohexyl]methyl}carbamate;
196. (3S)-Tetrahydrofuran-3-yl {[trans-4-({[(2-{6-[2-(dimethylamino)ethoxy]pyridin-3-yl}quinolin-4-yl)carbonyl]amino}methyl)cyclohexyl]methyl}carbamate;
197. Tetrahydro-2H-pyran-4-yl {[trans-4-({[(2-{6-[(2-hydroxyethyl)amino]pyridin-3-yl}quinolin-4-yl)carbonyl]amino}methyl)cyclohexyl]methyl}carbamate;
198. Tetrahydro-2H-pyran-4-yl ({trans-4-[({[2-(6-{[2-(dimethylamino)ethyl]amino}-pyridin-3-yl)quinolin-4-yl]carbonyl}amino)methyl]cyclohexyl}methyl)carbamate;
199. (3S)-Tetrahydrofuran-3-yl ({trans-4-[({[2-(6-{[2-(dimethylamino)ethyl]amino}-pyridin-3-yl)quinolin-4-yl]carbonyl}amino)methyl]cyclohexyl}methyl)carbamate;
200. (3S)-Tetrahydrofuran-3-yl {[trans-4-({[(2-{6-[(2-hydroxyethyl)amino]pyridin-3-yl}quinolin-4-yl)carbonyl]amino}methyl)cyclohexyl]methyl}carbamate;
201. (3S)-Tetrahydrofuran-3-yl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
202. Tetrahydro-2H-pyran-4-yl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
203. tert-Butyl {[trans-4-({[(2-{4-[2-(dimethylamino)ethoxy]phenyl}quinolin-4-yl)carbonyl]amino}methyl)cyclohexyl]methyl}carbamate;
204. tert-Butyl ({trans-4-[({2-[6-(4-methylpiperazin-1-yl)pyridin-3-yl]quinolin-4-yl}carbonyl)amino]cyclohexyl}methyl)carbamate;
205. tert-Butyl {[trans-4-({[(2-{4-[3-(dimethylamino)-2-hydroxypropyl]piperidin-1-yl}quinolin-4-yl)carbonyl]amino}methyl)cyclohexyl]methyl}carbamate;
206. tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;
207. (R) or (S) tert-Butyl [(trans-4-{[({2-[4-(2-hydroxy-3-morpholin-4-ylpropyl)piperidin-1-yl]quinolin-4-yl}-carbonyl)amino]methyl}cyclohexyl)methyl]-carbamate;
208. (S) or (R) tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;
209. tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;
210. tert-Butyl {[trans-4-({[(2-{4-[2-(dimethylamino)-2-oxoethoxy]piperidin-1-yl}quinolin-4-yl)carbonyl]amino}methyl)cyclohexyl]methyl}carbamate;
211. tert-Butyl {[trans-4-({[(2-{4-[2-(dimethylamino)ethoxy]piperidin-1-yl}quinolin-4-yl)carbonyl]amino}methyl)cyclohexyl]methyl}carbamate;
212. 2,2-Dimethylpropyl {[trans-4-({[(2-{4-[2-(dimethylamino)ethyl]piperidin-1-yl}quinolin-4-yl)carbonyl]amino}methyl)cyclohexyl]methyl}carbamate;
213. tert-Butyl [(trans-4-{[({2-[4-(morpholin-4-ylmethyl)piperidin-1-yl]quinolin-4-yl}carbonyl)amino]methyl}cyclohexyl)methyl]-carbamate;
214. tert-Butyl [(trans-4-{[({2-[4-(4-methylpiperazin-1-yl)piperidin-1-yl]quinolin-4-yl}carbonyl)amino]methyl}cyclohexyl)methyl]-carbamate;
215. tert-Butyl ({trans-4-[({[2-(pyridin-3-yloxy)quinolin-4-yl]carbonyl}amino)-methyl]cyclohexyl}methyl)carbamate;
216. tert-Butyl [(trans-4-{[({2-[(1-methylpiperidin-4-yl)methoxy]quinolin-4-yl}carbonyl)amino]methyl}cyclohexyl)methyl]-carbamate;
217. tert-Butyl ({trans-4-[({[2-(3-{[2-(dimethylamino)ethyl]carbamoyl}azetidin-1-yl)quinolin-4-yl]carbonyl}amino)methyl]cyclohexyl}methyl)carbamate;
218. tert-Butyl ({trans-4-[({[2-(4-aminophenyl)quinolin-4-yl]carbonyl}amino)-methyl]cyclohexyl}methyl)carbamate;
219. tert-Butyl {[trans-4-({[(2-{4-[2-(dimethylamino)ethyl]-3-oxopiperazin-1-yl}quinolin-4-yl)carbonyl]amino}methyl)cyclohexyl]methyl}carbamate;
220. tert-Butyl [(trans-4-{[({2-[4-(hydroxyacetyl)piperazin-1-yl]quinolin-4-yl}carbonyl)amino]methyl}cyclohexyl)methyl]carbamate;
221. tert-Butyl {[trans-4-({[(2-{4-[(4-fluorobenzyl)oxy]piperidin-1-yl}quinolin-4-yl)carbonyl]amino}methyl)cyclohexyl]methyl}carbamate;
222. tert-Butyl {[trans-4-({[(2-amino-1-benzothiophen-3-yl)carbonyl]amino}methyl)-cyclohexyl]methyl}carbamate;
223. tert-Butyl [(trans-4-{[({2-[(ethylcarbamoyl)amino]-1-benzothiophen-3-yl}carbonyl)amino]methyl}cyclohexyl)methyl]carbamate;
224. Tetrahydro-2H-pyran-4-yl {[trans-4-({[(2-{4-[2-(dimethylamino) ethyl]piperidin-1-yl}quinolin-4-yl)carbonyl]amino}methyl)cyclohexyl]methyl}carbamate;
225. tert-Butyl ({trans-4-[({[2-(1′-methyl-4,4′-bipiperidin-1-yl)quinolin-4-yl]carbonyl}amino)methyl]cyclohexyl}methyl)carbamate;
226. tert-Butyl ({trans-4-[({[2-(4-{2-[(2-methoxyethyl)amino]ethyl}piperidin-1-yl)quinolin-4-yl]carbonyl}amino)methyl]cyclohexyl}methyl)carbamate;
227. tert-Butyl ({trans-4-[({[2-(4-{2-[(2-methoxyethyl)(methyl)-amino]ethyl}piperidin-1-yl)quinolin-4-yl]carbonyl}amino)-methyl]cyclohexyl}-methyl)carbamate;
228. tert-Butyl {[trans-4-({[(2-{4-[2-(tetrahydro-2H-pyran-4-ylamino)ethyl]piperidin-1-yl}quinolin-4-yl)carbonyl]amino}-methyl)cyclohexyl]-methyl}carbamate;
229. tert-Butyl {[trans-4-({[(2-{4-[2-(3-methoxyazetidin-1-yl)ethyl]piperidin-1-yl}quinolin-4-yl)carbonyl]amino}-methyl)cyclohexyl]-methyl}carbamate;
230. tert-Butyl {[trans-4-({[(2-{4-[2-(3-hydroxypyrrolidin-1-yl)ethyl]piperidin-1-yl}quinolin-4-yl)carbonyl]amino}-methyl)cyclohexyl]-methyl}carbamate;
231. tert-Butyl [(trans-4-{[({2-[4-(2-pyrrolidin-1-ylethyl)piperidin-1-yl]quinolin-4-yl}carbonyl)amino]-methyl}cyclohexyl)-methyl]carbamate;
232. tert-Butyl ({trans-4-[({[2-(4-{2-[(2-hydroxyethyl)(methyl)-amino]ethyl}piperidin-1-yl)quinolin-4-yl]carbonyl}amino)-methyl]cyclohexyl}-methyl)carbamate;
233. tert-Butyl {[trans-4-({[(2-{4-[2-(3-hydroxyazetidin-1-yl)ethyl]piperidin-1-yl}quinolin-4-yl)carbonyl]amino}methyl)cyclohexyl]methyl}carbamate;
234. tert-Butyl [(trans-4-{[({2-[4-(2-azetidin-1-ylethyl)piperidin-1-yl]quinolin-4-yl}carbonyl)amino]methyl}cyclohexyl)methyl]carbamate;
235. 1-{2-[1-(4-{[(trans-4-{[(tert-Butoxycarbonyl)amino]methyl}cyclohexyl) methyl]-carbamoyl}quinolin-2-yl)piperidin-4-yl]ethyl}azetidine-3-carboxylic acid;
236. tert-Butyl {[trans-4-({[(2-{4-[2-(3-aminoazetidin-1-yl)ethyl]piperidin-1-yl}quinolin-4-yl)carbonyl]amino}methyl)cyclohexyl]methyl}carbamate;
237. tert-Butyl {[trans-4-({[(2-{4-[3-(dimethylamino)-2-fluoropropyl]piperidin-1-yl}quinolin-4-yl)carbonyl]amino}methyl)cyclohexyl]methyl}carbamate;
238. tert-Butyl {[trans-4-({[(2-{3-[2-(dimethylamino)ethoxy]azetidin-1-yl}quinolin-4-yl)carbonyl]amino}methyl)cyclohexyl]methyl}carbamate;
239. tert-Butyl [(trans-4-{[({2-[3-(aminomethyl)phenyl]-6-(1H-pyrazol-4-yl)pyridin-4-yl}carbonyl)amino]methyl}cyclohexyl)methyl]carbamate;
240. tert-Butyl [(trans-4-{[({6-[3-(aminomethyl)phenyl]-2,3′-bipyridin-4-yl}carbonyl)amino]methyl}cyclohexyl)methyl]carbamate;
241. tert-Butyl [(trans-4-{[({6′-amino-6-[3-(aminomethyl)phenyl]-2,3′-bipyridin-4-yl}carbonyl)amino]methyl}cyclohexyl)methyl]carbamate;
242. tert-Butyl {[trans-4-({[(2-{1-[2-(dimethylamino)ethyl]-1H-pyrazol-4-yl}quinolin-4-yl)carbonyl]amino}methyl)cyclohexyl]methyl}carbamate;
243. tert-Butyl [(trans-4-{[({2-[1-(2-hydroxyethyl)-1H-pyrazol-4-yl]quinolin-4-yl}carbonyl)amino]methyl}cyclohexyl)methyl]carbamate;
244. tert-Butyl ({trans-4-[({[2-(4-{2-[(2-fluoroethylamino]ethyl}piperidin-1-yl)quinolin-4-yl]carbonyl}amino)methyl]cyclohexyl}methyl)carbamate;
245. tert-Butyl ({trans-4-[({[2-(4-(2-((2-fluoroethyl)(methyl)amino)ethyl}piperidin-1-yl)quinolin-4-yl]carbonyl}amino)methyl]cyclohexyl}methyl)carbamate;
   or a pharmaceutically acceptable salt thereof,
   or an enantiomer thereof.

### Preparation

A compound of formula (I) or formula (II) and its salts may be prepared by any process known to be applicable to the preparation of chemically related compounds. Such processes, when used to prepare a compound of the formula (I) or formula (II), or a pharmaceutically-acceptable salt thereof, are provided as a further feature of the invention.

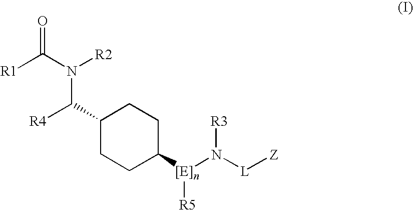

In a further aspect the present invention also provides that the compounds of the formula (I) or formula (II) and salts thereof, can be prepared by a process a) to f) as follows (wherein all variables are as hereinbefore defined for a compound of formula (I) or formula (II) unless otherwise stated).

a) Compounds of formula (2) can be coupled to compounds of formula (3) in a solvent such as DCM to form compounds of formula (I) or formula (II). For example, if L=C(O) and Y=OH, then using a solvent such as DCM and a coupling reagent such as TBTU in the presence of a base such as TEA or NMM or by using EDC, HOBT and NMM in a mixture of 2-methyltetrahydrofuran and water. If L=SO₂ and Y=Cl, then using a solvent such as DCM in the presence of a base such as TEA.

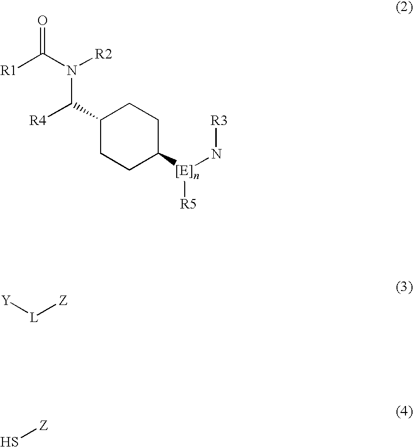

b) Alternatively, compounds of formula (2) can be coupled to thiols of formula (4) according to the procedure described in Wright, S., Hallstrom, K. N., J. Org. Chem., 2006, 71, p. 1080-1084 to form compounds of formula (I) where L=SO₂.

c) Compounds of formula (I) can also be prepared by reacting compounds of formula (5), where X is a suitable group such as a trifluoromethylsulfonyl group or a halo, such as chloro or bromo, with compounds of formula (6) where Z₁ and Z₂ are both H or Z₁ and Z₂ together represent —C(CH₃)₂C(CH₃)₂—, in the presence of a transition metal catalyst, preferably palladium. These reactions take place in solvents such as dioxane or ACN and water and in the presence of a base such as K₂CO₃ or NaHCO₃. Compounds of formula (5) where X is a suitable group such as a halo, such as chloro or fluoro can also be treated with compounds of formula (7) or (8) or nitrogen containing heterocycles, such as piperidine or piperazine, with or without a base such as KOH in a solvent such as THF and ACN or pyridine to give additional compounds of formula (I).

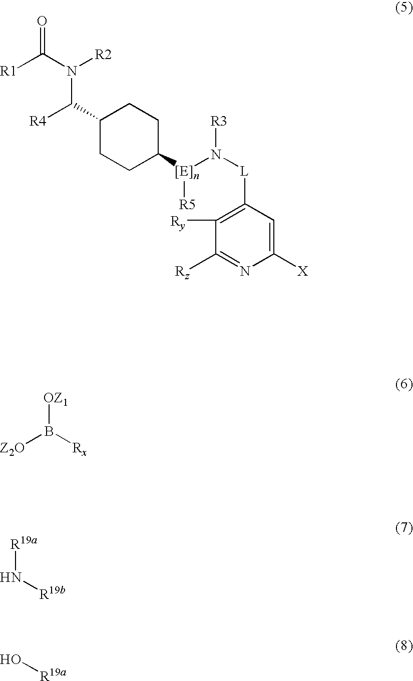

d) Compounds of formula (I) can also be formed by reacting an amine of formula (9) with a reagent of formula (10) and LG=a suitable leaving group such as chloro or 4-nitrophenoxy, in a solvent such as DCM or THF using a base such as TEA. Similarly, compounds of formula (I) can be formed by reacting an amine of formula (9) with an isocyanate of formula (11) in a solvent such as DCM or ACN using a base such as TEA, or with a carbamoyl chloride of formula (12) in a solvent such as DCM or ACN using a base such as TEA.

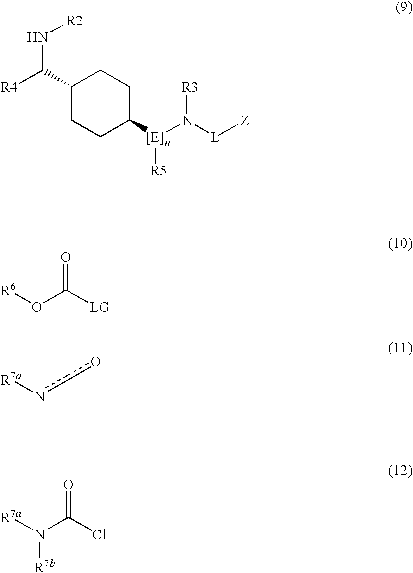

e) Furthermore, additional compounds of formula (I) can be prepared by treating compounds of formula (I) where R³=H with compounds of formula (13) where R³ is as defined herein and LG is a suitable leaving group known to someone skilled in the art, for example iodide. This reaction can take place in a solvent such as THF and/or DMF using a base such as K₂CO₃ and/or sodium hydride.

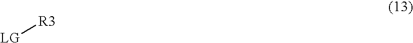

f) Additional compounds of formula (I) can also be prepared by treating compounds of formula (I), such as compounds of formula (14) where X is a suitable leaving group such as a halo, such as chloro or fluoro, with compounds of formula (7) or (8) or nitrogen containing heterocycles, such as piperidine or piperazine, with or without a base such as KOH in a solvent such as THF and ACN or pyridine.

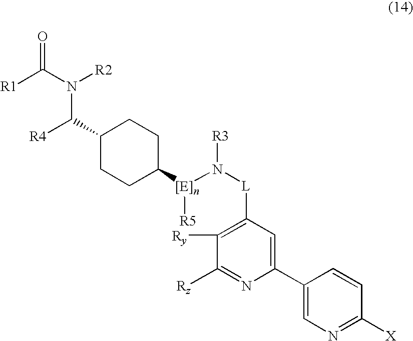

Compounds of formula (I) to (14) may be made by application of standard synthetic methods known to someone skilled in the art. The processes a)-f) can be used in different orders to form the compounds (I). Some of the described processes may involve the use of protecting groups as known to someone skilled in the art, for example, a Boc group may be used to protect an amino group. The protecting groups can then be removed according to the art, for example, using trifluoroacetic acid to remove a Boc group to give compounds of formula (I). Once a protecting group has been removed the released functional group may be manipulated further using standard synthetic methods known to someone skilled in the art. For example, an amino group may be transformed into an acetamide by treatment with an alkylating agent such as acetic anhydride.

The starting materials for the described processes a)-f) can be prepared as follows: Compounds of formula (I) and also compounds of formula (5) and (14) can be formed by reacting a compound of formula (2) with a carboxylic acid of formula (3), using a coupling agent such as TBTU in the presence of a base such as DIPEA in a solvent such as DMF or DCM, to form the amide bond. Alternately, EDC, HOBT and NMM in a mixture of 2-methyl tetrahydrofuran and water can be used in place of TBTU and DIPEA in DMF or DCM.

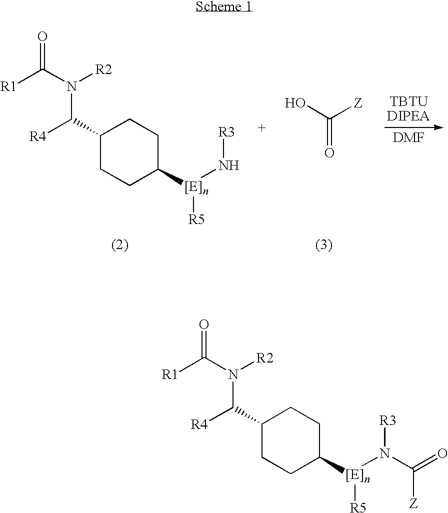

Alternatively, for compounds of formula (I), (5) and (14) where L=SO₂ then compounds of formula (2) can be treated with a sulfonyl chloride of formula (3), in the presence of a base such as TEA in a solvent such as DCM, to form the sulfonamide bond as shown in Scheme 2.

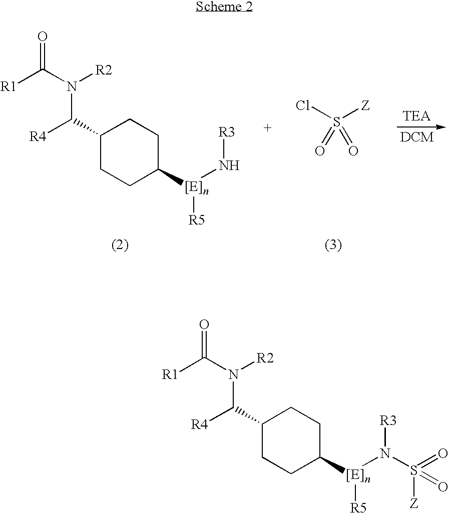

Compounds of formula (2) can be synthesised by the method described in Scheme 3. Amines of formula (15) can be treated with different acylating agents, such as compounds of formula (10) and (11) to form the Boc-protected compounds (16). For example, the amine (15) can be treated with chloroformates (10) or 4-nitrophenylcarbonates (10) in the presence of a base such as TEA to form carbamates or the amine (15) can be treated with isocyanates (11) using TEA as a base to form ureas. The Boc protecting group in compounds of formula (16) can then be removed using an acid, for example 1N HCl in EtOAc or TFA, to form amines of formula (2).

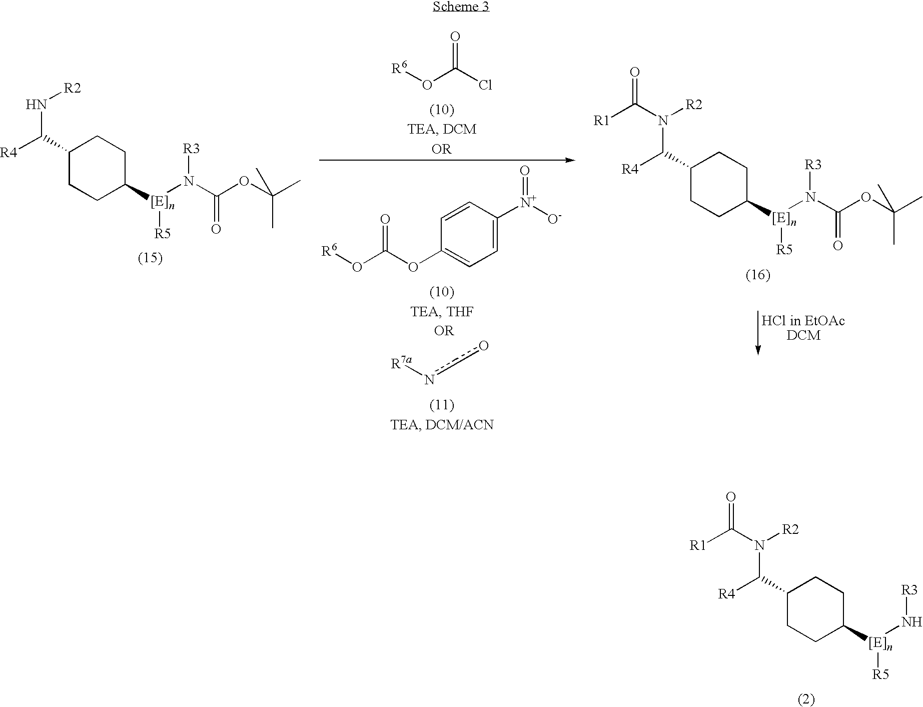

Compounds of formula (3) are either commercially available or can be synthesised. For example, carboxylic acids of formula (3) can be synthesised by a coupling reaction such as a palladium coupling or a nucleophilic aromatic substitution reaction. For example, the 2-chloro compound of general formula (3) shown in Scheme 4 can be treated with the boronic ester (6) and a palladium catalyst like [1,3-bis(2,6-diisopropylphenyl)imidazol-2-ylidene](3-chloropyridyl)-palladium(II) dichloride (PEPPSI) in the presence of an aqueous alkalimetal carbonate like K₂CO₃ using an organic solvent like dioxane to give further compounds of formula (3).

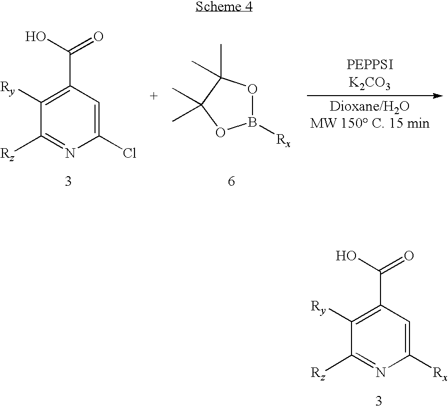

Alternatively, the 2-chloro containing starting material (3) shown in Scheme 4 can be treated with an amine (7) with pyridine as solvent or with an alcohol (8) using a base such as KOH in a solvent mixture of THF and ACN to give further compounds of formula (3). It is also understandable to someone skilled in the art that the metal mediated coupling reaction can be followed by the nucleophilic substitution reaction in the same molecule. An example of this is shown in Scheme 5.

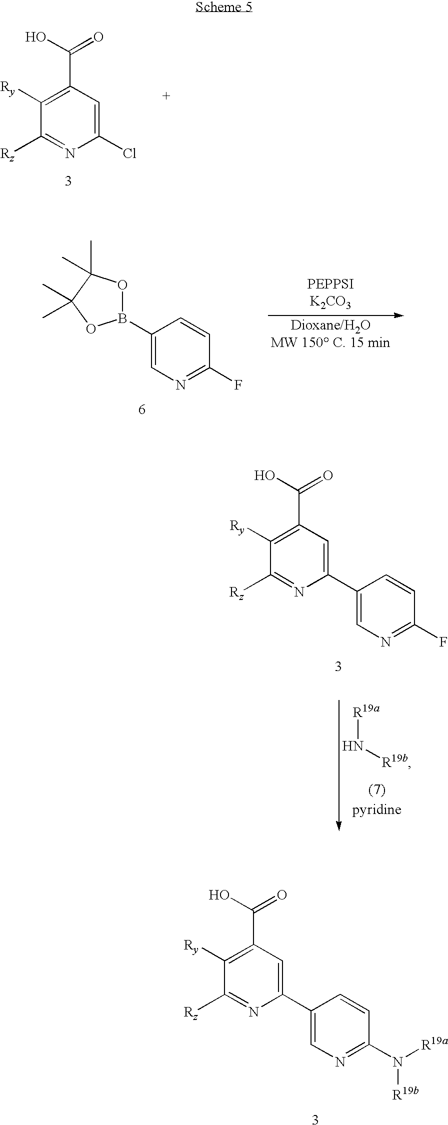

Further compounds of formula (3) may be synthesised as shown in Scheme 6. 2,3-Dihydro-indole-2,3-dione (isatin) (17) was treated with a methyl ketone (18) in the presence of KOH using EtOH as solvent to provide the quinoline compounds of formula (3).

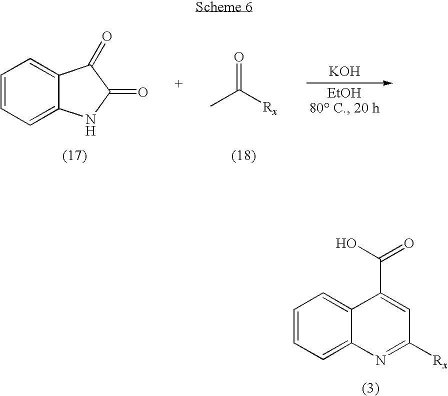

Thiol compounds of formula (4) required as starting material in process b) can be synthesised by refluxing an appropriate chloro-substituted derivative (19), where Z is a heteroaryl or an appropriately substituted aryl compound that will be known to someone skilled in the art, with sodium ethanethiolate (20) in a solvent such as DMF as shown in Scheme 7. For example 4-chloro-2-phenyl-quinoline (21) can be refluxed with sodium ethanethiolate (20) in a solvent such as DMF to give the quinoline compound of formula (4) as shown in Scheme 7.

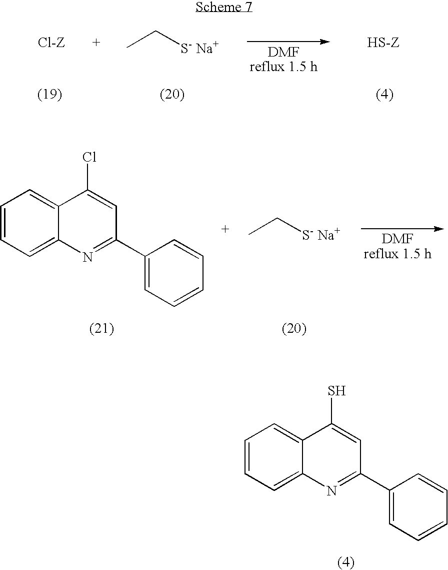

All processes discussed herein can be used in different orders to form the required intermediates, such as compounds of formula (5) and (14). This is known to someone skilled in the art. If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991, or P. J. Kocienski, Protecting Groups, Georg Thieme Verlag, 1994).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

It will be appreciated by those skilled in the art that certain compounds of formula I may be transformed into other compounds of formula I by functional group modifications known to those skilled in the art. For example a hydroxy group may be converted into a leaving group for example a mesylate and then displaced with an amine or an aliphatic carbon atom bearing a hydroxy group may be oxidised to an aldehyde and then reacted with an amine using reductive amination. Certain intermediates are believed to be novel and form a further part of the present invention.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-5 mg/kg body weight. Oral formulations are preferred, particularly tablets or capsules which may be formulated by methods known to those skilled in the art to provide doses of the active compound in the range of 0.5 mg to 500 mg.

According to a further aspect of the invention there is also provided a pharmaceutical formulation comprising a compound of formula (I), or pharmaceutically acceptable salt thereof, including the compound of the proviso, in a mixture with pharmaceutically acceptable adjuvants, diluents and/or carriers.

### Pharmacological Properties

The compounds of formula (I) or formula (II) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating), dyslipidaemia and the treatment of type 2 diabetes mellitus.

The present compounds of formula (I) or formula (II) are useful for the prophylaxis and/or treatment of clinical conditions associated with inherent or induced reduced sensitivity to insulin (insulin resistance) and associated metabolic disorders (also known as the metabolic syndrome). These clinical conditions will include, but will not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apoAI particle levels and high Apo B levels in the presence of small, dense, low density lipoproteins (LDL) particles, phenotype B.

The compounds of the present invention are expected to be useful in treating patients with combined or mixed hyperlipidemias or various degrees of hypertriglyceridemias and postprandial dyslipidemia with or without other manifestations of the metabolic syndrome.

Treatment with the present compounds is expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. Because of their insulin sensitizing effect the compounds of formula (I) or formula (II) are also expected to prevent or delay the development of type 2 diabetes from the metabolic syndrome and diabetes of pregnancy. Therefore the development of long-term complications associated with chronic hyperglycaemia in diabetes mellitus, such as the micro-angiopathies causing renal disease, retinal damage and peripheral vascular disease of the lower limbs, is expected to be delayed.

The compounds of formula (I) or formula (II) may also be useful in the treatment of metabolic syndrome and Prader-Willi syndrome.

In another aspect the present invention provides a compound of formula (I) or formula (II) as previously defined for use as a medicament.

In a further aspect the present invention provides the use of a compound of formula (I) or formula (II) in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating) and for the treatment or prophylaxis of dyslipidaemia and for the treatment or prophylaxis of type 2 diabetes mellitus.

In a still further aspect the present invention provides a method of treating obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating) dyslipidaemia and type 2 diabetes mellitus comprising administering a pharmacologically effective amount of a compound of formula (I) or formula (II), including the compound of the proviso b), to a patient in need thereof.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or gastrointestinal motility.

The compounds of the invention may further be combined with another therapeutic agent that is useful in the treatment of disorders associated with obesity such as hypertension, hyperlipidaemias, dyslipidaemias and diabetes. For example, a compound of the present invention may be used in combination with another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula (I) or formula (II), or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula (I) or formula (II), or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;

a cholesterol absorption antagonist;

a MTP (microsomal transfer protein) inhibitor;

a nicotinic acid derivative, including slow release and combination products;

a phytosterol compound;

probucol;

an anti-coagulant;

an omega-3 fatty acid;

another anti-obesity compound for example sibutramine, phentermine, orlistat, bupropion, ephedrine, thyroxine;

an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;

a CB1 receptor antagonist/inverse agonist;

a melanin concentrating hormone (MCH) modulator;

a melanocortin-4 receptor agonist;

an NPY receptor modulator;

an orexin receptor modulator;

a diacylglycerol acyltransferase-1 inhibitor;

a diacylglycerol acyltransferase-2 inhibitor;

a phosphoinositide-dependent protein kinase (PDK) modulator; or modulators of nuclear receptors for example LXR, FXR, RXR, GR, ERRα, β, PPARα, β, γ and RORalpha;

a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);

an antipsychotic agent for example olanzapine and clozapine;

a serotonin receptor modulator;

a leptin/leptin receptor modulator;

a ghrelin/ghrelin receptor modulator;

a DPP-IV inhibitor;

an SGLT-2 inhibitor;

a GLK activator;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for the treatment of obesity and its associated complications in a warm-blooded animal, such as man, in need of such treatment which comprises administering to said animal an effective amount of a compound of formula (I) or formula (II), or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

According to a further aspect of the invention there is provided a pharmaceutical composition which comprises a compound of formula (I) or formula (II), or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I) or formula (II), or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula (I) or formula (II), or a pharmaceutically acceptable salt thereof, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

According to a further aspect of the present invention there is provided a kit comprising:

a) a compound of formula (I) or formula (II), or a pharmaceutically acceptable salt thereof, together with a pharmaceutically acceptable diluent or carrier, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

Therefore in an additional feature of the invention, there is provided a method for the treatment of diabetes mellitus and obesity and its associated complications in a warm-blooded animal, such as man, in need of such treatment which comprises administering to said animal an effective amount of a compound of formula (I) or formula (II), or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula (I) or formula (II), or a pharmaceutically acceptable salt thereof, including the compounds of the provisos b) and c), in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula (I) or formula (II), or a pharmaceutically acceptable salt thereof, including the compounds of the proviso b), and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I) or formula (II), or a pharmaceutically acceptable salt thereof, including the compounds of the proviso b), and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula (I) or formula (II), or a pharmaceutically acceptable salt thereof, including the compounds of the proviso b), together with a pharmaceutically acceptable diluent or carrier, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of a compound of the formula (I) or formula (II), or a pharmaceutically acceptable salt thereof, including the compounds of the proviso b), and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to a further aspect of the present invention there is provided a combination treatment comprising the administration of an effective amount of a compound of the formula (I) or formula (II), or a pharmaceutically acceptable salt thereof, including the compounds of the proviso b), optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with other therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis).

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological ACTIVITY/BIOLOGICAL TESTS

### Assay Y

Human recombinant Acetyl-CoA Carboxylase 1 and Acetyl-CoA Carboxylase 2 (ACC1 and ACC2) were assayed by determining the amount of inorganic phosphate (Pᵢ) generated. Preincubation of 6 μL enzyme together with 0.3 μL compound for 15 min was done before the reaction was initiated by the addition of 4 μL substrate. The reaction was carried out in 384-well plates at room temperature containing the following constituents; 1 mM ATP, 0.4 mM Acetyl-CoA, 20 mM Citrate, 50 mM Hepes (pH 7.5), 12.5 mM NaHCO₃, mM MgAc₂×4H₂O, 1.5 mM MgSO₄×7H₂O, 1 mM TCEP, 0.025% BSA, compound at different concentrations, ACC1 (diluted 1:50) or ACC2 (diluted 1:15). After 3 hr the reaction was stopped by addition of 60 μL of water followed by 30 μL Malachite Green solution. The Malachite Green solution was prepared by mixing 16.4 mL ammonium molybdate solution (7.5%), 1.64 mL Tween (11%) with 82 mL Malachite Green/H₂SO₄-solution (0.7 g Malachite Green to 1000 mL of 10% (v/v) H₂SO₄). Plates were left for 10 min at room temperature before reading the absorbance at 670 nm.

All compounds were tested in triplicate at 10 different concentrations. Control compound (0, 50 and 100% effect) was included on each plate. The inhibitory effect of a compound was calculated by % inhibition=100*[(X−max)/(min−max)], where X is the readout value in the compound well. Min is the average signal for 100% inhibition and Max is the average signal for 0% inhibition in the control wells. An IC₅₀ value was derived by non-linear regression curve fitting using the following equation: y=A+((B−A)/1+((C/x)̂D))) where A is the non-specific binding, B is the total binding, C is the IC₅₀ and D is the slope. Saturation binding Kd and Bmax were derived using the equation y=((B1 max*x)/(Kd+x)).

### Assay X

Human recombinant Acetyl-CoA Carboxylase 1 and Acetyl-CoA Carboxylase 2 (ACC1 and ACC2) were assayed by determining the amount of inorganic phosphate (Pᵢ) generated. Pre-incubation of 15 μL enzyme together with 0.75 μL DMSO+/−compound for 5 min was done before the reaction was initiated by the addition of 10 μL substrate. The reaction was carried out in 384-well plates at 37° C. containing the following constituents (n=2); 1 mM ATP, 0.4 mM acetyl-CoA, 20 mM citrate, 50 mM Hepes (pH 7.5), 12.5 mM NaHCO₃, 20 mM MgAc₂×4H₂O, 1.5 mM MgSO₄×7H₂O, 1 mM TCEP, 0.025% BSA, compound at different concentrations, ACC1 and ACC2 diluted to generate signals resulting in assay Z′>0.5. After 1.5 h the reaction was stopped by addition of 74 μL of water followed by extensive mixing, split and transfer of 2×40 μL to a read-plate encompassing 30 μL distilled H₂O per well (n=2→n=4). Colour reaction was initiated by addition of 30 μL malachite green reagent per well and allowed 15 min before absorbance reading of the plates at 660 nm. The malachite green reagent was prepared by mixing 16.4 mL ammonium molybdate solution (7.5%), 1.64 mL Tween (11%) with 82 mL Malachite Green/H₂SO₄-solution (0.7 g Malachite Green to 1000 mL of 10% (v/v) H₂SO₄).

All compounds were tested in triplicate at 10 different concentrations. Control compound (0, 50 and 100% effect) was included on each plate. The inhibitory effect of a compound was calculated by % inhibition=100*[(X−Max)/(Min−Max)], where X is the readout value in the compound well. Min is the average signal for 100% inhibition and Max is the average signal for 0% inhibition in the control wells. Curve fitting was done using the following equation: y=(A+((B−A)/(1+((x/C)̂D)))) where A and B represents the curve bottom (% inhibition) and the total amplitude (% inhibition), respectively, whereas C and D are the apparent IC₅₀ and curve slope, respectively.

The compounds of the invention exhibit activity as ACC2 inhibitors. Further, some of the compounds do also, in addition, exhibit activity as ACC1 inhibitors. Compounds of the present invention typically show an IC₅₀ of less than 20 μM vs ACC2, preferably less than 10 μM. The following table shows the biological activities for the exemplified examples.

| Compound | | |
|---|---|---|
| number | ACC2 IC₅₀ (μM) | ACC1 IC₅₀ (μM) |
| Example 1 | 0.05 | 0.20 |
| Example 2 | 2.96 | >20 |
| Example 3 | 0.65 | 1.52 |
| Example 4 | 0.39 | 1.33 |
| Example 5 | 0.26 | 1.22 |
| Example 6 | 0.24 | 0.56 |
| Example 7 | 0.21 | 1.03 |
| Example 8 | 0.08 | 0.31 |
| Example 9 | 0.04 | 0.25 |
| Example 10 | 0.04 | 0.30 |
| Example 11 | 0.10 | 1.22 |
| Example 12 | 0.56 | >20 |
| Example 13 | 0.16 | 0.45 |
| Example 14 | 0.18 | 0.69 |
| Example 15 | 2.87 | >20 |
| Example 16 | 0.32 | 2.03 |
| Example 17 | 1.94 | >20 |
| Example 18 | 1.80 | 4.85 |
| Example 19 | 0.78 | 1.21 |
| Example 20 | 0.62 | 1.29 |
| Example 21 | 0.97 | 1.49 |
| Example 22 | 0.31 | 1.01 |
| Example 23 | 0.46 | 3.27 |
| Example 24 | 0.49 | 1.35 |
| Example 25 | 0.14 | 1.68 |
| Example 26 | 4.03 | >20 |
| Example 27 | 1.18 | 3.59 |
| Example 28 | 1.17 | 6.00 |
| Example 29 | 1.26 | 4.04 |
| Example 30 | 1.29 | >20 |
| Example 31 | 3.56 | >20 |
| Example 32 | 1.73 | >20 |
| Example 33 | 1.14 | 3.51 |
| Example 34 | 0.22 | 1.17 |
| Example 35 | 3.84 | 9.96 |
| Example 36 | 1.59 | 3.95 |
| Example 37 | 0.47 | 3.69 |
| Example 38 | 4.05 | 5.44 |
| Example 39 | 0.02 | 0.22 |
| Example 40 | 1.00 | 8.64 |
| Example 41 | 4.54 | 6.63 |
| Example 42 | 6.14 | 12.80 |
| Example 43 | 5.82 | 8.31 |
| Example 44 | 9.71 | 13.00 |
| Example 45 | 4.35 | >20 |
| Example 46 | 9.53 | >20 |
| Example 47 | 9.85 | 14.10 |
| Example 48 | 9.67 | >20 |
| Example 49 | 9.87 | 7.60 |
| Example 50 | 6.82 | 5.30 |
| Example 51 | 5.67 | >20 |
| Example 52 | 7.66 | 8.23 |
| Example 53 | 9.08 | >20 |
| Example 54 | 3.99 | >20 |
| Example 55 | 0.28 | 1.30 |
| Example 56 | 0.40 | 1.21 |
| Example 57 | 0.80 | 1.91 |
| Example 58 | 0.81 | 2.10 |
| Example 59 | 4.67 | >20 |
| Example 60 | 1.82 | 3.17 |
| Example 61 | 0.55 | 1.00 |
| Example 62 | 2.77 | 3.07 |
| Example 63 | 2.83 | 4.58 |
| Example 64 | 3.72 | >20 |
| Example 65 | 3.88 | >20 |
| Example 66 | 1.39 | 6.79 |
| Example 67 | 3.29 | >20 |
| Example 68 | 2.62 | >20 |
| Example 69 | 4.76 | 20.00 |
| Example 70 | 2.85 | >20 |
| Example 71 | 2.45 | 16.90 |
| Example 72 | 2.48 | >20 |
| Example 73 | 5.03 | >20 |
| Example 74 | 9.49 | >20 |
| Example 75 | 4.27 | >20 |
| Example 76 | 3.62 | >20 |
| Example 77 | 3.62 | 9.32 |
| Example 78 | 3.14 | 6.55 |
| Example 79 | 5.40 | >20 |
| Example 80 | 3.01 | >20 |
| Example 81 | 3.03 | >20 |
| Example 82 | 3.17 | >20 |
| Example 83 | 3.27 | >20 |
| Example 84 | 4.59 | >20 |
| Example 85 | 6.69 | >20 |
| Example 86 | 6.98 | >20 |
| Example 87 | 8.71 | >20 |
| Example 88 | 2.45 | >20 |
| Example 89 | 3.03 | >20 |
| Example 90 | 2.01 | >20 |
| Example 91 | 2.30 | 4.82 |
| Example 92 | 0.05 | 0.26 |
| Example 93 | 0.17 | 1.85 |
| Example 94 | 0.93 | 5.47 |
| Example 95 | 0.98 | 2.14 |
| Example 96 | 3.64 | >20 |
| Example 97 | 6.14 | 18.30 |
| Example 98 | 0.07 | 1.37 |
| Example 99 | 0.10 | 0.78 |
| Example 100 | 0.11 | 0.59 |
| Example 101 | 0.15 | 2.21 |
| Example 102 | 0.02 | 0.17 |
| Example 103 | 0.04 | 0.20 |
| Example 104 | 0.11 | 1.60 |
| Example 105 | 0.03 | 0.11 |
| Example 106 | 0.07 | 0.43 |
| Example 107 | 0.21 | 2.62 |
| Example 108 | 0.45 | 7.87 |
| Example 109 | 0.21 | 1.71 |
| Example 110 | 4.37 | >20 |
| Example 111 | 0.71 | 3.18 |
| Example 112 | 0.38 | 2.71 |
| Example 113 | 0.94 | 7.80 |
| Example 114 | 0.33 | 2.38 |
| Example 115 | 2.81 | 14.90 |
| Example 116 | 0.18 | 1.26 |
| Example 117 | 0.41 | 0.96 |
| Example 118 | 0.65 | 1.64 |
| Example 119 | 0.72 | 2.26 |
| Example 120 | 1.32 | 4.25 |
| Example 121 | 1.37 | 4.77 |
| Example 122 | 1.86 | 8.13 |
| Example 123 | 0.31 | 0.96 |
| Example 124 | 1.76 | 4.48 |
| Example 125 | 1.95 | 4.99 |
| Example 126 | 0.42 | 1.67 |
| Example 127 | 0.88 | 3.78 |
| Example 128 | 1.14 | 2.58 |
| Example 129 | 2.44 | 7.94 |
| Example 130 | 0.44 | 1.95 |
| Example 131 | 1.59 | 5.25 |
| Example 132 | 1.86 | 5.76 |
| Example 133 | 0.66 | 2.49 |
| Example 134 | 0.10 | 0.53 |
| Example 135 | 0.08 | 0.38 |
| Example 136 | 0.62 | 3.07 |
| Example 137 | 0.68 | 3.41 |
| Example 138 | 0.71 | 3.76 |
| Example 139 | 0.53 | 3.34 |
| Example 140 | 0.96 | 3.31 |
| Example 141 | 0.92 | 3.90 |
| Example 142 | 0.13 | >20 |
| Example 143 | 0.03 | 0.36 |
| Example 144 | 0.22 | 4.00 |
| Example 145 | 0.06 | 0.28 |
| Example 146 | 0.03 | 0.18 |
| Example 147 | 0.03 | 2.59 |
| Example 148 | 0.07 | 0.46 |
| Example 149 | 0.05 | 0.39 |
| Example 150 | 0.13 | 0.35 |
| Example 151 | 0.16 | 0.61 |
| Example 152 | 0.17 | 0.48 |
| Example 153 | 0.13 | 0.23 |
| Example 154 | 0.05 | 0.19 |
| Example 155 | 0.41 | 1.10 |
| Example 156 | 0.35 | 0.81 |
| Example 157 | 0.18 | 0.41 |
| Example 158 | 1.24 | 3.87 |
| Example 159 | 0.28 | 0.50 |
| Example 160 | 0.07 | 0.22 |
| Example 161 | 0.16 | 0.30 |
| Example 162 | 0.37 | 0.88 |
| Example 163 | 0.37 | 1.10 |
| Example 164 | 0.33 | 1.26 |
| Example 165 | 0.16 | 0.54 |
| Example 166 | 0.41 | 0.82 |
| Example 167 | 1.58 | 4.40 |
| Example 168 | 0.23 | 0.68 |
| Example 169 | 0.65 | 1.37 |
| Example 170 | 0.48 | 1.20 |
| Example 171 | 0.14 | 0.29 |
| Example 172 | 0.16 | 3.50 |
| Example 173 | 0.41 | — |
| Example 174 | 0.34 | 1.00 |
| Example 175 | 0.50 | 7.30 |
| Example 176 | 1.05 | 3.04 |
| Example 177 | 1.71 | 4.31 |
| Example 178 | 0.33 | 1.55 |
| Example 179 | 0.55 | 1.80 |
| Example 180 | 1.06 | 6.91 |
| Example 181 | 1.34 | 12.30 |
| Example 182 | 1.76 | 6.43 |
| Example 183 | 1.26 | >20 |
| Example 184 | 0.04 | 0.21 |
| Example 185 | 1.12 | >20 |
| Example 186 | 0.14 | 2.22 |
| Example 187 | 0.56 | 8.48 |
| Example 188 | 0.27 | 5.64 |
| Example 189 | 0.07 | 1.96 |
| Example 190 | 1.30 | 13.60 |
| Example 191 | 0.97 | 2.96 |
| Example 192 | 0.56 | 1.96 |
| Example 193 | 0.10 | 0.44 |
| Example 194 | 0.05 | 0.51 |
| Example 195 | 0.82 | 4.90 |
| Example 196 | 0.92 | 3.48 |
| Example 197 | 1.98 | 9.64 |
| Example 198 | 0.38 | 1.42 |
| Example 199 | 0.37 | 1.60 |
| Example 200 | 1.20 | 4.50 |
| Example 201 | 0.85 | 2.17 |
| Example 202 | 0.51 | 1.10 |
| Example 203 | 0.15 | 0.63 |
| Example 204 | 0.38 | 3.80 |
| Example 205 | 0.14 | 1.31 |
| Example 206 | 0.13 | 0.973 |
| Example 207 | 0.13 | 1.04 |
| Example 208 | 0.17 | 1.10 |
| Example 209 | 0.11 | 0.78 |
| Example 210 | 2.04 | 13.1 |
| Example 211 | 0.27 | 2.22 |
| Example 212 | 0.12 | 1.19 |
| Example 213 | 0.62 | 3.28 |
| Example 214 | 2.24 | 3.31 |
| Example 215 | 0.49 | >20 |
| Example 216 | 0.72 | 3.55 |
| Example 217 | 0.73 | 4.7 |
| Example 218 | 0.06 | 0.21 |
| Example 219 | 0.90 | 4.58 |
| Example 220 | 1.18 | 9.63 |
| Example 221 | 1.61 | >20 |
| Example 222 | 3.40 | 9.10 |
| Example 223 | 0.23 | 1.16 |
| Example 224 | 0.68 | 6.31 |
| Example 225 | 0.11 | 0.95 |
| Example 226 | 0.20 | 1.63 |
| Example 227 | 0.23 | 1.63 |
| Example 228 | 0.21 | 1.52 |
| Example 229 | 0.11 | 0.92 |
| Example 230 | 0.16 | 1.33 |
| Example 231 | 0.08 | 0.70 |
| Example 232 | 0.14 | 1.29 |
| Example 233 | 0.10 | 1.02 |
| Example 234 | 0.11 | 0.95 |
| Example 235 | 0.23 | 1.84 |
| Example 236 | 0.08 | 0.55 |
| Example 237 | 0.14 | 1.09 |
| Example 238 | 0.63 | 4.45 |
| Example 239 | 0.35 | 0.74 |
| Example 240 | 0.36 | 1.08 |
| Example 241 | 0.11 | 0.26 |
| Example 242 | 0.24 | 1.33 |
| Example 243 | 0.61 | 1.93 |
| Example 244 | 0.15 | 1.25 |
| Example 245 | 0.18 | 1.4 |

Examples 121-132, 150-179, 190-203 were tested using the assay conditions described in Assay X. All of the other Examples were tested using the assay conditions described in Assay Y. i) Examples 205-245 were tested using the assay conditions described in Assay X.

### EXAMPLES

### Abbreviations

AcOH Acetic acid
ACN Acetonitrile
DCM Dichloromethane
DIPEA N,N-Diisopropylethylamine
DMAP 4-Dimethylaminopyridine
DME Dimethoxyethane
DMF Dimethylformamide
DMSO Dimethylsulfoxide
EDC 1-Ethyl-3-[3-dimethylaminopropyl]carbodiimide hydrochloride
EtOAc Ethyl acetate
EtOH Ethanol
HOBT 1-Hydroxybenzotriazole
K₂CO₃ Potassium carbonate
KOH Potassium hydroxide
MeOH Methanol
MCPBA meta-Chloroperoxybenzoic acid”
MgAc₂ Magnesium acetate
NaHCO₃ Sodium hydrogencarbonate
NH₄Cl Ammonium chloride
NMM N-Methylmorpholine
NMP N-Methyl-2-pyrrolidone
Pd(PPh₃)₄ Tetrakis(triphenylphosphine)palladium(0)
PEPPSI 1,3-bis(2,6-Diisopropylphenyl)imidazol-2-ylidene](3-chloropyridyl) palladium(II)dichloride
TBTU O-(benzotriazol-1-yl)-N,N,N′,N′-tetramethyluronium tetrafluoroborate
TEA Triethylamine
TFA Trifluoroacetic acid
THF Tetrahydrofuran
TBAF tert-Butyl ammonium fluoride
aq. aqueous
eq. equivalents
h hour
min minutes
rt room temperature
HPLC Standard method A:
Column: Kromasil, C8, 10 μm, 21.2 mm×250 mm
Mobile phase A: 100% ACN
Mobile phase B: 5% ACN+95% 0.1M NH₄OAc
Gradient: ca 30-100% A over ca 30 min.

### HPLC Standard Method B:

Waters Fraction Lynx Purification System with Phenomenex Gemini C18 5 μm 21.2 mm×100 mm columns. The mobile phase used was varying gradients of ACN and 0.2% NH₃ buffer; flow rate 30 mL/min; MS-triggered fraction collection was used.

### HPLC Standard Method C:

Waters Fraction Lynx Purification System with Xbridge Prep C18 5 μm OBD 19 mm×150 mm columns. The mobile phase used was varying gradients of ACN and 0.2% NH₃ buffer; flow rate 30 mL/min; MS-triggered fraction collection was used.

### HPLC Standard Method D:

Column: Kromasil, C8, 10 μm, 50.8 mm×300 mm

Mobile phase A: 100% ACN

Mobile phase B: 5% ACN+95% 0.1M NH₄OAc

Gradient: ca 0-100% A over ca 40 min.

### HPLC Standard Method E:

Waters Fraction Lynx Purification System with Kromasil C8 5 μm 20 mm×100 mm columns. The mobile phase used was varying gradients of ACN and 100 mM ammonium acetate buffer; flow rate 30 mL/min; MS-triggered fraction collection was used.

### HPLC Standard Method F:

Waters Fraction Lynx Purification System with Xbridge Prep C18 5 μm OBD 19 mm×150 mm columns. The mobile phase used was varying gradients of ACN and 0.1 M HCO₂H buffer; flow rate 30 mL/min; MS-triggered fraction collection was used.

### HPLC Standard Method G:

Column: Kromasil, C8, 10 μm, 20 mm×50 mm

Mobile phase A: 100% ACN

Mobile phase B: 5% ACN+95% 0.1 M HCO₂H

Gradient: ca 20-100% A over ca 25 min.

### HPLC Standard Method H:

Column: Kromasil, C8, 10 μm, 19 mm×250 mm

Mobile phase A: 100% ACN

Mobile phase B: 5% ACN+95% 0.1M NH₃

Gradient: ca 30-100% A over ca 30 min.

* Wet ¹H NMR in DMSO/DMSO-d₆: The solutions are taken from a concentrated sample dissolved in (CH₃)₂SO and are diluted with (CD₃)₂SO. Since a substantial amount of (CH₃)₂SO is present in the sample, first a pre-scan is run and analysed to automatically suppress the (CH₃)₂SO (2.54 ppm) and H₂O (3.3 ppm) peaks. This means that in this so-called wet ID experiment the intensity of peaks that reside in these areas around 3.3 ppm and 2.54 ppm are reduced. Furthermore impurities are seen in the spectrum which give rise to a triplet at 1.12 ppm, a singlet at 2.96 and two multiplets between 2.76-2.70 ppm and 2.61-2.55 ppm. Most probably these impurities are dimethylsulfone and diethylsulfoxide.

Cation exchange resin CBA (carboxypropyl) commercially available from Biotage was used as its free acid.

Where a microwave is referred to it means a Biotage Initiator or Personal Chemistry [Biotage] Emrys Optimizer.

Drying organic solutions through a phase separator were performed using a IST/Biotage phase separator.

Flash column chromatography was performed using a Horizon/Biotage system with prepacked Biotage Si columns.

Where a lyophilizer is referred to it means a Flexi-Dry MP system from FTS Systems

In order that the invention disclosed herein may be more efficiently understood, examples are provided below. It should be understood that these examples are for illustrative purposes only and are not to be construed as limiting the invention in any manner.

### Example 1

### Method 1

### tert-Butyl [(trans-4-{[({2-[6-(4-methylpiperazin-1-yl)pyridin-3-yl]quinolin-4-yl}carbonyl)amino]methyl}cyclohexyl)methyl]carbamate

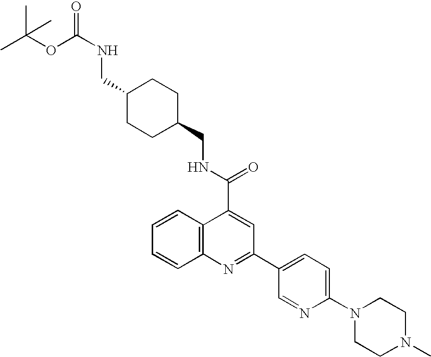

2-[6-(4-Methylpiperazin-1-yl)pyridin-3-yl]quinoline-4-carboxylic acid [Intermediate A] (50 mg, 0.14 mmol) was dissolved in DCM (3 mL) and tert-butyl {[trans-4-(amino-methyl)cyclohexyl]methyl}carbamate (38 mg, 0.16 mmol, 1.1 eq.), TBTU (48 mg, 0.15 mmol, 1.05 eq.) and TEA (58 mg, 0.57 mmol, 4.0 eq.) were added and the reaction mixture was stirred at rt for 15 h. The reaction mixture was concentrated in vacuo to leave a residue, which was dissolved in DMSO and purified by HPLC (Standard method A) to give the title compound (47 mg, 57%): ¹H NMR (400 MHz, DMSO-d₆) δ 9.10 (d, 1H), 8.82 (t, 1H), 8.50 (dd, 1H), 8.17-8.07 (m, 3H), 7.82 (t, 1H), 7.63 (t, 1H), 7.06 (d, 1H), 6.84 (d, 1H), 3.69 (t, 4H), 3.28 (t, 2H), 2.84 (t, 2H), 2.48 (t, 4H), 2.29 (s, 3H), 1.90 (d, 2H), 1.78 (d, 2H), 1.59 (s, 1H), 1.43 (s, 9H), 1.40 (s, 1H), 1.11-0.86 (m, 4H); m/z 573.4 (M+H)⁺.

The following Examples 2-16 were prepared from the Intermediate listed, in place of 2-[6-(4-methylpiperazin-1-yl)pyridin-3-yl]quinoline-4-carboxylic acid, and tert-butyl {[trans-4-(aminomethyl)cyclohexyl]methyl}carbamate using essentially the same conditions as described for Example 1 (Method 1).

### Example 2

### tert-Butyl ({trans-4-[({[2-(2-methyl-6-oxo-1,6-dihydropyridin-3-yl)quinolin-4-yl]carbonyl}amino)methyl]cyclohexyl}methyl)carbamate

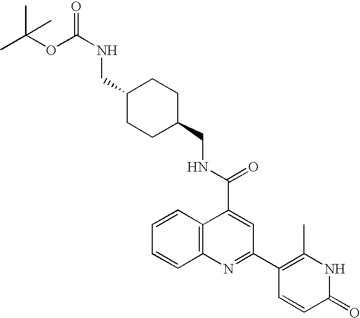

from Intermediate B

¹H NMR (400 MHz, DMSO-d₆) δ 11.81 (s, 1H), 8.68 (t, 1H), 8.06 (d, 1H), 7.99 (d, 1H), 7.80 (d, 1H), 7.75 (t, 1H), 7.62-7.56 (m, 2H), 6.74 (t, 1H), 6.30 (d, 1H), 3.16 (t, 2H), 2.73 (t, 2H), 2.43 (s, 3H), 1.77 (d, 2H), 1.67 (d, 2H), 1.53-1.42 (m, 1H), 1.33 (s, 9H), 1.29-1.21 (m, 1H), 0.98-0.75 (m, 4H); m/z 505.2 (M+H)⁺.

### Example 3

### tert-Butyl {[trans-4-({[(2-pyrazin-2-ylquinolin-4-yl)carbonyl]amino}methyl)cyclohexyl]methyl}carbamate

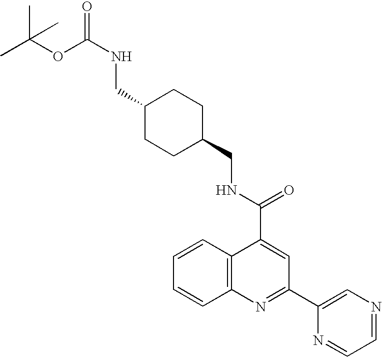

from Intermediate C

¹H NMR (400 MHz, DMSO-d₆) δ 9.73 (d, 1H), 8.87 (t, 1H), 8.82 (t, 1H), 8.78 (d, 1H), 8.41 (s, 1H), 8.18 (d, 1H), 8.13 (d, 1H), 7.89-7.82 (m, 1H), 7.73-7.67 (m, 1H), 6.75 (t, 1H), 3.20 (t, 2H), 2.74 (t, 2H), 1.79 (d, 2H), 1.69 (d, 2H), 1.57-1.44 (m, 1H), 1.33 (s, 9H), 1.31-1.22 (m, 1H), 1.01-0.74 (m, 4H); m/z 476.2 (M+H)⁺.

### Example 4

### tert-Butyl ({trans-4-[({[2-(4-ethoxyphenyl)quinolin-4-yl]carbonyl}amino)methyl]-cyclohexyl}methyl)carbamate

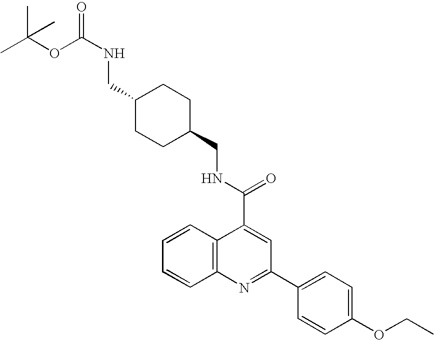

from Intermediate D

¹H NMR (400 MHz, DMSO-d₆) δ 8.75 (s, 1H), 8.22 (d, 2H), 8.10-7.98 (m, 3H), 7.74 (t, 1H), 7.56 (t, 1H), 7.07 (d, 2H), 6.75 (t, 1H), 4.09 (q, 2H), 3.18 (t, 2H), 2.74 (t, 2H), 1.79 (d, 2H), 1.68 (d, 2H), 1.57-1.42 (m, 1H), 1.34 (t, 3H), 1.33 (s, 9H), 1.30-1.21 (m, 1H), 1.01-0.75 (m, 4H); m/z 518.0 (M+H)⁺.

### Example 5

### tert-Butyl ({trans-4-[({[2-(6-carbamoylpyridin-3-yl)quinolin-4-yl]carbonyl}amino)-methyl]cyclohexyl}methyl)carbamate

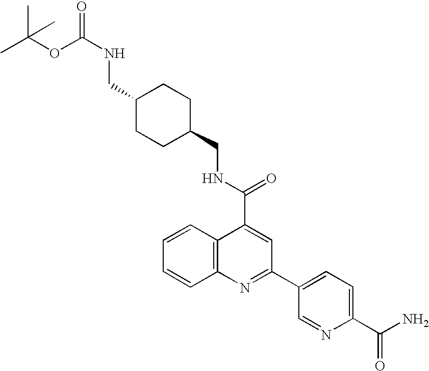

from Intermediate E

¹H NMR (500 MHz, DMSO-d₆) δ 9.50 (d, 1H), 8.85-8.80 (m, 2H), 8.26 (s, 1H), 8.22 (d, 2H), 8.17 (dd, 2H), 7.86 (td, 1H), 7.75 (s, 1H), 7.70 (t, 1H), 6.79 (t, 1H), 3.23 (t, 2H), 2.77 (t, 2H), 1.83 (d, 2H), 1.71 (d, 2H), 1.58-1.49 (m, 1H), 1.36 (s, 9H), 1.34-1.26 (m, 1H), 1.01-0.80 (m, 4H); m/z 518.3 (M+H)⁺.

### Example 6

### tert-Butyl {[trans-4-({[(2-phenylquinolin-4-yl)carbonyl]amino}methyl)-cyclohexyl]-methyl}carbamate

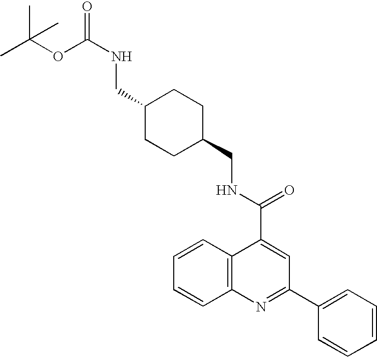

The title compound was prepared using 2-phenyl-quinoline-4-carboxylic acid in place of 2-[6-(4-methylpiperazin-1-yl)pyridin-3-yl]quinoline-4-carboxylic acid: ¹H NMR (400 MHz, DMSO-d₆) δ 8.77 (t, 1H), 8.27 (d, 2H), 8.15-8.02 (m, 3H), 7.78 (t, 1H), 7.61 (t, 1H), 7.57-7.45 (m, 3H), 6.75 (t, 1H), 3.19 (t, 2H), 2.74 (t, 2H), 1.80 (d, 2H), 1.68 (d, 2H), 1.58-1.43 (m, 1H), 1.33 (s, 9H), 1.31-1.23 (m, 1H), 1.01-0.75 (m, 4H); m/z 474.0 (M+H)⁺.

### Example 7

### tert-Butyl {[trans-4-({[(2-pyridin-4-ylquinolin-4-yl)carbonyl]amino}methyl)-cyclohexyl]methyl}carbamate

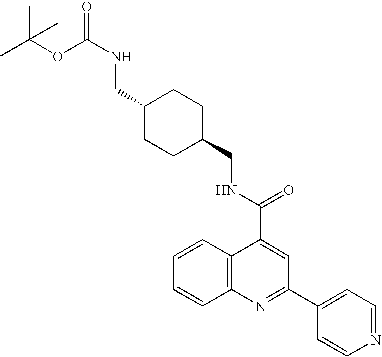

from Intermediate F

¹H NMR (400 MHz, DMSO-d₆) δ 8.79 (t, 1H), 8.75 (dd, 2H), 8.23 (dd, 2H), 8.18 (s, 1H), 8.14 (dd, 2H), 7.83 (t, 1H), 7.67 (t, 1H), 6.75 (t, 1H), 3.20 (t, 2H), 2.74 (t, 2H), 1.80 (d, 2H), 1.68 (d, 2H), 1.50 (s, 1H), 1.33 (s, 9H), 1.30 (s, 1H), 1.00-0.74 (m, 4H); m/z 475.1 (M+H)⁺.

### Example 8

### tert-Butyl ({trans-4-[({[2-(3-carbamoylphenyl)quinolin-4-yl]carbonyl}amino)methyl]-cyclohexyl}methyl)carbamate

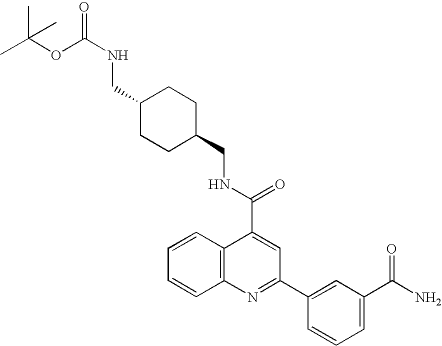

from Intermediate G

¹H NMR (400 MHz, DMSO-d₆) δ 8.80 (t, 1H), 8.73 (s, 1H), 8.43 (d, 1H), 8.20-8.04 (m, 4H), 7.98 (d, 1H), 7.79 (t, 1H), 7.62 (t, 2H), 7.46 (s, 1H), 6.75 (t, 1H), 3.20 (t, 2H), 2.74 (t, 2H), 1.80 (d, 2H), 1.68 (d, 2H), 1.51 (s, 1H), 1.33 (s, 9H), 1.30 (s, 1H), 1.01-0.73 (m, 4H); m/z 517.9 (M+H)⁺.

### Example 9

### tert-Butyl ({trans-4-[({[2-(4-carbamoylphenyl)quinolin-4-yl]carbonyl}amino)methyl]-cyclohexyl}methyl)carbamate

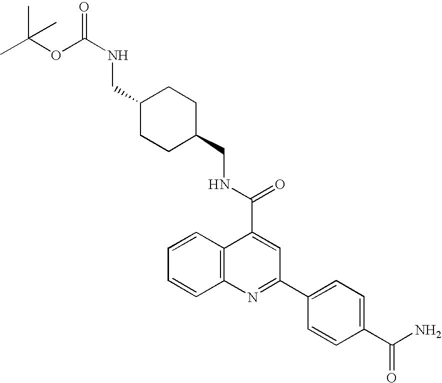

from Intermediate H

¹H NMR (500 MHz, DMSO-d₆) δ 8.83 (t, 1H), 8.40 (d, 2H), 8.18 (s, 1H), 8.16 (d, 2H), 8.11 (s, 1H), 8.07 (d, 2H), 7.84 (t, 1H), 7.67 (t, 1H), 7.48 (s, H), 6.80 (t, 1H), 3.24 (t, 2H), 2.79 (t, 2H), 1.85 (d, 2H), 1.73 (d, 2H), 1.55 (s, 1H), 1.38 (s, 9H), 1.34 (s, 1H), 1.04-0.82 (m, 4H); m/z 517.9 (M+H)⁺.

### Example 10

### tert-Butyl {[trans-4-({[(2-{6-[3-(dimethylamino)propoxy]pyridin-3-yl}quinolin-4-yl)carbonyl]amino}methyl)cyclohexyl]methyl}carbamate

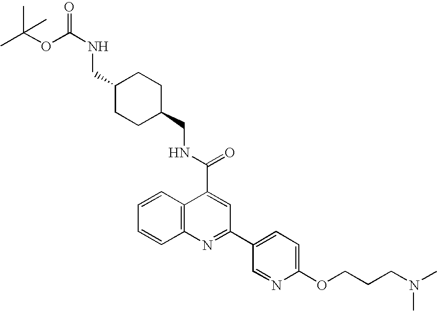

from Intermediate I

¹H NMR (400 MHz, DMSO-d₆) δ 9.04 (d, 1H), 8.74 (t, 1H), 8.57 (dd, 1H), 8.11-8.04 (m, 3H), 7.80-7.74 (m, 1H), 7.62-7.56 (m, 1H), 6.95 (d, 1H), 6.75 (t, 1H), 4.34 (t, 2H), 3.19 (t, 2H), 2.74 (t, 2H), 2.34 (t, 2H), 2.12 (s, 6H), 1.90-1.76 (m, 4H), 1.69 (d, 2H), 1.56-1.44 (m, 1H), 1.33 (s, 9H), 1.31-1.23 (m, 1H), 0.99-0.77 (m, 4H); m/z 576.5 (M+H)⁺.

### Example 11

### tert-Butyl [(trans-4-{[({2-[6-(dimethylamino)pyridin-3-yl]quinolin-4-yl}carbonyl)-amino]methyl}cyclohexyl)methyl]carbamate

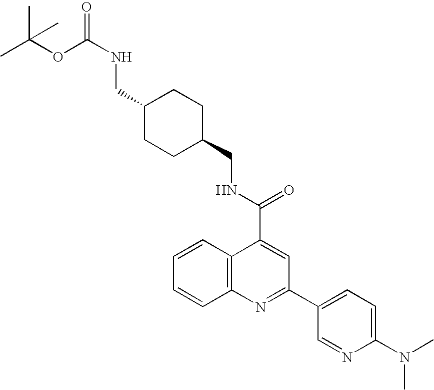

from Intermediate J

¹H NMR (400 MHz, DMSO-d₆) δ 9.00 (d, 1H), 8.72 (t, 1H), 8.39 (dd, 1H), 8.02 (t, 2H), 7.98 (s, 1H), 7.74-7.69 (m, 1H), 7.55-7.49 (m, 1H), 6.79-6.73 (m, 2H), 3.18 (t, 2H), 3.10 (s, 6H), 2.74 (t, 2H), 1.80 (d, 2H), 1.68 (d, 2H), 1.56-1.43 (m, 1H), 1.33 (s, 9H), 1.31-1.23 (m, 1H), 0.99-0.76 (m, 4H); m/z 518.4 (M+H)⁺.

### Example 12

### tert-Butyl [(trans-4-{[({2-[4-(trifluoromethyl)phenyl]quinolin-4-yl}carbonyl)amino]-methyl}cyclohexyl)methyl]carbamate

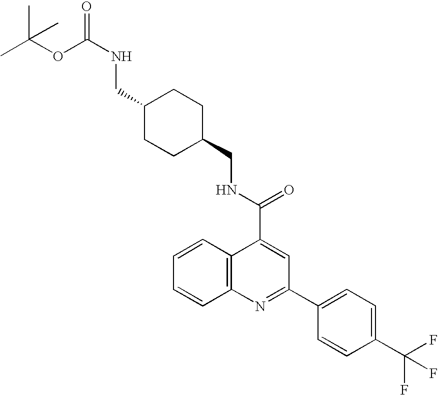

from Intermediate K

¹H NMR (400 MHz, DMSO-d₆) δ 8.80 (t, 1H), 8.49 (d, 2H), 8.16 (s, 1H), 8.13 (d, 2H), 7.91 (d, 2H), 7.82 (t, 1H), 7.65 (t, 1H), 6.75 (t, 1H), 3.20 (t, 2H), 2.74 (t, 2H), 1.80 (d, 2H), 1.68 (d, 2H), 1.58-1.45 (m, 1H), 1.33 (s, 9H), 1.31-1.22 (m, 1H), 1.00-0.77 (m, 4H); m/z 542.2 (M+H)⁺.

### Example 13

### tert-Butyl ({trans-4-[({[2-(5-acetyl-2-thienyl)quinolin-4-yl]carbonyl}amino)methyl]-cyclohexyl}methyl)carbamate

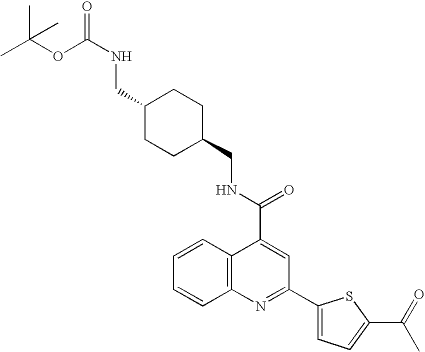

from Intermediate L

¹H NMR (400 MHz, DMSO-d₆) δ 8.79 (t, 1H), 8.15-8.12 (m, 2H), 8.02 (d, 2H), 7.98 (d, 1H), 7.82-7.76 (m, 1H), 7.65-7.59 (m, 1H), 6.75 (t, 1H), 3.19 (t, 2H), 2.74 (t, 2H), 2.55 (s, 3H), 1.80 (d, 2H), 1.68 (d, 2H), 1.54-1.44 (m, 1H), 1.33 (s, 9H), 1.31-1.22 (m, 1H), 1.00-0.76 (m, 4H); m/z 522.8 (M+H)⁺.

### Example 14

### tert-Butyl ({trans-4-[({[2-(4-fluorophenyl)quinolin-4-yl]carbonyl}amino)methyl]-cyclohexyl}methyl)carbamate

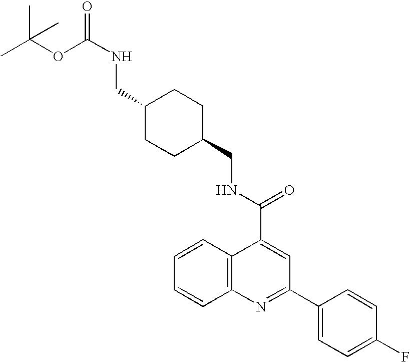

from Intermediate M

¹H NMR (400 MHz, DMSO-d₆) δ 8.77 (t, 1H), 8.34 (dd, 2H), 8.14-8.04 (m, 3H), 7.78 (dd, 1H), 7.61 (t, 1H), 7.37 (t, 2H), 6.75 (t, 1H), 3.19 (t, 2H), 2.74 (t, 2H), 1.80 (d, 2H), 1.68 (d, 2H), 1.57-1.44 (m, 1H), 1.33 (s, 9H), 1.30-1.25 (m, 1H), 1.01-0.76 (m, 4H); m/z 493.0 (M+H)⁺.

### Example 15

### tert-Butyl ({trans-4-[({[2-(3,5-dimethylisoxazol-4-yl)quinolin-4-yl]carbonyl}amino)-methyl]cyclohexyl}methyl)carbamate

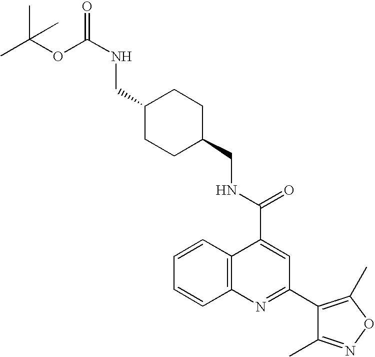

from Intermediate N

¹H NMR (400 MHz, DMSO-d₆) δ 8.73 (t, 1H), 8.04 (t, 2H), 7.79 (t, 1H), 7.67-7.57 (m, 2H), 6.79-6.71 (m, 1H), 3.18 (t, 2H), 2.73 (t, 2H), 2.65 (s, 3H), 2.52 (s, 3H), 1.77 (d, 2H), 1.68 (d, 2H), 1.56-1.43 (m, 1H), 1.33 (s, 9H), 1.30-1.22 (m, 1H), 1.02-0.75 (m, 4H); m/z 494.0 (M+H)⁺.

### Example 16

### tert-Butyl {[trans-4-({[(2-pyridin-3-ylquinolin-4-yl)carbonyl]amino}methyl)cyclo-hexyl]methyl}carbamate

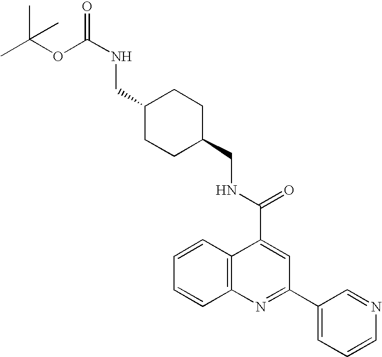

from Intermediate 0

¹H NMR (400 MHz, DMSO-d₆) δ 9.44 (d, 1H), 8.77 (t, 1H), 8.68 (dd, 1H), 8.62 (dt, 1H), 8.16 (s, 1H), 8.15-8.10 (m, 2H), 7.84-7.78 (m, 1H), 7.67-7.61 (m, 1H), 7.60-7.55 (m, 1H), 6.75 (t, 1H), 3.20 (t, 2H), 2.74 (t, 2H), 1.81 (d, 2H), 1.69 (d, 2H), 1.57-1.45 (m, 1H), 1.33 (s, 9H), 1.30-1.23 (m, 1H), 1.00-0.72 (m, 4H); m/z 475.3 (M+H)⁺.

The following Examples 17-19 were prepared from the Intermediate listed and ethyl {[trans-4-(aminomethyl)cyclohexyl]methyl}carbamate hydrochloride [Intermediate P] in place of tert-butyl {[trans-4-(aminomethyl)cyclohexyl]methyl}carbamate, using essentially the same conditions as described for Example 1 (Method 1).

### Example 17

### Ethyl ({trans-4-[({[2-(4-fluorophenyl)quinolin-4-yl]carbonyl}amino)methyl]cyclo-hexyl}methyl)carbamate

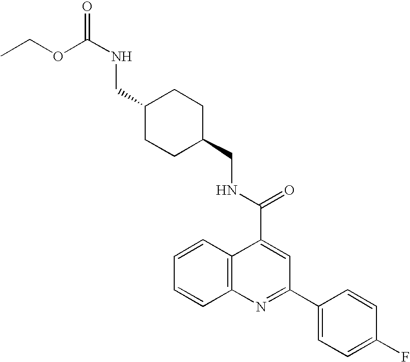

from Intermediate M

¹H NMR (400 MHz, DMSO-d₆) δ 8.77 (t, 1H), 8.34 (dd, 2H), 8.13-8.04 (m, 3H), 7.78 (t, 1H), 7.61 (t, 1H), 7.37 (t, 2H), 7.03 (t, 1H), 3.92 (q, 2H), 3.19 (t, 2H), 2.79 (t, 2H), 1.80 (d, 2H), 1.69 (d, 2H), 1.57-1.45 (m, 1H), 1.36-1.24 (m, 1H), 1.11 (t, 3H), 1.01-0.78 (m, 4H); m/z 464.9 (M+H)⁺.

### Example 18

### Ethyl [(trans-4-{[({2-[4-(dimethylcarbamoyl)phenyl]quinolin-4-yl}carbonyl)amino]-methyl}cyclohexyl)methyl]carbamate

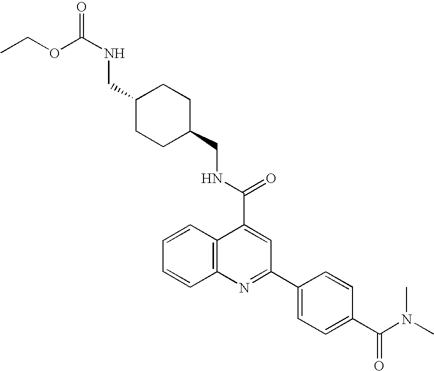

from Intermediate Q

¹H NMR (400 MHz, DMSO-d₆) δ 8.79 (t, 1H), 8.32 (d, 2H), 8.14-8.07 (m, 2H), 7.80 (t, 1H), 7.05-6.99 (m, 1H), 7.65-7.48 (m, 4H), 3.92 (q, 2H), 3.20 (t, 2H), 3.01-2.91 (m, 6H), 2.79 (t, 2H), 1.84-1.65 (m, 4H), 1.56-1.45 (m, 1H), 1.36-1.26 (m, 1H), 1.10 (t, 3H), 1.00-0.78 (m, 4H); m/z 517.9 (M+H)⁺.

### Example 19

### Ethyl ({trans-4-[({[2-(3-carbamoylphenyl)quinolin-4-yl]carbonyl}amino)methyl]-cyclohexyl}methyl)carbamate

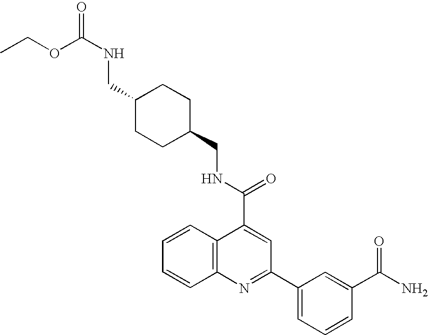

from Intermediate G

¹H NMR (400 MHz, DMSO-d₆) δ 8.81 (t, 1H), 8.73 (s, 1H), 8.43 (d, 1H), 8.17-8.07 (m, 2H), 7.98 (d, 1H), 7.79 (t, 1H), 7.66-7.44 (m, 5H), 7.02 (t, 1H), 3.92 (q, 2H), 3.20 (t, 2H), 2.79 (t, 2H), 1.80 (d, 2H), 1.69 (d, 2H), 1.57-1.44 (m, 1H), 1.36-1.23 (m, 1H), 1.10 (t, 3H), 1.00-0.71 (m, 4H); m/z 489.9 (M+H)⁺.

The following Examples 20-25 were prepared from the Intermediate listed in place of 2-[6-(4-methylpiperazin-1-yl)pyridin-3-yl]quinoline-4-carboxylic acid and tert-butyl {[trans-4-(aminomethyl)cyclohexyl]methyl}carbamate, using essentially the same conditions as described for Example 1 (Method 1), and were purified by HPLC (Standard method B).

### Example 20

### tert-Butyl ({trans-4-[({[2-(4-methylphenyl)quinolin-4-yl]carbonyl}amino)methyl]-cyclohexyl}methyl)carbamate

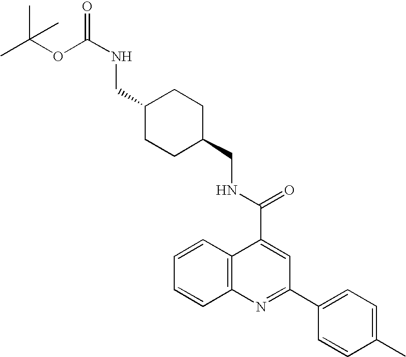

from Intermediate R

¹H NMR (400 MHz, DMSO-d₆) δ 8.76 (t, 1H), 8.17 (d, 2H), 8.12-7.99 (m, 2H), 7.81-7.71 (m, 2H), 7.62-7.54 (m, 2H), 7.35 (d, 1H), 6.79-6.71 (m, 1H), 3.19 (t, 2H), 2.74 (t, 2H), 2.36 (s, 3H), 1.79 (d, 2H), 1.68 (d, 2H), 1.56-1.43 (m, 1H), 1.33 (s, 9H), 1.31-1.21 (m, 1H), 1.00-0.75 (m, 4H); m/z 488.0 (M+H)⁺.

### Example 21

### tert-Butyl ({trans-4-[({[2-(4-chlorophenyl)quinolin-4-yl]carbonyl}amino)methyl]cyclo-hexyl}methyl)carbamate

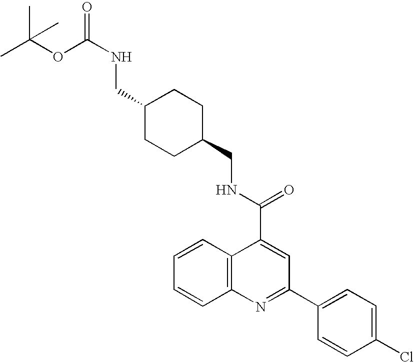

from Intermediate S

¹H NMR (400 MHz, DMSO-d₆) δ 8.77 (t, 1H), 8.31 (d, 2H), 8.14-8.05 (m, 3H), 7.79 (t, 1H), 7.65-7.56 (m, 3H), 6.75 (t, 1H), 3.19 (t, 2H), 2.74 (t, 2H), 1.80 (d, 2H), 1.68 (d, 2H), 1.57-1.43 (m, 1H), 1.33 (s, 9H), 1.30-1.22 (m, 1H), 1.01-0.75 (m, 4H); m/z 508.0 (M+H)⁺.

### Example 22

### tert-Butyl ({trans-4-[({[2-(3-methoxyphenyl)quinolin-4-yl]carbonyl}amino)-methyl]cyclohexyl}methyl)carbamate

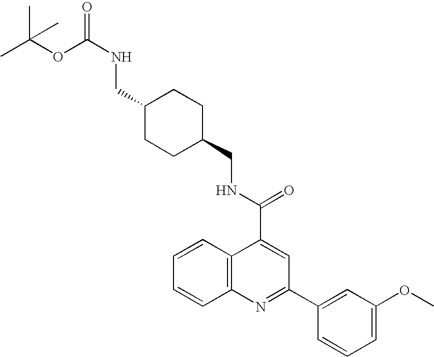

from Intermediate T

¹H NMR (400 MHz, DMSO-d₆) δ 8.76 (t, 1H), 8.15-8.02 (m, H), 7.87-7.74 (m, 3H), 7.61 (t, 1H), 7.45 (t, 1H), 7.07 (dd, 1H), 6.75 (t, 1H), 3.85 (s, 3H), 3.19 (t, 2H), 2.74 (t, 2H), 1.80 (d, 2H), 1.68 (d, 2H), 1.58-1.43 (m, 1H), 1.33 (s, 9H), 1.30-1.23 (m, 1H), 1.02-0.75 (m, 4H); m/z 504.6 (M+H)⁺.

### Example 23

### tert-Butyl ({trans-4-[({[2-(2-methoxyphenyl)quinolin-4-yl]carbonyl}amino)methyl]-cyclohexyl}methyl)carbamate

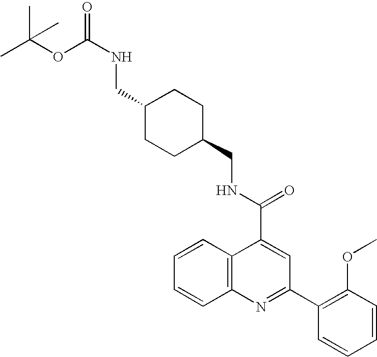

from Intermediate U

¹H NMR (600 MHz, DMSO/DMSO-d₆*) δ 8.69 (t, 1H), 8.07 (t, 2H), 7.89 (s, 1H), 7.80 (dd, 1H), 7.78-7.75 (m, 1H), 7.63-7.59 (m, 1H), 7.48-7.45 (m, 1H), 7.19 (d, 1H), 7.12-7.09 (m, 1H), 6.76 (t, 1H), 3.83 (s, 3H), 3.17 (t, 2H), 2.74 (t, 2H), 1.77 (d, 2H), 1.68 (d, 2H), 1.53-1.45 (m, 1H), 1.33 (s, 9H), 1.31-1.24 (m, 1H), 0.97-0.79 (m, 4H); m/z 504.7 (M+H)⁺.

### Example 24

### tert-Butyl [(trans-4-{[({2-[4-(methylthio)phenyl]quinolin-4-yl}carbonyl)amino]-methyl}cyclohexyl)methyl]carbamate

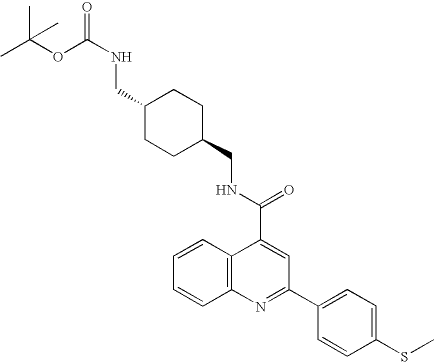

from Intermediate V

¹H NMR (600 MHz, DMSO/DMSO-d₆*) δ 8.78 (t, 1H), 8.25-8.22 (m, 2H), 8.09-8.05 (m, 2H), 8.04 (s, 1H), 7.78-7.75 (m, 1H), 7.60-7.57 (m, 1H), 7.42-7.39 (m, 2H), 6.76 (t, 1H), 3.19 (t, 2H), 2.74 (t, 2H), 1.80 (d, 2H), 1.69 (d, 2H), 1.54-1.47 (m, H), 1.34 (s, 9H), 1.31-1.25 (m, 1H), 0.97-0.79 (m, 4H); m/z 520.6 (M+H)⁺.

### Example 25

### tert-Butyl ({trans-4-[({[2-(2,4-dimethyl-1,3-thiazol-5-yl)quinolin-4-yl]carbonyl}amino)-methyl]cyclohexyl}methyl)carbamate

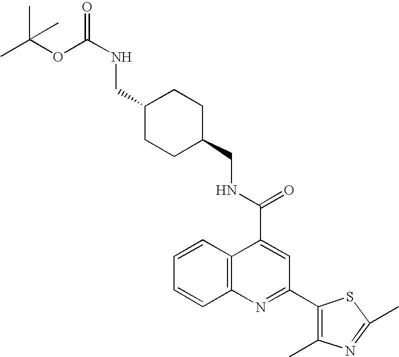

from Intermediate AA

¹H NMR (600 MHz, CDCl₃) δ 8.15-8.09 (m, 2H), 7.76 (t, 1H), 7.67 (s, 1H), 7.59 (t, 1H), 6.11-6.06 (m, 1H), 4.61-4.56 (m, 1H), 3.44 (t, 2H), 2.99 (t, 2H), 2.78 (s, 3H), 2.74 (s, 3H), 1.89 (d, 2H), 1.84 (d, 2H), 1.67-1.58 (m, 1H), 1.75-1.67 (m, 1H), 1.44 (s, 9H), 1.13-0.94 (m, 4H); m/z 509.5 (M+H)⁺.

The following Examples 26-32 were prepared from ethyl {[trans-4-(aminomethyl)cyclo-hexyl]methyl}carbamate hydrochloride [Intermediate P] and the Intermediate listed in place of 2-[6-(4-methylpiperazin-1-yl)pyridin-3-yl]quinoline-4-carboxylic acid, using essentially the same conditions as described for Example 1 (Method 1), and were purified by HPLC (Standard method C).

### Example 26

### Ethyl {[trans-4-({[(2-pyrazin-2-ylquinolin-4-yl)carbonyl]amino}methyl)cyclohexyl]-methyl}carbamate

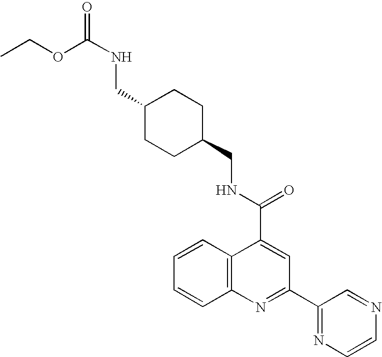

from Intermediate C

¹H NMR (600 MHz, DMSO/DMSO-d₆*) δ 9.75-9.75 (m, 1H), 8.89 (t, 1H), 8.84-8.82 (m, 1H), 8.79 (d, 1H), 8.42 (s, 1H), 8.20 (d, 1H), 8.14 (d, 1H), 7.87 (t, 1H), 7.71 (t, 1H), 7.05 (t, 1H), 3.93 (q, 2H), 3.21 (t, 2H), 2.81 (t, 2H), 1.80 (d, 2H), 1.71 (d, 2H), 1.56-1.48 (m, 1H), 1.36-1.28 (m, 1H), 1.12 (t, 3H), 0.99-0.81 (m, 4H); m/z 448.2 (M+H)⁺.

### Example 27

### Ethyl ({trans-4-[({[2-(3-methoxyphenyl)quinolin-4-yl]carbonyl}amino)methyl]cyclo-hexyl}methyl)carbamate

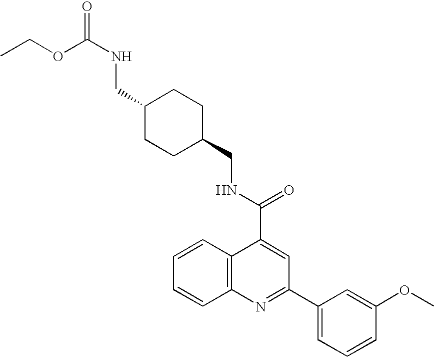

from Intermediate T

¹H NMR (600 MHz, DMSO-d₆) δ 8.79 (t, 1H), 8.10 (t, 2H), 8.07 (s, 1H), 7.85-7.77 (m, 3H), 7.62 (t, 1H), 7.47 (t, 1H), 7.09-7.07 (m, 1H), 7.04 (t, 1H), 3.93 (q, 2H), 3.86 (s, 3H), 3.20 (t, 2H), 2.80 (t, 2H), 1.81 (d, 2H), 1.70 (d, 2H), 1.56-1.48 (m, 1H), 1.35-1.27 (m, 1H), 1.11 (t, 3H), 0.98-0.81 (m, 4H); m/z 476.2 (M+H)⁺.

### Example 28

### Ethyl ({trans-4-[({[2-(4-methylphenyl)quinolin-4-yl]carbonyl}amino)methyl]cyclo-hexyl}methyl)carbamate

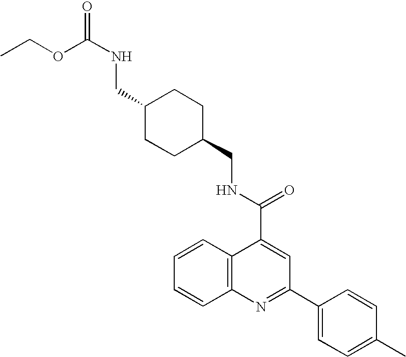

from Intermediate R

¹H NMR (400 MHz, DMSO-d₆) δ 8.76 (t, 1H), 8.17 (d, 2H), 8.12-8.01 (m, 3H), 7.76 (t, 1H), 7.57 (t, 1H), 7.35 (d, 2H), 7.03 (t, 1H), 3.92 (q, 2H), 3.19 (t, 2H), 2.79 (t, 2H), 2.37 (s, 3H), 1.80 (d, 2H), 1.69 (d, 2H), 1.58-1.43 (m, 1H), 1.38-1.24 (m, 1H), 1.11 (t, 3H), 1.01-0.76 (m, 4H); m/z 460.3 (M+H)⁺.

### Example 29

### Ethyl [(trans-4-{[({2-[4-(methylthio)phenyl]quinolin-4-yl}carbonyl)amino]methyl}-cyclohexyl)methyl]carbamate

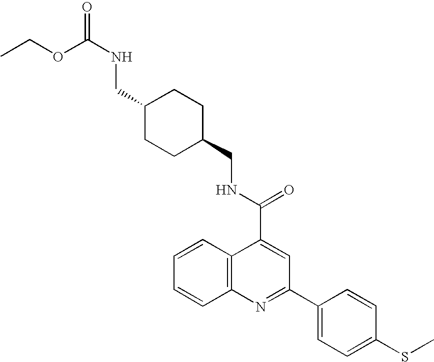

from Intermediate V

¹H NMR (400 MHz, DMSO-d₆) δ 8.77 (t, 1H), 8.23 (d, 2H), 8.10-8.02 (m, 3H), 7.76 (t, H), 7.59 (t, 1H), 7.40 (d, 2H), 7.03 (t, 1H), 3.92 (q, 2H), 3.19 (t, 2H), 3.10-3.01 (m, 1H), 2.79 (t, 2H), 1.80 (d, 2H), 1.69 (d, 2H), 1.57-1.45 (m, 1H), 1.37-1.25 (m, 1H), 1.16-1.07 (m, 5H), 1.00-0.78 (m, 4H); m/z 492.2 (M+H)⁺.

### Example 30

### Ethyl ({trans-4-[({[2-(4-ethoxyphenyl)quinolin-4-yl]carbonyl}amino)methyl]-cyclohexyl}methyl)carbamate

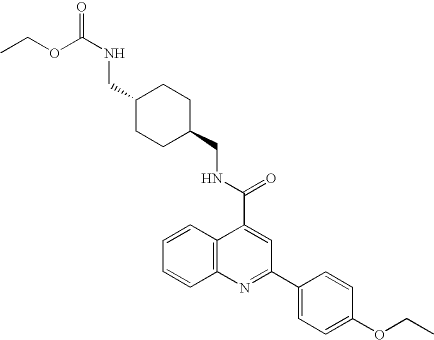

from Intermediate D

¹H NMR (400 MHz, DMSO-d₆) δ 8.75 (t, 1H), 8.22 (d, 2H), 8.05 (dd, 2H), 8.00 (s, 1H), 7.77-7.71 (m, 1H), 7.56 (t, 1H), 7.07 (d, 2H), 7.04-6.99 (m, 1H), 4.09 (q, 2H), 3.92 (q, 2H), 3.18 (t, 2H), 2.79 (t, 2H), 1.80 (d, 2H), 1.69 (d, 2H), 1.56-1.45 (m, 1H), 1.34 (t, 3H), 1.32-1.26 (m, 1H), 1.10 (t, 3H), 1.00-0.77 (m, 4H); m/z 490.2 (M+H)⁺.

### Example 31

### Ethyl ({trans-4-[({[2-(2-methoxyphenyl)quinolin-4-yl]carbonyl}amino)methyl]-cyclohexyl}methyl)carbamate

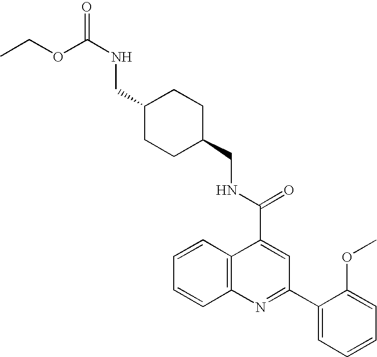

from Intermediate U

m/z 476.2 (M+H)⁺, retention time 5.0 min.

### Example 32

### Ethyl ({trans-4-[({[2-(4-chlorophenyl)quinolin-4-yl]carbonyl}amino)methyl]cyclo-hexyl}methyl)carbamate

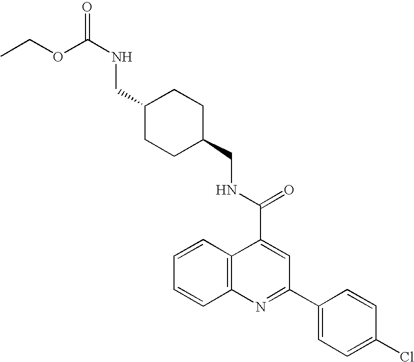

from Intermediate S

¹H NMR (400 MHz, DMSO-d₆) δ 8.77 (t, 1H), 8.31 (d, 2H), 8.13-8.06 (m, 3H), 7.82-7.76 (m, 1H), 7.65-7.58 (m, 3H), 7.03 (t, 1H), 3.92 (q, 2H), 3.19 (t, 2H), 2.79 (t, 2H), 1.80 (d, 2H), 1.69 (d, 2H), 1.57-1.44 (m, 1H), 1.36-1.25 (m, 1H), 1.10 (t, 3H), 1.00-0.78 (m, 4H); m/z 480.3 (M+H)⁺.

### Example 33

### tert-Butyl {[trans-4-({[(2-pyrimidin-5-ylquinolin-4-yl)carbonyl]amino}methyl)cyclo-hexyl]methyl}carbamate

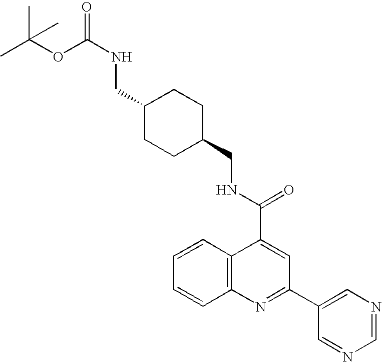

from Intermediate BB

The title compound was prepared using essentially the same method as described for Example 1 (Method 1), but using DMF as solvent and NMM as base instead of TEA. ¹H NMR (500 MHz, DMSO-d₆) δ 9.66 (s, 2H), 9.34 (s, 1H), 8.81 (t, 1H), 8.30 (s, 1H), 8.18 (t, 2H), 7.91-7.85 (m, 1H), 7.75-7.69 (m, 1H), 6.80 (t, 1H), 3.25 (t, 2H), 2.78 (t, 2H), 1.85 (d, 2H), 1.73 (d, 2H), 1.59-1.51 (m, 1H), 1.37 (s, 9H), 1.35-1.29 (m, 1H), 1.04-0.82 (m, 4H); m/z 476.3 (M+H)⁺.

### Example 34

### tert-Butyl ({trans-4-[({[2-(4-cyanophenyl)quinolin-4-yl]carbonyl}amino)methyl]cyclo-hexyl}methyl)carbamate

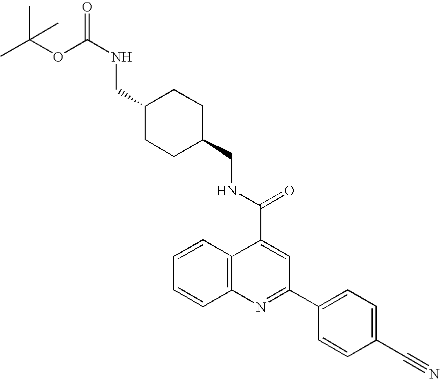

from Intermediate CC

The title compound was prepared using essentially the same method as described for Example 1 (Method 1), but using DMF as solvent and NMM as base instead of TEA. ¹H NMR (500 MHz, CDCl₃) δ 8.31 (d, 2H), 8.21 (dd, 2H), 7.93 (s, 1H), 7.87-7.79 (m, 3H), 7.65 (dd, 1H), 6.18 (t, 1H), 4.66-4.57 (m, 1H), 3.46 (t, 2H), 3.00 (t, 2H), 1.91 (d, 2H), 1.85 (d, 2H), 1.70-1.60 (m, 1H), 1.45 (s, 9H), 1.51-1.40 (m, 1H), 1.16-0.94 (m, 4H); m/z 499.2 (M+H)⁺.

### Example 35

### tert-Butyl {[trans-4-({[(2-chloroquinolin-4-yl)carbonyl]amino}methyl)cyclohexyl]-methyl}carbamate

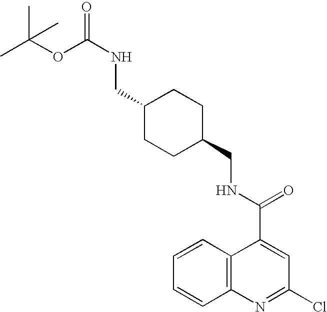

N-Methyl morpholine (5.9 mL, 54 mmol) and tert-butyl {[trans-4-(aminomethyl)cyclo-hexyl]methyl}carbamate (6.7 g, 28 mmol) were added to a solution of 2-chloroquinoline-4-carboxylic acid (5.6 g, 27 mmol) in a mixture of 2-methyltetrahydrofuran (50 mL) and water (34 mL) at rt. An aqueous solution (9.1 mL) of HOBt (20% w/w) and N-methyl morpholine (15% w/w) was added to the stirred solution followed by the addition of EDC (6.7 g, 35 mmol). The reaction mixture was stirred vigorously at rt for 4 days. The mixture was filtered and the collected solid was washed with water containing 10% methanol to leave the title compound (6.6 g, 57%): ¹H NMR (400 MHz, DMSO-d₆) δ 8.81 (t, 1H), 8.07 (d, 1H), 7.99 (d, 1H), 7.89-7.81 (m, 1H), 7.73-7.66 (m, 1H), 7.59 (s, 1H), 6.77 (t, 1H), 3.21-3.12 (m, 2H), 2.79-2.72 (m, 2H), 1.83-1.74 (m, 2H), 1.72-1.64 (m, 2H), 1.56-1.43 (m, 1H), 1.35 (s, 9H), 1.33-1.24 (m, 1H), 0.90-0.75 (m, 4H); m/z 432.1 (M+H)⁺.

### Example 36

### Method 2

### tert-Butyl ({trans-4-[({2-[4-(aminomethyl)phenyl]-6-methoxyisonicotinoyl}-amino)methyl]cyclohexyl}methyl)carbamate

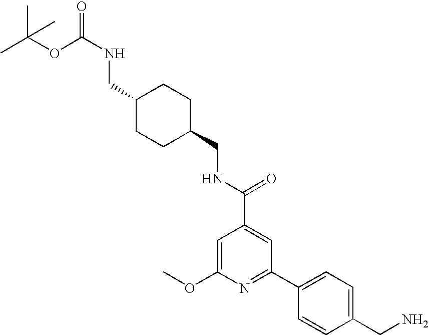

tert-Butyl [(trans-4-{[(2-chloro-6-methoxyisonicotinoyl)amino]methyl}cyclohexyl)-methyl]carbamate [Intermediate EE] (200 mg, 0.49 mmol), [4-(aminomethyl)phenyl]-boronic acid hydrochloride (109 mg, 0.58 mmol, 1.2 eq.), Pd(PPh₃)₄ (28 mg, 0.024 mmol, 0.05 eq.), and K₂CO₃ (208 mg, 1.5 mmol, 3.1 eq.) were diluted with dioxane (1.5 mL) and water (1.5 mL). The reaction mixture was degassed and heated to 60° C. under a nitrogen atmosphere for 16 h. The reaction mixture was then diluted with water and extracted with a mixture of DCM and MeOH. The organic phase was concentrated in vacuo to leave a residue which was purified with a cation-exchange resin CBA eluting with NH₃ in MeOH to give the title compound as a white solid (141 mg, 60%): ¹H NMR (400 MHz, DMSO-d₆) δ 8.75-8.68 (m, 1H), 8.08 (d, 2H), 7.88 (s, 1H), 7.46 (d, 2H), 7.10 (s, 1H), 6.81-6.75 (m, 1H), 3.99 (s, 3H), 3.78 (s, 2H), 3.13 (t, 2H), 2.76 (t, 2H), 1.76 (d, 2H), 1.69 (d, 2H), 1.56-1.44 (m, 1H), 1.37 (s, 9H), 1.33-1.25 (m, 1H), 0.98-0.76 (m, 4H); m/z 483.2 (M+H)⁺.

### Example 37

### tert-Butyl ({trans-4-[({2-[4-(aminomethyl)phenyl]-5-chloroisonicotinoyl}amino)-methyl]cyclohexyl}methyl)carbamate

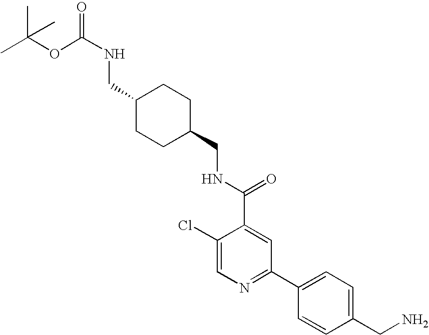

The title compound was prepared using essentially the same conditions as described for Example 36 (Method 2), starting from tert-butyl [(trans-4-{[(2,5-dichloroisonicotinoyl)-amino]methyl}cyclohexyl)methyl]carbamate [Intermediate FF] in place of tert-butyl [(trans-4-{[(2-chloro-6-methoxyisonicotinoyl)amino]methyl}cyclohexyl)-methyl]-carbamate: ¹H NMR (400 MHz, DMSO-d₆) δ 8.72 (s, 1H), 8.67-8.60 (m, 1H), 8.05 (d, 2H), 7.95 (s, 1H), 7.45 (d, 2H), 6.81-6.74 (m, 1H), 3.76 (s, 2H), 3.11 (t, 2H), 2.76 (t, 2H), 1.80 (d, 2H), 1.69 (d, 2H), 1.51-1.40 (m, 1H), 1.36 (s, 9H), 1.33-1.24 (m, 1H), 0.99-0.77 (m, 4H); m/z 487.1 (M+H)⁺.

### Example 38

### tert-Butyl ({trans-4-[({2-[4-(aminomethyl)phenyl]-6-methylisonicotinoyl}amino)-methyl]cyclohexyl}methyl)carbamate

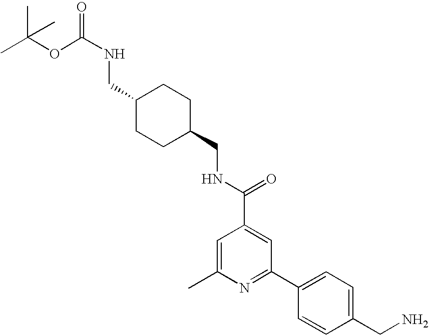

The title compound was prepared using essentially the same conditions as described for Example 36 (Method 2), starting from tert-butyl [(trans-4-{[(2-chloro-6-methylisonicotinoyl)amino]methyl}cyclohexyl)methyl]carbamate [Intermediate GG] in place of tert-butyl [(trans-4-{[(2-chloro-6-methoxyisonicotinoyl)amino]methyl}-cyclohexyl)methyl]carbamate: ¹H NMR (400 MHz, DMSO-d₆) δ 8.77-8.69 (m, 1H), 8.09-8.02 (m, 3H), 7.55 (s, 1H), 7.46 (d, 2H), 6.83-6.74 (m, 1H), 3.78 (s, 2H), 3.14 (t, 2H), 2.76 (t, 2H), 2.59 (s, 3H), 1.77 (d, 2H), 1.70 (d, 2H), 1.56-1.44 (m, 1H), 1.37 (s, 9H), 1.33-1.24 (m, 1H), 0.97-0.76 (m, 4H); m/z 467.2 (M+H)⁺.

### Example 39

### tert-Butyl [(trans-4-{[({2-[3-(aminomethyl)phenyl]quinolin-4-yl}carbonyl)amino]-methyl}cyclohexyl)methyl]carbamate

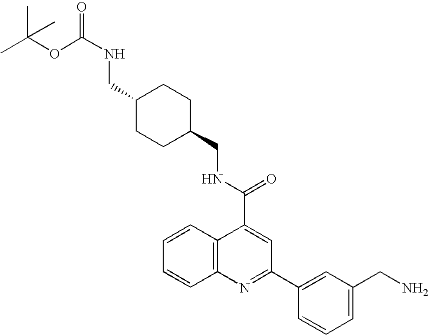

The title compound was prepared using essentially the same conditions as described for Example 36 (Method 2), starting from tert-butyl {[trans-4-({[(2-chloroquinolin-4-yl)carbonyl]amino}methyl)cyclohexyl]methyl}carbamate [Example 35] in place of tert-butyl [(trans-4-{[(2-chloro-6-methoxyisonicotinoyl)amino]methyl}cyclohexyl)-methyl]-carbamate. The compound was purified by flash column chromatography using

EtOAc:MeOH:TEA (100:10:1) as eluent: ¹H NMR (400 MHz, MeOH-d₄) δ 8.21-8.11 (m, 3H), 8.08 (d, 1H), 8.02 (s, 1H), 7.84-7.77 (m, 1H), 7.67-7.60 (m, 1H), 7.57-7.48 (m, 2H), 3.96 (s, 2H), 3.35 (d, 2H), 2.90 (d, 2H), 1.94 (d, 2H), 1.83 (d, 2H), 1.71-1.58 (m, 1H), 1.43 (s, 10H), 1.15-0.91 (m, 4H); m/z 503.1 (M+H)⁺.

### Example 40

### Method 3

### [3-(4-{[(trans-4-{[(tert-Butoxycarbonyl)amino]methyl}cyclohexyl)methyl]-carbamoyl}quinolin-2-yl)phenyl]acetic Acid

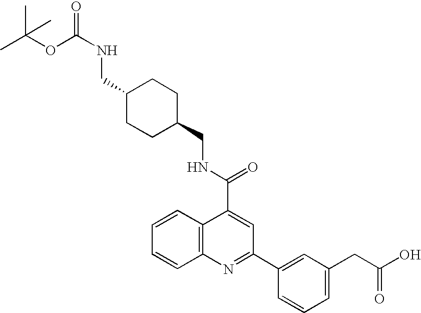

tert-Butyl {[trans-4-({[(2-chloroquinolin-4-yl)carbonyl]amino}methyl)cyclohexyl]-methyl}carbamate [Example 35] (200 mg, 0.46 mmol), [3-(4,4,5,5-tetramethyl-1,3,2-dioxaborolan-2-yl)phenyl]acetic acid (133 mg, 0.51 mmol, 1.1 eq.), Pd(PPh₃)₄ (26 mg, 0.023 mmol, 0.05 eq.), K₂CO₃ (198 mg, 1.43 mmol, 3.1 eq.), water (2 mL), and dioxane (2 mL) were added to a vial and degassed. The reaction mixture was heated to 60° C. for 16 h under a nitrogen atmosphere and then the reaction mixture was diluted with DCM, MeOH and a mixture of water and citric acid and the organic phase was separated and concentrated in vacuo to leave a residue. The residue was washed with MeOH to give the title compound as a white solid (188 mg, 76%): ¹H NMR (300 MHz, DMSO-d₆) δ 12.38 (s, 1H), 8.80 (t, 1H), 8.13 (m, 5H), 7.80 (t, 1H), 7.63 (t, 1H), 7.51 (t, 1H), 7.41 (d, 1H), 6.82-6.74 (m, 1H), 3.72 (s, 2H), 3.22 (t, 2H), 2.77 (t, 2H), 1.83 (d, 2H), 1.71 (d, 2H), 1.61-1.46 (m, 1H), 1.36 (s, 10H), 1.07-0.78 (m, 4H); m/z 532.3 (M+H)⁺.

### Example 41

### Method 4

### tert-Butyl [(trans-4-{[(2-phenylisonicotinoyl)amino]methyl}cyclohexyl)methyl]-carbamate

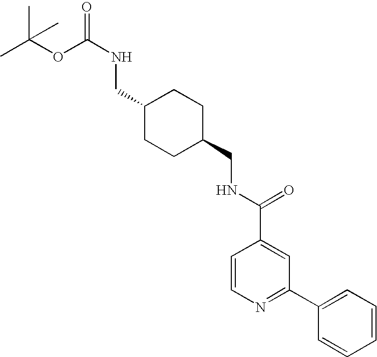

tert-Butyl [(trans-4-{[(2-bromoisonicotinoyl)amino]methyl}cyclohexyl)methyl] carbamate [Intermediate HH] (62 mg, 0.15 mmol) was dissolved in ACN (3 mL) and treated with phenyl boronic acid (32 mg, 0.26 mmol, 1.8 eq.), Pd(OAc)₂ (14 mg, 0.06 mmol, 0.4 eq.), and a 1M aqueous solution of NaHCO₃ (1 mL). The reaction mixture was sealed, degassed with nitrogen for 15 min, and heated in the microwave at 150° C. for 10 min. The solvent was concentrated in vacuo to leave a residue. The residue was dissolved in DCM and washed with a saturated aqueous solution of NaHCO₃ and then dried using a phase separator. The solvent was concentrated in vacuo to leave a residue which was purified by flash column chromatography, using EtOAc:heptane (2:1)+1% TEA as eluent, to give the title compound (33 mg, 53%): ¹H NMR (500 MHz, CDCl₃) δ 8.80 (d, 1H), 8.09-8.02 (m, 3H), 7.53-7.44 (m, 4H), 6.43-6.34 (m, 1H), 4.64-4.56 (m, 1H), 3.36 (t, 2H), 2.99 (t, 2H), 1.90-1.79 (m, 4H), 1.66-1.55 (m, 2H), 1.45 (s, 9H), 1.10-0.91 (m, 4H); m/z 424.2 (M+H)⁺.

### Example 42

### tert-Butyl {[trans-4-({[2-(4-methoxyphenyl)isonicotinoyl]amino}methyl)cyclo-hexyl]methyl}carbamate

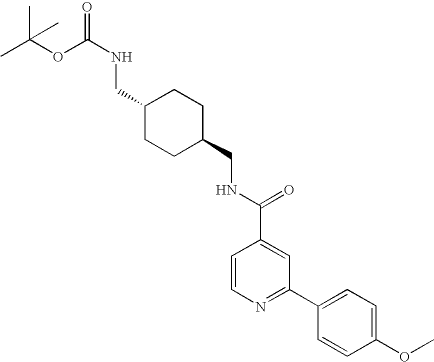

The title compound was prepared using essentially the same procedure as described for Example 41 (Method 4), starting from 4-methoxyphenyl boronic acid in place of phenyl boronic acid: ¹H NMR (500 MHz, CDCl₃) δ 8.76 (d, 1H), 8.04-8.00 (m, 3H), 7.42 (dd, 1H), 7.05-7.01 (m, 2H), 6.33-6.27 (m, 1H), 4.63-4.57 (m, 1H), 3.89 (s, 3H), 3.37 (t, 2H), 3.00 (t, 2H), 1.85 (dd, 4H), 1.64-1.55 (m, 2H), 1.46 (s, 9H), 1.11-0.93 (m, 4H).

### Example 43

### tert-Butyl [(trans-4-{[(2-methyl-6-phenylisonicotinoyl)amino]methyl}cyclohexyl)-methyl]carbamate

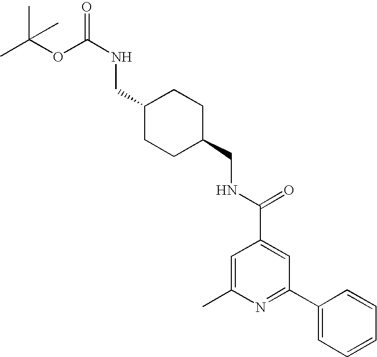

The title compound was prepared using essentially the same procedure as described for Example 41 (Method 4), starting from tert-butyl [(trans-4-{[(2-chloro-6-methylisonicotinoyl)amino]methyl}cyclohexyl)methyl]carbamate [Intermediate GG] in place of tert-butyl [(trans-4-{[(2-bromoisonicotinoyl)amino]methyl}cyclohexyl)-methyl]carbamate. The compound was purified by flash column chromatography, using EtOAc:heptane (2:1)+1% TEA as eluent, and then by HPLC (Standard method D). ¹H NMR (500 MHz, CDCl₃) δ 7.89 (d, 2H), 7.69 (s, 1H), 7.37-7.30 (m, 3H), 7.23 (s, 1H), 6.13 (s, 1H), 4.45 (s, 1H), 3.22 (t, 2H), 2.85 (t, 2H), 2.56 (s, 3H), 1.74-1.67 (m, 4H), 1.31 (s, 9H), 1.21-1.13 (m, 2H), 0.96-0.73 (m, 4H); m/z 438.2 (M+H)⁺.

### Example 44

### tert-Butyl {[trans-4-({[(5-phenylpyridin-3-yl)carbonyl]amino}methyl)cyclohexyl]-methyl}carbamate

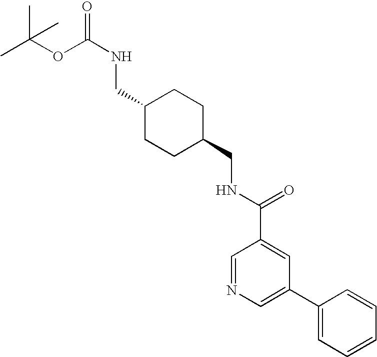

The title compound was prepared using essentially the same procedure as described for Example 41 (Method 4), starting from tert-butyl {[trans-4-({[(5-bromopyridin-3-yl)carbonyl]amino}methyl)cyclohexyl]methyl}carbamate [Intermediate II] in place of tert-butyl [(trans-4-{[(2-bromoisonicotinoyl)amino]methyl}cyclohexyl)methyl]-carbamate. The compound was purified by flash column chromatography, using EtOAc:heptane (2:1)+1% TEA as eluent, and then by HPLC (Standard method D). ¹H NMR (500 MHz, CDCl₃) δ 8.93 (d, 2H), 8.31 (s, 1H), 7.62 (d, 2H), 7.52-7.44 (m, 3H), 6.53 (s, 1H), 4.63 (s, 1H), 3.37 (t, 2H), 2.98 (t, 2H), 1.87 (d, 2H), 1.81 (d, 2H), 1.64-1.56 (m, 1H), 1.45 (s, 9H), 1.35-1.24 (m, 1H), 1.06-0.89 (m, 4H); m/z 424.1 (M+H)⁺.

The Examples 45-53 were prepared using essentially the same method as described for Example 41 (Method 4) using tert-butyl [(trans-4-{[(2-bromoisonicotinoyl)-amino]-methyl}cyclohexyl)methyl]carbamate [Intermediate HH] in place of tert-butyl [(trans-4-{[(2-bromoisonicotinoyl)amino]methyl}cyclohexyl)methyl]carbamate, and the appropriate boronic acid. The compounds were purified by HPLC (Standard method B).

### Example 45

### tert-Butyl {[trans-4-({[2-(1-benzothiophen-2-yl)isonicotinoyl]amino}methyl)cyclo-hexyl]methyl}carbamate

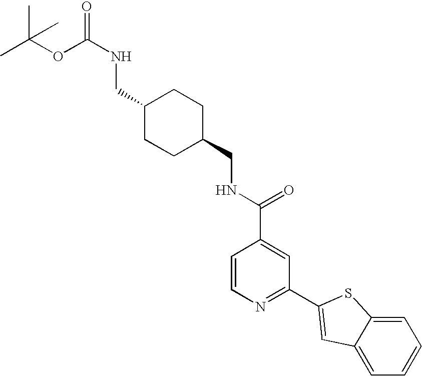

¹H NMR (600 MHz, DMSO/DMSO-d₆*) δ 8.79 (t, 1H), 8.68 (d, 1H), 8.39 (s, 1H), 8.20 (s, 1H), 7.98-7.95 (m, 1H), 7.91-7.87 (m, 1H), 7.65 (d, 1H), 7.40-7.35 (m, 2H), 6.75 (t, 1H), 3.13 (t, 2H), 2.73 (t, 2H), 1.74 (d, 2H), 1.66 (d, 2H), 1.52-1.44 (m, 1H), 1.33 (s, 9H), 1.29-1.23 (m, 1H), 0.93-0.76 (m, 4H); m/z 480.7 (M+H)⁺.

### Example 46

### tert-Butyl {[trans-4-({[(6′-ethoxy-2,3′-bipyridin-4-yl)carbonyl]amino}methyl)cyclohexyl]methyl}carbamate

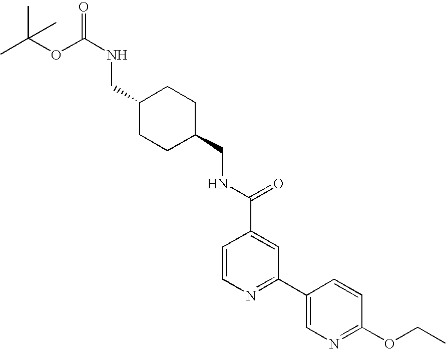

¹H NMR (600 MHz, DMSO/DMSO-d₆*) δ 8.86 (d, 1H), 8.74 (t, 1H), 8.72 (d, 1H), 8.37-8.34 (m, 1H), 8.21 (s, 1H), 7.65-7.62 (m, 1H), 6.90 (d, 1H), 6.74 (t, 1H), 4.34 (q, 2H), 3.11 (t, 2H), 2.72 (t, 2H), 1.73 (d, 2H), 1.65 (d, 2H), 1.50-1.42 (m, 1H), 1.32 (s, 9H), 1.31 (t, 3H), 1.29-1.22 (m, 1H), 0.92-0.75 (m, 4H); m/z 469.5 (M+H)⁺.

### Example 47

### tert-Butyl {[trans-4-({[2-(2,4-dimethoxyphenyl)isonicotinoyl]amino}methyl)cyclohexyl]methyl}carbamate

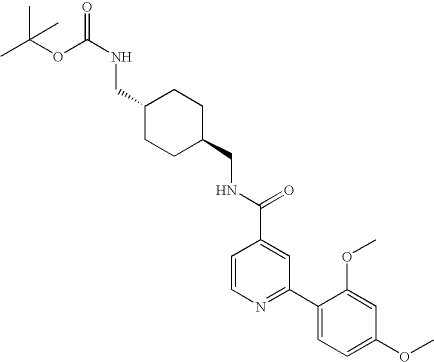

¹H NMR (600 MHz, DMSO/DMSO-d₆*) δ 8.67 (d, 1H), 8.65 (t, 1H), 8.11 (s, 1H), 7.69 (d, 1H), 7.55-7.53 (m, 1H), 6.74 (t, 1H), 6.67-6.65 (m, 1H), 6.64-6.61 (m, 1H), 3.80 (s, 3H), 3.79 (s, 3H), 3.08 (t, 2H), 2.72 (t, 2H), 1.70 (d, 2H), 1.65 (d, 2H), 1.49-1.41 (m, 1H), 1.32 (s, 9H), 1.29-1.21 (m, 1H), 0.90-0.74 (m, 4H); m/z 484.3 (M+H)⁺.

### Example 48

### tert-Butyl {[trans-4-({[(2′,6′-dimethoxy-2,3′-bipyridin-4-yl)carbonyl]amino}methyl)-cyclohexyl]methyl}carbamate

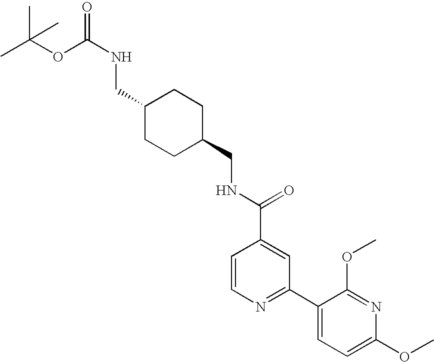

¹H NMR (600 MHz, DMSO/DMSO-d₆*) δ 8.70-8.66 (m, 2H), 8.26-8.23 (m, 2H), 7.58-7.55 (m, 1H), 6.74 (t, 1H), 6.52 (d, 1H), 3.96 (s, 3H), 3.90 (s, 3H), 3.09 (t, 2H), 2.72 (t, 2H), 1.71 (d, 2H), 1.65 (d, 2H), 1.50-1.41 (m, 1H), 1.32 (s, 9H), 1.28-1.21 (m, 1H), 0.91-0.74 (m, 4H); m/z 485.5 (M+H)⁺.

### Example 49

### tert-Butyl {[trans-4-({[(6′-methoxy-2,3′-bipyridin-4-yl)carbonyl]amino}methyl)cyclohexyl]methyl}carbamate

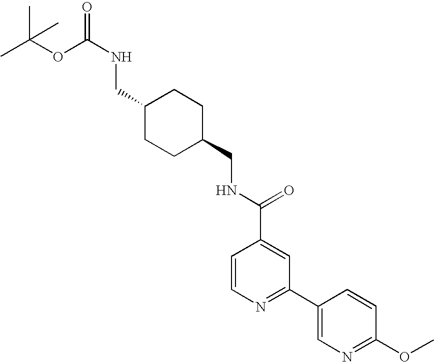

¹H NMR (600 MHz, DMSO/DMSO-d₆*) δ 8.88 (d, 1H), 8.75 (t, 1H), 8.73 (d, 1H), 8.38-8.36 (m, 1H), 8.22 (s, 1H), 7.65-7.63 (m, 1H), 6.94 (d, 1H), 6.74 (t, 1H), 3.89 (s, 3H), 3.11 (t, 2H), 2.72 (t, 2H), 1.73 (d, 2H), 1.65 (d, 2H), 1.51-1.42 (m, 1H), 1.32 (s, 9H), 1.29-1.21 (m, 1H), 0.92-0.74 (m, 4H); m/z 455.6 (M+H)⁺.

### Example 50

### tert-Butyl {[trans-4-({[2-(4-carbamoylphenyl)isonicotinoyl]amino}methyl)cyclohexyl]-methyl}carbamate

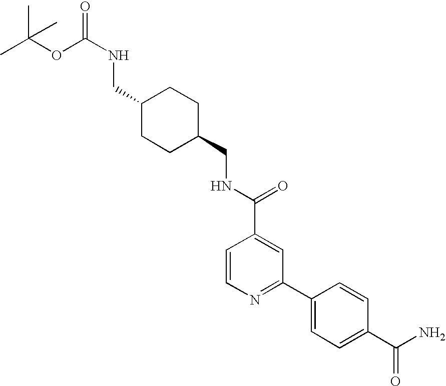

m/z 465.5 (M−H)⁻.

### Example 51

### tert-Butyl {[trans-4-({[2-(3-chlorophenyl)isonicotinoyl]amino}methyl)cyclohexyl]-methyl}carbamate

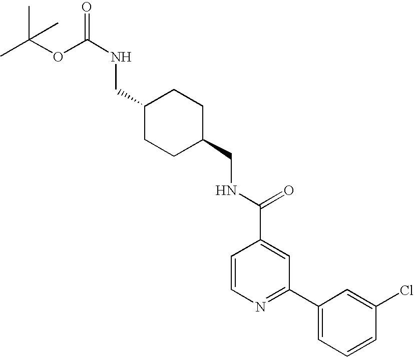

¹H NMR (600 MHz, DMSO/DMSO-d₆*) δ 8.80 (t, 1H), 8.76 (d, 1H), 8.28 (s, 1H), 8.14 (s, 1H), 8.07 (d, 1H), 7.71 (d, 1H), 7.55-7.49 (m, 2H), 6.74 (t, 1H), 3.12 (t, 2H), 2.72 (t, 2H), 1.73 (d, 2H), 1.65 (d, 2H), 1.51-1.43 (m, 1H), 1.32 (s, 9H), 1.29-1.22 (m, 1H), 0.92-0.74 (m, 4H); m/z 458.2 (M+H)⁺.

### Example 52

### tert-Butyl ({trans-4-[({2-[4-(aminomethyl)phenyl]isonicotinoyl}amino)methyl]cyclo-hexyl}methyl)carbamate

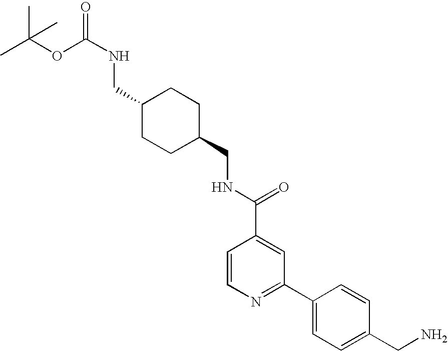

¹H NMR (400 MHz, DMSO-d₆) δ 8.79 (t, 1H), 8.75 (d, 1H), 8.25 (s, 1H), 8.07 (d, 2H), 7.68-7.64 (m, 1H), 7.47 (d, 2H), 6.77 (t, 1H), 3.77 (s, 2H), 3.18-3.12 (m, 2H), 2.75 (t, 2H), 2.02-1.89 (m, 2H), 1.76 (d, 2H), 1.69 (d, 2H), 1.55-1.44 (m, 1H), 1.36 (s, 9H), 1.33-1.24 (m, 1H), 0.97-0.75 (m, 4H); m/z 453.2 (M+H)⁺.

### Example 53

### tert-Butyl {[trans-4-({[2-(3,4-difluorophenyl)isonicotinoyl]amino}methyl)cyclohexyl]-methyl}carbamate

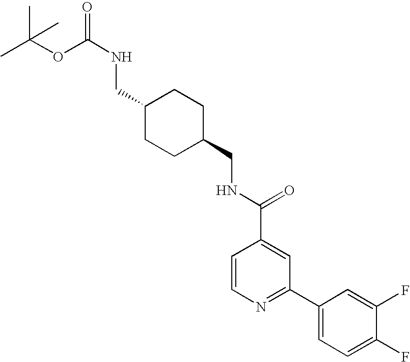

¹H NMR (600 MHz, DMSO/DMSO-d₆*) δ 8.77 (t, 1H), 8.75 (d, 1H), 8.27 (s, 1H), 8.16-8.11 (m, 1H), 8.00-7.96 (m, 1H), 7.69 (d, 1H), 7.56 (q, 1H), 6.74 (t, 1H), 3.12 (t, 2H), 2.72 (t, 2H), 1.73 (d, 2H), 1.65 (d, 2H), 1.51-1.42 (m, 1H), 1.32 (s, 9H), 1.29-1.23 (m, 1H), 0.92-0.74 (m, 4H); m/z 460.5 (M+H)⁺.

### Example 54

### Method 5

### Ethyl [(trans-4-{[(2-naphthylsulfonyl)amino]methyl}cyclohexyl)methyl]carbamate

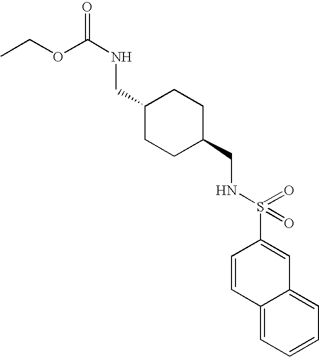

N-{[trans-4-(Aminomethyl)cyclohexyl]methyl}naphthalene-2-sulfonamide [Intermediate DD] (30 mg, 0.077 mmol) and TEA (23 mg, 0.23 mmol, 3.0 eq.) were dissolved in DCM (1 mL) and a few drops of DMSO were added. The reaction mixture was cooled to 0° C. A solution of ethyl chloroformate (8.4 mg, 0.077 mmol, 1.0 eq.) in DCM (1 mL) was then added dropwise and the reaction mixture was stirred at 0° C. whilst warming to rt overnight. The reaction mixture was concentrated in vacuo to leave a residue which was purified by HPLC (Standard method A) to give the title compound (20 mg, 63%): ¹H NMR (400 MHz, CDCl₃) δ 8.40 (s, 1H), 7.25 (d, 2H), 7.89 (d, 1H), 7.79 (dd, 1H), 7.61 (ddd, 2H), 4.64-4.57 (m, 1H), 4.41 (t, 1H), 4.06 (q, 2H), 2.96 (t, 2H), 2.80 (t, 2H), 1.72 (d, 4H), 1.42-1.26 (m, 2H), 1.20 (t, 3H), 0.92-0.76 (m, 4H); m/z 405.0 (M+H)⁺.

The following Examples 55-60 were prepared from N-{[trans-4-(aminomethyl)cyclo-hexyl]methyl}-2-pyridin-4-ylquinoline-4-carboxamide hydrochloride [Intermediate JJ] in place of N-{[trans-4-(aminomethyl)cyclohexyl]methyl}naphthalene-2-sulfonamide and the appropriate alkyl chloroformate, using essentially the same method as described for Example 54 (Method 5).

### Example 55

### 2,2-Dimethylpropyl {[trans-4-({[(2-pyridin-4-ylquinolin-4-yl)carbonyl]amino}methyl)-cyclohexyl]methyl}carbamate

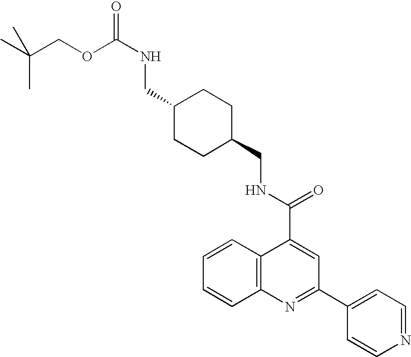

¹H NMR (500 MHz, DMSO-d₆) δ 8.84 (t, 1H), 8.80 (d, 2H), 8.27 (d, 2H), 8.23 (s, 1H), 8.18 (t, 2H), 7.88 (t, 1H), 7.72 (t, 1H), 7.08 (t, 1H), 3.65 (s, 2H), 3.25 (t, 2H), 2.85 (t, 2H), 1.85 (d, 2H), 1.75 (d, 2H), 1.61-1.50 (m, 1H), 1.42-1.32 (m, 1H), 1.04-0.82 (m, 13H); m/z 489.3 (M+H)⁺.

### Example 56

### Cyclopentyl {[trans-4-({[(2-pyridin-4-ylquinolin-4-yl)carbonyl]amino}methyl)cyclo-hexyl]methyl}carbamate

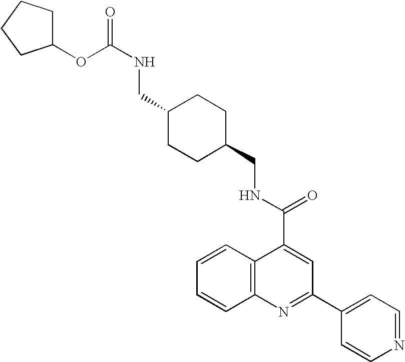

¹H NMR (400 MHz, DMSO-d₆) δ 8.85-8.72 (m, 3H), 8.26-8.20 (m, 2H), 8.20-8.09 (m, 2H), 7.87-7.79 (m, 1H), 7.71-7.63 (m, 1H), 6.99-6.91 (m, 1H), 4.93-4.84 (m, 1H), 3.22-3.15 (m, 2H), 2.82-2.72 (m, 2H), 1.85-1.63 (m, 6H), 1.63-1.43 (m, 7H), 1.36-1.23 (m, 2H), 1.01-0.76 (m, 4H); m/z 487.3 (M+H)⁺.

### Example 57

### Isopropyl {[trans-4-({[(2-pyridin-4-ylquinolin-4-yl)carbonyl]amino}methyl)cyclo-hexyl]methyl}carbamate

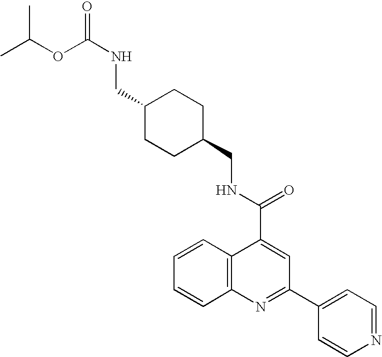

¹H NMR (400 MHz, DMSO-d₆) δ 8.80 (t, 1H), 8.76 (d, 2H), 8.23 (d, 2H), 8.18 (s, 1H), 8.16-8.10 (m, 2H), 7.86-7.80 (m, 1H), 7.71-7.65 (m, 1H), 6.95 (t, 1H), 4.74-4.63 (m, 1H), 3.20 (t, 2H), 2.79 (t, 2H), 1.81 (d, 2H), 1.69 (d, 2H), 1.56-1.44 (m, 1H), 1.37-1.24 (m, 1H), 1.11 (d, 6H), 1.01-0.77 (m, 4H); m/z 461.2 (M+H)⁺.

### Example 58

### Propyl {[trans-4-({[(2-pyridin-4-ylquinolin-4-yl)carbonyl]amino}methyl)cyclohexyl]-methyl}carbamate

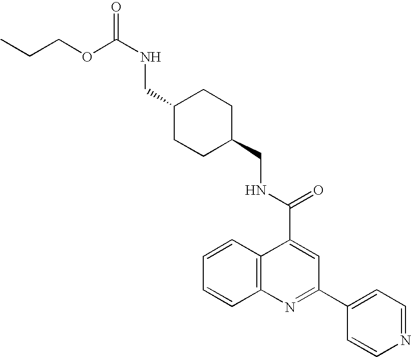

¹H NMR (500 MHz, DMSO-d₆) δ 8.84 (t, 1H), 8.80 (d, 2H), 8.28 (d, 2H), 8.23 (s, 1H), 8.18 (t, 2H), 7.88 (t, 1H), 7.72 (t, 1H), 7.08 (t, 1H), 3.88 (t, 2H), 3.25 (t, 2H), 2.84 (t, 2H), 1.85 (d, 2H), 1.74 (d, 2H), 1.54 (m, 2H), 1.60-1.48 (m, 1H), 1.41-1.30 (m, 1H), 1.04-0.90 (m, 4H), 0.88 (t, 3H); m/z 461.3 (M+H)⁺.

### Example 59

### 2-Methoxyethyl {[trans-4-({[(2-pyridin-4-ylquinolin-4-yl)carbonyl]amino}-methyl)cyclohexyl]methyl}carbamate

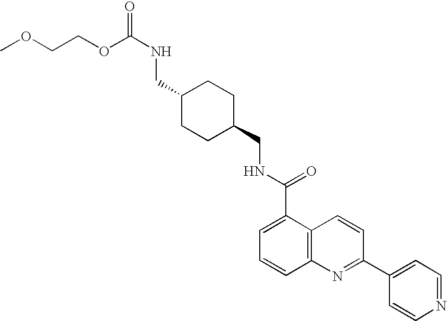

¹H NMR (500 MHz, DMSO-d₆) δ 8.84 (t, 1H), 8.80 (d, 2H), 8.28 (d, 2H), 8.23 (s, 1H), 8.18 (t, 2H), 7.88 (t, 1H), 7.72 (t, 1H), 7.20 (t, 1H), 4.04 (t, 2H), 3.48 (t, 2H), 3.27-3.22 (m, 5H), 2.84 (t, 2H), 1.85 (d, 2H), 1.74 (d, 2H), 1.59-1.51 (m, 1H), 1.40-1.31 (m, 1H), 1.04-0.83 (m, 4H); m/z 477.3 (M+H)⁺.

### Example 60

### Ethyl {[trans-4-({[(2-pyridin-4-ylquinolin-4-yl)carbonyl]amino}methyl)cyclo-hexyl]methyl}carbamate

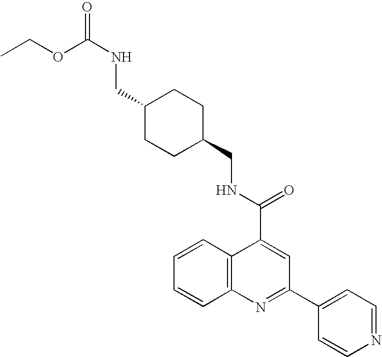

¹H NMR (400 MHz, CDCl₃) δ 8.77 (dd, 2H), 8.19 (dd, 2H), 8.04 (dd, 2H), 7.90 (s, 1H), 7.84-7.76 (m, 1H), 7.67-7.59 (m, 1H), 6.13 (t, 1H), 4.66 (s, 1H), 4.08 (q, 2H), 3.44 (t, 2H), 3.03 (t, 2H), 1.86 (dd, 4H), 1.69-1.54 (m, 1H), 1.51-1.35 (m, 1H), 1.21 (t, 3H), 1.14-0.90 (m, 4H); m/z 447.1 (M+H)⁺.

### Example 61

### Ethyl ({trans-4-[({[2-(4-carbamoylphenyl)quinolin-4-yl]carbonyl}amino)methyl]-cyclohexyl}methyl)carbamate

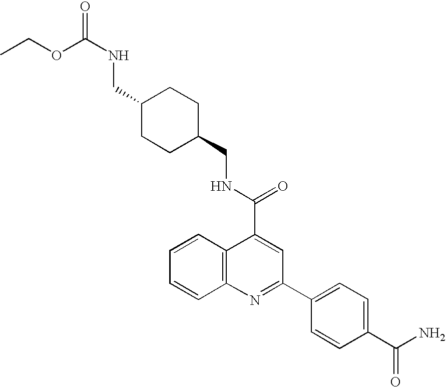

The title compound was prepared from N-{[trans-4-(aminomethyl)cyclohexyl]methyl}-2-(4-carbamoylphenyl)quinoline-4-carboxamide hydrochloride [Intermediate KK] in place of N-{[trans-4-(aminomethyl)cyclohexyl]methyl}naphthalene-2-sulfonamide, using essentially the same method as described for Example 54 (Method 5). ¹H NMR (400 MHz, DMSO-d₆) δ 8.79 (t, 1H), 8.35 (d, 2H), 8.16-7.98 (m, 6H), 7.80 (dd, 1H), 7.63 (dd, 1H), 7.43 (s, 1H), 7.03 (t, 1H), 3.92 (q, 2H), 3.20 (t, 2H), 2.80 (t, 2H), 1.80 (d, 2H), 1.70 (d, 2H), 1.57-1.44 (m, 1H), 1.38-1.24 (m, 1H), 1.11 (t, 3H), 1.01-0.76 (m, 4H); m/z 489.9 (M+H)⁺.

### Example 62

### Ethyl {[trans-4-({[(2-phenylquinolin-4-yl)carbonyl]amino}methyl)cyclohexyl]methyl}-carbamate

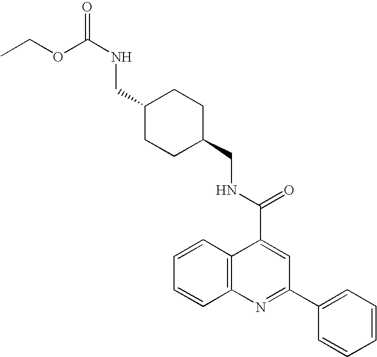

The title compound was prepared from N-{[trans-4-(aminomethyl)cyclohexyl]methyl}-2-phenylquinoline-4-carboxamide [Intermediate LL] in place of N-{[trans-4-(aminomethyl)-cyclohexyl]methyl}naphthalene-2-sulfonamide, using essentially the same method as described for Example 54 (Method 5). ¹H NMR (500 MHz, CDCl₃) δ 8.16 (t, 1H), 8.12 (d, 2H), 7.86 (s, 1H), 7.74 (t, 1H), 7.58-7.49 (m, 3H), 7.49-7.43 (m, 1H), 6.11 (s, 1H), 4.66 (s, 1H), 4.07 (q, 3H), 3.41 (t, 2H), 3.02 (t, 2H), 1.88 (d, 2H), 1.81 (d, 2H), 1.64-1.58 (m, 1H), 1.47-1.38 (m, 1H), 1.21 (t, 3H), 1.11-0.91 (m, 4H); m/z 446.3 (M+H)⁺.

### Example 63

### Method 6

### tert-Butyl [(trans-4-{[(biphenyl-3-ylsulfonyl)amino]methyl}cyclohexyl)methyl]-carbamate

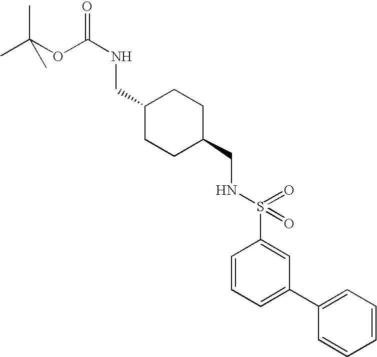

tert-Butyl {[trans-4-(aminomethyl)cyclohexyl]methyl}carbamate (96 mg, 0.40 mmol) was dissolved in DCM (4 mL) and treated with 3-biphenylsulfonyl chloride (99 mg, 0.40 mmol, 1.0 eq.) and TEA (44 mg, 0.44 mmol, 1.1 eq.). The reaction mixture was stirred at rt for 15 h and then diluted with a 1M aqueous solution of HCl. The layers were separated and the organic layer was concentrated in vacuo to leave a residue. The residue was dissolved in DMSO (1 mL) and purified by HPLC (Standard method E) to give the title compound (77 mg, 42%): ¹H NMR (500 MHz, DMSO-d₆) δ 8.03 (s, 1H), 7.93 (d, 1H), 7.77 (d, 1H), 7.74-7.65 (m, 3H), 7.65-7.60 (m, 1H), 7.53 (t, 2H), 7.44 (t, 1H), 6.78-6.73 (m, 1H), 2.72 (t, 2H), 2.60 (t, 2H), 1.71-1.59 (m, 4H), 1.36 (s, 9H), 1.30-1.17 (m, 2H), 0.83-0.68 (m, 4H); m/z 457.2 (M−H)⁻.

The following Examples 64-78 were prepared by essentially the same method as described for Example 63 (Method 6), starting from tert-butyl {[trans-4-(aminomethyl)cyclohexyl]-methyl}carbamate and the appropriate sulphonyl chloride in place of 3-biphenylsulfonyl chloride.

### Example 64

### tert-Butyl {[trans-4-({[(3,4-dibromophenyl)sulfonyl]amino}methyl)cyclohexyl]-methyl}carbamate

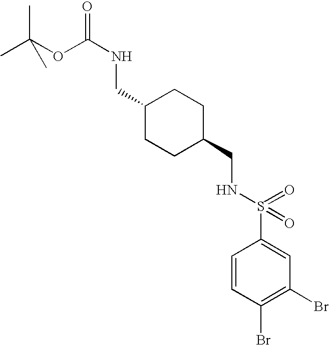

¹H NMR (500 MHz, DMSO-d₆) δ 8.05 (s, 1H), 8.00 (d, 1H), 7.80-7.75 (m, 1H), 7.67 (d, 1H), 6.79-6.74 (m, 1H), 2.73 (t, 2H), 2.60 (t, 2H), 1.70-1.61 (m, 4H), 1.37 (s, 9H), 1.30-1.20 (m, 2H), 0.83-0.71 (m, 4H); m/z 536.9 (M−H)⁻.

### Example 65

### tert-Butyl {[trans-4-({[(3,4-dichlorophenyl)sulfonyl]amino}methyl)cyclohexyl]methyl}-carbamate

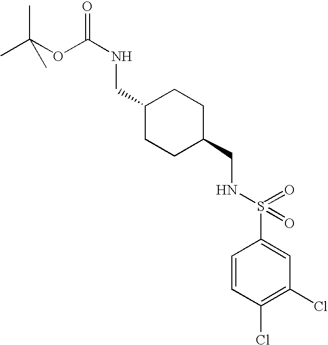

¹H NMR (500 MHz, DMSO-d₆) δ 7.96 (s, 1H), 7.89 (d, 1H), 7.81-7.76 (m, 1H), 7.74 (d, 1H), 6.79-6.74 (m, 1H), 2.73 (t, 2H), 2.60 (t, 2H), 1.70-1.60 (m, 4H), 1.37 (s, 9H), 1.30-1.19 (m, 2H), 0.82-0.72 (m, 4H); m/z 451.0 (M+H)⁺.

### Example 66

### tert-Butyl [(trans-4-{[(quinolin-8-ylsulfonyl)amino]methyl}cyclohexyl)methyl]-carbamate

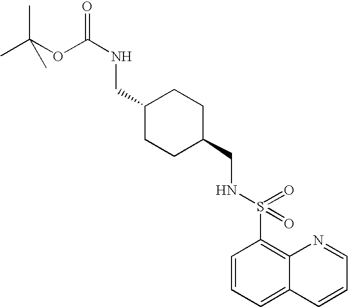

¹H NMR (500 MHz, DMSO-d₆) δ 9.08 (m, 1H), 8.56 (d, 1H), 8.3 (d, 2H), 7.78-7.71 (m, 2H), 7.12 (t, 1H), 6.72 (t, 1H), 2.69 (t, 2H), 2.6 (t, 2H), 1.57 (t, 4H), 1.35 (s, 9H), 1.27-1.1 (m, 2H), 0.72-0.60 (m, 4H); m/z 434.1 (M+H)⁺.

### Example 67

### tert-Butyl [(trans-4-{[(biphenyl-4-ylsulfonyl)amino]methyl}cyclohexyl)methyl]-carbamate

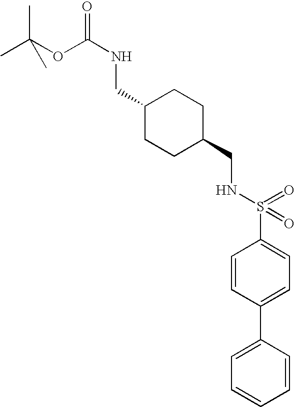

¹H NMR (500 MHz, DMSO-d₆) δ 7.87 (dd, 4H), 7.74 (d, 2H), 7.63-7.59 (m, 1H), 7.52 (t, 2H), 7.44 (t, 1H), 6.78-6.73 (m, 1H), 2.73 (t, 2H), 2.60 (t, 2H), 1.73-1.60 (m, 4H), 1.36 (s, 9H), 1.32-1.19 (m, 2H), 0.84-0.70 (m, 4H); m/z 457.2 (M−H)⁻.

### Example 68

### tert-Butyl {[trans-4-({[(4-isopropylphenyl)sulfonyl]amino}methyl)cyclohexyl]methyl}-carbamate

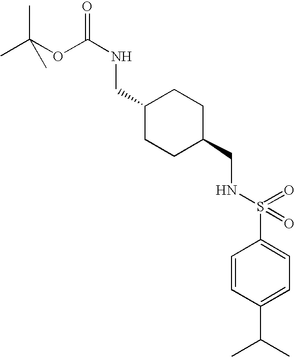

¹H NMR (500 MHz, DMSO-d₆) δ 7.69 (d, 2H), 7.50-7.43 (m, 3H), 6.78-6.73 (m, 1H), 3.31-3.29 (m, 2H), 3.01-2.94 (m, 1H), 2.72 (t, 2H), 1.69-1.59 (m, 4H), 1.36 (s, 9H), 1.27-1.18 (m, 2H), 1.22 (d, 6H), 0.78-0.71 (m, 4H); m/z 423.3 (M−H)⁻.

### Example 69

### tert-Butyl [(trans-4-{[(2,3-dihydro-1,4-benzodioxin-6-ylsulfonyl)amino]methyl}cyclo-hexyl)methyl]carbamate

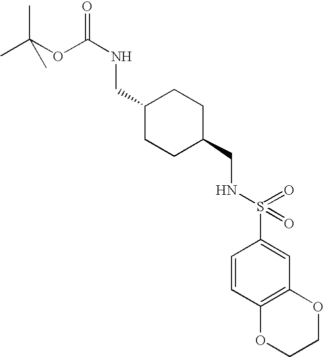

¹H NMR (500 MHz, DMSO-d₆) δ 7.40 (t, 1H), 7.25-7.21 (m, 2H), 7.03 (d, 1H), 6.79-6.74 (m, 1H), 4.34-4.28 (m, 4H), 2.73 (t, 2H), 2.53-2.52 (m, 2H), 1.71-1.60 (m, 4H), 1.37 (s, 9H), 1.29-1.18 (m, 2H), 0.82-0.71 (m, 4H); m/z 439.2 (M−H)⁻.

### Example 70

### tert-Butyl ({trans-4-[({[5-(2-methyl-1,3-thiazol-4-yl)-2-thienyl]sulfonyl}amino)-methyl]cyclohexyl}methyl)carbamate

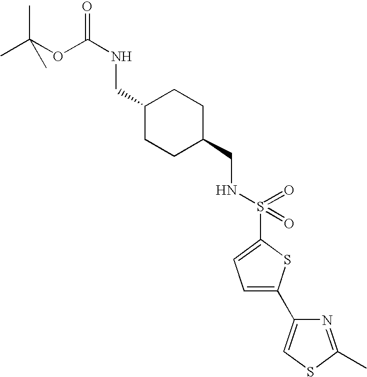

¹H NMR (500 MHz, DMSO-d₆) δ 8.03 (s, 1H), 7.79 (t, 1H), 7.56 (d, 1H), 7.51 (d, 1H), 6.75 (s, 1H), 2.72 (t, 2H), 2.70-2.65 (m, 5H), 1.73-1.58 (m, 4H), 1.35 (s, 9H), 1.32-1.26 (m, 1H), 1.26-1.20 (m, 1H), 0.84-0.70 (m, 4H); m/z 486.0 (M+H)⁺.

### Example 71

### tert-Butyl {[trans-4-({[(5-acetamido-1-naphthyl)sulfonyl]amino}methyl)cyclohexyl]-methyl}carbamate

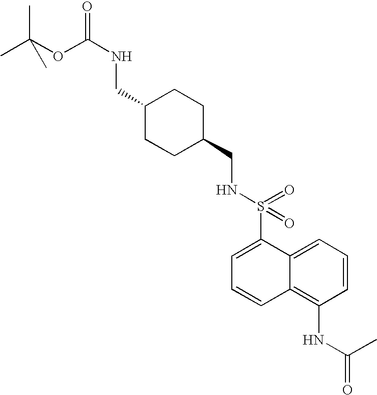

m/z 490.1 (M+H)⁺.

### Example 72

### tert-Butyl {[trans-4-({[(5-isoxazol-5-yl-2-thienyl)sulfonyl]amino}methyl)cyclo-hexyl]methyl}carbamate

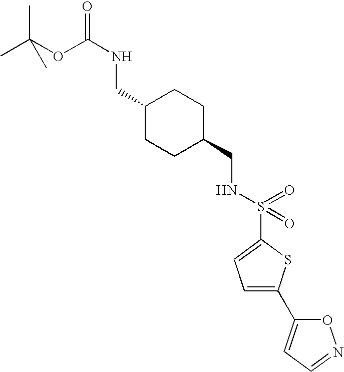

¹H NMR (500 MHz, DMSO-d₆) δ 8.73 (d, 1H), 8.03 (t, 1H), 7.74 (d, 1H), 7.65 (d, 1H), 7.10 (d, 1H), 6.76 (t, 1H), 2.76-2.69 (m, 4H), 1.73-1.62 (m, 4H), 1.36 (s, 9H), 1.34-1.28 (m, 1H), 1.28-1.20 (m, 1H), 0.86-0.72 (m, 4H); m/z 456.0 (M+H)⁺.

### Example 73

### tert-Butyl {[trans-4-({[(4-acetamidophenyl)sulfonyl]amino}methyl)cyclohexyl]-methyl}carbamate

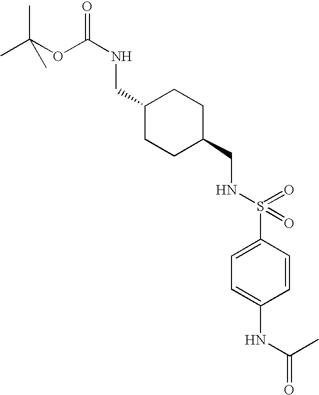

¹H NMR (500 MHz, DMSO-d₆) δ 7.73 (d, 2H), 7.68 (d, 2H), 7.39 (t, 1H), 6.74 (t, 1H), 2.71 (t, 2H), 2.53-2.46 (m, 2H), 2.07 (s, 3H), 1.70-1.58 (m, 4H), 1.35 (s, 9H), 1.28-1.16 (m, 2H), 0.78-0.68 (m, 4H); m/z 440.1 (M+H)⁺.

### Example 74

### tert-Butyl [(trans-4-{[(3-thienylsulfonyl)amino]methyl}cyclohexyl)methyl]-carbamate

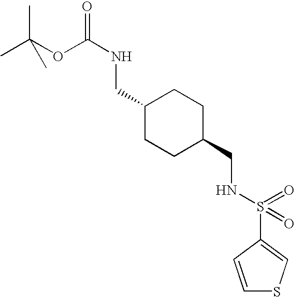

¹H NMR (500 MHz, DMSO-d₆) δ 8.11 (dd, 1H), 7.74 (dd, 1H), 7.52-7.48 (m, 1H), 7.31 (dd, 1H), 6.76 (t, 1H), 2.73 (t, 2H), 2.60 (t, 2H), 1.70-1.61 (m, 4H), 1.37 (s, 9H), 1.30-1.19 (m, 2H), 0.82-0.70 (m, 4H); m/z 387.2 (M−H)⁻.

### Example 75

### tert-Butyl ({trans-4-[({[3-(5-methyl-1,2,4-oxadiazol-3-yl)phenyl]sulfonyl}amino)-methyl]cyclohexyl}methyl)carbamate

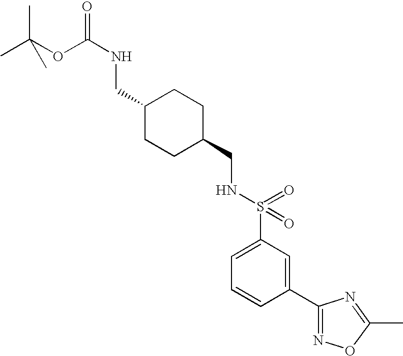

¹H NMR (500 MHz, DMSO-d₆) δ 8.37 (t, 1H), 8.22-8.17 (m, 1H), 7.97-7.93 (m, 1H), 7.78 (t, 1H), 6.74 (t, 1H), 2.72-2.67 (m, 4H), 2.63-2.55 (m, 3H), 1.70-1.57 (m, 4H), 1.34 (s, 9H), 1.29-1.15 (m, 2H), 0.80-0.68 (m, 4H); m/z 465.1 (M+H)⁺.

### Example 76

### tert-Butyl [(trans-4-{[(isoquinolin-5-ylsulfonyl)amino]methyl}cyclohexyl)-methyl]carbamate

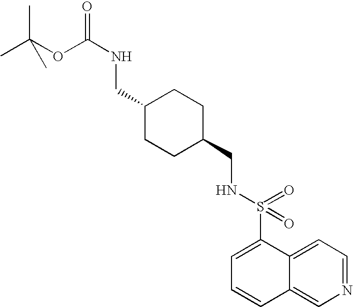

¹H NMR (500 MHz, DMSO-d₆) δ 9.44 (s, 1H), 8.68 (d, 1H), 8.44-8.40 (m, 2H), 8.3 (dd, 1H), 8.25 (m, 1H), 7.81 (t, 1H), 6.72 (m, 1H), 2.67 (t, 2H), 2.64-2.58 (m, 2H), 1.54 (d, 4H), 1.34 (s, 9H), 1.20-1.10 (m, 2H), 0.70-0.58 (m, 4H); m/z 434.1 (M+H)⁺.

### Example 77

### tert-Butyl {[trans-4-({[(4-acetamido-2-methyl-1,3-thiazol-5-yl)sulfonyl]amino}methyl)-cyclohexyl]methyl}carbamate

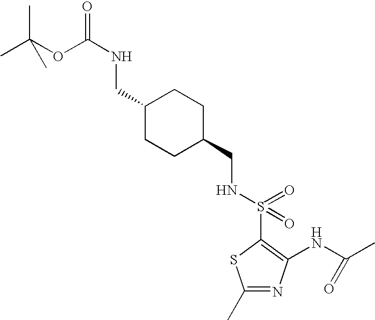

¹H NMR (500 MHz, DMSO-d₆) δ 7.78 (m, 1H), 6.78 (m, 1H), 2.75-2.72 (m, 2H), 2.65-2.62 (m, 2H), 2.42 (s, 3H), 2.15 (s, 3H), 1.71-1.66 (m, 4H), 1.35 (s, 9H), 1.31-1.20 (m, 2H), 0.8-0.72 (m, 4H); m/z 461.0 (M+H)⁺.

### Example 78

### tert-Butyl [(trans-4-{[(1,3-benzothiazol-6-ylsulfonyl)amino]methyl}cyclohexyl)-methyl]carbamate

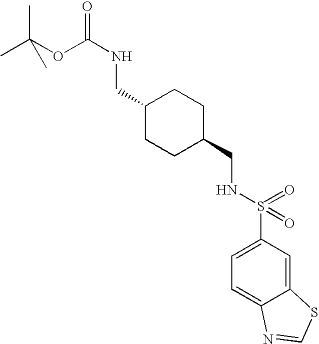

¹H NMR (500 MHz, DMSO-d₆) δ 9.61 (s, 1H), 8.68 (d, 1H), 8.27 (d, 1H), 7.91 (dd, 1H), 7.71-7.66 (m, 1H), 6.75 (t, 1H), 2.72 (t, 2H), 2.60 (d, 2H), 1.70-1.59 (m, 4H), 1.36 (s, 9H), 1.30-1.18 (m, 2H), 0.81-0.69 (m, 4H); m/z 440.0 (M+H)⁺.

### Example 79

### Method 7

### tert-Butyl [(trans-4-{[ethyl(2-naphthylsulfonyl)amino]methyl}cyclohexyl)methyl]-carbamate

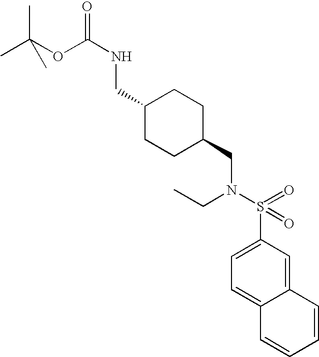

tert-Butyl [(trans-4-{[(2-naphthylsulfonyl)amino]methyl}cyclohexyl)methyl] carbamate [Intermediate DD, Step i)] (0.10 g, 0.23 mmol) was dissolved in a mixture of THF (2 mL) and DMF (2 mL) and treated with ethyl iodide (0.14 g, 1.2 mmol, 5.0 eq.) and K₂CO₃ (0.64 g, 9.2 mmol, 40 eq.). The reaction mixture was stirred at rt for 24 h. The reaction mixture was partitioned between water and DCM and the layers were separated and then the organic layer was washed with brine, dried (phase separator) and the solvent was concentrated in vacuo to leave a residue. The residue was dissolved in DMSO and purified by HPLC (Standard method E) to give the title compound (43 mg, 41%): ¹H NMR (500 MHz, DMSO-d₆) δ 8.48 (s, 1H), 8.18 (d, 1H), 8.11 (d, 1H), 8.03 (d, 1H), 7.89-7.77 (dd, 1H), 7.71-7.65 (m, 2H), 6.77 (t, 1H), 3.19-3.15 (dd, 2H), 2.95 (d, 2H), 2.75 (t, 2H), 1.76-1.61 (m, 4H), 1.55-1.43 (m, 1H), 1.36 (s, 9H), 1.32-1.21 (m, 1H), 1.00-0.94 (m, 3H), 0.89-0.75 (m, 4H); m/z 459.2 (M−H)⁻.

The Examples 80-87 were prepared using essentially the same conditions as described for Example 79 (Method 7) using the appropriate alkyl iodide in place of ethyl iodide.

### Example 80

### tert-Butyl [(trans-4-{[(2-naphthylsulfonyl)(2-phenylethyl)amino]methyl}cyclohexyl)-methyl]carbamate

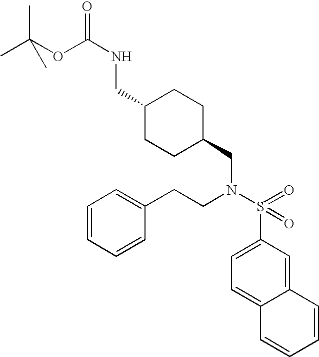

¹H NMR (500 MHz, DMSO-d₆) δ 8.50 (s, 1H), 8.22 (d, 1H), 8.13 (d, 1H), 8.05 (d, 1H), 7.81 (dd, 1H), 7.73-7.65 (m, 2H), 7.25 (t, 2H), 7.20-7.15 (m, 3H), 6.78-6.74 (m, 1H), 3.00-2.97 (m, 2H), 2.77-2.72 (m, 4H), 2.53-2.52 (m, 2H), 1.70-1.61 (m, 4H), 1.50-1.43 (m, 1H), 1.37 (s, 9H), 1.30-1.22 (m, 1H), 0.85-0.73 (m, 4H); m/z 535.8 (M−H)⁻.

### Example 81

### Ethyl N-[(trans-4-{[(tert-butoxycarbonyl)amino]methyl}cyclohexyl)methyl]-N-(2-naphthylsulfonyl)glycinate

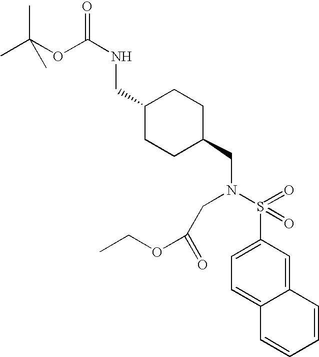

¹H NMR (500 MHz, DMSO-d₆) δ 8.47 (s, 1H), 8.18 (d, 1H), 8.12 (d, 1H), 8.05 (d, 1H), 7.81 (dd, 1H), 7.74-7.65 (m, 2H), 6.75 (t, 1H), 4.07 (s, 2H), 3.93 (q, 2H), 3.05 (d, 2H), 2.73 (t, 2H), 1.75-1.59 (m, 4H), 1.48-1.39 (m, 1H), 1.36 (s, 9H), 1.30-1.20 (m, 1H), 1.04 (t, 3H), 0.82-0.71 (m, 4H); m/z 519.4 (M+H)⁺.

### Example 82

### tert-Butyl [(trans-4-{[(2-naphthylsulfonyl)(tetrahydro-2H-pyran-4-ylmethyl)amino]-methyl}cyclohexyl)methyl]carbamate

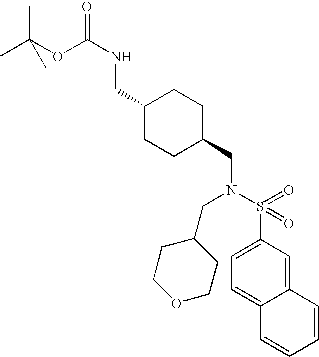

¹H NMR (500 MHz, DMSO-d₆) δ 8.47 (s, 1H), 8.20 (d, 1H), 8.11 (d, 1H), 8.04 (d, 1H), 7.81-7.78 (m, 1H), 7.70-7.66 (m, 2H), 6.76-6.72 (m, 1H), 3.81-3.77 (m, 2H), 3.23-3.17 (m, 2H), 2.97-2.90 (m, 4H), 2.74-2.71 (m, 2H), 1.84-1.79 (m, 1H), 1.68-1.59 (m, 4H), 1.54-1.48 (m, 3H), 1.35 (s, 9H), 1.25-1.20 (m, 1H), 1.15-1.04 (m, 2H), 0.82-0.72 (m, 4H); m/z 529.3 (M−H)⁻.

### Example 83

### tert-Butyl [(trans-4-{[(cyanomethyl)(2-naphthylsulfonyl)amino]methyl}cyclohexyl)-methyl]carbamate

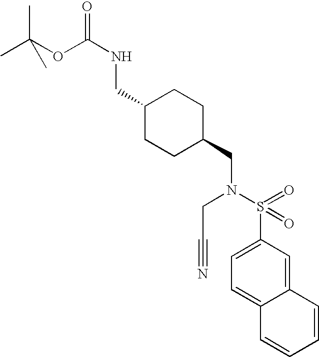

¹H NMR (500 MHz, DMSO-d₆) δ 8.55 (d, 1H), 8.19-8.15 (m, 2H), 8.06 (d, 1H), 7.85-7.83 (dd, 1H), 7.74-7.69 (m, 2H), 6.77 (3, 1H), 4.48 (s, 2H), 3.0 (d, 2H), 2.75 (t, 2H), 1.71-1.66 (m, 4H), 1.60-1.55 (m, 1H), 1.36 (s, 9H), 1.31-1.25 (m, 1H), 0.90-0.75 (m, 4H); m/z 470.5 (M−H)⁻.

### Example 84

### tert-Butyl [(trans-4-{[allyl(2-naphthylsulfonyl)amino]methyl}cyclohexyl)methyl]-carbamate

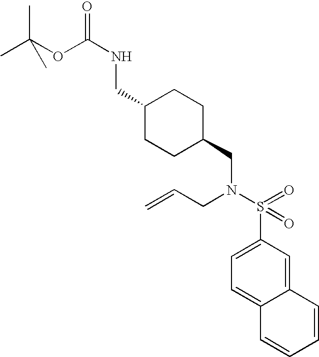

¹H NMR (500 MHz, DMSO-d₆) δ 8.48 (s, 1H), 8.18 (d, 1H), 8.12 (d, 1H), 8.05 (d, 1H), 7.81-7.79 (dd, 1H), 7.73 (m, 2H), 6.75 (t, 1H), 5.59-5.50 (m, 1H), 5.20-5.16 (dd, 1H), 5.07-5.05 (dd, 1H), 3.79 (d, 2H), 2.96 (d, 2H), 2.73 (t, 2H), 1.70-1.60 (m, 4H), 1.55-1.45 (m, 1H), 1.36 (s, 9H), 1.30-1.20 (m, 1H), 0.85-0.72 (m, 4H); m/z 471.1 (M−H)⁻.

### Example 85

### tert-Butyl [(trans-4-{[butyl(2-naphthylsulfonyl)amino]methyl}cyclohexyl)methyl]-carbamate

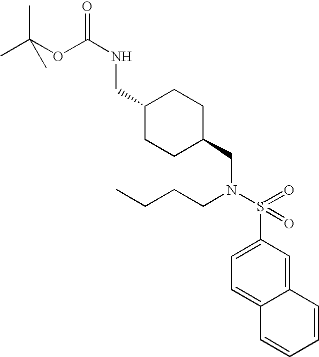

¹H NMR (500 MHz, DMSO-d₆) δ 8.45 (s, 1H), 8.19 (d, 1H), 8.11 (d, 1H), 8.04 (d, 1H), 7.78-7.77 (dd, 1H), 7.71-7.64 (m, 2H), 6.75 (t, 1H), 3.07 (t, 2H), 2.93 (d, 2H), 2.74 (t, 2H), 1.74-1.60 (m, 4H), 1.48 (s, 1H), 1.42-1.36 (m, 2H), 1.36 (s, 9H), 1.31-1.20 (m, 1H), 1.24-1.15 (m, 2H), 0.85-0.75 (m, 7H); m/z 487.2 (M−H)⁻.

### Example 86

### tert-Butyl [(trans-4-{[(2-naphthylsulfonyl)(2,2,2-trifluoroethyl)amino]methyl}cyclo-hexyl)methyl]carbamate

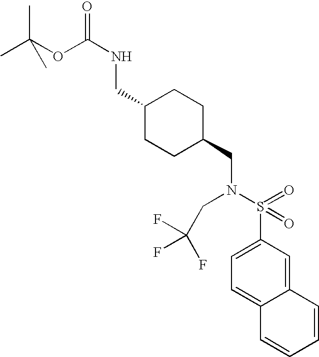

¹H NMR (500 MHz, DMSO-d₆) δ 8.59 (s, 1H), 8.21 (d, 1H), 8.15 (d, 1H), 8.07 (d, 1H), 7.88 (dd, 1H), 7.76-7.65 (m, 2H), 6.74 (t, 1H), 4.14 (q, 2H), 2.99 (d, 2H), 2.72 (t, 2H), 1.65-1.47 (m, 4H), 1.36 (s, 9H), 1.32-1.18 (m, 2H), 0.80-0.68 (m, 4H); m/z 513.1 (M−H)⁻.

### Example 87

### tert-Butyl [(trans-4-{[(2-naphthylsulfonyl)(propyl)amino]methyl}cyclohexyl)methyl]-carbamate

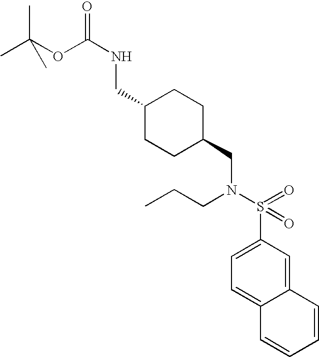

¹H NMR (500 MHz, DMSO-d₆) δ 8.45 (s, 1H), 8.19 (d, 1H), 8.11 (d, 1H), 8.04 (d, 1H), 7.78-7.77 (dd, 1H), 7.71-7.64 (m, 2H), 6.75 (t, 1H), 3.03 (t, 2H), 2.94 (d, 2H), 2.74 (t, 2H), 1.74-1.60 (m, 4H), 1.55-1.38 (m, 3H), 1.36 (s, 9H), 1.31-1.20 (m, 1H), 0.85-0.75 (m, 7H); m/z 473.3 (M−H)⁻.

### Example 88

### tert-Butyl [(trans-4-{[methyl(2-naphthylsulfonyl)amino]methyl}cyclohexyl)methyl]-carbamate

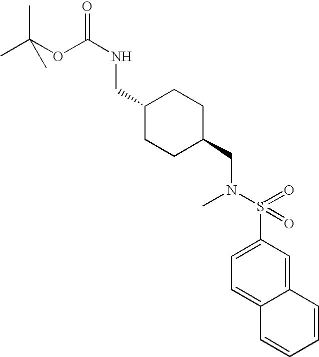

The title compound was prepared using essentially the same conditions as described for Example 79 (Method 7) with methyl iodide in place of ethyl iodide. The compound was purified by HPLC (Standard method A): ¹H NMR (500 MHz, DMSO-d₆) δ 8.43 (s, 1H), 8.19 (d, 1H), 8.13 (d, 1H), 8.05 (d, 1H), 7.76 (d, 1H), 7.69 (dt, 2H), 6.77 (t, 1H), 2.81-2.72 (m, 4H), 2.67 (s, 3H), 1.74-1.64 (m, 4H), 1.53-1.42 (m, 1H), 1.36 (s, 9H), 1.32-1.23 (m, 1H), 0.89-0.76 (m, 4H); m/z 391.1 (M-tert-butyl+H)⁺.

The following Examples 89-90 were prepared from tert-butyl ({trans-4-[({[5-(2-methyl-1,3-thiazol-4-yl)-2-thienyl]sulfonyl}amino)methyl]cyclohexyl}methyl)carbamate (Example 70) in place of tert-butyl [(trans-4-{[(2-naphthylsulfonyl)amino]methyl}cyclo-hexyl)methyl]carbamate and the appropriate alkyl iodide using essentially the same conditions as described for Example 79 (Method 7).

### Example 89

### tert-Butyl ({trans-4-[(methyl {[5-(2-methyl-1,3-thiazol-4-yl)-2-thienyl]sulfonyl}-amino)methyl]cyclohexyl}methyl)carbamate

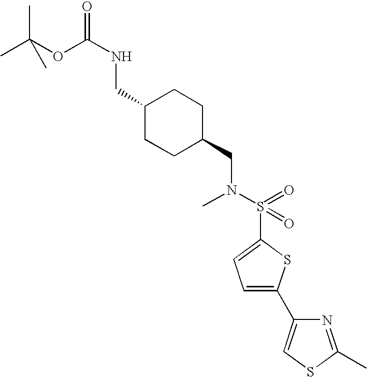

¹H NMR (500 MHz, DMSO-d₆) δ 8.09 (s, 1H), 7.66 (d, 1H), 7.61 (d, 1H), 6.78 (t, 1H), 2.80-2.74 (m, 4H), 2.70 (s, 6H), 1.75-1.67 (m, 4H), 1.55-1.48 (m, 1H), 1.37 (s, 9H), 1.33-1.26 (m, 1H), 0.91-0.79 (m, 4H); m/z 498.1 (M−H)⁻.

### Example 90

### tert-Butyl ({trans-4-[(ethyl {[5-(2-methyl-1,3-thiazol-4-yl)-2-thienyl]sulfonyl}amino)-methyl]cyclohexyl}methyl)carbamate

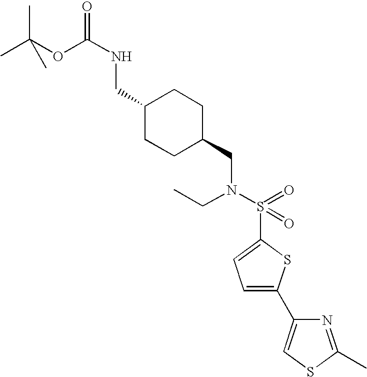

¹H NMR (500 MHz, DMSO-d₆) δ 8.08 (s, 1H), 7.63-7.60 (m, 2H), 6.78 (t, 1H), 3.17 (q, 2H), 2.91 (d, 2H), 2.77 (t, 2H), 2.70 (s, 3H), 1.79-1.65 (m, 4H), 1.57-1.48 (m, 1H), 1.37 (s, 9H), 1.34-1.25 (m, 1H), 1.05 (t, 3H), 0.90-0.79 (m, 4H); m/z 512.1 (M−H)⁻.

### Example 91

### Method 8

### tert-Butyl {[trans-4-({methyl[(2-phenylquinolin-4-yl)carbonyl]amino}methyl)cyclo-hexyl]methyl}carbamate

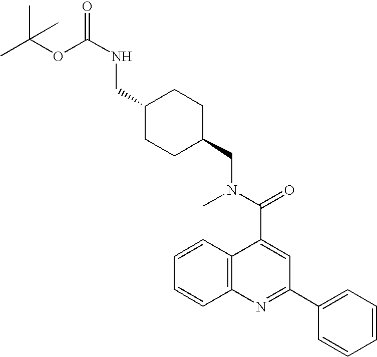

tert-Butyl {[trans-4-({[(2-phenylquinolin-4-yl)carbonyl]amino}methyl)cyclohexyl]-methyl}carbamate (Example 6) (25 mg, 0.053 mmol) was dissolved in DMF (2 mL) and treated with sodium hydride (2.1 mg, 0.053 mmol, 1.0 eq.), K₂CO₃ (36 mg, 0.26 mmol, 5.0 eq.) and methyl iodide (75 mg, 0.53 mmol, 10 eq.). The reaction mixture was diluted with DMSO (0.5 mL) and purified by HPLC (Standard method A) to give the title compound (20 mg, 78%). ¹H NMR (400 MHz, DMSO-d₆) (In this solvent at rt the molecule occurs in two rotameric forms, that are in slow exchange) δ 8.31-8.24 (m, 2H), 8.13-8.06 (m, 1H), 8.05-8.01 (m, 1H), 7.83-7.76 (m, 1H), 7.72-7.45 (m, 5H), 6.81-6.75 (m, 0.6H), 6.67-6.61 (m, 0.4H), 3.47-3.40 (m, 1H), 3.09 (s, 1.3H), 3.00-2.81 (m, 1H), 2.80-2.72 (m, 2.7H), 2.65-2.59 (m, 1H), 1.86-1.68 (m, 3H), 1.62-1.40 (m, 2H), 1.35 (s, 5H), 1.28 (s, 4H), 1.09-0.82 (m, 3H), 0.76-0.62 (m, 1H), 0.55-0.24 (m, 1H); m/z 488.2 (M+H)⁺.

### Example 92

### Method 9

### tert-Butyl [(trans-4-{[({2-[4-(aminomethyl)phenyl]quinolin-4-yl}carbonyl)amino]-methyl}cyclohexyl)methyl]carbamate acetate

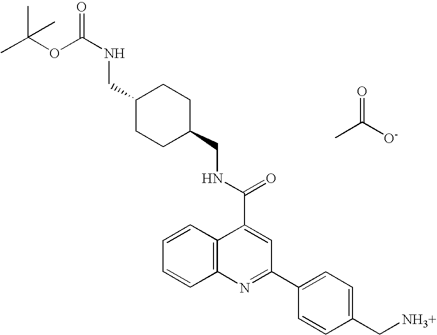

2-Chloroquinoline-4-carboxylic acid (0.15 g, 0.72 mmol), 4-(aminomethylphenyl) boronic acid (0.16 g, 0.87 mmol, 1.2 eq.) and Pd(PPh₃)₄ (42 mg, 0.036 mmol, 0.05 eq.) were added to a mixture of dioxane (2 mL) and a 1M aq. solution of K₂CO₃ (2 mL). The reaction mixture was degassed, sealed, and heated in the microwave at 140° C. for 15 min. The reaction mixture was cooled to 0° C. and a solution of (9-fluorenylmethyl)chloroformate (0.46 g, 1.8 mmol) in dioxane (1 mL) was added dropwise. The reaction mixture was warmed to rt and stirred for 36 h. The reaction mixture was filtered and the solid was washed with DMSO, water and MeOH to give 2-[4-({[(9H-fluoren-9-ylmeth-oxy)carbonyl]amino}methyl)phenyl]-quinoline-4-carboxylic acid (0.22 g, 60% over two steps) as a crude solid, which was used directly in the next step without further purification. m/z 501.3 (M+H)⁺.

The crude 2-[4-({[(9H-fluoren-9-ylmethoxy)carbonyl]-amino}methyl)phenyl] quinoline-4-carboxylic acid was reacted with tert-butyl {[trans-4-(aminomethyl)cyclohexyl]methyl}-carbamate using essentially the same procedure as described for Example 1 (Method 1) to give the crude 9H-fluoren-9-ylmethyl [4-(4-{[(trans-4-{[(tert-butoxycarbonyl)amino]methyl}cyclohexyl)methyl]carbamoyl}quinolin-2-yl)benzyl]carbamate, which was used in the next step with no further purification. m/z 725.4 (M+H)⁺.

The crude 9H-fluoren-9-ylmethyl [4-(4-{[(trans-4-{[(tert-butoxycarbonyl)amino]methyl}-cyclohexyl)methyl]carbamoyl}quinolin-2-yl)benzyl]carbamate (0.14 g, 0.19 mmol) was dissolved in DCM (4 mL) and treated with piperidine (1 mL). The reaction mixture was stirred at rt for 5 min. The reaction mixture was concentrated in vacuo to leave a residue. The residue was dissolved in DMSO and purified by HPLC (Standard method D) to give the title compound (35 mg, 32%). ¹H NMR (400 MHz, DMSO-d₆) δ 8.77 (t, 1H), 8.21 (d, 2H), 8.10-8.05 (m, 1H), 8.03 (s, 1H), 7.77 (t, 1H), 7.61-7.46 (m, 4H), 6.74 (t, 1H), 3.77 (s, 2H), 3.19 (t, 2H), 2.74 (t, 2H), 1.83 (s, 2H), 1.79 (d, 2H), 1.68 (d, 2H), 1.55-1.45 (m, 1H), 1.33 (s, 9H), 1.30-1.23 (m, 1H), 1.00-0.76 (m, 4H); m/z 503.4 (M+H)⁺.

### Example 93

### Method 10

4-(4-{[(trans-4-{[(tert-Butoxycarbonyl)amino]methyl}cyclohexyl)methyl]carbamoyl}-quinolin-2-yl)benzoic acid

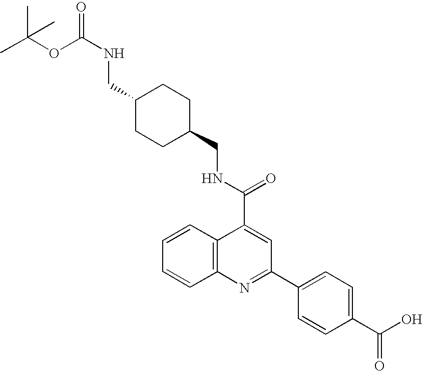

Methyl 4-(4-{[(trans-4-{[(tert-butoxycarbonyl)amino]methyl}cyclohexyl)methyl]-carbamoyl}quinolin-2-yl)benzoate [Intermediate NN] (12.5 mg, 0.024 mmol) was dissolved in THF (1 mL) and treated with a 1M aqueous solution of lithium hydroxide (0.5 mL). The reaction mixture was stirred at rt for 3 h and then concentrated in vacuo to leave a residue. The residue was dissolved in DMSO and purified by HPLC (Standard method A) to give the title compound (4.7 mg, 39%): ¹H NMR (400 MHz, DMSO-d₆) δ 13.07 (s, 1H), 8.83 (t, 1H), 8.44 (d, 2H), 8.18-8.11 (m, 4H), 7.88-7.82 (m, 1H), 7.71-7.65 (m, 1H), 6.79 (t, 1H), 3.62-3.57 (m, 1H), 3.24 (t, 2H), 2.78 (t, 2H), 1.88-1.81 (m, 2H), 1.79-1.69 (m, 2H), 1.60-1.50 (m, 1H), 1.37 (s, 9H), 1.35-1.27 (m, 1H), 1.04-0.81 (m, 4H); m/z 518.2 (M+H)⁺.

### Example 94

### Method 11

### tert-Butyl ({trans-4-[({[2-(1-oxidopyridin-4-yl)quinolin-4-yl]carbonyl}amino)methyl]-cyclohexyl}methyl)carbamate

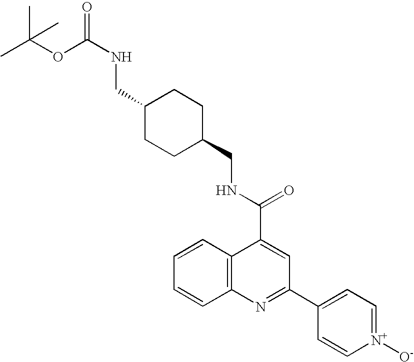

tert-Butyl {[trans-4-({[(2-pyridin-4-ylquinolin-4-yl)carbonyl]amino}methyl)cyclohexyl]-methyl}carbamate [Example 7] (50 mg, 0.10 mmol) was dissolved in DCM (5 mL), cooled to 0° C. and treated with MCPBA (20 mg, 0.12 mmol, 1.1 eq.). The reaction mixture was warmed to rt with stirring overnight. The reaction mixture was concentrated in vacuo to leave a residue which was dissolved in DMSO and purified by HPLC (Standard method A) to give the title compound (26 mg, 50%): ¹H NMR (600 MHz, DMSO-d₆) δ 8.79 (t, 1H), 8.36 (d, 2H), 8.33 (d, 2H), 8.19 (s, 1H), 8.11 (m, 2H), 7.82 (m, 1H), 7.66 (td, 1H), 6.78 (t, 1H), 3.21 (t, 2H), 2.75 (t, 2H), 1.82 (d, 2H), 1.70 (d, 2H), 1.55-1.47 (m, 1H), 1.35 (s, 9H), 1.32-1.26 (m, 1H), 1.00-0.79 (m, 4H); m/z 491.3 (M+H)⁺.

### Example 95

### Method 12

### tert-Butyl {[trans-4-({[(2-phenylquinolin-4-yl)sulfonyl]amino}methyl)cyclohexyl]-methyl}carbamate

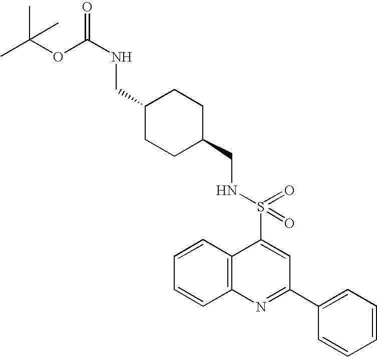

The title compound was prepared from 2-phenyl-quinoline-4-thiol [Intermediate OO] (200 mg, 0.84 mmol) and tert-butyl {[trans-4-(aminomethyl)cyclohexyl]methyl} carbamate (0.51 g, 2.1 mmol, 2.5 eq.) using the procedure described in Wright, S. W.; Hallstrom, K. N., J. Org. Chem., 2006, 71, p. 1080-1084. The crude product was purified by HPLC (Standard method B) to give the title compound (114 mg, 27%). ¹H NMR (400 MHz, DMSO-d₆) δ 8.62 (d, 1H), 8.36 (s, 1H), 8.31-8.15 (m, 4H), 7.88 (t, 1H), 7.74 (t, 1H), 7.61-7.50 (m, 3H), 6.69 (t, 1H), 2.76-2.69 (m, 2H), 2.65 (t, 2H), 1.57 (dd, 4H), 1.31 (s, 9H), 1.28-1.06 (m, 2H), 0.77-0.56 (m, 4H); m/z 510.8 (M+H)⁺.

### Example 96

### Method 13

### tert-Butyl ({trans-4-[(1-naphthoylamino)methyl]cyclohexyl}methyl)carbamate

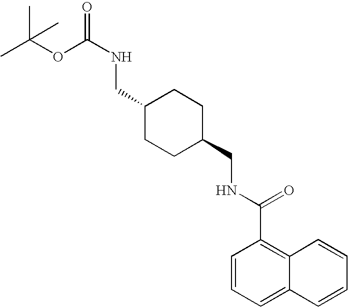

tert-Butyl {[trans-4-(aminomethyl)cyclohexyl]methyl}carbamate (60 mg, 0.25 mmol) was dissolved in DCM (5 mL) and treated with DIPEA (0.16 g, 1.2 mmol, 5.0 eq.). The mixture was cooled to 0° C. and then a solution of naphthalene-1-carbonyl chloride (57 mg, 0.3 mmol, 1.2 eq.) in DCM (0.5 mL) was added dropwise. The reaction mixture was stirred and warmed to rt for 45 min and then washed with a 1M aqueous solution of HCl and a saturated aqueous solution of NaHCO₃. The organic layer was dried (phase separator) and the solvent was concentrated in vacuo to give a residue. The residue was dissolved in DMSO and purified by HPLC (Standard method A) to give the title compound (49 mg, 50%). ¹H NMR (500 MHz, DMSO-d₆) δ 8.49 (t, 1H), 8.18-8.14 (m, 1H), 8.02-7.95 (m, 2H), 7.59-7.51 (m, 4H), 6.79 (t, 1H), 3.18 (t, 2H), 2.78 (t, 2H), 1.82 (d, 2H), 1.72 (d, 2H), 1.57-1.48 (m, 1H), 1.38 (s, 9H), 1.35-1.28 (m, 1H), 1.00-0.81 (m, 4H); m/z 341.1 (M-tert-butyl+H)⁺.

### Example 97

### Method 14

### N-[(trans-4-{[(tert-Butylcarbamoyl)amino]methyl}cyclohexyl)methyl]-2-pyridin-4-ylquinoline-4-carboxamide

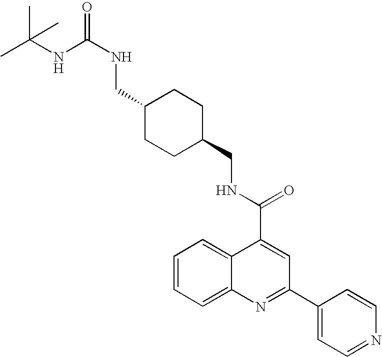

N-{[trans-4-(Aminomethyl)cyclohexyl]methyl}-2-pyridin-4-ylquinoline-4-carboxamide hydrochloride [Intermediate JJ] (30 mg, 0.080 mmol) was dissolved in a mixture of DCM (2 mL) and ACN (2 mL) and treated with TEA (12 mg, 0.12 mmol, 1.5 eq.) and tert-butyl isocyanate (9.5 mg, 0.096 mmol, 1.2 eq.). The reaction mixture was stirred at rt for 20 h and then concentrated in vacuo to leave a residue. The residue was dissolved in MeOH/water and purified by HPLC (Standard method A) to give the title compound (8.7 mg, 23%). ¹H NMR (400 MHz, DMSO-d₆) δ 8.84-8.71 (m, 3H), 8.26-8.07 (m, 5H), 7.83 (t, 1H), 7.68 (t, 1H), 5.66-5.57 (m, 1H), 5.50 (s, 1H), 3.23-3.10 (m, 2H), 2.81-2.74 (m, 2H), 1.81 (d, 2H), 1.68 (d, 2H), 1.56-1.45 (m, 1H), 1.28-1.20 (m, 1H), 1.16 (s, 9H), 1.03-0.77 (m, 4H); m/z 474.8 (M+H)⁺.

### Example 98

### Method 15

### tert-Butyl ({trans-4-[({[2-(3-{[(methylsulfonyl)amino]methyl}phenyl)quinolin-4-yl]carbonyl}amino)methyl]cyclohexyl}methyl)carbamate

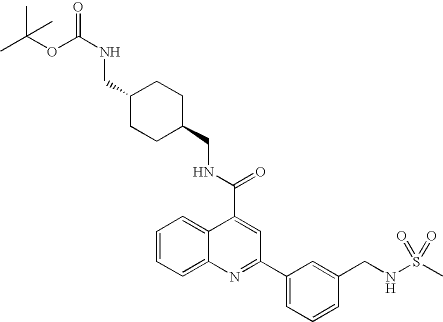

DIPEA (42 μL, 0.24 mmol, 2.0 eq.), and methanesulfonyl chloride (14 μL, 0.18 mmol, 1.5 eq.) were added to a solution of tert-butyl [(trans-4-{[({2-[3-(aminomethyl)phenyl]-quinolin-4-yl}carbonyl)amino]methyl}cyclohexyl)methyl]carbamate (Example 39) (60 mg, 0.12 mmol) in DCM (1 mL) and the reaction mixture was stirred for 16 h at rt. The reaction mixture was diluted with water, DCM, and MeOH and extracted with a mixture of DCM and MeOH. The organic layer was separated and concentrated in vacuo to leave a residue which was purified by flash column chromatography, using increasingly polar mixtures of DCM and EtOAc as eluent, to give the title compound (17 mg, 24%). ¹H NMR (400 MHz, CDCl₃) δ 8.10-8.01 (m, 2H), 7.98 (s, 1H), 7.91 (d, 1H), 7.78 (s, 1H), 7.66 (t, 1H), 7.48 (t, 1H), 7.44-7.34 (m, 2H), 4.27 (s, 2H), 3.82 (s, 2H), 3.28-3.21 (m, 3H), 2.86-2.80 (m, 2H), 2.78 (s, 3H), 1.78 (d, 2H), 1.70 (d, 2H), 1.57-1.45 (m, 1H), 1.31 (s, 10H), 1.02-0.79 (m, 4H); m/z 581.2 (M+H)⁺.

### Example 99

### Method 16

### tert-Butyl {[trans-4-({[(2-{3-[(carbamoylamino)methyl]phenyl}quinolin-4-yl)-carbonyl]amino}methyl)cyclohexyl]methyl}carbamate

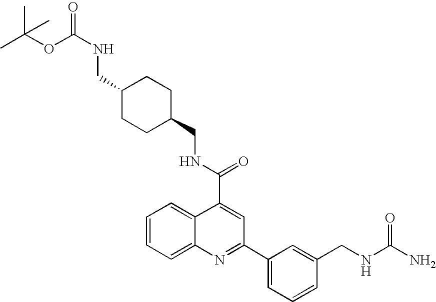

tert-Butyl [(trans-4-{[({2-[3-(aminomethyl)phenyl]quinolin-4-yl}carbonyl)amino]methyl}-cyclohexyl)methyl]carbamate (Example 39) (100 mg, 0.2 mmol) and potassium cyanate (24 mg, 0.3 mmol, 1.5 eq.) were dissolved in a solution of MeOH (2 mL) and AcOH (0.1 mL) and heated to 60° C. for 1 h. The reaction mixture was diluted with a small amount of MeOH and water to leave a precipitate which was collected by filtration. The precipitate was washed with a solution of MeOH and water and dried to give the title compound (115 mg, 95%). ¹H NMR (400 MHz, DMSO-d₆) δ 8.85-8.78 (m, 1H), 8.19 (s, 1H), 8.16-8.09 (m, 3H), 8.05 (s, 1H), 7.81 (t, 1H), 7.63 (t, 1H), 7.51 (t, 1H), 7.40 (d, 1H), 6.82-6.74 (m, 1H), 6.57-6.50 (m, 1H), 5.55 (s, 2H), 4.30 (d, 2H), 3.26-3.18 (m, 2H), 2.81-2.73 (m, 2H), 1.83 (d, 2H), 1.71 (d, 2H), 1.59-1.48 (m, 1H), 1.36 (s, 9H), 1.34-1.25 (m, 1H), 1.03-0.79 (m, 4H); m/z 546.2 (M+H)⁺.

### Example 100

### Method 17

### tert-Butyl [(trans-4-{[({2-[3-(acetamidomethyl)phenyl]quinolin-4-yl}carbonyl)amino]-methyl}cyclohexyl)methyl]carbamate

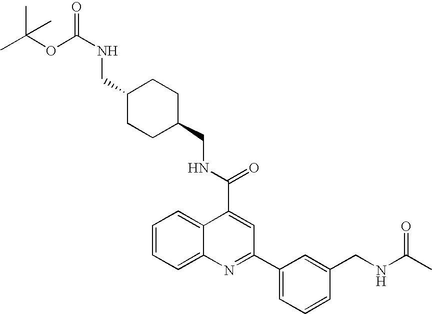

DIPEA (42 μL, 0.24 mmol, 2.0 eq.), DMAP (1 mg, 0.008 mmol, 0.07 eq.) and acetic acid anhydride (23 μL, 0.24 mmol, 2.0 eq.) were added to a solution of tert-butyl [(trans-4-{[({2-[3-(aminomethyl)phenyl]quinolin-4-yl}carbonyl)amino]methyl}cyclohexyl)-methyl]carbamate (Example 39) (60 mg, 0.12 mmol) in DCM (1 mL). The reaction mixture was stirred for 16 h at rt and then diluted with water and MeOH and then DCM and MeOH were added. The layers were separated and the organic layer was concentrated in vacuo to leave a residue. The residue was purified by flash column chromatography using increasingly polar mixtures of DCM and EtOAc as eluent to give the title compound (65 mg, 99%). ¹H NMR (400 MHz, CDCl₃) δ 8.03 (t, 2H), 7.89-7.80 (m, 2H), 7.74 (s, 1H), 7.67-7.60 (m, 1H), 7.46 (t, 1H), 7.35 (t, 1H), 7.28 (s, 1H), 4.34 (s, 2H), 3.90 (s, 3H), 3.22 (d, 2H), 2.80 (d, 2H), 1.89 (s, 3H), 1.76 (d, 2H), 1.68 (d, 2H), 1.55-1.43 (m, 1H), 1.29 (s, 10H), 1.00-0.77 (m, 4H); m/z 545.2 (M+H)⁺.

### Example 101

### Method 18

### Methyl [3-(4-{[(trans-4-{[(tert-butoxycarbonyl)amino]methyl}cyclohexyl)methyl]-carbamoyl}quinolin-2-yl)benzyl]carbamate

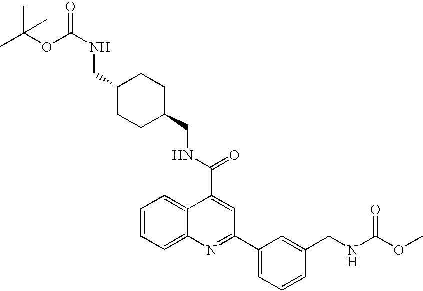

DIPEA (42 μL, 0.24 mmol, 2.0 eq.) and methyl chloroformate (23 mg, 0.24 mmol, 2.0 eq.) were added to a solution of tert-butyl [(trans-4-{[({2-[3-(aminomethyl)phenyl] quinolin-4-yl}carbonyl)amino]methyl}cyclohexyl)methyl]carbamate (Example 39) (60 mg, 0.12 mmol) in DCM (1 mL). The reaction mixture was stirred for 16 h at rt and then diluted with water and MeOH and a mixture of DCM and MeOH were added. The layers were separated and the organic phase was concentrated in vacuo to leave a residue. The residue was purified by flash column chromatography using increasingly polar mixtures of DCM and EtOAc as eluent to give the title compound (59 mg, 88%). ¹H NMR (400 MHz, CDCl₃) δ 8.18-8.07 (m, 2H), 8.00 (s, 1H), 7.95 (d, 1H), 7.78 (s, 1H), 7.72 (t, 1H), 7.52 (t, 1H), 7.43 (t, 1H), 7.39-7.33 (m, 1H), 6.54 (s, 1H), 5.32 (s, 1H), 4.64 (s, 1H), 4.45-4.37 (m, 2H), 3.68 (s, 3H), 3.36 (t, 2H), 2.94 (t, 2H), 1.89-1.72 (m, 4H), 1.63-1.49 (m, 1H), 1.42 (s, 10H), 1.09-0.83 (m, 4H); m/z 561.2 (M+H)⁺.

### Example 102

### tert-Butyl [(trans-4-{[({2-[4-(2-aminoethyl)phenyl]quinolin-4-yl}carbonyl)amino]-methyl}cyclohexyl)methyl]carbamate

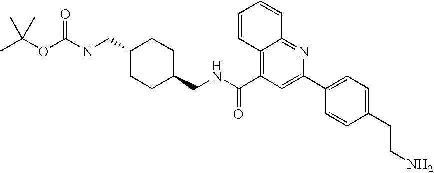

tert-Butyl {[trans-4-({[(2-chloroquinolin-4-yl)carbonyl]amino}methyl)cyclohexyl]-methyl}carbamate (Example 35) (200 mg, 0.46 mmol), [4-(2-aminoethyl)phenyl]-boronic acid hydrochloride (102 mg, 0.51 mmol), Pd(PPh₃)₄ (26 mg, 0.023 mmol), K₂CO₃ (198 mg, 1.43 mmol), water (2 mL), and dioxane (2 mL) were added to a vial and degassed. The reaction mixture was heated at 60° C. for 16 h under a nitrogen atmosphere and then cooled to rt. Water was added and the mixture was filtered. The collected solid was purified with cation exchange resin (CBA) followed by preparative HPLC, using ACN/Water/0.2% AcOH as eluent, to give the title compound (182 mg, 76%). ¹H NMR (400 MHz, DMSO-d₆) δ 8.80 (t, 1H), 8.22 (d, 2H), 8.15-8.08 (m, 2H), 8.06 (s, 1H), 7.84-7.77 (m, 1H), 7.66-7.59 (m, 1H), 7.40 (d, 2H), 6.79 (t, 1H), 3.31 (s, 2H), 3.23 (t, 2H), 2.87-2.69 (m, 6H), 1.83 (d, 2H), 1.72 (d, 2H), 1.61-1.48 (m, 1H), 1.37 (s, 10H), 1.04-0.79 (m, 4H); m/z 530.2 (M+H)⁺.

### Example 103

### tert-Butyl [(trans-4-{[({2-[4-(acetamidomethyl)phenyl]quinolin-4-yl}carbonyl)-amino]methyl}cyclohexyl)methyl]carbamate

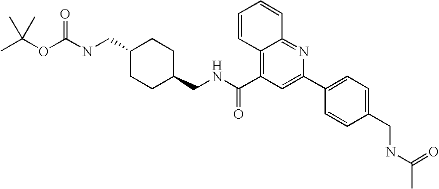

DIPEA (51 mg, 0.39 mmol), DMAP (2 mg) and acetic anhydride (40 mg, 0.40 mmol) were added sequentially to a solution of tert-butyl [(trans-4-{[({2-[4-(aminomethyl)-phenyl]quinolin-4-yl}carbonyl)amino]methyl}cyclohexyl)methyl]carbamate acetate (Example 92 (Method 9)) (100 mg, 0.2 mmol) in DCM (2 mL) and the reaction mixture was stirred at rt for 16 h. The reaction mixture was concentrated in vacuo to leave a residue, which was purified by preparative HPLC, using ACN/water/AcOH 0.2% as eluent, to give the title compound (51 mg, 47%). ¹H NMR (400 MHz, DMSO-d₆) δ 8.84-8.76 (m, 1H), 8.47-8.39 (m, 1H), 8.26 (d, 2H), 8.14-8.09 (m, 2H), 8.08 (s, 1H), 7.84-7.78 (m, 1H), 7.66-7.60 (m, 1H), 7.44 (d, 2H), 6.82-6.76 (m, 1H), 4.35 (d, 2H), 3.23 (t, 2H), 2.78 (t, 2H), 1.91 (s, 3H), 1.83 (d, 2H), 1.73 (d, 2H), 1.60-1.49 (m, 1H), 1.37 (s, 10H), 1.04-0.80 (m, 4H); m/z 545.2 (M+H)⁺.

### Example 104

### Methyl [4-(4-{[(trans-4-{[(tert-butoxycarbonyl)amino]methyl}cyclohexyl)methyl]-carbamoyl}quinolin-2-yl)benzyl]carbamate

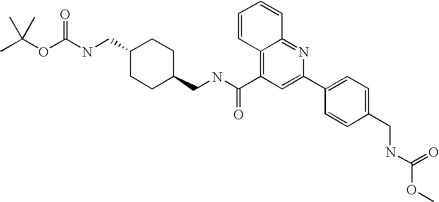

DIPEA (51 mg, 0.40 mmol), and methyl chloroformate (37 mg, 0.40 mmol) were added to a solution of tert-butyl [(trans-4-{[({2-[4-(aminomethyl)phenyl]quinolin-4-yl}carbonyl)amino]-methyl}cyclohexyl)methyl]carbamate acetate (Example 92 (Method 9)) (100 mg, 0.2 mmol) in DCM (2 mL) and the reaction mixture was stirred for 16 h at rt. The reaction mixture was then concentrated in vacuo to leave a residue which was purified by preparative HPLC, using ACN/water/AcOH 0.2% as eluent, to give the title compound (59 mg, 52%). ¹H NMR (400 MHz, DMSO-d₆) δ 8.77 (t, 1H), 8.21 (d, 2H), 8.11-8.05 (m, 2H), 8.03 (s, 1H), 7.77 (t, 1H), 7.59 (t, 2H), 7.49 (d, 2H), 6.77-6.71 (m, 1H), 3.77 (s, 2H), 3.23-3.16 (m, 2H), 2.78-2.68 (m, 2H), 1.87-1.61 (m, 7H), 1.56-1.41 (m, 1H), 1.32 (s, 10H), 1.01-0.74 (m, 4H); m/z 561.2 (M+H)⁺.

### Example 105

### tert-Butyl {[trans-4-({[(2-{4-[(carbamoylamino)methyl]phenyl}quinolin-4-yl)carbonyl]amino}methyl)cyclohexyl]methyl}carbamate

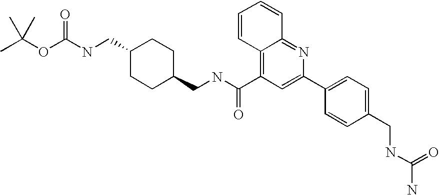

Potassium cyanate (48 mg, 0.60 mmol) was added to a solution of tert-butyl [(trans-4-{[({2-[4-(aminomethyl)phenyl]quinolin-4-yl}carbonyl)amino]-methyl}cyclohexyl)-methyl]carbamate (Example 92 (Method 9) (0.10 g, 0.20 mmol) in a mixture of AcOH (0.1 mL) and MeOH (3 mL) and the reaction mixture was heated at 60° C. for 15 h. The reaction mixture was cooled to rt and water was added. The mixture was filtered and the solid was dissolved in DMSO and purified by preparative HPLC, using ACN/water/AcOH 0.2% as eluent, to give the title compound (23 mg, 21%). ¹H NMR (400 MHz, CDCl₃) δ 8.08 (d, 2H), 7.98 (d, 2H), 7.79 (s, 1H), 7.70 (t, 1H), 7.52 (t, 1H), 7.36 (d, 1H), 5.39-5.29 (m, 1H), 4.17 (s, 2H), 3.32-3.26 (m, 2H), 2.92-2.84 (m, 2H), 1.89-1.71 (m, 4H), 1.63-1.51 (m, 1H), 1.39-1.32 (m, 1H), 1.38 (s, 9H), 1.07-0.84 (m, 4H); m/z 546.3 (M+H)⁺.

### Example 106

### tert-Butyl ({trans-4-[({[2-(4-{[(methylsulfonyl)amino]methyl}phenyl)quinolin-4-yl]carbonyl}amino)methyl]cyclohexyl}methyl)carbamate

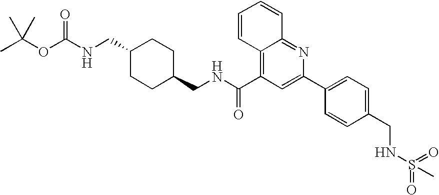

Methanesulfonyl chloride (46 mg, 0.40 mmol) and DIPEA (51 mg, 0.40 mmol) were added sequentially to a solution of tert-butyl [(trans-4-{[({2-[4 (aminomethyl)-phenyl]quinolin-4-yl}carbonyl)amino]methyl}cyclohexyl)methyl]carbamate (Example 92 (Method 9)) (0.10 g, 0.20 mmol) in DCM (2 mL) and the reaction mixture was stirred at rt for 15 h. The reaction mixture was then concentrated in vacuo to leave a residue, which was dissolved in DMSO and purified by preparative HPLC, using ACN/water/AcOH 0.2% as eluent, to give the title compound (5 mg, 4%). ¹H NMR (400 MHz, CDCl₃) δ 8.10 (t, 2H), 8.04 (d, 2H), 7.80 (s, 1H), 7.71 (t, 1H), 7.53 (t, 1H), 7.45 (d, 2H), 7.13 (t, 1H), 4.31 (s, 2H), 3.37-3.30 (m, 2H), 2.94 (s, 3H), 2.92-2.86 (m, 2H), 1.89-1.72 (m, 4H), 1.62-1.50 (m, 1H), 1.38 (s, 9H), 1.38-1.29 (m, 1H), 1.08-0.84 (m, 4H); m/z 581.2 (M+H)⁺.

### Example 107

### [4-(4-{[(trans-4-{[(tert-Butoxycarbonyl)amino]methyl}cyclohexyl)methyl]-carbamoyl}quinolin-2-yl)phenyl]acetic Acid

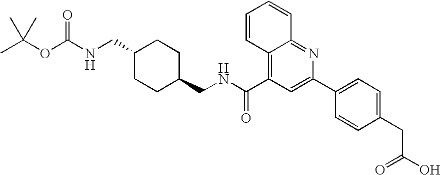

tert-Butyl {[trans-4-({[(2-chloroquinolin-4-yl)carbonyl]amino}methyl)cyclohexyl]-methyl}carbamate (Example 35) (150 mg, 0.34 mmol), [4-(4,4,5,5-tetramethyl-1,3,2-dioxaborolan-2-yl)phenyl]acetic acid (100 mg, 0.38 mmol), Pd(PPh₃)₄ (20 mg, 0.017 mmol), K₂CO₃ (148 mg, 1.07 mmol), water (2 mL) and dioxane (2 mL) were added to a vial and the vial was degassed. The reaction mixture was heated at 60° C. for 16 h under a nitrogen atmosphere and then cooled to rt. DCM, MeOH and a mixture of water and citric acid were added and the layers were separated. The organic layer was concentrated in vacuo to leave a solid, which was washed with MeOH to give the title compound (166 mg, 89%). ¹H NMR (400 MHz, DMSO-d₆) δ 12.40 (s, 1H), 8.81 (t, 1H), 8.25 (d, 2H), 8.15-8.09 (m, 2H), 8.08 (s, 1H), 7.84-7.78 (m, 1H), 7.66-7.61 (m, 1H), 7.46 (d, 2H), 6.79 (t, 1H), 3.68 (s, 2H), 3.23 (t, 2H), 2.78 (t, 2H), 1.84 (d, 2H), 1.73 (d, 2H), 1.61-1.48 (m, 1H), 1.37 (s, 10H), 1.04-0.80 (m, 4H); m/z 532.2 (M+H)⁺.

### Example 108

### 3-(4-{[(trans-4-{[(tert-Butoxycarbonyl)amino]methyl}cyclohexyl)methyl]-carbamoyl}quinolin-2-yl)benzoic Acid

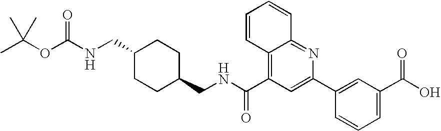

The title compound was prepared as described in Example 107 using 3-(dihydroxyboryl)benzoic acid in place of [4-(4,4,5,5-tetramethyl-1,3,2-dioxaborolan-2-yl)phenyl]acetic acid. ¹H NMR (400 MHz, DMSO-d₆) δ 13.19 (s, 1H), 8.89-8.80 (m, 2H), 8.52 (d, 1H), 8.20-8.13 (m, 3H), 8.09 (d, 1H), 7.87-7.80 (m, 1H), 7.73-7.64 (m, 2H), 6.83-6.75 (m, 1H), 3.24 (t, 2H), 2.78 (t, 2H), 1.89-1.67 (m, 4H), 1.60-1.48 (m, 1H), 1.37 (s, 10H), 1.05-0.79 (m, 4H); m/z 518.2 (M+H)⁺.

### Example 109

### Method 19

### Tetrahydro-2H-pyran-4-yl [(trans-4-{[({2-[6-(4-methylpiperazin-1-yl)pyridin-3-yl]quinolin-4-yl}carbonyl)amino]methyl}cyclohexyl)methyl]carbamate

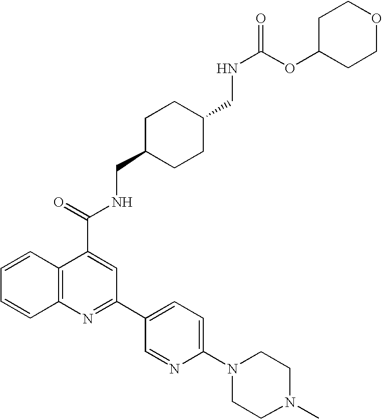

4-Nitrophenyl tetrahydro-2H-pyran-4-yl carbonate (Intermediate QQ) (81 mg, 0.30 mmol, 1.2 eq.) and TEA (0.32 mL, 2.3 mmol, 9.2 eq.) were added sequentially to a stirred solution of N-{[trans-4-(aminomethyl)cyclohexyl]methyl}-2-[6-(4-methylpiperazin-1-yl)pyridin-3-yl]quinoline-4-carboxamide (Intermediate PP) (120 mg, 0.25 mmol) in THF (2 mL) and the reaction mixture was stirred at rt for 12 h. The reaction mixture was concentrated in vacuo to leave a residue which was dissolved in DMSO and purified by reverse phase HPLC (Standard method A) to afford the title compound (16 mg, 11%). ¹H NMR (300 MHz, CDCl₃) δ 8.88 (d, 1H), 8.35 (dd, 1H), 8.13-8.06 (m, 2H), 7.76 (s, 1H), 7.75-7.67 (m, 1H), 7.54-7.47 (m, 1H), 6.75 (d, 1H), 6.31-6.21 (m, 1H), 4.89-4.71 (m, 2H), 3.95-3.83 (m, 2H), 3.74-3.64 (m, 4H), 3.58-3.46 (m, 2H), 3.45-3.38 (m, 2H), 3.10-2.98 (m, 2H), 2.59-2.49 (m, 4H), 2.37 (s, 3H), 1.99-1.78 (m, 6H), 1.73-1.55 (m, 3H), 1.53-1.37 (m, 1H), 1.18-0.94 (m, 4H); m/z (M+H)⁺ 601.3.

### Examples 110-132

The following Examples 110-132 were prepared by the general procedure of Example 109 (Method 19) by using the appropriate Intermediate RR-OOO in place of Intermediate QQ.

| | | | MS |
|---|---|---|---|
| | | ¹H NMR (600 MHz, | m/z |
| Example | Name | DMSO/DMSO-d₆)* δ | (M + H)⁺ |
| 110 | 1-Methylpiperidin-4-yl- | 9.01 (d, 1H), 8.74 (t, 1H), 8.41 (dd, | 614.3 |
| | [(trans-4-{[({2-[6-(4- | 1H), 8.06-7.99 (m, 3H), | |
| | methylpiperazin-1- | 7.76-7.72 (m, 1H), 7.56-7.52 (m, 1H), 7.04 (t, | |
| | yl)pyridin-3-yl]quinolin-4- | 1H), 6.97 (d, 1H), 4.47-4.35 (m, 1H), | |
| | yl}carbonyl)amino]methyl}cyclohexyl)methyl]carbamate | 3.62-3.58 (m, 4H), 3.21-3.17 (m, | |
| | | 2H), 2.82-2.78 (m, 2H), | |
| | | 2.41-2.38 (m, 4H), 2.21-2.20 (m, 3H), 2.13 (s, | |
| | | 3H), 1.84-1.66 (m, 6H), | |
| | | 1.53-1.44 (m, 3H), 1.35-1.27 (m, 1H), | |
| | | 0.98-0.80 (m, 4H) | |
| 111 | Oxetan-2-ylmethyl- | 9.01 (d, 1H), 8.74 (t, 1H), 8.42 (dd, | 587.2 |
| | [(trans-4-{[({2-[6-(4- | 1H), 8.06-7.99 (m, 3H), | |
| | methylpiperazin-1- | 7.75-7.72 (m, 1H), 7.56-7.52 (m, 1H), 7.24 (t, | |
| | yl)pyridin-3-yl]quinolin-4- | 1H), 6.97 (d, 1H), 4.81-4.76 (m, 1H), | |
| | yl}carbonyl)amino]methyl}cyclohexyl)methyl]carbamate | 4.48-4.42 (m, 2H), 4.41-4.36 (m, | |
| | | 2H), 4.09-3.98 (m, 2H), 3.60 (s, 4H), | |
| | | 3.19 (t, 2H), 2.82 (t, 2H), | |
| | | 2.39-2.32 (m, 4H), 2.21 (s, 3H), 1.86-1.79 (m, | |
| | | 2H), 1.75-1.68 (m, 2H), | |
| | | 1.56-1.48 (m, 1H), 1.38-1.29 (m, 1H), | |
| | | 1.00-0.82 (m, 4H) | |
| 112 | (1S)-2-Methoxy-1- | 9.01 (d, 1H), 8.76-8.72 (m, 1H), | 589.3 |
| | methylethyl[(trans-4- | 8.42 (dd, 1H), 8.06-7.99 (m, 3H), | |
| | {[({2-[6-(4- | 7.76-7.71 (m, 1H), 7.57-7.52 (m, 1H), | |
| | methylpiperazin-1- | 7.07 (t, 1H), 6.97 (d, 1H), | |
| | yl)pyridin-3-yl]quinolin-4- | 4.79-4.71 (m, 1H), 3.60 (s, 4H), 3.22 (s, 3H), | |
| | yl}carbonyl)amino]methyl}cyclohexyl)methyl]carbamate | 3.20-3.17 (m, 2H), 2.82-2.78 (m, | |
| | | 2H), 2.42-2.39 (m, 4H), 2.22 (s, 3H), | |
| | | 1.84-1.77 (m, 2H), 1.72-1.66 (m, | |
| | | 2H), 1.56-1.46 (m, 1H), | |
| | | 1.36-1.27 (m, 1H), 1.08 (d, 3H), 0.98-0.81 (m, | |
| | | 4H) | |
| 113 | (1R)-2-Methoxy-1- | 9.01 (d, 1H), 8.76-8.72 (m, 1H), | 589.3 |
| | methylethyl[(trans-4- | 8.42 (dd, 1H), 8.06-7.99 (m, 3H), | |
| | {[({2-[6-(4- | 7.76-7.71 (m, 1H), 7.57-7.52 (m, 1H), | |
| | methylpiperazin-1- | 7.07 (t, 1H), 6.97 (d, 1H), | |
| | yl)pyridin-3-yl]quinolin-4- | 4.79-4.71 (m, 1H), 3.60 (s, 4H), 3.22 (s, 3H), | |
| | yl}carbonyl)amino]methyl}cyclohexyl)methyl]carbamate | 3.20-3.17 (m, 2H), 2.82-2.78 (m, | |
| | | 2H), 2.42-2.39 (m, 4H), 2.22 (s, 3H), | |
| | | 1.84-1.77 (m, 2H), 1.72-1.66 (m, | |
| | | 2H), 1.56-1.46 (m, 1H), | |
| | | 1.36-1.27 (m, 1H), 1.08 (d, 3H), 0.98-0.81 (m, | |
| | | 4H) | |
| 114 | Tetrahydrofuran-3-yl- | 9.01 (d, 1H), 8.77-8.72 (m, 1H), | 587.2 |
| | [(trans-4-{[({2-[6-(4- | 8.42 (dd, 1H), 8.06-7.98 (m, 3H), | |
| | methylpiperazin-1- | 7.76-7.72 (m, 1H), 7.57-7.52 (m, 1H), | |
| | yl)pyridin-3-yl]quinolin-4- | 7.17 (t, 1H), 6.97 (d, 1H), | |
| | yl}carbonyl)amino]methyl}cyclohexyl)methyl]carbamate | 5.08-5.03 (m, 1H), 3.76-3.64 (m, 4H), | |
| | | 3.63-3.56 (m, 4H), 3.21-3.17 (m, 2H), | |
| | | 2.83-2.78 (m, 2H), 2.21 (s, 3H), | |
| | | 2.09-2.01 (m, 2H), 1.84-1.77 (m, | |
| | | 2H), 1.72-1.66 (m, 2H), | |
| | | 1.56-1.46 (m, 1H), 1.35-1.27 (m, 1H), | |
| | | 0.98-0.80 (m, 4H) | |
| 115 | 2-(2-Oxopyrrolidin-1- | 9.01 (d, 1H), 8.77-8.73 (m, 1H), | 627.4 |
| | yl)ethyl[(trans-4-{[({2-[6- | 8.41 (dd, 1H), 8.06-7.99 (m, 3H), | |
| | (4-methylpiperazin-1- | 7.76-7.72 (m, 1H), 7.57-7.52 (m, 1H), | |
| | yl)pyridin-3-yl]quinolin-4- | 7.14 (t, 1H), 6.97 (d, 1H), 4.00 (t, | |
| | yl}carbonyl)amino]methyl}cyclohexyl)methyl]carbamate | 2H), 3.62-3.57 (m, 4H), | |
| | | 3.21-3.17 (m, 2H), 2.82-2.78 (m, 2H), | |
| | | 2.41-2.37 (m, 4H), 2.20 (s, 3H), | |
| | | 2.17-2.13 (m, 2H), 1.89-1.83 (m, 2H), | |
| | | 1.83-1.78 (m, 2H), 1.72-1.66 (m, 2H), | |
| | | 1.55-1.47 (m, 1H), 1.36-1.27 (m, | |
| | | 1H), 0.98-0.80 (m, 4H) | |
| 116 | (3S)-Tetrahydrofuran-3-yl- | 9.01 (d, 1H), 8.77-8.72 (m, 1H), | 587.2 |
| | [(trans-4-{[({2-[6-(4- | 8.42 (dd, 1H), 8.06-7.98 (m, 3H), | |
| | methylpiperazin-1- | 7.76-7.72 (m, 1H), 7.57-7.52 (m, 1H), | |
| | yl)pyridin-3-yl]quinolin-4- | 7.17 (t, 1H), 6.97 (d, 1H), | |
| | yl}carbonyl)amino]methyl}cyclohexyl)methyl]carbamate | 5.08-5.03 (m, 1H), 3.76-3.64 (m, 4H), | |
| | | 3.63-3.56 (m, 4H), 3.21-3.17 (m, 2H), | |
| | | 2.83-2.78 (m, 2H), 2.21 (s, 3H), | |
| | | 2.09-2.01 (m, 2H), 1.84-1.77 (m, | |
| | | 2H), 1.72-1.66 (m, 2H), | |
| | | 1.56-1.46 (m, 1H), 1.35-1.27 (m, 1H), | |
| | | 0.98-0.80 (m, 4H) | |
| 117 | 2,2-Difluoroethyl[(trans- | 9.01 (d, 1H), 8.77-8.72 (m, 1H), | 581.2 |
| | 4-{[({2-[6-(4- | 8.41 (dd, 1H), 8.06-7.99 (m, 3H), | |
| | methylpiperazin-1- | 7.76-7.72 (m, 1H), 7.57-7.52 (m, 1H), | |
| | yl)pyridin-3-yl]quinolin-4- | 7.48-7.43 (m, 1H), 6.97 (d, 1H), | |
| | yl}carbonyl)amino]methyl}cyclohexyl)methyl]carbamate | 6.28-6.05 (m, 1H), 4.23-4.13 (m, | |
| | | 2H), 3.63-3.54 (m, 4H), | |
| | | 3.21-3.17 (m, 2H), 2.86-2.82 (m, 2H), | |
| | | 2.41-2.38 (m, 4H), 2.20 (s, 3H), | |
| | | 1.84-1.78 (m, 2H), 1.73-1.67 (m, 2H), | |
| | | 1.56-1.46 (m, 1H), 1.37-1.30 (m, 1H), | |
| | | 0.99-0.81 (m, 4H) | |
| 118 | 2-Fluoroethyl[(trans-4- | 9.01 (d, 1H), 8.77-8.72 (m, 1H), | 563.2 |
| | {[({2-[6-(4- | 8.41 (dd, 1H), 8.06-7.99 (m, 3H), | |
| | methylpiperazin-1- | 7.76-7.72 (m, 1H), 7.57-7.52 (m, 1H), | |
| | yl)pyridin-3-yl]quinolin-4- | 7.27 (t, 1H), 6.97 (d, 1H), | |
| | yl}carbonyl)amino]methyl}cyclohexyl)methyl]carbamate | 4.59-4.56 (m, 1H), 4.51-4.47 (m, 1H), | |
| | | 4.19-4.15 (m, 1H), 4.13-4.10 (m, 1H), | |
| | | 3.63-3.57 (m, 4H), 3.21-3.17 (m, | |
| | | 2H), 2.85-2.80 (m, 2H), | |
| | | 2.42-2.38 (m, 4H), 2.21 (s, 3H), 1.84-1.78 (m, | |
| | | 2H), 1.73-1.67 (m, 2H), | |
| | | 1.56-1.47 (m, 1H), 1.36-1.29 (m, 1H), | |
| | | 0.98-0.80 (m, 4H) | |
| 119 | Ethyl[(trans-4-{[({2-[6- | 9.01 (d, 1H), 8.77-8.72 (m, 1H), | 545.2 |
| | (4-methylpiperazin-1- | 8.42 (dd, 1H), 8.06-7.99 (m, 3H), | |
| | yl)pyridin-3-yl]quinolin-4- | 7.76-7.72 (m, 1H), 7.57-7.52 (m, 1H), | |
| | yl}carbonyl)amino]methyl}cyclohexyl)methyl]carbamate | 7.04 (t, 1H), 6.97 (d, 1H), 3.93 (q, | |
| | | 2H), 3.62-3.58 (m, 4H), | |
| | | 3.20-3.17 (m, 2H), 2.82-2.79 (m, 2H), | |
| | | 2.42-2.38 (m, 4H), 2.21 (s, 3H), | |
| | | 1.84-1.77 (m, 2H), 1.73-1.66 (m, 2H), | |
| | | 1.56-1.47 (m, 1H), 1.36-1.27 (m, 1H), | |
| | | 1.11 (t, 3H), 0.98-0.80 (m, 4H) | |
| 120 | 2-Methoxyethyl[(trans-4- | 9.01 (d, 1H), 8.77-8.72 (m, 1H), | 574.3 |
| | {[({2-[6-(4- | 8.41 (dd, 1H), 8.06-7.99 (m, 3H), | |
| | methylpiperazin-1- | 7.76-7.71 (m, 1H), 7.56-7.52 (m, 1H), | |
| | yl)pyridin-3-yl]quinolin-4- | 7.17 (t, 1H), 6.97 (d, 1H), | |
| | yl}carbonyl)amino]methyl}cyclohexyl)methyl]carbamate | 4.02-3.99 (m, 2H), 3.62-3.58 (m, 4H), | |
| | | 3.46-3.42 (m, 2H), 3.21 (s, 3H), | |
| | | 3.20-3.17 (m, 2H), 2.82-2.79 (m, 2H), | |
| | | 2.42-2.38 (m, 4H), 2.21 (s, 3H), | |
| | | 1.84-1.78 (m, 2H), 1.73-1.67 (m, 2H), | |
| | | 1.55-1.46 (m, 1H), 1.35-1.27 (m, 1H), | |
| | | 0.98-0.79 (m, 4H) | |
| 121 | 1-Cyanoethyl[(trans-4- | 9.01 (d, 1H), 8.76-8.73 (m, 1H), | 569.3 |
| | {[({2-[6-(4- | 8.43-8.40 (m, 1H), 8.05-8.01 (m, | |
| | methylpiperazin-1- | 3H), 7.75-7.72 (m, 1H), | |
| | yl)pyridin-3-yl]quinolin-4- | 7.61-7.52 (m, 2H), 6.97 (d, 1H), 5.32 (m, 1H), | |
| | yl}carbonyl)amino]methyl}cyclohexyl)methyl]carbamate | 3.62-3.58 (m, 4H), 3.20-3.17 (m, | |
| | | 2H), 2.86-2.83 (m, 2H), | |
| | | 2.41-2.38 (m, 4H), 2.21 (s, 3H), 1.84-1.79 (m, | |
| | | 2H), 1.73-1.68 (m, 2H), | |
| | | 1.53-1.51 (m, 1H), 1.49 (d, 3H), 1.38-1.30 (m, | |
| | | 1H), 0.99-0.83 (m, 4H) | |
| 122 | 2-Acetamidoethyl[(trans- | 9.02-9.01 (m, 1H), 8.76-8.72 (m, | 601.3 |
| | 4-{[({2-[6-(4- | 1H), 8.43-8.40 (m, 1H), | |
| | methylpiperazin-1- | 8.06-8.01 (m, 3H), 7.91-7.87 (m, 1H), | |
| | yl)pyridin-3-yl]quinolin-4- | 7.75-7.72 (m, 1H), 7.56-7.52 (m, 1H), | |
| | yl}carbonyl)amino]methyl}cyclohexyl)methyl]carbamate | 7.12-7.09 (m, 1H), 6.97 (d, 1H), | |
| | | 3.91-3.88 (m, 2H), 3.62-3.57 (m, | |
| | | 4H), 3.20-3.17 (m, 4H), | |
| | | 2.82-2.79 (m, 2H), 2.39 (s, 4H), 2.23-2.20 (m, | |
| | | 3H), 1.83-1.78 (m, 2H), 1.76 (s, 3H), | |
| | | 1.73-1.68 (m, 2H), 1.54-1.47 (m, | |
| | | 1H), 1.35-1.28 (m, 1H), | |
| | | 0.98-0.80 (m, 4H) | |
| 123 | (3-Methyloxetan-3- | 9.02-9.00 (m, 1H), 8.76-8.73 (m, | 600.3 |
| | yl)methyl[(trans-4-{[({2- | 1H), 8.43-8.40 (m, 1H), | |
| | [6-(4-methylpiperazin-1- | 8.06-8.01 (m, 3H), 7.75-7.72 (m, 1H), | |
| | yl)pyridin-3-yl]quinolin-4- | 7.56-7.52 (m, 1H), 7.20-7.17 (m, 1H), | |
| | yl}carbonyl)amino]methyl}cyclohexyl)methyl]carbamate | 6.97 (d, 1H), 4.35 (d, 2H), 4.18 (d, | |
| | | 2H), 4.00 (s, 2H), 3.62-3.58 (m, 4H), | |
| | | 3.21-3.17 (m, 2H), 2.84-2.80 (m, | |
| | | 2H), 2.39 (s, 4H), 2.39 (s, 3H), | |
| | | 1.83-1.78 (m, 2H), 1.73-1.68 (m, 2H), | |
| | | 1.55-1.47 (m, 1H), 1.37-1.30 (m, | |
| | | 1H), 1.21 (s, 3H), 0.98-0.81 (m, 4H) | |
| 124 | (3R)-5-Oxopyrrolidin-3-yl- | 9.02-9.01 (m, 1H), 8.76-8.73 (m, | 599.3 |
| | [(trans-4-{[({2-[6-(4- | 1H), 8.42-8.40 (m, 1H), | |
| | methylpiperazin-1- | 8.05-8.01 (m, 3H), 7.75-7.72 (m, 1H), | |
| | yl)pyridin-3-yl]quinolin-4- | 7.67-7.65 (m, 1H), 7.56-7.53 (m, 1H), | |
| | yl}carbonyl)amino]methyl}cyclohexyl)methyl]carbamate | 7.26-7.23 (m, 1H), 6.97 (d, 1H), | |
| | | 5.11-5.07 (m, 1H), 3.62-3.58 (m, | |
| | | 4H), 3.55-3.51 (m, 2H), | |
| | | 3.20-3.17 (m, 2H), 3.11 (d, 1H), 2.82-2.79 (m, | |
| | | 2H), 2.41-2.38 (m, 4H), 2.20 (s, 3H), | |
| | | 1.82-1.78 (m, 2H), 1.71-1.67 (m, | |
| | | 2H), 1.53-1.47 (m, 1H), | |
| | | 1.35-1.27 (m, 1H), 0.98-0.79 (m, 4H) | |
| 125 | (3S)-5-Oxopyrrolidin-3-yl- | 9.02-9.01 (m, 1H), | 599.3 |
| | [(trans-4-{[({2-[6-(4- | 8.76-8.73 (m, 1H), | |
| | methylpiperazin-1- | 8.42-8.40 (m, 1H), 8.05-8.01 (m, | |
| | yl)pyridin-3-yl]quinolin-4- | 3H), 7.75-7.72 (m, 1H), | |
| | yl}carbonyl)amino]methyl}cyclohexyl)methyl]carbamate | 7.67-7.65 (m, 1H), | |
| | | 7.56-7.53 (m, 1H), 7.26-7.23 (m, | |
| | | 1H), 6.97 (d, 1H), | |
| | | 5.11-5.07 (m, 1H), 3.62-3.58 (m, | |
| | | 4H), 3.55-3.51 (m, 2H), | |
| | | 3.20-3.17 (m, 2H), | |
| | | 3.11 (d, 1H), 2.82-2.79 (m, 2H), | |
| | | 2.41-2.38 (m, 4H), 2.20 (s, | |
| | | 3H), 1.82-1.78 (m, 2H), | |
| | | 1.71-1.67 (m, 2H), | |
| | | 1.53-1.47 (m, 1H), | |
| | | 1.35-1.27 (m, 1H), 0.98-0.79 (m, 4H) | |
| 126 | Tetrahydrofuran-3- | 9.02-9.01 (m, 1H), 8.76-8.73 (m, | 600.3 |
| | ylmethyl[(trans-4-{[({2- | 1H), 8.43-8.40 (m, 1H), | |
| | [6-(4-methylpiperazin-1- | 8.05-8.00 (m, 3H), 7.75-7.72 (m, 1H), | |
| | yl)pyridin-3-yl]quinolin-4- | 7.56-7.52 (m, 1H), 7.13-7.09 (m, 1H), | |
| | yl}carbonyl)amino]methyl}cyclohexyl)methyl]carbamate | 6.97 (d, 1H), 3.90-3.87 (m, 1H), | |
| | | 3.82-3.78 (m, 1H), 3.70-3.64 (m, | |
| | | 2H), 3.62-3.55 (m, 6H), | |
| | | 3.20-3.17 (m, 2H), 2.82-2.78 (m, 2H), | |
| | | 2.41-2.38 (m, 4H), 2.21 (s, 3H), | |
| | | 1.92-1.85 (m, 2H), 1.83-1.78 (m, 2H), | |
| | | 1.72-1.67 (m, 2H), 1.54-1.46 (m, 2H), | |
| | | 1.36-1.28 (m, 1H), 0.97-0.79 (m, 4H) | |
| 127 | Tetrahydrofuran-2- | 9.02-9.00 (m, 1H), 8.75-8.73 (m, | 600.3 |
| | ylmethyl[(trans-4-{[({2- | 1H), 8.42-8.40 (m, 1H), | |
| | [6-(4-methylpiperazin-1- | 8.05-8.01 (m, 3H), 7.76-7.72 (m, 1H), | |
| | yl)pyridin-3-yl]quinolin-4- | 7.56-7.53 (m, 1H), 7.16-7.13 (m, 1H), | |
| | yl}carbonyl)amino]methyl}cyclohexyl)methyl]carbamate | 6.97 (d, 1H), 3.94-3.87 (m, 1H), | |
| | | 3.85-3.81 (m, 1H), 3.72-3.68 (m, | |
| | | 1H), 3.62-3.57 (m, 6H), | |
| | | 3.21-3.17 (m, 2H), 2.82-2.79 (m, 2H), | |
| | | 2.40-2.39 (m, 4H), 2.21 (s, 3H), | |
| | | 1.90-1.68 (m, 6H), 1.55-1.45 (m, 3H), | |
| | | 1.36-1.29 (m, 1H), 0.97-0.80 (m, 4H) | |
| 128 | (5-Methylisoxazol-3- | 9.02-9.00 (m, 1H), 8.76-8.73 (m, | 611.3 |
| | yl)methyl[(trans-4-{[({2- | 1H), 8.43-8.40 (m, 1H), | |
| | [6-(4-methylpiperazin-1- | 8.06-8.00 (m, 3H), 7.75-7.72 (m, 1H), | |
| | yl)pyridin-3-yl]quinolin-4- | 7.56-7.52 (m, 1H), 7.35-7.32 (m, 1H), | |
| | yl}carbonyl)amino]methyl}cyclohexyl)methyl]carbamate | 6.97 (d, 1H), 6.16 (s, 1H), 5.18 (s, | |
| | | 2H), 3.63-3.55 (m, 4H), | |
| | | 3.21-3.16 (m, 2H), 2.85-2.82 (m, 2H), | |
| | | 2.41-2.38 (m, 4H), 2.35 (s, 3H), 2.21 (s, | |
| | | 3H), 1.83-1.78 (m, 2H), | |
| | | 1.73-1.68 (m, 2H), 1.55-1.47 (m, 1H), | |
| | | 1.36-1.30 (m, 1H), 0.98-0.82 (m, 4H) | |
| 129 | 2-(1H-Pyrazol-1-yl)ethyl- | 9.02-9.01 (m, 1H), 8.76-8.73 (m, | 610.3 |
| | [(trans-4-{[({2-[6-(4- | 1H), 8.43-8.40 (m, 1H), | |
| | methylpiperazin-1- | 8.06-8.01 (m, 3H), 7.75-7.72 (m, 1H), | |
| | yl)pyridin-3-yl]quinolin-4- | 7.67-7.66 (m, 1H), 7.55-7.52 (m, 1H), | |
| | yl}carbonyl)amino]methyl}cyclohexyl)methyl]carbamate | 7.41-7.40 (m, 1H), 7.17-7.14 (m, | |
| | | 1H), 6.97 (d, 1H), 6.20 (s, 1H), | |
| | | 4.30-4.22 (m, 4H), 3.63-3.57 (m, 4H), | |
| | | 3.20-3.16 (m, 2H), 2.81-2.77 (m, | |
| | | 2H), 2.42-2.38 (m, 4H), 2.21 (s, 3H), | |
| | | 1.83-1.76 (m, 2H), 1.70-1.65 (m, | |
| | | 2H), 1.35-1.25 (m, 1H), | |
| | | 0.96-0.78 (m, 4H), 1.53-1.42 (m, 1H) | |
| 130 | 1,3-Thiazol-2-ylmethyl- | 9.02-9.00 (m, 1H), 8.76-8.73 (m, | 613.3 |
| | [(trans-4-{[({2-[6-(4- | 1H), 8.43-8.40 (m, 1H), | |
| | methylpiperazin-1- | 8.05-8.01 (m, 3H), 7.75-7.72 (m, 3H), | |
| | yl)pyridin-3-yl]quinolin-4- | 7.56-7.53 (m, 1H), 7.47-7.44 (m, 1H), | |
| | yl}carbonyl)amino]methyl}cyclohexyl)methyl]carbamate | 6.97 (d, 1H), 5.75 (s, 2H), | |
| | | 3.62-3.58 (m, 4H), 3.20-3.17 (m, 2H), | |
| | | 2.87-2.83 (m, 2H), 2.40-2.37 (m, 4H), | |
| | | 2.20 (s, 3H), 1.84-1.78 (m, 2H), | |
| | | 1.74-1.69 (m, 2H), 1.55-1.47 (m, | |
| | | 1H), 1.39-1.31 (m, 1H), | |
| | | 0.98-0.84 (m, 4H) | |
| 131 | Pyrazin-2-ylmethyl- | 9.02-9.01 (m, 1H), 8.76-8.73 (m, | 608.3 |
| | [(trans-4-{[({2-[6-(4- | 1H), 8.63-8.56 (m, 3H), | |
| | methylpiperazin-1- | 8.43-8.40 (m, 1H), 8.06-8.01 (m, 3H), | |
| | yl)pyridin-3-yl]quinolin-4- | 7.75-7.72 (m, 1H), 7.56-7.52 (m, 1H), | |
| | yl}carbonyl)amino]methyl}cyclohexyl)methyl]carbamate | 7.41-7.39 (m, 1H), 6.97 (d, 1H), | |
| | | 5.11 (s, 2H), 3.62-3.58 (m, 4H), | |
| | | 3.21-3.17 (m, 2H), 2.87-2.83 (m, 2H), | |
| | | 2.40-2.38 (m, 4H), 2.22 (s, 3H), | |
| | | 1.84-1.78 (m, 2H), 1.74-1.68 (m, 2H), | |
| | | 1.56-1.47 (m, 1H), 1.39-1.30 (m, 1H), | |
| | | 1.00-0.81 (m, 4H) | |
| 132 | 2-Cyanoethyl[(trans-4- | 9.02-9.00 (m, 1H), 8.76-8.72 (m, | 569.3 |
| | {[({2-[6-(4- | 1H), 8.42-8.40 (m, 1H), | |
| | methylpiperazin-1- | 8.06-8.01 (m, 3H), 7.75-7.72 (m, 1H), | |
| | yl)pyridin-3-yl]quinolin-4- | 7.56-7.52 (m, 1H), 7.34-7.31 (m, 1H), | |
| | yl}carbonyl)amino]methyl}cyclohexyl)methyl]carbamate | 6.97 (d, 1H), 4.08 (t, 2H), | |
| | | 3.62-3.57 (m, 4H), 3.20-3.17 (m, 2H), | |
| | | 2.84-2.78 (m, 4H), 2.41-2.38 (m, 4H), | |
| | | 2.21 (s, 3H), 1.83-1.79 (m, 2H), | |
| | | 1.73-1.68 (m, 2H), 1.55-1.46 (m, | |
| | | 1H), 1.37-1.30 (m, 1H), | |
| | | 0.97-0.81 (m, 4H) | |

### Example 133

### (1S)-2-Hydroxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate

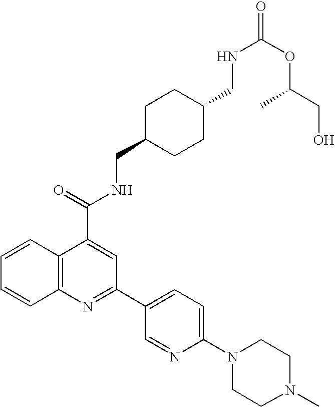

TFA (1.3 mL) was added to a solution of (1S)-2-tert-butoxy-1-methylethyl [(trans-4-{[({2-[6-(4-methylpiperazin-1-yl)pyridin-3-yl]quinolin-4-yl}carbonyl)amino]-methyl}cyclohexyl)methyl]carbamate (Intermediate PPP) (17 mg, 27 μmol) in DCM (3 mL) and the reaction mixture was stirred for 1.5 h at rt. The reaction mixture was concentrated in vacuo to leave a residue, which was dissolved in DCM and a saturated aq. solution of NaHCO₃ was added. The layers were separated and the organic layer was dried (phase separator) and concentrated in vacuo to leave a residue, which was purified by flash column chromatography, using a mixture of 0→75% EtOAc:MeOH:TEA (1:0.2:0.02) in EtOAc as eluent, to give the title compound (4.0 mg, 26%). ¹H NMR (400 MHz, CDCl₃) δ 8.85 (d, 1H), 8.31 (dd, 1H), 8.04 (d, 2H), 7.72 (s, 1H), 7.68-7.62 (m, 2H), 7.49-7.42 (m, 1H), 6.71 (d, 1H), 6.19 (t, 1H), 4.89-4.70 (m, 2H), 3.81-3.70 (m, 4H), 3.61 (dd, 1H), 3.51 (dd, 1H), 3.38-3.32 (m, 2H), 3.02-2.94 (m, 2H), 2.75-2.62 (m, 4H), 2.44 (s, 3H), 1.87-1.72 (m, 4H), 1.63-1.49 (m, 1H), 1.45-1.31 (m, 1H), 1.16 (d, 3H), 1.07-0.84 (m, 4H); m/z (M+H)⁺ 575.2.

### Examples 134-135

The following Examples 134-135 were prepared by the general procedure of Example 1 (Method 1) using the appropriate Intermediate QQQ-RRR in place of 2-[6-(4-methylpiperazin-1-yl)pyridin-3-yl]quinoline-4-carboxylic acid and using DMF in place of DCM.

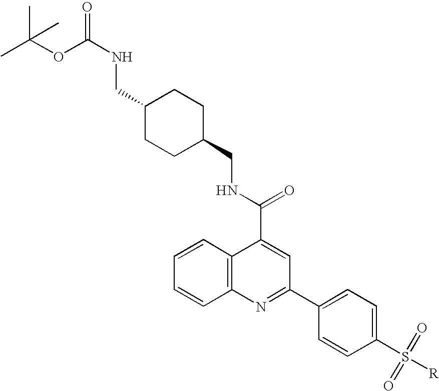

| | | | ¹H NMR (400 MHz, | MS |
|---|---|---|---|---|
| | | | DMSO- | m/z |
| Example | Name | R | d6) δ | (M + H)⁺ |
| 134 | tert-Butyl{[trans-4- | NHMe | 8.79 (t, 1H), | 567.2 |
| | ({[(2-{4- | | 8.50-8.46 (m, 2H), 8.16-8.11 (m, | |
| | [(methylamino)sulfonyl]- | | 3H), 7.95-7.91 (m, 2H), | |
| | phenyl}quinolin- | | 7.85-7.79 (m, 1H), | |
| | 4-yl)carbonyl]amino}methyl)cyclohexyl]methyl}carbamate | | 7.68-7.63 (m, 1H), | |
| | | | 7.56-7.52 (m, 1H), 6.76 (t, 1H), | |
| | | | 3.20 (t, 2H), 2.74 (t, 2H), | |
| | | | 2.45-2.42 (m, 3H), | |
| | | | 1.84-1.77 (m, 2H), | |
| | | | 1.72-1.65 (m, 2H), 1.55-1.46 (m, | |
| | | | 1H), 1.35-1.24 (m, 1H), | |
| | | | 1.33 (s, 9H), | |
| | | | 0.99-0.77 (m, 4H) | |
| 135 | tert-Butyl[(trans-4- | NH₂ | 8.79 (t, 1H), | 553.1 |
| | {[({2-[4- | | 8.48-8.44 (m, 2H), 8.16-8.10 (m, | |
| | (aminosulfonyl)phenyl]- | | 3H), 7.99-7.96 (m, 2H), | |
| | quinolin-4-yl}carbonyl)amino]- | | 7.84-7.79 (m, 1H), | |
| | methyl}-cyclohexyl)methyl]carbamate | | 7.67-7.63 (m, 1H), 7.45 (s, | |
| | | | 2H), 6.76 (t, 1H), 3.20 (t, | |
| | | | 2H), 2.74 (t, 2H), | |
| | | | 1.84-1.77 (m, 2H), | |
| | | | 1.72-1.65 (m, 2H), 1.56-1.46 (m, | |
| | | | 1H), 1.34-1.24 (m, 1H), | |
| | | | 1.33 (s, 9H), | |
| | | | 1.00-0.78 (m, 4H) | |

### Examples 136-141

The following Examples 136-141 were prepared by the general procedure of Example 109 (Method 19) using the appropriate Intermediate SSS-UUU in place of N-{[trans-4-(aminomethyl)cyclohexyl]methyl}-2-[6-(4-methylpiperazin-1-yl)pyridin-3-yl]quinoline-4-carboxamide and 4-nitrophenyl tetrahydro-2H-pyran-4-yl carbonate (Intermediate QQ) or 4-nitrophenyl (3S)-tetrahydrofuran-3-yl carbonate (Intermediate XX).

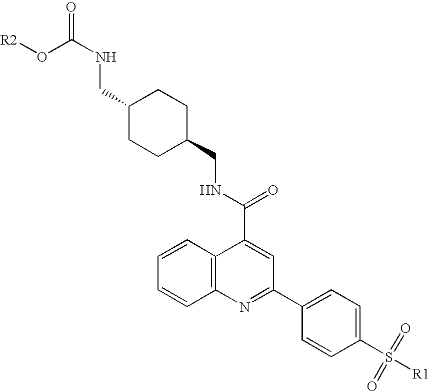

| | | | MS |
|---|---|---|---|
| | | ¹H NMR (600 MHz, | m/z |
| Example | Name | DMSO/DMSO-d6)* δ | (M + H)⁺ |
| 136 | Tetrahydro-2H-pyran- | 8.81 (t, 1H), 8.50-8.47 (m, 2H), | 595.1 |
| | 4-yl{[trans-4-({[(2-{4- | 8.16-8.12 (m, 3H), 7.95-7.92 (m, | |
| | [(methylamino)sulfonyl]phenyl}quinolin- | 2H), 7.85-7.81 (m, 1H), | |
| | 4- | 7.68-7.65 (m, 1H), 7.55 (q, 1H), 7.09 (t, 1H), | |
| | yl)carbonyl]amino}methyl)cyclohexyl]methyl}- | 4.65-4.60 (m, 1H), 3.78-3.74 (m, | |
| | carbamate | 2H), 3.22-3.19 (m, 2H), | |
| | | 2.82-2.79 (m, 2H), 2.44 (d, 3H), | |
| | | 1.83-1.77 (m, 4H), 1.72-1.67 (m, 2H), | |
| | | 1.55-1.41 (m, 3H), 1.35-1.28 (m, 1H), | |
| | | 0.98-0.81 (m, 4H) | |
| 137 | (3S)-Tetrahydrofuran- | 8.81 (t, 1H), 8.50-8.47 (m, 2H), | 581.1 |
| | 3-yl{[trans-4-({[(2-{4- | 8.16-8.12 (m, 3H), 7.95-7.92 (m, | |
| | [(methylamino)sulfonyl]phenyl}quinolin- | 2H), 7.85-7.81 (m, 1H), | |
| | 4- | 7.68-7.65 (m, 1H), 7.17 (t, 1H), | |
| | yl)carbonyl]amino}methyl)cyclohexyl]methyl}- | 5.07-5.04 (m, 1H), 3.75-3.64 (m, 3H), | |
| | carbamate | 3.62-3.58 (m, 1H), 3.22-3.19 (m, 2H), | |
| | | 2.82-2.78 (m, 2H), 2.44 (s, 3H), | |
| | | 2.09-2.02 (m, 1H), 1.83-1.78 (m, | |
| | | 3H), 1.72-1.67 (m, 2H), | |
| | | 1.56-1.47 (m, 1H), 1.35-1.27 (m, 1H), | |
| | | 0.98-0.81 (m, 4H) | |
| 138 | Tetrahydro-2H-pyran- | 8.81 (t, 1H), 8.48-8.45 (m, 2H), | 581.1 |
| | 4-yl[(trans-4-{[({2-[4- | 8.16-8.11 (m, 3H), 7.99-7.97 (m, | |
| | (aminosulfonyl)phenyl]- | 2H), 7.84-7.81 (m, 1H), | |
| | quinolin-4- | 7.68-7.64 (m, 1H), 7.46 (s, 2H), 7.09 (t, 1H), | |
| | yl}carbonyl)amino]- | 4.65-4.60 (m, 1H), 3.78-3.74 (m, | |
| | methyl}cyclohexyl)- | 2H), 3.23-3.20 (m, 2H), | |
| | methyl]carbamate | 2.83-2.79 (m, 2H), 1.84-1.77 (m, 4H), | |
| | | 1.73-1.68 (m, 2H), 1.56-1.41 (m, 3H), | |
| | | 1.36-1.28 (m, 1H), 0.99-0.82 (m, | |
| | | 4H) | |
| 139 | (3S)-Tetrahydrofuran- | 8.81 (t, 1H), 8.48-8.45 (m, 2H), | 567.1 |
| | 3-yl[(trans-4-{[({2-[4- | 8.15-8.11 (m, 3H), 8.00-7.97 (m, | |
| | (aminosulfonyl)phenyl]- | 2H), 7.84-7.81 (m, 1H), | |
| | quinolin-4- | 7.68-7.64 (m, 1H), 7.46 (s, 2H), 7.17 (t, 1H), | |
| | yl}carbonyl)amino]- | 5.07-5.04 (m, 1H), 3.75-3.64 (m, | |
| | methyl}cyclohexyl)- | 3H), 3.62-3.59 (m, 1H), | |
| | methyl]carbamate | 3.23-3.20 (m, 2H), 2.82-2.79 (m, 2H), | |
| | | 2.09-2.03 (m, 1H), 1.84-1.79 (m, 3H), | |
| | | 1.72-1.68 (m, 2H), 1.55-1.48 (m, | |
| | | 1H), 1.35-1.28 (m, 1H), | |
| | | 0.99-0.82 (m, 4H) | |
| 140 | Tetrahydro-2H-pyran- | 8.82 (t, 1H), 8.53-8.51 (m, 2H), | 609.1 |
| | 4-yl{[trans-4-({[(2-{4- | 8.17-8.13 (m, 3H), 7.93-7.90 (m, | |
| | [(dimethylamino)sulfonyl]phenyl}quinolin- | 2H), 7.85-7.82 (m, 1H), | |
| | 4- | 7.69-7.66 (m, 1H), 7.09 (t, 1H), | |
| | yl)carbonyl]amino}- | 4.65-4.60 (m, 1H), 3.78-3.74 (m, 2H), | |
| | methyl)cyclohexyl]- | 3.23-3.19 (m, 2H), 2.83-2.79 (m, 2H), | |
| | methyl}carbamate | 2.64 (s, 6H), 1.84-1.77 (m, 4H), | |
| | | 1.73-1.67 (m, 2H), 1.55-1.41 (m, | |
| | | 3H), 1.36-1.28 (m, 1H), | |
| | | 0.99-0.81 (m, 4H) | |
| 141 | (3S)-Tetrahydrofuran- | 8.82 (t, 1H), 8.53-8.51 (m, 2H), | 595.1 |
| | 3-yl{[trans-4-({[(2-{4- | 8.17-8.13 (m, 3H), 7.93-7.90 (m, | |
| | [(dimethylamino)sulfonyl]phenyl}quinolin- | 2H), 7.85-7.82 (m, 1H), | |
| | 4- | 7.69-7.66 (m, 1H), 7.17 (t, 1H), | |
| | yl)carbonyl]amino}- | 5.07-5.04 (m, 1H), 3.75-3.64 (m, 3H), | |
| | methyl)cyclohexyl]- | 3.62-3.59 (m, 1H), 3.23-3.19 (m, 2H), | |
| | methyl}carbamate | 2.82-2.79 (m, 2H), 2.64 (s, 6H), | |
| | | 2.09-2.02 (m, 1H), 1.84-1.78 (m, | |
| | | 3H), 1.72-1.67 (m, 2H), | |
| | | 1.55-1.48 (m, 1H), 1.35-1.28 (m, 1H), | |
| | | 0.99-0.81 (m, 4H) | |

### Example 142

### Method 20

### tert-Butyl [(trans-4-{[({2-[6-(4-acetylpiperazin-1-yl)pyridin-3-yl]quinolin-4-yl}carbonyl)amino]methyl}cyclohexyl)methyl]carbamate

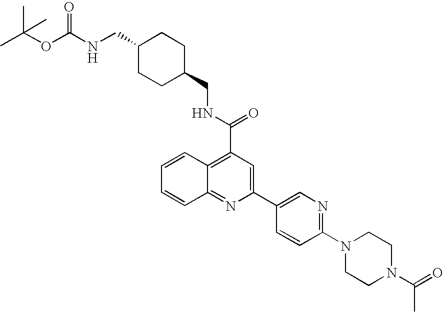

1-Acetylpiperazine (0.98 g, 7.6 mmol, 30 eq.) was added to a solution of tert-butyl ({trans-4-[({[2-(6-fluoropyridin-3-yl)quinolin-4-yl]carbonyl}amino)methyl]cyclohexyl} methyl)-carbamate (Intermediate VVV) (0.13 g, 0.25 mmol) in pyridine (2 mL) in a microwave vial. The reaction mixture was heated at 130° C. in a microwave for 30 min. The reaction mixture was concentrated in vacuo to leave a residue, which was dissolved in THF and filtered through a plug of silica using THF as eluent. The filtrate was concentrated in vacuo to leave a residue, which was dissolved in DMSO and purified by preparative HPLC (Standard method C) to give the title compound (67 mg, 44%). ¹H NMR (600 MHz, DMSO/DMSO-d₆*) δ 9.03 (s, 1H), 8.78-8.73 (m, 1H), 8.44 (d, 1H), 8.06-7.98 (m, 3H), 7.77-7.71 (m, 1H), 7.58-7.51 (m, 1H), 6.99 (d, 1H), 6.78-6.74 (m, 1H), 3.70-3.63 (m, 2H), 3.61-3.57 (m, 2H), 3.56-3.52 (m, 4H), 3.21-3.16 (m, 2H), 2.77-2.72 (m, 2H), 2.03 (s, 3H), 1.83-1.77 (m, 2H), 1.71-1.66 (m, 2H), 1.54-1.47 (m, 1H), 1.33 (s, 9H), 1.32-1.27 (m, 1H), 0.97-0.79 (m, 4H); m/z (M+H)⁺ 601.2.

### Examples 143-172

The following Examples 143-172 were prepared by the general procedure of Example 142 (Method 20) using the appropriate amine as starting material in place of 1-acetylpiperazine.

| | | | MS |
|---|---|---|---|
| | | ¹H NMR δ (600 MHz, | m/z |
| Example | Name | DMSO/DMSO-d₆)* | (M + H)⁺ |
| 143 | tert-Butyl{[trans- | 8.88 (s, 1H), 8.76-8.71 (m, 1H), | 534.2 |
| | 4-({[(2-{6-[(2- | 8.27 (d, 1H), 8.03 (d, 1H), 7.98 (d, 1H), | |
| | hydroxyethyl)amino]pyridin- | 7.93 (s, 1H), 7.74-7.70 (m, 1H), | |
| | 3- | 7.55-7.48 (m, 1H), 7.03-6.96 (m, | |
| | yl}quinolin-4- | 1H), 6.78-6.71 (m, 1H), 6.63 (d, | |
| | yl)carbonyl]amino}methyl)cyclohexyl]methyl}carbamate | 1H), 3.56-3.50 (m, 2H), | |
| | | 3.20-3.16 (m, 2H), 2.77-2.72 (m, 2H), | |
| | | 1.83-1.76 (m, 2H), 1.71-1.66 (m, 2H), | |
| | | 1.54-1.46 (m, 1H), 1.34 (s, 9H), | |
| | | 1.32-1.27 (m, 1H), 0.97-0.79 (m, | |
| | | 4H). | |
| 144 | tert-Butyl({trans- | 8.97 (s, 1H), 8.77-8.73 (m, 1H), | 544.2 |
| | 4-[({[2-(6- | 8.41 (d, 1H), 8.03 (d, 1H), 8.00 (d, 1H), | |
| | pyrrolidin-1- | 7.97 (s, 1H), 7.75-7.71 (m, 1H), | |
| | ylpyridin-3-yl)quinolin- | 7.55-7.50 (m, 1H), 6.78-6.74 (m, | |
| | 4- | 1H), 6.67-6.58 (m, 1H), | |
| | yl]carbonyl}amino)methyl]cyclohexyl}methyl)carbamate | 3.20-3.16 (m, 2H), 2.76-2.72 (m, 2H), | |
| | | 1.97-1.93 (m, 4H), 1.83-1.77 (m, 2H), | |
| | | 1.72-1.66 (m, 2H), 1.54-1.47 (m, | |
| | | 1H), 1.34 (s, 9H), 1.30-1.25 (m, 1H), | |
| | | 0.98-0.80 (m, 4H). | |
| 145 | tert-Butyl({trans- | 9.03 (s, 1H), 8.77-8.73 (m, 1H), | 560.2 |
| | 4-[({[2-(6- | 8.44 (d, 1H), 8.04 (d, 1H), 8.02 (d, 1H), | |
| | morpholin-4- | 8.00 (d, 1H), 7.76-7.72 (m, 1H), | |
| | ylpyridin-3-yl)- | 7.57-7.52 (m, 1H), 6.97 (d, 1H), | |
| | quinolin-4- | 6.78-6.73 (m, 1H), 3.71-3.68 (m, | |
| | yl]carbonyl}amino)- | 4H), 3.58-3.54 (m, 4H), | |
| | methyl]cyclohexyl}methyl)carbamate | 3.21-3.16 (m, 2H), 2.76-2.72 (m, 2H), | |
| | | 1.83-1.76 (m, 2H), 1.72-1.65 (m, 2H), | |
| | | 1.54-1.46 (m, 1H), 1.33 (s, 9H), | |
| | | 1.31-1.26 (m, 1H), 0.97-0.79 (m, | |
| | | 4H). | |
| 146 | tert-Butyl({trans- | 8.89 (s, 1H), 8.77-8.70 (m, 1H), | 561.3 |
| | 4-[({[2-(6-{[2- | 8.27 (dd, 1H), 8.03 (d, 1H), 7.98 (d, 1H), | |
| | (dimethylamino)ethyl]- | 7.94 (d, 1H), 7.74-7.70 (m, 1H), | |
| | amino}pyridin- | 7.54-7.50 (m, 1H), 6.92-6.86 (m, | |
| | 3-yl)quinolin-4- | 1H), 6.78-6.73 (m, 1H), 6.63 (d, | |
| | yl]carbonyl}amino)- | 1H), 3.20-3.16 (m, 2H), | |
| | methyl]cyclohexyl}- | 2.76-2.72 (m, 2H), 2.19 (s, 6H), 1.83-1.77 (m, | |
| | methyl)carbamate | 2H), 1.72-1.65 (m, 2H), | |
| | | 1.56-1.47 (m, 1H), 1.33 (s, 9H), 1.32-1.27 (m, | |
| | | 1H), 0.97-0.79 (m, 4H). | |
| 147 | tert-Butyl{[trans- | 8.98 (s, 1H), 8.78-8.72 (m, 1H), | 560.2 |
| | 4-({[(2-{6-[(3R)-3- | 8.39 (d, 1H), 8.02 (d, 1H), 7.99 (d, 1H), | |
| | hydroxypyrrolidin- | 7.97 (s, 1H), 7.74-7.70 (m, 1H), | |
| | 1-yl]pyridin-3- | 7.55-7.50 (m, 1H), 6.78-6.73 (m, | |
| | yl}quinolin-4- | 1H), 6.59 (d, 1H), 4.40 (s, 1H), | |
| | yl)carbonyl]amino}- | 3.58-3.47 (m, 4H), 3.21-3.16 (m, 2H), | |
| | methyl)cyclohexyl]- | 2.77-2.72 (m, 2H), 2.06-1.99 (m, | |
| | methyl}carbamate | 1H), 1.94-1.87 (m, 1H), | |
| | | 1.83-1.76 (m, 2H), 1.72-1.64 (m, 2H), | |
| | | 1.55-1.45 (m, 1H), 1.33 (s, 9H), | |
| | | 1.32-1.26 (m, 1H), 0.97-0.78 (m, 4H). | |
| 148 | tert-Butyl({trans- | 8.88 (s, 1H), 8.75-8.70 (m, 1H), | 548.2 |
| | 4-[({[2-(6-{[(1S)-2- | 8.26 (d, 1H), 8.02 (d, 1H), 7.98 (d, 1H), | |
| | hydroxy-1- | 7.93 (s, 1H), 7.74-7.69 (m, 1H), | |
| | methylethyl]amino}- | 7.54-7.49 (m, 1H), 6.80-6.73 (m, | |
| | pyridin-3- | 2H), 6.61 (d, 1H), 4.06-3.97 (m, | |
| | yl)quinolin-4- | 1H), 3.51-3.44 (m, 1H), | |
| | yl]carbonyl}amino)- | 3.33-3.29 (m, 1H), 3.20-3.16 (m, 2H), | |
| | methyl]cyclohexyl}- | 2.76-2.73 (m, 2H), 1.83-1.76 (m, 2H), | |
| | methyl)carbamate | 1.72-1.65 (m, 2H), 1.54-1.45 (m, | |
| | | 1H), 1.34 (s, 9H), 1.31-1.28 (m, 1H), | |
| | | 1.13 (d, 3H), 0.97-0.79 (m, 4H). | |
| 149 | tert-Butyl({trans- | 8.89 (s, 1H), 8.75-8.70 (m, 1H), | 548.2 |
| | 4-[({[2-(6-{[(1R)-2- | 8.26 (d, 1H), 8.02 (d, 1H), 7.98 (d, 1H), | |
| | hydroxy-1- | 7.93 (s, 1H), 7.74-7.69 (m, 1H), | |
| | methylethyl]amino}- | 7.54-7.49 (m, 1H), 6.80-6.73 (m, | |
| | pyridin-3- | 2H), 6.61 (d, 1H), 4.06-3.97 (m, | |
| | yl)quinolin-4- | 1H), 3.51-3.44 (m, 1H), | |
| | yl]carbonyl}amino)- | 3.33-3.29 (m, 1H), 3.20-3.17 (m, 2H), | |
| | methyl]cyclohexyl}- | 2.76-2.73 (m, 2H), 1.83-1.76 (m, 2H), | |
| | methyl)carbamate | 1.72-1.65 (m, 2H), 1.54-1.44 (m, | |
| | | 1H), 1.34 (s, 9H), 1.31-1.28 (m, 1H), | |
| | | 1.12 (d, 3H), 0.97-0.78 (m, 4H). | |
| 150 | tert-Butyl{[trans- | 8.89-8.88 (m, 1H), 8.74-8.71 (m, | 548.4 |
| | 4-({[(2-{6-[(3- | 1H), 8.28-8.26 (m, 1H), | |
| | hydroxypropyl)amino]pyridin- | 8.04-7.97 (m, 3H), 7.74-7.70 (m, 1H), | |
| | 3- | 7.54-7.50 (m, 1H), 7.01-6.98 (m, 1H), | |
| | yl}quinolin-4- | 6.78-6.74 (m, 1H), 6.59 (d, 1H), | |
| | yl)carbonyl]amino}- | 4.53 (t, 1H), 3.48 (q, 2H), | |
| | methyl)cyclohexyl]methyl}carbamate | 3.20-3.16 (m, 2H), 2.76-2.72 (m, 2H), | |
| | | 1.82-1.77 (m, 2H), 1.71-1.66 (m, 4H), | |
| | | 1.53-1.46 (m, 1H), 1.34 (s, 9H), | |
| | | 1.31-1.25 (m, 1H), 0.97-0.79 (m, | |
| | | 4H) | |
| 151 | tert-Butyl[(trans-4- | 8.99-8.98 (m, 1H), 8.75-8.72 (m, | 574.4 |
| | {[({2-[6-(4- | 1H), 8.39-8.36 (m, 1H), | |
| | hydroxypiperidin-1- | 8.05-7.99 (m, 3H), 7.75-7.71 (m, 1H), | |
| | yl)pyridin-3-yl]- | 7.55-7.51 (m, 1H), 6.97 (d, 1H), | |
| | quinolin-4-yl}- | 6.78-6.74 (m, 1H), 4.75-4.73 (m, 1H), | |
| | carbonyl)amino]methyl}cyclohexyl)methyl]carbamate | 4.12-4.07 (m, 3H), 3.75-3.69 (m, | |
| | | 2H), 3.22-3.15 (m, 4H), | |
| | | 2.77-2.72 (m, 2H), 1.83-1.75 (m, 4H), | |
| | | 1.71-1.66 (m, 2H), 1.55-1.46 (m, 1H), | |
| | | 1.33 (s, 9H), 1.31-1.25 (m, 1H), | |
| | | 0.98-0.78 (m, 4H) | |
| 152 | tert-Butyl{[trans- | 8.97-8.95 (m, 1H), 8.75-8.71 (m, | 578.4 |
| | 4-({[(2-{6-[bis(2- | 1H), 8.38-8.35 (m, 1H), | |
| | hydroxyethyl)amino]pyridin- | 8.05-7.98 (m, 3H), 7.74-7.70 (m, 1H), | |
| | 3- | 7.54-7.50 (m, 1H), 6.80 (d, 1H), | |
| | yl}quinolin-4- | 6.78-6.74 (m, 1H), 4.84-4.80 (m, 2H), | |
| | yl)carbonyl]amino}- | 3.65-3.61 (m, 4H), 3.61-3.57 (m, | |
| | methyl)cyclohexyl]methyl}carbamate | 4H), 3.20-3.16 (m, 2H), | |
| | | 2.76-2.73 (m, 2H), 1.83-1.77 (m, 2H), | |
| | | 1.71-1.66 (m, 2H), 1.54-1.47 (m, 1H), | |
| | | 1.33 (s, 9H), 1.31-1.25 (m, 1H), | |
| | | 0.97-0.79 (m, 4H) | |
| 153 | tert-Butyl[(trans-4- | 9.01-8.99 (m, 1H), 8.76-8.72 (m, | 601.4 |
| | {[({2-[6-(4- | 1H), 8.41-8.38 (m, 1H), | |
| | carbamoylpiperidin- | 8.05-7.99 (m, 3H), 7.75-7.71 (m, 1H), | |
| | 1-yl)pyridin-3-yl]- | 7.56-7.51 (m, 1H), 7.30-7.27 (m, 1H), | |
| | quinolin-4- | 6.98 (d, 1H), 6.78-6.74 (m, 2H), | |
| | yl}carbonyl)amino]- | 4.43-4.38 (m, 2H), 3.21-3.16 (m, | |
| | methyl}cyclohexyl)methyl]carbamate | 2H), 2.95-2.89 (m, 4H), | |
| | | 2.77-2.73 (m, 2H), 1.83-1.74 (m, 4H), | |
| | | 1.72-1.65 (m, 2H), 1.55-1.47 (m, 2H), | |
| | | 1.33 (s, 9H), 1.31-1.24 (m, 1H), | |
| | | 0.98-0.77 (m, 4H) | |
| 154 | tert-Butyl({trans- | 9.01-8.99 (m, 1H), 8.76-8.72 (m, | 559.4 |
| | 4-[({[2-(6- | 1H), 8.42-8.37 (m, 1H), | |
| | piperazin-1- | 8.05-7.99 (m, 3H), 7.75-7.71 (m, 1H), | |
| | ylpyridin-3-yl)- | 7.55-7.52 (m, 1H), 6.93 (d, 1H), | |
| | quinolin-4-yl]- | 6.78-6.74 (m, 1H), 3.54-3.50 (m, 4H), | |
| | carbonyl}amino)methyl]cyclohexyl}methyl)carbamate | 3.21-3.16 (m, 2H), 2.78-2.73 (m, | |
| | | 6H), 1.83-1.77 (m, 2H), | |
| | | 1.71-1.65 (m, 2H), 1.54-1.46 (m, 1H), 1.34 (s, | |
| | | 9H), 1.31-1.25 (m, 1H), | |
| | | 0.98-0.77 (m, 4H) | |
| 155 | tert-Butyl({trans- | 8.90-8.89 (m, 1H), 8.75-8.71 (m, | 578.4 |
| | 4-[({[2-(6-{[2-(2- | 1H), 8.29-8.26 (m, 1H), | |
| | hydroxyethoxy)ethyl]amino}pyridin- | 8.05-7.96 (m, 3H), 7.75-7.68 (m, 1H), | |
| | 3- | 7.54-7.51 (m, 1H), 7.06-7.03 (m, 1H), | |
| | yl)quinolin-4-yl]- | 6.78-6.75 (m, 1H), 6.63 (d, 1H), | |
| | carbonyl}amino)- | 4.61 (t, 1H), 3.57-3.54 (m, 2H), | |
| | methyl]cyclohexyl}methyl)carbamate | 3.51-3.46 (m, 2H), 3.45-3.42 (m, | |
| | | 2H), 3.20-3.16 (m, 2H), | |
| | | 2.77-2.72 (m, 2H), 1.83-1.76 (m, 2H), | |
| | | 1.71-1.66 (m, 2H), 1.53-1.46 (m, 1H), | |
| | | 1.33 (s, 9H), 1.31-1.24 (m, 1H), | |
| | | 0.97-0.79 (m, 4H) | |
| 156 | tert-Butyl{[trans- | 8.90-8.88 (m, 1H), 8.75-8.71 (m, | 548.4 |
| | 4-({[(2-{6-[(2- | 1H), 8.28-8.25 (m, 1H), | |
| | methoxyethyl)amino]pyridin- | 8.04-7.97 (m, 3H), 7.74-7.69 (m, 1H), | |
| | 3- | 7.54-7.50 (m, 1H), 7.09-7.04 (m, 1H), | |
| | yl}quinolin-4- | 6.78-6.74 (m, 1H), 6.64 (d, 1H), | |
| | yl)carbonyl]amino}methyl)cyclohexyl]methyl}carbamate | 3.50-3.45 (m, 2H), 3.26 (s, 3H), | |
| | | 3.20-3.16 (m, 2H), 2.77-2.72 (m, | |
| | | 2H), 1.82-1.77 (m, 2H), | |
| | | 1.71-1.66 (m, 2H), 1.54-1.47 (m, 1H), 1.34 (s, | |
| | | 9H), 1.31-1.25 (m, 1H), | |
| | | 0.97-0.78 (m, 4H) | |
| 157 | tert-Butyl[(trans-4- | 8.99-8.97 (m, 1H), 8.76-8.72 (m, | 574.4 |
| | {[({2-[6-(3- | 1H), 8.39-8.36 (m, 1H), | |
| | hydroxypiperidin-1- | 8.05-7.98 (m, 3H), 7.75-7.71 (m, 1H), | |
| | yl)pyridin-3-yl]- | 7.55-7.51 (m, 1H), 6.93 (d, 1H), | |
| | quinolin-4- | 6.78-6.75 (m, 1H), 4.91-4.89 (m, 1H), | |
| | yl}carbonyl)amino]methyl}cyclohexyl)methyl]carbamate | 4.27-4.23 (m, 1H), 4.06-4.02 (m, | |
| | | 2H), 3.52-3.46 (m, 2H), | |
| | | 3.20-3.16 (m, 2H), 2.88-2.82 (m, 2H), | |
| | | 2.76-2.72 (m, 2H), 1.92-1.86 (m, 2H), | |
| | | 1.83-1.77 (m, 2H), 1.71-1.66 (m, | |
| | | 2H), 1.54-1.47 (m, 1H), 1.34 (s, 9H), | |
| | | 1.31-1.26 (m, 1H), 0.98-0.79 (m, | |
| | | 4H) | |
| 158 | 3-{[5-(4-{[(trans-4- | 8.90-8.88 (m, 1H), 8.75-8.71 (m, | 576.4 |
| | {[(tert- | 1H), 8.28-8.25 (m, 1H), | |
| | Butoxycarbonyl)amino]methyl}cyclohexyl)methyl]- | 8.05-7.97 (m, 3H), 7.74-7.69 (m, 1H), | |
| | carbamoyl}quinolin-2- | 7.54-7.50 (m, 1H), 7.15-7.09 (m, 1H), | |
| | yl)pyridin-2- | 6.78-6.74 (m, 1H), 6.63 (d, 1H), | |
| | yl]amino}-2- | 3.57-3.48 (m, 1H), 3.19-3.16 (m, | |
| | methyl-propanoic | 2H), 2.76-2.73 (m, 2H), | |
| | acid | 1.82-1.77 (m, 2H), 1.70-1.65 (m, 2H), | |
| | | 1.54-1.46 (m, 1H), 1.34 (s, 9H), | |
| | | 1.31-1.25 (m, 1H), 1.08 (d, 3H), 0.97-0.80 (m, | |
| | | 4H) | |
| 159 | tert-Butyl({trans- | 8.88-8.86 (m, 1H), 8.72-8.69 (m, | 596.4 |
| | 4-[({[2-(6-{[(5- | 1H), 8.48-8.45 (m, 2H), | |
| | methylpyrazin-2- | 8.33-8.30 (m, 1H), 8.05-7.97 (m, 3H), | |
| | yl)methyl]amino}pyridin- | 7.74-7.70 (m, 1H), 7.70-7.67 (m, 1H), | |
| | 3- | 7.55-7.51 (m, 1H), 6.77-6.71 (m, | |
| | yl)quinolin-4- | 2H), 4.66-4.63 (m, 2H), | |
| | yl]carbonyl}amino)methyl]cyclohexyl}methyl)carbamate | 3.19-3.15 (m, 2H), 2.76-2.72 (m, 2H), 2.03 (s, | |
| | | 3H), 1.82-1.77 (m, 2H), | |
| | | 1.70-1.65 (m, 2H), 1.52-1.45 (m, 1H), 1.33 (s, | |
| | | 9H), 1.30-1.23 (m, 1H), | |
| | | 0.97-0.77 (m, 4H) | |
| 160 | tert-Butyl{[trans- | 9.00-8.97 (m, 1H), 8.76-8.73 (m, | 587.4 |
| | 4-({[(2-{6-[(3S)-3- | 1H), 8.41-8.38 (m, 1H), | |
| | (dimethylamino)- | 8.04-7.98 (m, 3H), 7.74-7.70 (m, 1H), | |
| | pyrrolidin-1- | 7.54-7.50 (m, 1H), 6.78-6.74 (m, 1H), | |
| | yl]pyridin-3- | 6.61 (d, 1H), 3.78-3.62 (m, 1H), | |
| | yl}quinolin-4-yl)- | 3.20-3.16 (m, 2H), 2.77-2.73 (m, | |
| | carbonyl]amino}methyl)cyclohexyl]methyl}carbamate | 2H), 2.19 (s, 6H), 1.83-1.78 (m, 4H), | |
| | | 1.71-1.66 (m, 2H), 1.53-1.47 (m, | |
| | | 1H), 1.34 (s, 9H), 1.31-1.25 (m, | |
| | | 1H), 0.97-0.79 (m, 4H) | |
| 161 | tert-Butyl{[trans- | 8.99-8.97 (m, 1H), 8.75-8.72 (m, | 588.4 |
| | 4-({[(2-{6-[4- | 1H), 8.39-8.36 (m, 1H), | |
| | (hydroxy | 8.05-7.98 (m, 3H), 7.75-7.71 (m, 1H), | |
| | methyl)piperidin-1- | 7.55-7.51 (m, 1H), 6.95 (d, 1H), | |
| | yl]pyridin-3-yl}- | 6.77-6.74 (m, 1H), 4.52-4.48 (m, 1H), | |
| | quinolin-4-yl)- | 4.45-4.40 (m, 2H), 3.27-3.23 (m, | |
| | carbonyl]amino}methyl)cyclohexyl]methyl}carbamate | 2H), 3.20-3.16 (m, 2H), | |
| | | 2.89-2.84 (m, 2H), 2.76-2.72 (m, 2H), | |
| | | 1.82-1.77 (m, 2H), 1.75-1.61 (m, 7H), | |
| | | 1.54-1.46 (m, 1H), 1.33 (s, 9H), | |
| | | 1.32-1.26 (m, 1H), 0.99-0.79 (m, | |
| | | 4H) | |
| 162 | tert-Butyl{[trans- | 8.89-8.86 (m, 1H), 8.74-8.71 (m, | 564.3 |
| | 4-({[(2-{6-[(2,3- | 1H), 8.29-8.26 (m, 1H), | |
| | dihydroxypropyl)amino]pyridin- | 8.04-7.97 (m, 3H), 7.74-7.70 (m, 1H), | |
| | 3- | 7.54-7.50 (m, 1H), 7.00-6.95 (m, 1H), | |
| | yl}quinolin-4- | 6.78-6.75 (m, 1H), 6.67 (d, 1H), | |
| | yl)carbonyl]amino}- | 4.91 (d, 1H), 4.66 (t, 1H), | |
| | methyl)cyclohexyl]- | 3.65-3.60 (m, 2H), 3.49-3.44 (m, 1H), | |
| | methyl}carbamate | 3.21-3.16 (m, 2H), 2.76-2.72 (m, 2H), | |
| | | 2.47-2.45 (m, 2H), 1.83-1.77 (m, | |
| | | 2H), 1.72-1.65 (m, 2H), | |
| | | 1.53-1.46 (m, 1H), 1.33 (s, 9H), 1.32-1.24 (m, | |
| | | 1H), 0.96-0.78 (m, 4H) | |
| 163 | tert-Butyl{[trans- | 8.98-8.95 (m, 1H), 8.76-8.72 (m, | 587.4 |
| | 4-({[(2-{6-[(2S)-2- | 1H), 8.42-8.38 (m, 1H), | |
| | carbamoylpyrrolidin- | 8.05-7.97 (m, 3H), 7.74-7.71 (m, 1H), | |
| | 1-yl]pyridin-3- | 7.55-7.51 (m, 1H), 7.40-7.37 (m, 1H), | |
| | yl}-quinolin-4-yl)- | 6.95-6.91 (m, 1H), 6.78-6.74 (m, | |
| | carbonyl]amino}methyl)cyclohexyl]methyl}- | 1H), 6.58-6.55 (m, 1H), | |
| | carbamate | 4.39-4.35 (m, 1H), 3.69-3.64 (m, 2H), | |
| | | 3.20-3.16 (m, 2H), 2.76-2.71 (m, 2H), | |
| | | 2.21-2.14 (m, 2H), 1.99-1.93 (m, | |
| | | 2H), 1.83-1.77 (m, 2H), | |
| | | 1.71-1.66 (m, 2H), 1.53-1.46 (m, 1H), 1.33 (s, | |
| | | 9H), 1.31-1.24 (m, 1H), | |
| | | 0.97-0.79 (m, 4H) | |
| 164 | tert-Butyl({trans- | 8.88-8.87 (m, 1H), 8.75-8.71 (m, | 564.3 |
| | 4-[({[2-(6-{[2- | 1H), 8.28-8.25 (m, 1H), | |
| | hydroxy-1- | 8.04-7.97 (m, 3H), 7.74-7.70 (m, 1H), | |
| | (hydroxymethyl)- | 7.54-7.50 (m, 1H), 6.78-6.75 (m, 1H), | |
| | ethyl]amino}pyridin- | 6.72-6.69 (m, 1H), 6.68 (d, 1H), | |
| | 3-yl)quinolin-4- | 4.69 (t, 2H), 4.01-3.95 (m, 1H), | |
| | yl]- | 3.55-3.47 (m, 4H), 3.20-3.15 (m, | |
| | carbonyl}amino)methyl]cyclohexyl}methyl)- | 2H), 2.77-2.72 (m, 2H), | |
| | carbamate | 1.82-1.77 (m, 2H), 1.71-1.66 (m, 2H), | |
| | | 1.54-1.46 (m, 1H), 1.34 (s, 9H), | |
| | | 1.31-1.25 (m, 1H), 0.97-0.79 (m, 4H) | |
| 165 | tert-Butyl[(trans-4- | 9.05-9.04 (m, 1H), 8.76-8.73 (m, | 573.4 |
| | {[({2-[6-(3- | 1H), 8.48-8.45 (m, 1H), | |
| | oxopiperazin-1-yl)- | 8.13-8.11 (m, 1H), 8.06-8.02 (m, 3H), | |
| | pyridin-3- | 7.76-7.73 (m, 1H), 7.57-7.53 (m, 1H), | |
| | yl]quinolin-4- | 6.96 (d, 1H), 6.78-6.75 (m, 1H), | |
| | yl}carbonyl)amino]- | 4.11 (s, 2H), 3.83-3.80 (m, 2H), | |
| | methyl}cyclohexyl)methyl]carbamate | 3.20-3.17 (m, 2H), 2.76-2.73 (m, | |
| | | 2H), 1.83-1.78 (m, 2H), | |
| | | 1.71-1.67 (m, 2H), 1.54-1.47 (m, 1H), 1.34 (s, | |
| | | 9H), 1.31-1.26 (m, 1H), | |
| | | 0.98-0.79 (m, 4H) | |
| 166 | tert-Butyl({trans- | 8.94-8.92 (m, 1H), 8.75-8.71 (m, | 584.3 |
| | 4-[({[2-(6-{[(1- | 1H), 8.29-8.26 (m, 1H), | |
| | methyl-1H-pyrazol- | 8.05-7.97 (m, 3H), 7.74-7.70 (m, 1H), | |
| | 4- | 7.54-7.50 (m, 2H), 7.25-7.23 (m, 2H), | |
| | yl)methyl]amino}- | 6.78-6.74 (m, 1H), 6.62 (d, 1H), | |
| | pyridin-3- | 4.34 (d, 2H), 3.75 (s, 3H), | |
| | yl)quinolin-4- | 3.20-3.16 (m, 2H), 2.76-2.72 (m, 2H), | |
| | yl]carbonyl}amino)- | 1.82-1.77 (m, 2H), 1.71-1.66 (m, 2H), | |
| | methyl]cyclohexyl}methyl)carbamate | 1.53-1.46 (m, 1H), 1.34 (s, 9H), | |
| | | 1.31-1.25 (m, 1H), 0.97-0.78 (m, | |
| | | 4H) | |
| 167ⁱ | tert-Butyl{[trans- | — | 561.3 |
| | 4-({[(2-{6-[(3- | | |
| | amino-3- | | |
| | oxopropyl)amino]pyridin- | | |
| | 3- | | |
| | yl}quinolin-4-yl)- | | |
| | carbonyl]amino}methyl)cyclohexyl]methyl}carbamate | | |
| 168ⁱ | tert-Butyl[(trans-4- | 8.97-8.95 (m, 1H), 8.76-8.73 (m, | 546.3 |
| | {[({2-[6-(3- | 1H), 8.40-8.37 (m, 1H), | |
| | hydroxyazetidin-1- | 8.05-7.99 (m, 3H), 7.75-7.71 (m, 1H), | |
| | yl)-pyridin-3- | 7.55-7.52 (m, 1H), 6.78-6.74 (m, 1H), | |
| | yl]quinolin-4- | 6.50 (d, 1H), 5.72 (d, 1H), | |
| | yl}carbonyl)amino]- | 4.61-4.56 (m, 1H), 4.26-4.21 (m, 2H), | |
| | methyl}cyclohexyl)methyl]carbamate | 3.77-3.74 (m, 2H), 3.20-3.16 (m, 2H), | |
| | | 2.76-2.71 (m, 2H), 1.82-1.76 (m, | |
| | | 2H), 1.71-1.65 (m, 2H), | |
| | | 1.53-1.46 (m, 1H), 1.34 (s, 9H), 1.32-1.25 (m, | |
| | | 1H), 0.98-0.79 (m, 4H) | |
| 169 | tert-Butyl({trans- | 8.88-8.86 (m, 1H), 8.74-8.71 (m, | 575.4 |
| | 4-[({[2-(6-{[(1S)-1- | 1H), 8.29-8.26 (m, 1H), | |
| | carbamoylpropyl]amino}pyridin- | 8.04-7.97 (m, 3H), 7.74-7.70 (m, 1H), | |
| | 3- | 7.55-7.51 (m, 1H), 7.38-7.37 (m, 1H), | |
| | yl)-quinolin-4-yl]- | 7.02 (d, 1H), 6.96-6.93 (m, 1H), | |
| | carbonyl}amino)methyl]cyclohexyl}methyl)carbamate | 6.77-6.72 (m, 2H), 4.39-4.32 (m, | |
| | | 1H), 3.20-3.16 (m, 2H), | |
| | | 2.76-2.72 (m, 2H), 1.82-1.63 (m, 6H), | |
| | | 1.53-1.46 (m, 1H), 1.34 (s, 9H), | |
| | | 1.31-1.26 (m, 1H), 0.97-0.79 (m, 4H), 0.91 (t, | |
| | | 3H) | |
| 170ⁱ | tert-Butyl({trans- | 8.89-8.88 (m, 1H), 8.73-8.71 (m, | 586.4 |
| | 4-[({[2-(6-{[(5- | 1H), 8.34-8.31 (m, 1H), | |
| | methyl-1,2,4- | 8.06-7.98 (m, 3H), 7.74-7.71 (m, 1H), | |
| | oxadiazol-3- | 7.63-7.60 (m, 1H), 7.55-7.52 (m, 1H), | |
| | yl)methyl]amino}- | 6.77-6.74 (m, 1H), 6.72 (d, 1H), | |
| | pyridin-3- | 4.64 (d, 2H), 3.19-3.16 (m, 2H), | |
| | yl)quinolin-4- | 2.76-2.73 (m, 2H), 2.27 (s, 3H), | |
| | yl]carbonyl}amino)- | 1.82-1.77 (m, 2H), 1.71-1.66 (m, | |
| | methyl]cyclohexyl}- | 2H), 1.54-1.46 (m, 1H), 1.34 (s, 9H), | |
| | methyl)carbamate | 1.32-1.25 (m, 1H), 0.97-0.79 (m, | |
| | | 4H) | |
| 171 | tert-Butyl{[trans- | 9.04-9.02 (m, 1H), 8.76-8.73 (m, | 590.4 |
| | 4-({[(2-{6-[2- | 1H), 8.46-8.43 (m, 1H), | |
| | (hydroxymethyl)morpholin- | 8.05-8.01 (m, 3H), 7.76-7.72 (m, 1H), | |
| | 4-yl]- | 7.56-7.53 (m, 1H), 6.97 (d, 1H), | |
| | pyridin-3- | 6.78-6.75 (m, 1H), 4.83 (t, 1H), | |
| | yl}quinolin-4- | 4.34-4.31 (m, 1H), 4.17-4.14 (m, 1H), | |
| | yl)carbonyl]amino}- | 3.96-3.93 (m, 2H), 3.57-3.51 (m, 1H), | |
| | methyl)cyclohexyl]- | 3.21-3.16 (m, 2H), 2.97-2.89 (m, | |
| | methyl}carbamate | 2H), 2.76-2.72 (m, 2H), | |
| | | 2.69-2.61 (m, 2H), 1.83-1.77 (m, 2H), | |
| | | 1.71-1.66 (m, 2H), 1.53-1.47 (m, 1H), | |
| | | 1.34 (s, 9H), 1.31-1.26 (m, 1H), | |
| | | 0.97-0.79 (m, 4H) | |
| 172 | tert-Butyl[(trans-4- | 9.06-9.04 (m, 1H), 8.77-8.74 (m, | 559.4 |
| | {[({2-[6-(4- | 1H), 8.69-8.68 (m, 1H), | |
| | oxoimidazolidin-1- | 8.50-8.47 (m, 1H), 8.06-8.02 (m, 3H), | |
| | yl)-pyridin-3- | 7.76-7.73 (m, 1H), 7.57-7.54 (m, 1H), | |
| | yl]quinolin-4- | 6.78-6.75 (m, 1H), 6.67 (d, 1H), | |
| | yl}carbonyl)amino]- | 4.88 (s, 2H), 3.94 (s, 2H), | |
| | methyl}cyclohexyl)- | 3.20-3.16 (m, 2H), 2.77-2.72 (m, 2H), | |
| | methyl]carbamate | 1.83-1.77 (m, 2H), 1.71-1.67 (m, 2H), | |
| | | 1.54-1.47 (m, 1H), 1.33 (s, 9H), | |
| | | 1.31-1.26 (m, 1H), 0.97-0.79 (m, | |
| | | 4H) | |

ⁱA salt of the amine was used. Hence, 20 eq of DIPEA was added to the reaction mixture

### Example 173

### Method 21

### tert-Butyl [(trans-4-{[({2-[6-(tetrahydrofuran-3-ylmethoxy)pyridin-3-yl]quinolin-4-yl}carbonyl)amino]methyl}cyclohexyl)methyl]carbamate

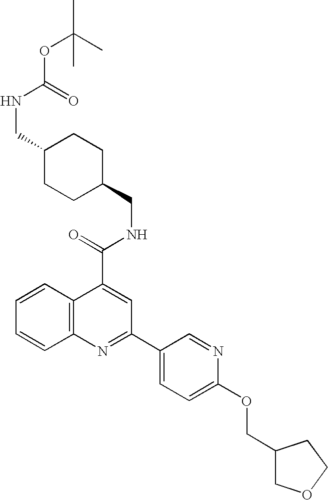

Tetrahydro-3-furanmethanol (83 mg, 0.81 mmol, 5.0 eq.) and KOH (freshly ground, 64 mg, 1.14 mmol, 7.0 eq.) were added sequentially to a solution of tert-butyl({trans-4-[({[2-(6-fluoropyridin-3-yl)quinolin-4-yl]carbonyl}amino)methyl]cyclohexyl}methyl)carbamate (Intermediate VVV) (80 mg, 0.162 mmol) in a mixture of THF (1 mL) and ACN (3 mL) and the reaction mixture was heated to 65° C. for 12 h. The reaction mixture was concentrated in vacuo to leave a residue, which was taken up in DMSO and purified by reversed phase HPLC (Standard method F) to afford the title compound (12 mg, 13%). ¹H NMR (400 MHz, DMSO-d₆) δ 9.05-9.03 (m, 1H), 8.78-8.75 (m, 1H), 8.59-8.57 (m, 1H), 8.10-8.05 (m, 3H), 7.80-7.76 (m, 1H), 7.62-7.58 (m, 1H), 6.98 (d, 1H), 6.78-6.75 (m, 1H), 4.33-4.21 (m, 2H), 3.79-3.73 (m, 2H), 3.66-3.62 (m, 2H), 3.55-3.52 (m, 1H), 3.21-3.18 (m, 2H), 2.76-2.73 (m, 2H), 2.05-1.98 (m, 2H), 1.83-1.79 (m, 2H), 1.71-1.66 (m, 2H), 1.55-1.47 (m, 1H), 1.34 (s, 9H), 1.32-1.25 (m, 1H), 0.97-0.79 (m, 4H); m/z (M+H)⁺ 575.5.

### Examples 174-179

The following Examples 174-179 were prepared by the general procedure of Example 173 (Method 21) using the appropriate alcohol as starting material in place of 2-methanesulfonylethanol.

| | | ¹H NMR (600 MHz, DMSO/DMSO- | MS m/z |
|---|---|---|---|
| Example | Name | d₆) δ | (M + H)⁺ |
| 174 | tert-Butyl[(trans-4- | 9.05-9.03 (m, 1H), 8.78-8.75 (m, 1H), | 535.4 |
| | {[({2-[6-(2- | 8.60-8.57 (m, 1H), 8.10-8.06 (m, 3H), | |
| | hydroxyethoxy)- | 7.80-7.76 (m, 1H), 7.62-7.58 (m, 1H), | |
| | pyridin-3-yl]- | 6.98 (d, 1H), 6.78-6.75 (m, 1H), | |
| | quinolin-4-yl}- | 4.87 (t, 1H), 4.35 (t, 2H), 3.73 (q, 2H), | |
| | carbonyl)amino]- | 3.21-3.18 (m, 2H), 2.76-2.73 (m, 2H), | |
| | methyl}cyclohexyl)methyl]carbamate | 1.82-1.78 (m, 2H), 1.71-1.66 (m, 2H), | |
| | | 1.53-1.48 (m, 1H), 1.34 (s, 9H), | |
| | | 1.31-1.26 (m, 1H), 0.97-0.79 (m, 4H) | |
| 175 | tert-Butyl[(trans-4- | 9.05-9.03 (m, 1H), 8.78-8.74 (m, 1H), | 575.5 |
| | {[({2-[6-(tetrahydro- | 8.59-8.57 (m, 1H), 8.10-8.05 (m, 3H), | |
| | 2H-pyran-4-yloxy)- | 7.80-7.76 (m, 1H), 7.62-7.59 (m, 1H), | |
| | pyridin-3-yl]- | 6.97 (d, 1H), 6.78-6.75 (m, 1H), | |
| | quinolin-4-yl}- | 5.30-5.24 (m, 1H), 3.89-3.84 (m, 4H), | |
| | carbonyl)amino]- | 3.53-3.48 (m, 4H), 3.21-3.17 (m, 2H), | |
| | methyl}cyclohexyl)methyl]carbamate | 2.76-2.73 (m, 2H), 1.83-1.78 (m, 2H), | |
| | | 1.71-1.66 (m, 2H), 1.54-1.46 (m, 1H), | |
| | | 1.34 (s, 9H), 1.31-1.24 (m, 1H), | |
| | | 0.97-0.80 (m, 4H) | |
| 176 | tert-Butyl[(trans-4- | 9.04-9.01 (m, 1H), 8.78-8.74 (m, 1H), | 563.4 |
| | {[({2-[6-(2-hydroxy- | 8.57-8.54 (m, 1H), 8.10-8.04 (m, 3H), | |
| | 1-methylpropoxy)- | 7.81-7.75 (m, 1H), 7.61-7.58 (m, 1H), | |
| | pyridin-3-yl]- | 6.92 (d, 1H), 6.78-6.74 (m, 1H), | |
| | quinolin-4-yl}- | 4.81-4.78 (m, 1H), 3.80-3.74 (m, 2H), | |
| | carbonyl)amino]- | 3.21-3.17 (m, 2H), 2.77-2.72 (m, 2H), | |
| | methyl}cyclohexyl)methyl]carbamate | 1.84-1.78 (m, 2H), 1.71-1.67 (m, 2H), | |
| | | 1.54-1.47 (m, 1H), 1.34 (s, 9H), | |
| | | 1.32-1.29 (m, 1H), 1.25 (d, 3H), 1.10 (d, 3H), | |
| | | 0.98-0.80 (m, 4H) | |
| 177 | tert-Butyl{[trans-4- | 8.79-8.76 (m, 1H), 8.64-8.63 (m, 1H), | 588.4 |
| | ({[(2-{6-[(2- | 8.38-8.35 (m, 1H), 8.02-7.99 (m, 3H), | |
| | oxopyrrolidin-1- | 7.78-7.74 (m, 1H), 7.60-7.56 (m, 1H), | |
| | yl)methoxy]pyridin- | 6.78-6.74 (m, 1H), 6.59 (d, 1H), | |
| | 3-yl}quinolin-4- | 5.40 (s, 2H), 3.51 (t, 2H), 3.20-3.17 (m, | |
| | yl)carbonyl]amino}- | 2H), 2.76-2.73 (m, 2H), 2.25 (t, 2H), | |
| | methyl)cyclohexyl]- | 1.93-1.87 (m, 2H), 1.82-1.78 (m, 2H), | |
| | methyl}carbamate | 1.71-1.66 (m, 2H), 1.53-1.47 (m, 1H), | |
| | | 1.34 (s, 9H), 1.31-1.25 (m, 1H), | |
| | | 0.97-0.79 (m, 4H) | |
| 178 | tert-Butyl[(trans-4- | 9.96-9.95 (m, 1H), 9.23-9.22 (m, 1H), | 548.4 |
| | {[({2-[6-(2-amino-2- | 8.80-8.76 (m, 1H), 8.70-8.67 (m, 1H), | |
| | oxoethoxy)pyridin-3- | 8.27-8.25 (m, 1H), 8.11-8.07 (m, 3H), | |
| | yl]quinolin-4- | 7.82-7.78 (m, 1H), 7.64-7.61 (m, 1H), | |
| | yl}carbonyl)amino]- | 6.78-6.74 (m, 1H), 5.78-5.72 (m, 1H), | |
| | methyl}cyclohexyl)methyl]carbamate | 4.09-4.06 (m, 2H), 3.22-3.17 (m, 2H), | |
| | | 2.76-2.72 (m, 2H), 1.83-1.77 (m, 2H), | |
| | | 1.72-1.66 (m, 2H), 1.54-1.48 (m, 1H), | |
| | | 1.34 (s, 9H), 1.31-1.25 (m, 1H), | |
| | | 0.98-0.79 (m, 4H) | |
| 179 | tert-Butyl{[trans-4- | 9.05-9.03 (m, 1H), 8.78-8.75 (m, 1H), | 617.5 |
| | ({[(2-{6-[2-(4- | 8.59-8.56 (m, 1H), 8.10-8.05 (m, 3H), | |
| | methylpiperazin-1- | 7.80-7.76 (m, 1H), 7.62-7.58 (m, 1H), | |
| | yl)ethoxy]pyridin-3- | 6.97 (d, 1H), 6.78-6.75 (m, 1H), | |
| | yl}quinolin-4- | 4.35 (t, 2H), 3.21-3.18 (m, 2H), | |
| | yl)carbonyl]amino}- | 2.76-2.73 (m, 2H), 2.70-2.68 (m, 2H), 2.14 (s, | |
| | methyl)cyclohexyl]- | 3H), 1.83-1.78 (m, 2H), 1.71-1.66 (m, | |
| | methyl}carbamate | 2H), 1.55-1.47 (m, 1H), 1.34 (s, 9H), | |
| | | 1.32-1.26 (m, 1H), 0.98-0.79 (m, 4H) | |

### Example 180

### tert-Butyl [(trans-4-{[({2-[3-(4-methylpiperazin-1-yl)propoxy]quinolin-4-yl}carbonyl)amino]methyl}cyclohexyl)methyl]carbamate

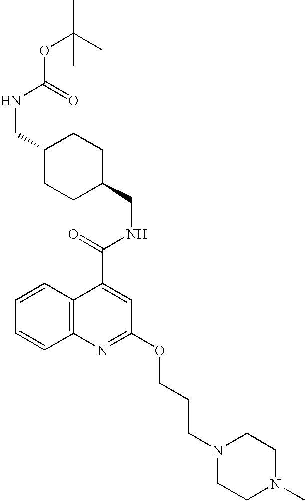

4-Methyl-1-(hydroxypropyl)-piperazine (0.27 g, 1.7 mmol) and KOH (freshly ground, 0.15 g, 2.7 mmol) were added sequentially to a solution of tert-butyl {[trans-4-({[(2-chloroquinolin-4-yl)carbonyl]amino}methyl)cyclohexyl]-methyl}carbamate (Example 35) (0.15 g, 0.35 mmol) in a mixture of THF (1 mL) and ACN (3 mL). The reaction mixture was heated at 50° C. for 12 h and then DMSO was added and the mixture purified by HPLC (Standard method G). The fractions containing the title compound were partly concentrated in vacuo and then a saturated aq. solution of NaHCO₃ was added. DCM was added and the layers were separated. The organic layer was dried (phase separator) and concentrated in vacuo to give the title compound (90 mg, 47%). ¹H NMR (400 MHz, CDCl₃) δ 8.05 (d, 1H), 7.82 (d, 1H), 7.63 (t, 1H), 7.40 (t, 1H), 6.92 (s, 1H), 6.16-6.02 (m, 1H), 4.65-4.56 (m, 1H), 4.52 (t, 2H), 3.37 (t, 2H), 2.97 (t, 2H), 2.62-2.36 (m, 10H), 2.28 (s, 3H), 2.09-1.96 (m, 2H), 1.91-1.75 (m, 4H), 1.63-1.52 (m, 1H), 1.44 (s, 9H), 1.42-1.35 (m, 1H), 1.13-0.85 (m, 4H); m/z (M+H)⁺ 554.1.

### Examples 181-184

The following examples were prepared by the procedure described for Example 180 using the appropriate alcohol as starting material in place of 4-methyl-1-(hydroxypropyl)piperazine.

| | | | MS |
|---|---|---|---|
| | | | m/z |
| Example | Name | ¹H NMR (400 MHz, CDCl₃) δ | (M + H)⁺ |
| 181 | tert-Butyl[(trans-4- | 8.04 (d, 1H), 7.81 (d, 1H), | 499.2 |
| | {[({2-[3- | 7.64-7.57 (m, 1H), 7.42-7.34 (m, 1H), 6.91 (s, | |
| | (dimethylamino)- | 1H), 6.09-5.99 (m, 1H), | |
| | propoxy]quinolin-4- | 4.62-4.53 (m, 1H), 4.50 (t, 2H), 3.35 (t, 2H), | |
| | yl}carbonyl)amino]- | 3.00-2.90 (m, 2H), 2.48-2.40 (m, | |
| | methyl}cyclohexyl)- | 2H), 2.24 (s, 6H), 2.03-1.93 (m, 2H), | |
| | methyl]carbamate | 1.89-1.74 (m, 4H), 1.61-1.49 (m, | |
| | | 1H), 1.41 (s, 9H), 1.41-1.35 (m, 1H), | |
| | | 1.11-0.87 (m, 4H). | |
| 182 | tert-Butyl[(trans-4- | 8.06 (d, 1H), 7.82 (d, 1H), | 540.1 |
| | {[({2-[2-(4- | 7.68-7.59 (m, 1H), 7.46-7.36 (m, 1H), 6.97 (s, | |
| | methylpiperazin-1- | 1H), 6.16-6.07 (m, 1H), 4.62 (t, 3H), | |
| | yl)ethoxy]quinolin-4- | 3.36 (t, 2H), 3.00-2.92 (m, 2H), | |
| | yl}carbonyl)amino]methyl}cyclohexyl)methyl]carbamate | 2.84 (t, 2H), 2.73-2.38 (m, 8H), 2.28 (s, | |
| | | 3H), 1.93-1.75 (m, 4H), | |
| | | 1.66-1.51 (m, 1H), 1.43 (s, 9H), 1.43-1.34 (m, | |
| | | 1H), 1.13-0.80 (m, 4H). | |
| 183^{a} | tert-Butyl[(trans-4- | 8.05 (d, 1H), 7.82 (d, 1H), | 582.4 |
| | {[({2-[3-(4- | 7.70-7.59 (m, 1H), 7.46-7.37 (m, 1H), 6.92 (s, | |
| | acetylpiperazin-1- | 1H), 6.22-6.09 (m, 1H), | |
| | yl)propoxy]quinolin-4- | 4.67-4.58 (m, 1H), 4.54 (t, 2H), 3.62 (t, 2H), | |
| | yl}carbonyl)amino]- | 3.47 (t, 2H), 3.37 (t, 2H), | |
| | methyl}cyclohexyl)- | 3.02-2.93 (m, 2H), 2.60-2.52 (m, 2H), | |
| | methyl]carbamate | 2.51-2.40 (m, 4H), 2.07 (s, 3H), | |
| | | 2.06-1.97 (m, 2H), 1.94-1.75 (m, 4H), | |
| | | 1.65-1.52 (m, 1H), 1.44 (s, 9H), | |
| | | 1.43-1.38 (m, 1H), 1.12-0.88 (m, 4H). | |
| 184^{b} | tert-Butyl{[trans-4- | 9.04 (s, 1H), 8.79-8.73 (m, 1H), | 562.3 |
| | ({[(2-{6-[2- | 8.57 (d, 1H), 8.11-8.05 (m, 3H), | |
| | (dimethylamino)ethoxy]pyridin- | 7.80-7.75 (m, 1H), 7.62-7.58 (m, 1H), | |
| | 3- | 6.97 (d, 1H), 6.78-6.73 (m, 1H), | |
| | yl}quinolin-4- | 4.42 (t, 2H), 3.22-3.17 (m, 2H), | |
| | yl)carbonyl]amino}- | 2.76-2.72 (m, 2H), 2.23 (s, 6H), | |
| | methyl)cyclohexyl]- | 1.83-1.77 (m, 2H), 1.71-1.65 (m, | |
| | methyl}carbamate | 2H), 1.54-1.47 (m, 1H), 1.33 (s, 9H), | |
| | | 1.31-1.26 (m, 1H), 0.98-0.79 (m, | |
| | | 4H).^{c} | |

^{a}Prepared using 3-(4-acetylpiperazin-1-yl)propan-1-ol (Intermediate WWW) in place of 4-methyl-1-(hydroxypropyl)piperazine
^{b}Prepared by using tert-Butyl({trans-4-[({[2-(6-fluoropyridin-3-yl)quinolin-4-yl]carbonyl}amino)-methyl]cyclohexyl}methyl)carbamate (Intermediate VVV) in place of tert-Butyl{[trans-4-({[(2-chloroquinolin-4-yl)carbonyl]amino}methyl)cyclo-hexyl]-methyl}carbamate (Example 35)
^{c}¹H NMR (600 MHz, DMSO/DMSO-d₆)*

### Example 185

### Method 22

### tert-Butyl [(trans-4-{[({2-[4-(2-hydroxyethyl)piperazin-1-yl]quinolin-4-yl}carbonyl)amino]methyl}cyclohexyl)methyl]carbamate

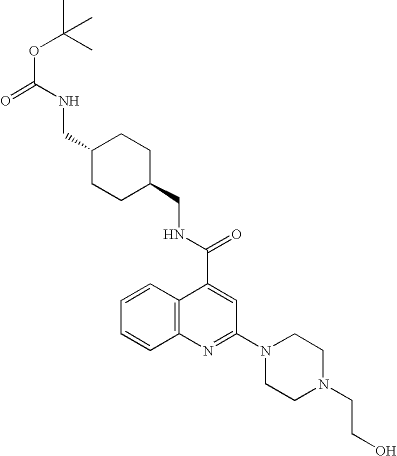

1-piperazineethanol (0.43 g, 3.3 mmol, 17 eq.) was added to a solution of tert-butyl {[trans-4-({[(2-chloroquinolin-4-yl)carbonyl]amino}methyl)cyclohexyl]methyl}-carbamate (Example 35) (80 mg, 0.19 mmol) in pyridine (2 mL) in a microwave vial and the reaction vessel was sealed and heated in a microwave at 135° C. for 30 min. The reaction mixture was then concentrated in vacuo to leave a residue which was dissolved in THF and passed through a plug of silica using THF as eluent. The mixture was concentrated in vacuo to leave a residue which was dissolved in DMSO and purified by HPLC (HPLC Standard method C) to give the title compound (50 mg, 51%). ¹H NMR (600 MHz, DMSO/DMSO-d₆)* δ 8.58 (t, 1H), 7.77 (d, 1H), 7.57-7.54 (m, 1H), 7.52-7.48 (m, 1H), 7.22-7.18 (m, 1H), 7.14 (s, 1H), 6.76 (t, 1H), 4.43 (t, 2H), 3.68-3.63 (m, 4H), 3.52 (q, 2H), 3.12 (t, 2H), 2.74 (t, 2H), 2.43-2.39 (m, 4H), 1.80-1.63 (m, 4H), 1.51-1.42 (m, 1H), 1.34 (s, 9H), 1.30-1.25 (m, 1H), 0.94-0.77 (m, 4H); m/z (M+H)⁺ 526.3.

### Examples 186-189

The following examples were prepared by the method described in Example 185 (Method 22) using the appropriate amine as starting material in place of 1-piperazineethanol.

| | | | MS |
|---|---|---|---|
| | | ¹H NMR (600 MHz, | m/z |
| Example | Name | DMSO/DMSO-d₆)* δ | (M + H)⁺ |
| 186 | tert-Butyl{[trans-4- | 8.58 (t, 1H), 7.77 (d, 1H), | 553.4 |
| | ({[(2-{4-[2- | 7.57-7.54 (m, 1H), | |
| | (dimethylamino)ethyl]- | 7.52-7.49 (m, 1H), 7.22-7.19 (m, | |
| | piperazin-1-yl}quinolin- | 1H), 7.14 (s, 1H), 6.76 (t, | |
| | 4-yl)carbonyl]amino}- | 1H), 3.69-3.63 (m, 4H), | |
| | methyl)cyclohexyl]- | 3.12 (t, 2H), 2.74 (t, 2H), | |
| | methyl}carbamate | 2.42-2.39 (m, 4H), 2.14 (s, | |
| | | 6H), 1.81-1.64 (m, 4H), | |
| | | 1.50-1.43 (m, 1H), 1.34 (s, | |
| | | 9H), 1.30-1.25 (m, 1H), | |
| | | 0.95-0.78 (m, 4H). | |
| 187 | tert-Butyl({trans-4- | 8.57 (t, 1H), 7.75 (d, 1H), | 497.3 |
| | [({[2-(4- | 7.55-7.52 (m, 1H), | |
| | hydroxypiperidin-1- | 7.51-7.47 (m, 1H), 7.20-7.16 (m, | |
| | yl)quinolin-4- | 1H), 7.14 (s, 1H), 6.76 (t, | |
| | yl]carbonyl}amino)- | 1H), 4.72 (d, 1H), | |
| | methyl]cyclohexyl}- | 4.22-4.15 (m, 2H), 3.76-3.68 (m, 1H), | |
| | methyl)carbamate | 3.25-3.19 (m, 2H), 3.12 (t, | |
| | | 2H), 2.74 (t, 2H), | |
| | | 1.83-1.74 (m, 4H), 1.71-1.65 (m, 2H), | |
| | | 1.50-1.43 (m, 1H), | |
| | | 1.42-1.34 (m, 2H), 1.34 (s, 9H), | |
| | | 1.29-1.25 (m, 1H), | |
| | | 0.94-0.76 (m, 4H). | |
| 188 | tert-Butyl[(trans-4- | 8.57 (t, 1H), 7.75 (d, 1H), | 511.3 |
| | {[({2-[4- | 7.55-7.51 (m, 1H), | |
| | (hydroxymethyl)- | 7.50-7.46 (m, 1H), 7.19-7.15 (m, | |
| | piperidin-1-yl]quinolin- | 1H), 7.13 (s, 1H), 6.76 (t, | |
| | 4-yl}carbonyl)amino]- | 1H), 4.55-4.50 (m, 2H), | |
| | methyl}cyclohexyl)- | 4.47 (t, 1H), 3.25 (t, 2H), | |
| | methyl]carbamate | 3.12 (t, 2H), 2.91-2.84 (m, | |
| | | 2H), 2.74 (t, 2H), | |
| | | 1.80-1.71 (m, 4H), 1.70-1.61 (m, 3H), | |
| | | 1.51-1.42 (m, 1H), 1.34 (s, | |
| | | 9H), 1.30-1.25 (m, 1H), | |
| | | 1.16-1.07 (m, 2H), | |
| | | 0.95-0.77 (m, 4H). | |
| 189 | tert-Butyl[(trans-4- | 8.57 (t, 1H), 7.74 (d, 1H), | 525.3 |
| | {[({2-[4-(2- | 7.54-7.5 1 (m, 1H), | |
| | hydroxyethyl)piperidin- | 7.50-7.46 (m, 1H), 7.19-7.15 (m, | |
| | 1-yl]quinolin-4- | 1H), 7.11 (s, 1H), | |
| | yl}carbonyl)amino]- | 6.77-6.73 (m, 1H), 4.52-4.46 (m, 2H), | |
| | methyl}cyclohexyl)- | 4.43-4.36 (m, 1H), 3.12 (t, | |
| | methyl]carbamate | 2H), 2.90-2.83 (m, 2H), | |
| | | 2.74 (t, 2H), 1.79-1.71 (m, | |
| | | 4H), 1.69-1.62 (m, 3H), | |
| | | 1.50-1.42 (m, 1H), | |
| | | 1.38-1.33 (m, 2H), 1.33 (s, 9H), | |
| | | 1.30-1.24 (m, 1H), | |
| | | 1.14-1.05 (m, 2H), 0.94-0.76 (m, | |
| | | 4H). | |

### Example 190

### tert-Butyl {[trans-4-({[(2-piperazin-1-ylquinolin-4-yl)carbonyl]amino}methyl)-cyclohexyl]methyl}carbamate

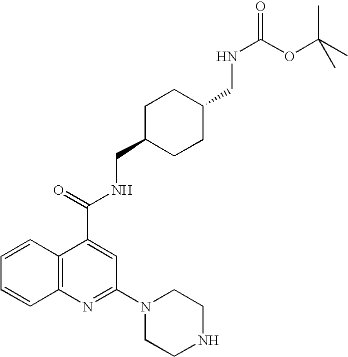

Piperazine (239 mg, 2.78 mmol) was added to a solution of tert-butyl {[trans-4-({[(2-chloroquinolin-4-yl)carbonyl]amino}methyl)cyclohexyl]methyl}carbamate (Example 35) (100 mg, 0.23 mmol) in pyridine (2 mL) and the reaction mixture was heated to 130° C. for 30 min. The reaction mixture was concentrated in vacuo to leave a residue which was purified by flash column chromatography, using a gradient of 5-70% MeOH (2% TEA) in DCM as eluent, and then preparative HPLC (Standard method H) to give the title compound (43 mg, 39%). ¹H NMR (300 MHz, CDCl₃) δ 8.61-8.56 (m, 1H), 7.80-7.77 (m, 1H), 7.57-7.48 (m, 3H), 7.23-7.18 (m, 1H), 6.79-6.74 (m, 1H), 3.63-3.58 (m, 4H), 3.16-3.10 (m, 2H), 2.81-2.73 (m, 6H), 1.81-1.75 (m, 2H), 1.72-1.66 (m, 2H), 1.52-1.44 (m, 1H), 1.36 (s, 9H), 1.32-1.26 (m, 1H), 0.96-0.80 (m, 4H); m/z (M+H)⁺ 482.5.

### Examples 191-192

The following Examples 191-192 were prepared by the same procedure as described for Example 190 using the appropriate amine, Intermediate YYY or Intermediate ZZZ, as starting material in place of piperazine and DIPEA (37 eq.) was added to the reaction mixtures.

| | | | MS |
|---|---|---|---|
| | | | m/z |
| Example | Name | ¹H NMR (400 MHz, CDCl₃) δ | (M + H)⁺ |
| 191 | tert-Butyl[(trans-4- | 8.60-8.56 (m, 1H), 7.88-7.83 (m, | 552.5 |
| | {[({2-[4- | 1H), 7.76-7.73 (m, 1H), | |
| | (acetamidomethyl)- | 7.55-7.47 (m, 2H), 7.21-7.14 (m, 2H), | |
| | piperidin-1-yl]- | 6.78-6.73 (m, 1H), 4.53-4.48 (m, 2H), | |
| | quinolin-4-yl}- | 3.14-3.10 (m, 2H), 2.97-2.84 (m, | |
| | carbonyl)amino]methyl}cyclohexyl)methyl]- | 6H), 2.75-2.72 (m, 2H), 1.78 (s, 3H), | |
| | carbamate | 1.77-1.64 (m, 6H), 1.49-1.43 (m, | |
| | | 1H), 1.33 (s, 9H), 1.29-1.23 (m, 1H), | |
| | | 1.14-1.05 (m, 1H), 0.94-0.77 (m, 4H) | |
| 192 | tert-Butyl[(trans-4- | 8.59-8.55 (m, 1H), 7.81-7.77 (m, | 566.6 |
| | {[({2-[4-(2- | 1H), 7.75-7.73 (m, 1H), | |
| | acetamidoethyl)- | 7.54-7.47 (m, 2H), 7.19-7.16 (m, 1H), 7.12 (s, | |
| | piperidin-1-yl]- | 1H), 6.77-6.73 (m, 1H), | |
| | quinolin-4-yl}- | 4.51-4.47 (m, 2H), 3.14-3.10 (m, 2H), | |
| | carbonyl)amino]methyl}cyclohexyl)methyl]- | 3.08-3.04 (m, 2H), 2.89-2.83 (m, 4H), | |
| | carbamate | 2.75-2.72 (m, 2H), 1.75 (s, 3H), | |
| | | 1.79-1.70 (m, 4H), 1.70-1.65 (m, | |
| | | 2H), 1.59-1.53 (m, 2H), | |
| | | 1.48-1.43 (m, 1H), 1.33 (s, 9H), 1.29-1.24 (m, | |
| | | 1H), 1.15-1.04 (m, 1H), | |
| | | 0.94-0.77 (m, 4H) | |

### Example 193

### tert-Butyl {[trans-4-({[(2-{4-[2-(dimethylamino)ethyl]piperidin-1-yl}quinolin-4-yl)carbonyl]amino}methyl)cyclohexyl]methyl}carbamate

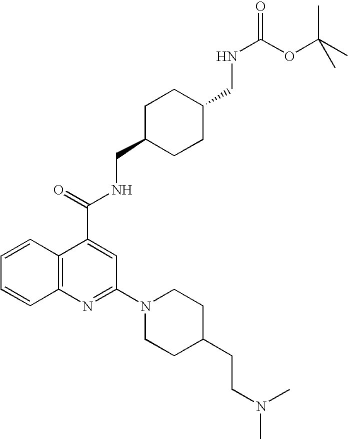

Dimethylamine (2 M in MeOH, 1.66 mL, 3.3 mmol) was added to a solution of 2-[1-(4-{[(trans-4-{[(tert-butoxycarbonyl)amino]methyl}cyclohexyl)methyl]carbamoyl}-quinolin-2-yl)piperidin-4-yl]ethyl methanesulfonate (Intermediate XXX Step i)) (600 mg, 0.17 mmol) in DMF (1 mL) and the reaction mixture was stirred for 20 h at rt. The reaction mixture was then purified by flash column chromatography, using a gradient of 5-70% MeOH (2% TEA) in DCM as eluent, to give the title compound (30 mg, 33%). ¹H NMR (300 MHz, CDCl₃) δ 7.83-7.80 (m, 1H), 7.68-7.64 (m, 1H), 7.54-7.49 (m, 1H), 7.24-7.14 (m, 2H), 6.96 (s, 1H), 6.28-6.24 (m, 1H), 4.65-4.60 (m, 1H), 4.49-4.44 (m, 2H), 3.35 (t, 2H), 2.99-2.87 (m, 5H), 2.43-2.39 (m, 2H), 2.30 (s, 6H), 1.89-1.76 (m, 6H), 1.64-1.54 (m, 1H), 1.49-1.40 (m, 2H), 1.43 (s, 9H), 1.31-1.19 (m, 2H), 1.09-0.89 (m, 4H); m/z (M+H)⁺ 552.3.

### Example 194

### tert-Butyl [(trans-4-{[({2-[4-(2-aminoethyl)piperidin-1-yl]quinolin-4-yl}carbonyl)amino]methyl}cyclohexyl)methyl]carbamate

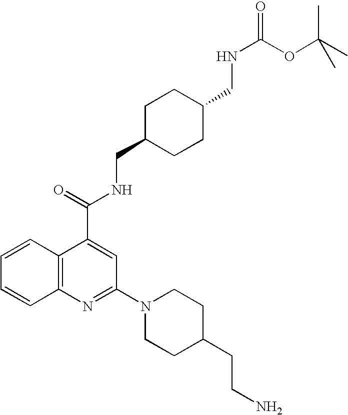

Tin(II) chloride (118 mg, 0.62 mmol), TEA (0.26 mL, 1.86 mmol) and thiophenol (0.26 mL, 2.5 mmol) were added to a solution of tert-butyl [(trans-4-{[({2-[4-(2-azidoethyl)piperidin-1-yl]quinolin-4-yl}carbonyl)amino]methyl}cyclohexyl)methyl]-carbamate (Intermediate XXX) (93 mg, 0.17 mmol) in THF (4 mL) and the reaction mixture was stirred for 10 min at rt. The reaction mixture was concentrated in vacuo to leave a residue, which was purified by flash column chromatography, using a gradient of 0-10% MeOH:DCM and then a gradient of 10-75% MeOH (2% TEA) in DCM as eluent, to give the title compound (17 mg, 19%). ¹H NMR (300 MHz, CDCl₃) δ 8.27-8.24 (m, 1H), 8.10-8.07 (m, 1H), 7.97-7.93 (m, 1H), 7.83 (s, 1H), 7.67-7.63 (m, 2H), 7.59-7.56 (m, 1H), 4.94-4.88 (m, 2H), 3.78-3.71 (m, 3H), 3.61-3.57 (m, 1H), 3.41-3.34 (m, 4H), 3.23-3.19 (m, 2H), 2.76 (s, 1H), 2.32-2.20 (m, 6H), 2.11-1.95 (m, 1H), 1.94-1.79 (m, 2H), 1.85 (s, 9H), 1.74-1.66 (m, 3H), 1.51-1.33 (m, 4H); m/z (M+H)⁺ 524.3.

### Example 195

### Tetrahydro-2H-pyran-4-yl {[trans-4-({[(2-{6-[2-(dimethylamino)ethoxy]pyridin-3-yl}quinolin-4-yl)carbonyl]amino}methyl)cyclohexyl]methyl}carbamate

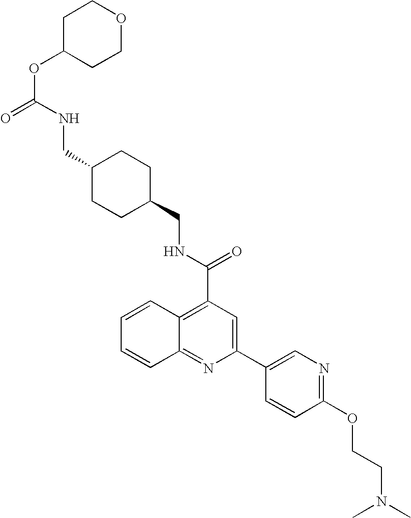

A solution of 2-dimethylaminoethanol (68 mg, 0.77 mmol) in THF (2 mL) and KOH (freshly ground, 43 mg, 0.77 mmol) were added to a solution of tetrahydro-2H-pyran-4-yl({trans-4-[({[2-(6-fluoropyridin-3-yl)quinolin-4-yl]carbonyl}amino)methyl]cyclohexyl} methyl)carbamate (Intermediate AAAA) (100 mg, 0.19 mmol) in ACN (2 mL). The reaction mixture was heated at 60° C. for 1 h and then filtered through a plug of silica using THF as eluent. The solvent was concentrated in vacuo and the residue was dissolved in DMSO and purified by preparative HPLC (Standard method C) to give the title compound (7 mg, 6%). ¹H NMR (600 MHz, DMSO/DMSO-d₆*) δ 9.05-9.04 (m, 1H), 8.78-8.75 (m, 1H), 8.59-8.56 (m, 1H), 8.10-8.05 (m, 3H), 7.80-7.76 (m, 1H), 7.62-7.58 (m, 1H), 7.11-7.07 (m, 1H), 6.97 (d, 1H), 4.65-4.59 (m, 1H), 4.41 (t, 2H), 3.78-3.74 (m, 4H), 3.21-3.18 (m, 2H), 2.83-2.79 (m, 2H), 2.64-2.61 (m, 2H), 2.20 (s, 6H), 1.84-1.77 (m, 4H), 1.72-1.67 (m, 2H), 1.55-1.48 (m, 1H), 1.47-1.40 (m, 2H), 1.35-1.29 (m, 1H), 0.98-0.82 (m, 4H); m/z (M+H)⁺ 590.3.

### Example 196

### (3S)-Tetrahydrofuran-3-yl {[trans-4-({[(2-{6-[2-(dimethylamino)ethoxy]pyridin-3-yl}quinolin-4-yl)carbonyl]amino}methyl)cyclohexyl]methyl}carbamate

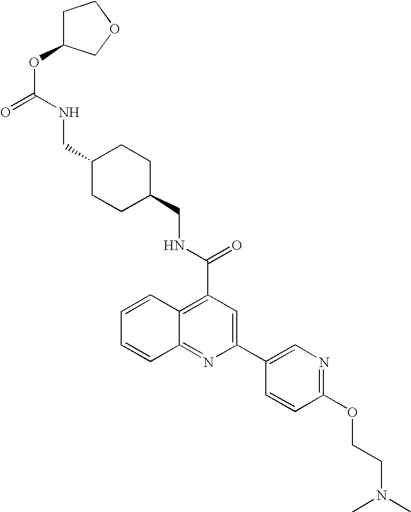

Example 196 was prepared using the procedure described for Example 195 using (3S)-tetrahydrofuran-3-yl ({trans-4-[({[2-(6-fluoropyridin-3-yl)quinolin-4-yl]carbonyl}-amino)methyl]cyclohexyl}methyl)carbamate (Intermediate BBBB) as starting material in place of tetrahydro-2H-pyran-4-yl ({trans-4-[({[2-(6-fluoropyridin-3-yl)quinolin-4-yl]carbonyl}amino)methyl]cyclohexyl}-methyl)carbamate to give the title compound (7 mg, 6%). m/z (M+H)⁺ 576.4.

### Example 197

### Tetrahydro-2H-pyran-4-yl {[trans-4-({[(2-{6-[(2-hydroxyethyl)amino]pyridin-3-yl}quinolin-4-yl)carbonyl]amino}methyl)cyclohexyl]methyl}carbamate

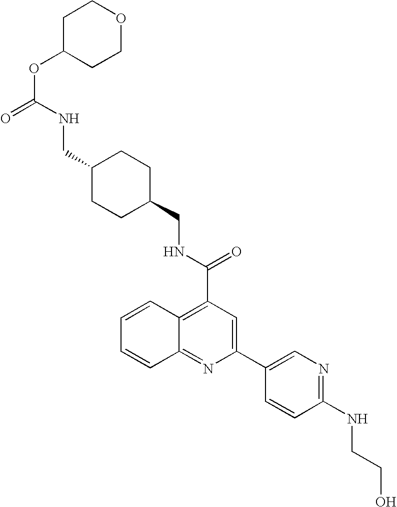

Ethanolamine (188 mg, 3.1 mmol) was added to a solution of tetrahydro-2H-pyran-4-yl({trans-4-[({[2-(6-fluoropyridin-3-yl)quinolin-4-yl]-carbonyl}amino)methyl]cyclo-hexyl}methyl)carbamate (Intermediate AAAA) (80 mg, 0.15 mmol) in pyridine (2 mL) and the reaction mixture was heated to 130° C. in a sealed microwave vial using microwave irradiation for 30 min. The reaction mixture was concentrated in vacuo to leave a residue which was dissolved in THF and washed through a plug of silica using THF as eluent. Concentration in vacuo left a residue which was dissolved in DMSO and purified by preparative HPLC (Standard method C) to afford the title compound (60 mg, 69%). ¹H NMR (600 MHz, DMSO/DMSO-d₆*) δ 8.89-8.88 (m, 1H), 8.75-8.72 (m, 1H), 8.29-8.26 (m, 1H), 8.04-7.97 (m, 3H), 7.74-7.70 (m, 1H), 7.54-7.50 (m, 1H), 7.11-7.07 (m, 1H), 7.03-6.99 (m, 1H), 6.63 (d, 1H), 4.76 (t, 2H), 4.65-4.60 (m, 1H), 3.78-3.74 (m, 4H), 3.54 (q, 2H), 3.20-3.16 (m, 2H), 2.83-2.79 (m, 2H), 1.83-1.76 (m, 4H), 1.72-1.66 (m, 2H), 1.54-1.40 (m, 3H), 1.34-1.28 (m, 1H), 0.97-0.80 (m, 4H); m/z (M+H)⁺ 562.3.

### Examples 198-200

The following Examples 198-200 were prepared using the procedure described for Example 197. Example 198 used Intermediate AAAA as starting material and N,N′-dimethylethylenediamine in place of ethanolamine. Examples 199-200 used Intermediate BBBB as starting material and N,N′-dimethylethylenediamine or ethanolamine as the required amine respectively.

| | | | MS |
|---|---|---|---|
| | | ¹H NMR δ (600 MHz, | m/z |
| Example | Name | DMSO/DMSO-d₆)* | (M + H⁺) |
| 198 | Tetrahydro-2H-pyran-4- | 8.90-8.89 (m, 1H), 8.74-8.71 (m, | 589.3 |
| | yl({trans-4-[({[2-(6- | 1H), 8.28-8.25 (m, 1H), | |
| | {[2-(dimethylamino)- | 8.04-7.97 (m, 3H), 7.74-7.70 (m, 1H), | |
| | ethyl]amino}pyridin-3- | 7.53-7.50 (m, 1H), 7.11-7.07 (m, | |
| | yl)quinolin-4- | 1H), 6.89-6.85 (m, 1H), 6.63 (d, | |
| | yl]carbonyl}amino)- | 1H), 4.66-4.59 (m, 1H), | |
| | methyl]cyclohexyl}- | 3.79-3.74 (m, 4H), 3.42-3.38 (m, 2H), | |
| | methyl)carbamate | 3.20-3.17 (m, 2H), 2.82-2.78 (m, | |
| | | 2H), 2.44-2.39 (m, 2H), 2.17 (s, | |
| | | 6H), 1.83-1.77 (m, 4H), | |
| | | 1.73-1.68 (m, 2H), 1.53-1.39 (m, 3H), | |
| | | 1.35-1.29 (m, 1H), 0.97-0.81 (m, | |
| | | 4H) | |
| 199 | (3S)-Tetrahydrofuran-3- | 8.90-8.89 (m, 1H), 8.74-8.72 (m, | 575.4 |
| | yl({trans-4-[({[2-(6- | 1H), 8.28-8.25 (m, 1H), | |
| | {[2- | 8.04-7.97 (m, 3H), 7.74-7.70 (m, 1H), | |
| | (dimethylamino)ethyl]- | 7.53-7.50 (m, 1H), 7.18-7.15 (m, | |
| | amino}pyridin-3- | 1H), 6.89-6.86 (m, 1H), 6.62 (d, | |
| | yl)quinolin-4- | 1H), 5.08-5.03 (m, 1H), | |
| | yl]carbonyl}amino)- | 3.75-3.63 (m, 4H), 3.42-3.38 (m, 2H), | |
| | methyl]cyclohexyl}- | 3.20-3.16 (m, 2H), 2.82-2.79 (m, | |
| | methyl)carbamate | 2H), 2.43-2.38 (m, 2H), 2.16 (s, | |
| | | 6H), 2.10-2.02 (m, 2H), | |
| | | 1.82-1.77 (m, 2H), 1.72-1.66 (m, 2H), | |
| | | 1.53-1.47 (m, 1H), 1.34-1.27 (m, | |
| | | 1H), 0.98-0.80 (m, 4H) | |
| 200 | (3S)-Tetrahydrofuran-3- | 8.90-8.88 (m, 1H), 8.75-8.72 (m, | 548.3 |
| | yl{[trans-4-({[(2-{6- | 1H), 8.29-8.26 (m, 1H), | |
| | [(2-hydroxyethyl)amino]pyridin- | 8.04-7.97 (m, 3H), 7.73-7.70 (m, 1H), | |
| | 3-yl}quinolin- | 7.54-7.50 (m, 1H), 7.18-7.15 (m, | |
| | 4-yl)carbonyl]amino}methyl)cyclohexyl]methyl}- | 1H), 7.02-7.00 (m, 1H), 6.63 (d, | |
| | carbamate | 1H), 5.07-5.04 (m, 1H), 4.76 (t, | |
| | | 1H), 3.75-3.64 (m, 4H), 3.54 (q, | |
| | | 2H), 3.42-3.37 (m, 2H), | |
| | | 3.20-3.16 (m, 2H), 2.82-2.78 (m, 2H), | |
| | | 2.10-2.02 (m, 2H), 1.83-1.76 (m, | |
| | | 2H), 1.72-1.66 (m, 2H), | |
| | | 1.54-1.47 (m, 1H), 1.34-1.27 (m, 1H), | |
| | | 0.98-0.81 (m, 4H) | |

### Example 201

### (3S)-Tetrahydrofuran-3-yl {[trans-4-({[(2-{6-[(3R)-3-hydroxypyrrolidin-1-yl]pyridin-3-yl}quinolin-4-yl)carbonyl]amino}methyl)cyclohexyl]methyl}-carbamate

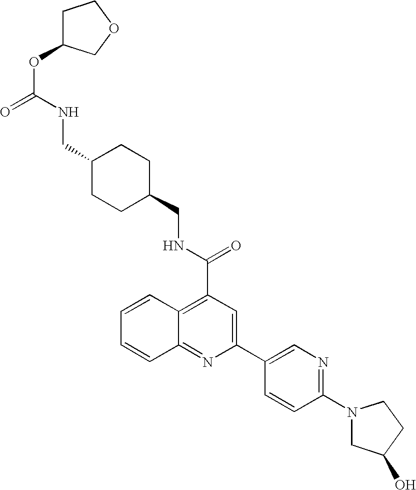

TFA (4 mL, 52 mmol) was added to a solution of tert-butyl {[trans-4-({[(2-{6-[(3R)-3-hydroxypyrrolidin-1-yl]pyridin-3-yl}quinolin-4-yl)carbonyl]amino}methyl)cyclohexyl]-methyl}carbamate (Example 147) (243 mg, 0.43 mmol) in DCM (10 mL) and the reaction mixture was stirred for 1 h at rt. The solvent was concentrated in vacuo to leave the crude amine which was used in the next step without further purification. m/z (M+H)⁺ 460.3.

A solution of 4-nitrophenyl (3S)-tetrahydrofuran-3-yl carbonate (Intermediate XX) (71 mg, 0.28 mmol) in THF (3 mL) and TEA (0.27 mL, 1.94 mmol) was added to a solution of the crude amine (99 mg, 0.22 mmol) in THF (2 mL) and the reaction mixture was stirred at 40° C. for 3 h and then at rt for 16 h. The reaction mixture was concentrated in vacuo to leave a residue which was purified by flash column chromatography, using a mixture of 2-25% MeOH in DCM as eluent, to give the title compound (16 mg, 13%). ¹H NMR (400 MHz, DMSO-d₆) δ 9.00-8.96 (m, 1H), 8.76-8.70 (m, 1H), 8.41-8.36 (m, 1H), 8.05-7.98 (m, 3H), 7.74-7.68 (m, 1H), 7.54-7.48 (m, 1H), 7.19-7.13 (m, 1H), 6.59-6.55 (m, 1H), 5.07-5.03 (m, 1H), 4.42-4.35 (m, 1H), 3.76-3.47 (m, 7H), 3.20-3.15 (m, 3H), 2.82-2.77 (m, 2H), 2.09-1.99 (m, 2H), 1.93-1.86 (m, 1H), 1.84-1.77 (m, 3H), 1.72-1.66 (m, 2H), 1.54-1.47 (m, 1H), 1.31 (s, 2H), 0.99-0.78 (m, 4H); m/z (M+H)⁺ 574.3.

### Example 202

### Tetrahydro-2H-pyran-4-yl {[trans-4-({[(2-{6-[(3R)-3-hydroxypyrrolidin-1-yl]pyridin-3-yl}quinolin-4-yl)carbonyl]amino}methyl)cyclohexyl]methyl}-carbamate

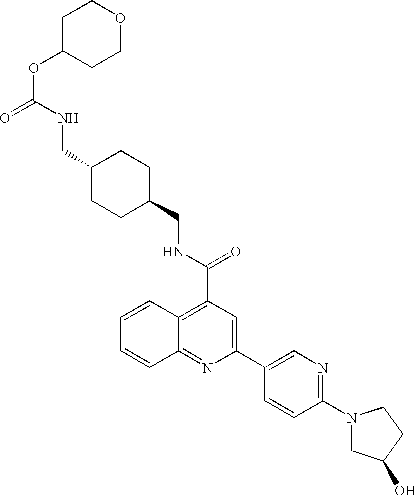

Example 202 was prepared according to the procedure described for Example 201 using 4-nitrophenyl tetrahydro-2H-pyran-4-yl carbonate (Intermediate QQ) in place of 4-nitrophenyl (3S)-tetrahydrofuran-3-yl carbonate. ¹H NMR (400 MHz, DMSO-d₆) δ 9.00-8.98 (m, 1H), 8.76-8.71 (m, 1H), 8.41-8.37 (m, 1H), 8.05-7.99 (m, 3H), 7.74-7.68 (m, 1H), 7.54-7.49 (m, 1H), 7.10-7.05 (m, 1H), 6.57 (d, 1H), 5.15-4.90 (m, 1H), 4.68-4.58 (m, 1H), 4.42-4.37 (m, 1H), 3.79-3.73 (m, 2H), 3.57-3.48 (m, 3H), 3.42-3.33 (m, 3H), 3.21-3.16 (m, 2H), 2.48-2.45 (m, 2H), 2.07-1.96 (m, 1H), 1.93-1.87 (m, 1H), 1.83-1.75 (m, 4H), 1.72-1.66 (m, 2H), 1.53-1.38 (m, 3H), 1.35-1.27 (m, 1H), 0.98-0.77 (m, 4H); m/z (M+H)⁺ 588.4.

### Example 203

### tert-Butyl {[trans-4-({[(2-{4-[2-(dimethylamino)ethoxy]phenyl}quinolin-4-yl)carbonyl]amino}methyl)cyclohexyl]methyl}carbamate

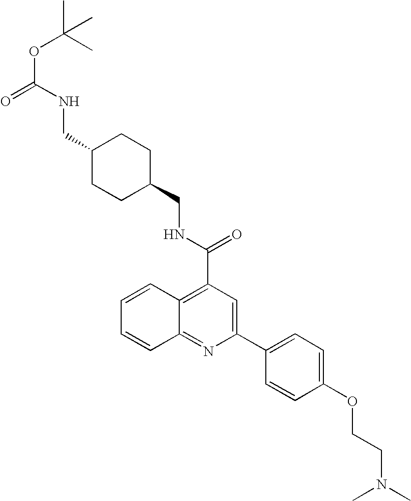

Diisopropyl azodicarboxylate (0.05 mL, 0.26 mmol) was added to a stirred solution of tert-butyl({trans-4-[({[2-(4-hydroxyphenyl)quinolin-4-yl]carbonyl}amino)methyl]cyclohexyl}methyl)carbamate (Intermediate CCCC) (86 mg, 0.18 mmol), 2-dimethyl-aminoethanol (0.022 mL, 0.22 mmol) and triphenylphosphine (69 mg, 0.26 mmol) in THF (2 mL) and the reaction mixture was stirred at rt for 3 h. EtOAc and a saturated aqueous solution of NaHCO₃ were added. The layers were separated and the aqueous phase was extracted with EtOAc. The combined organic layers were dried (Na₂SO₄) and concentrated in vacuo to leave a residue which was purified by preparative HPLC (Standard method F) to give the title compound (40 mg, 41%). ¹H NMR (400 MHz, DMSO/DMSO-d₆*) δ 8.78-8.76 (m, 1H), 8.25-8.21 (m, 2H), 8.08-8.03 (m, 3H), 7.77-7.74 (m, 1H), 7.58-7.55 (m, 1H), 7.11-7.07 (m, 2H), 6.78-6.75 (m, 1H), 4.12 (t, 2H), 3.20-3.17 (m, 2H), 2.76-2.73 (m, 2H), 2.65-2.62 (m, 2H), 2.20 (s, 6H), 1.82-1.77 (m, 2H), 1.71-1.66 (m, 2H), 1.54-1.48 (m, 1H), 1.34 (s, 9H), 1.31-1.26 (m, 1H), 0.97-0.79 (m, 4H); m/z (M+H)⁺ 561.4.

### Example 204

### tert-Butyl ({trans-4-[({2-[6-(4-methylpiperazin-1-yl)pyridin-3-yl]quinolin-4-yl}carbonyl)amino]cyclohexyl}methyl)carbamate

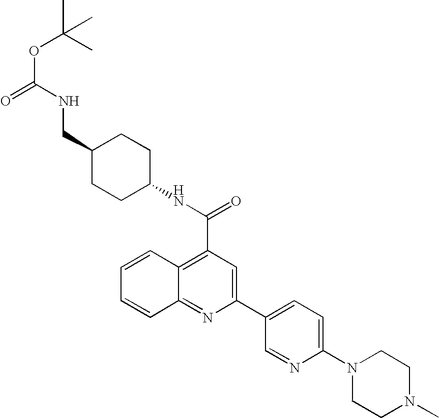

tert-Butyl [(trans-4-aminocyclohexyl)methyl]carbamate (470 mg, 2.1 mmol), DIPEA (0.9 mL, 5.2 mmol) and TBTU (716 mg, 2.2 mmol) were added to a solution of 2-[6-(4-methylpiperazin-1-yl)pyridin-3-yl]quinoline-4-carboxylic acid (Intermediate A) (598 mg, 1.7 mmol) in DMF (3 mL) and the reaction mixture was stirred for 16 h at rt. Water and MeOH were added to give a precipitate and the mixture was filtered. The collected solid was washed with a mixture of MeOH and water (1:3) and purified by flash column chromatography, using a gradient of 3-40% MeOH:TEA (10:1) in DCM, to give the title compound (220 mg, 23%). ¹H NMR (400 MHz, CDCl₃) δ 8.85-8.82 (m, 1H), 8.28-8.24 (m, 1H), 8.05-7.98 (m, 2H), 7.68-7.63 (m, 2H), 7.46-7.40 (m, 1H), 6.70 (d, 1H), 6.22-6.18 (m, 1H), 4.70-4.61 (m, 1H), 4.05-3.95 (m, 1H), 3.68-3.62 (m, 4H), 3.00-2.95 (m, 2H), 2.53-2.48 (m, 4H), 2.33 (s, 3H), 2.21-2.15 (m, 2H), 1.86-1.80 (m, 2H), 1.42 (s, 10H), 1.32-1.04 (m, 4H); m/z (M+H)⁺ 559.2.

### Example 205

### tert-Butyl {[trans-4-({[(2-{4-[3-(dimethylamino)-2-hydroxypropyl]piperidin-1-yl}quinolin-4-yl)carbonyl]amino}methyl)cyclohexyl]methyl}carbamate

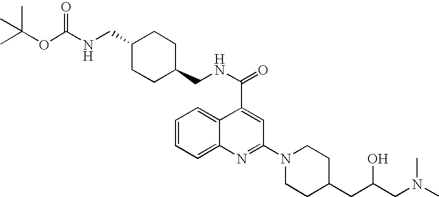

tert-Butyl [(trans-4-{[({2-[4-(oxiran-2-ylmethyl)piperidin-1-yl]quinolin-4-yl}carbonyl)amino]methyl}cyclohexyl)methyl]carbamate (Intermediate DDDD) (77 mg, 0.14 mmol) was dissolved in a solution of dimethylamine in MeOH (2M, 2 mL) and the reaction mixture was heated in the microwave at 80° C. for 5 min. The solvent was evaporated and the crude product was purified with HPLC chromatography, using a Kromasil C8 column, 10 μm, 250×20 ID mm, 10-90% mobilephase B over 25 min (Mobilephase A=water:ACN:formic acid 95:5:0.2, mobilephase B=ACN). The required fractions were concentrated in vacuo to give the title compound (57 mg, 68%). ¹H NMR (500 MHz, CD₃OD) δ 8.55 (s, 1H), 7.84-7.78 (m, 1H), 7.67-7.61 (m, 1H), 7.57-7.50 (m, 1H), 7.27-7.19 (m, 1H), 7.15 (s, 1H), 4.61-4.51 (m, 2H), 4.09-4.01 (m, 1H), 3.30-3.26 (m, 2H), 3.05-2.86 (m, 6H), 2.80 (s, 6H), 1.98-1.77 (m, 7H), 1.65-1.54 (m, 1H), 1.49-1.38 (m, 2H), 1.43 (s, 9H), 1.36-1.17 (m, 3H), 1.10-0.9 (m, 4H); m/z (M+H)⁺ 582.5.

### Examples 206-207

The following Examples were prepared using the method described in Example 205 using the appropriate amine as starting material in place of dimethylamine.

| | | | MS |
|---|---|---|---|
| | | ¹H NMR (500 MHz, | m/z |
| Example | Name | CD₃OD) δ | (M + H)⁺ |
| 206ⁱ | tert-Butyl[(trans-4- | 8.52 (s, 1H), 7.84-7.79 (m, | 608.5 |
| | {[({2-[4-(2-hydroxy-3- | 1H), 7.66-7.62 (m, 1H), | |
| | pyrrolidin-1-ylpropyl)- | 7.56-7.51 (m, 1H), | |
| | piperidin-1-yl]quinolin- | 7.26-7.20 (m, 1H), 7.15 (s, 1H), | |
| | 4-yl}carbonyl)amino]- | 4.60-4.50 (m, 2H), 4.07-3.99 (m, | |
| | methyl}cyclohexyl)methyl]carbamate | 1H), 3.42-3.33 (m, 4H), | |
| | | 3.30-3.26 (m, 2H), | |
| | | 3.18-3.07 (m, 2H), 3.05-2.93 (m, 2H), | |
| | | 2.91-2.86 (m, 2H), | |
| | | 2.10-2.03 (m, 4H), 1.97-1.77 (m, 7H), | |
| | | 1.65-1.54 (m, 1H), | |
| | | 1.50-1.37 (m, 2H), 1.43 (s, 9H), | |
| | | 1.36-1.17 (m, 3H), 1.10-0.89 (m, | |
| | | 4H) | |
| 207 | tert-Butyl[(trans-4- | 8.52 (s, 1H), 7.84-7.78 (m, | 624.5 |
| | {[({2-[4-(2-hydroxy-3- | 1H), 7.66-7.61 (m, 1H), | |
| | morpholin-4- | 7.56-7.50 (m, 1H), | |
| | ylpropyl)piperidin-1- | 7.26-7.19 (m, 1H), 7.14 (s, 1H), | |
| | yl]quinolin-4-yl}- | 4.59-4.50 (m, 2H), 4.02-3.94 (m, | |
| | carbonyl)amino]methyl}cyclohexyl)methyl]- | 1H), 3.80-3.70 (m, 4H), | |
| | carbamate | 3.30-3.25 (m, 2H), | |
| | | 3.04-2.93 (m, 2H), 2.92-2.86 (m, 2H), | |
| | | 2.82-2.67 (m, 4H), | |
| | | 2.57-2.52 (m, 2H), 1.97-1.77 (m, 7H), | |
| | | 1.65-1.54 (m, 1H), | |
| | | 1.49-1.37 (m, 2H), 1.43 (s, 9H), | |
| | | 1.37-1.16 (m, 3H), 1.10-0.90 (m, | |
| | | 4H) | |

ⁱ The product of Example 206 was separated by chiral chromatography, using a Chiralcel OJ column, 5 μm, 250×20 mm, mobile phase heptane/EtOH/TEA 90/10/0.1 to give the two enantiomers, Example 208 and Example 209. Example 208 eluted first with a retention time of 12 min and Example 209 eluted second with a retention time of 16 min.

### Example 208

### (R) or (S) tert-Butyl [(trans-4-{[({2-[4-(2-hydroxy-3-morpholin-4-ylpropyl)piperidin-1-yl]quinolin-4-yl}-carbonyl)amino]methyl}cyclohexyl)methyl]-carbamate;

### Example 209

### (S) or (R) tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;

### Example 210

### tert-Butyl {[trans-4-({[(2-{4-[2-(dimethylamino)-2-oxoethoxy]piperidin-1-yl}quinolin-4-yl)carbonyl]amino}methyl)cyclohexyl]methyl}carbamate

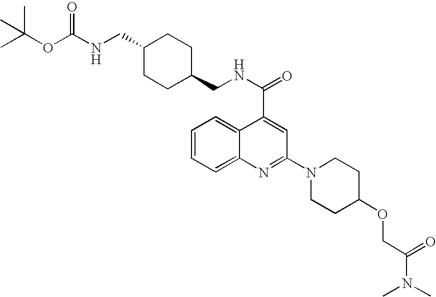

A suspension of tert-butyl {[trans-4-({[(2-chloroquinolin-4-yl)carbonyl]amino}-methyl)cyclohexyl]methyl}carbamate (Example 35) (75 mg, 0.17 mmol) and N,N-dimethyl-2-(piperidin-4-yloxy)acetamide trifluoroacetate (Intermediate EEEE) (155 mg, 0.52 mmol, 3 eq.) in pyridine (1 mL) was heated in a microwave at 140° C. for 80 min. The reaction mixture was concentrated in vacuo to leave a residue, which was purified by HPLC chromatography, using a Kromasil C8 column, 10 μm, 250×20 ID mm, 10-90% mobilephase B over 20 min (Mobilephase A=water:ACN:formic acid 95:5:0.2, mobilephase B=ACN) to give the title compound (20 mg, 20%). ¹H NMR (500 MHz, CD₃OD) δ 7.85-7.80 (m, 1H), 7.67-7.51 (m, 2H), 7.33-7.21 (m, 1H), 7.17 (s, 1H), 4.29 (s, 2H), 4.23-4.16 (m, 2H), 3.75-3.67 (m, 1H), 3.47-3.38 (m, 2H), 3.30-3.26 (m, 2H), 3.05 (s, 3H), 2.95 (s, 3H), 2.92-2.87 (m, 2H), 2.08-1.99 (m, 2H), 1.95-1.86 (m, 2H), 1.86-1.78 (m, 2H), 1.74-1.55 (m, 3H), 1.49-1.37 (m, 1H), 1.43 (s, 9H), 1.11-0.91 (m, 4H); m/z (M+H)⁺ 582.4.

### Example 211

### tert-Butyl {[trans-4-({[(2-{4-[2-(dimethylamino)ethoxy]piperidin-1-yl}quinolin-4-yl)carbonyl]amino}methyl)cyclohexyl]methyl}carbamate

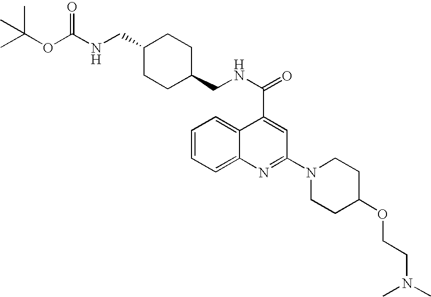

A suspension of tert-butyl {[trans-4-({[(2-chloroquinolin-4-yl)carbonyl]amino}-methyl)cyclohexyl]methyl}carbamate (100 mg, 0.23 mmol), N,N-dimethyl-2-(piperidin-4-yloxy)ethanamine bis(trifluoroacetate) (Intermediate FFFF) (395 mg, 0.99 mmol, 4.3 eq.) and K₂CO₃ (160 mg, 1.16 mmol, 5 eq.) in pyridine (2 mL) was heated in a microwave at 140° C. for 80 min. The reaction mixture was concentrated in vacuo to leave a residue, which was purified by HPLC chromatography, using a Kromasil C8 column, 10 μm, 250×20 ID mm, 10-90% mobilephase B over 20 min (Mobilephase A=water:ACN:formic acid 95:5:0.2, mobilephase B=ACN) to give the title compound (30 mg, 23%). ¹H NMR (500 MHz, CD₃OD) δ 7.84-7.79 (m, 1H), 7.67-7.63 (m, 1H), 7.57-7.51 (m, 1H), 7.27-7.21 (m, 1H), 7.16 (s, 1H), 4.24-4.13 (m, 2H), 3.76-3.61 (m, 3H), 3.46-3.37 (m, 2H), 3.30-3.25 (m, 2H), 2.92-2.86 (m, 2H), 2.78-2.72 (m, 2H), 2.43 (s, 6H), 2.07-1.97 (m, 2H), 1.94-1.86 (m, 2H), 1.85-1.77 (m, 2H), 1.69-1.56 (m, 3H), 1.48-1.37 (m, 1H), 1.43 (s, 9H), 1.11-0.90 (m, 4H); m/z (M+H)⁺ 568.5.

### Example 212

### 2,2-Dimethylpropyl {[trans-4-({[(2-{4-[2-(dimethylamino)ethyl]piperidin-1-yl}quinolin-4-yl)carbonyl]amino}methyl)cyclohexyl]methyl}carbamate

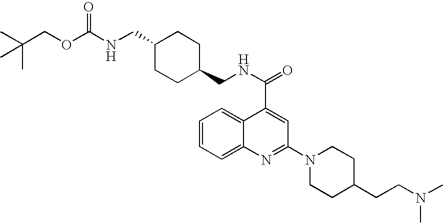

2,2-Dimethylpropyl chlorocarbonate (32 μL, 0.21 mmol, 1.2 eq.) was added to a solution of N-{[trans-4-(aminomethyl)cyclohexyl]methyl}-2-{4-[2-(dimethylamino)-ethyl]piperidin-1-yl}quinoline-4-carboxamide trihydrochloride (Intermediate GGGG) (100 mg, 0.18 mmol) and DIPEA (0.31 mL, 1.8 mmol, 10 eq.) in DCM (5 mL) and the reaction mixture was stirred at rt for 10 min. The reaction mixture was concentrated in vacuo to leave a residue, which was purified with HPLC chromatography, using a Kromasil C8 column, 10 μm, 250×20 ID mm, 10-90% mobilephase B over 20 min (Mobilephase A=water:ACN:formic acid 95:5:0.2, mobilephase B=ACN) to give the title compound (75 mg, 74%). ¹H NMR (500 MHz, CDCl₃) δ 7.88-7.80 (m, 1H), 7.71-7.64 (m, 1H), 7.57-7.49 (m, 1H), 7.28-7.19 (m, 1H), 7.01 (s, 1H), 6.20-6.12 (m, 1H), 4.80-4.70 (m, 1H), 4.55-4.46 (m, 2H), 3.79-3.70 (m, 2H), 3.42-3.33 (m, 2H), 3.08-2.99 (m, 2H), 2.98-2.86 (m, 2H), 2.72-2.62 (m, 2H), 2.48 (s, 6H), 1.93-1.75 (m, 6H), 1.68-1.53 (m, 4H), 1.50-1.39 (m, 1H), 1.34-1.21 (m, 2H), 1.11-0.88 (m, 4H), 0.92 (s, 9H); m/z (M+H)⁺ 566.5.

### Examples 213-214

The following examples were prepared by the method described in Example 185 (Method 22) using the appropriate amine as starting material in place of 1-piperazineethanol.

| | | ¹H NMR (600 MHz, | MS |
|---|---|---|---|
| Example | Name | DMSO/DMSO-d₆)* δ | m/z (M + H)⁺ |
| 213 | tert-Butyl[(trans-4- | 8.57 (t, 1H), 7.75 (d, 1H), | 580.4 |
| | {[({2-[4-(morpholin- | 7.56-7.45 (m, 2H), 7.17 (t, 2H), | |
| | 4-ylmethyl)piperidin- | 7.12 (s, 1H), 6.76 (t, 1H), 4.50 (d, | |
| | 1-yl]quinolin-4- | 2H), 3.58-3.49 (m, 4H), 3.12 (t, | |
| | yl}carbonyl)amino]methyl}cyclohexyl)methyl]- | 2H), 2.93-2.84 (m, 2H), 2.74 (t, | |
| | carbamate | 2H), 2.36-2.26 (m, 4H), 2.11 (d, | |
| | | 2H), 1.86-1.72 (m, 4H), 1.68 (d, | |
| | | 2H), 1.52-1.41 (m, 1H), 1.34 (s, | |
| | | 9H), 1.31-1.23 (m, 1H), | |
| | | 1.14-1.02 (m, 2H), 0.96-0.76 (m, 4H) | |
| 214 | tert-Butyl[(trans-4- | 8.57 (t, 1H), 8.12 (s, 1H), | 579.4 |
| | {[({2-[4-(4- | 7.76 (d, 1H), 7.55-7.47 (m, 2H), | |
| | methylpiperazin-1- | 7.19 (t, 1H), 7.14 (s, 1H), 6.76 (t, | |
| | yl)piperidin-1-yl]- | 1H), 4.52 (d, 2H), 3.13 (t, 2H), | |
| | quinolin-4- | 2.89 (t, 2H), 2.74 (t, 2H), | |
| | yl}carbonyl)amino]methyl}cyclohexyl)methyl]- | 2.12 (s, 3H), 1.86-1.81 (m, 2H), | |
| | carbamate | 1.80-1.73 (m, 2H), 1.71-1.65 (m, 2H), | |
| | | 1.50-1.43 (m, 1H), 1.41-1.34 (m, | |
| | | 2H), 1.34 (s, 9H), 1.31-1.24 (m, | |
| | | 1H), 0.96-0.77 (m, 4H) | |

### Examples 215-216

Examples 215 and 216 was prepared by the method described in Example 180 using the appropriate alcohol as starting material in place of 4-methyl-1-(hydroxy-propyl)piperazine. For Example 215 cesium carbonate was used in place of KOH.

| | | ¹H NMR (600 MHz, | MS |
|---|---|---|---|
| Example | Name | DMSO/DMSO-d₆)* δ | m/z (M + H)⁺ |
| 215 | tert-Butyl({trans-4- | 8.80 (t, 1H), 8.55 (d, 1H), | 491.4 |
| | [({[2-(pyridin-3- | 8.49 (d, 1H), 8.01 (d, 1H), | |
| | yloxy)quinolin-4- | 7.77-7.73 (m, 1H), 7.69-7.61 (m, 2H), | |
| | yl]carbonyl}amino)- | 7.55-7.48 (m, 2H), 7.32 (s, 1H), | |
| | methyl]cyclohexyl}methyl)carbamate | 6.77 (t, 1H), 3.18 (t, 2H), 2.75 (t, | |
| | | 2H), 1.79 (d, 2H), 1.69 (d, 2H), | |
| | | 1.54-1.45 (m, 1H), 1.34 (s, 9H), | |
| | | 1.33-1.24 (m, 1H), 0.98-0.78 (m, | |
| | | 4H) | |
| 216 | tert-Butyl[(trans-4- | 8.68 (t, 1H), 7.94 (d, 1H), | 525.3 |
| | {[({2-[(1- | 7.76 (d, 1H), 7.65 (t, 1H), 7.42 (t, | |
| | methylpiperidin-4- | 1H), 6.95 (s, 1H), 6.76 (t, 1H), | |
| | yl)methoxy]quinolin- | 4.26 (d, 2H), 3.13 (t, 2H), | |
| | 4- | 2.74 (t, 2H), 2.24-2.13 (m, 2H), | |
| | yl}carbonyl)amino]methyl}cyclohexyl)methyl]- | 2.04 (s, 3H), 1.81-1.72 (m, 4H), | |
| | carbamate | 1.71-1.64 (m, 2H), 1.51-1.42 (m, 1H), | |
| | | 1.34 (s, 9H), 1.33-1.24 (m, 4H), | |
| | | 0.95-0.77 (m, 4H) | |

### Example 217

### tert-Butyl ({trans-4-[({[2-(3-{[2-(dimethylamino)ethyl]carbamoyl}azetidin-1-yl)quinolin-4-yl]carbonyl}amino)methyl]cyclohexyl}methyl)carbamate

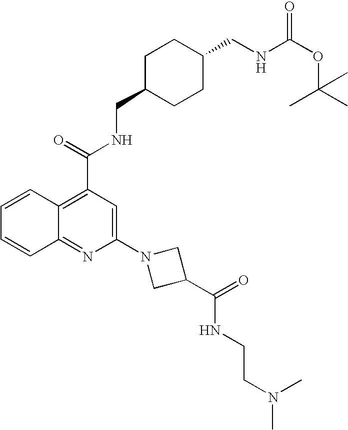

Example 217 was prepared by the method described for Example 1 (Method 1) using Intermediate HHHH in place of Intermediate A and tert-butyl {[trans-4-(aminomethyl)cyclohexyl]methyl}carbamate. ¹H NMR (600 MHz, DMSO/DMSO-d₆*) δ 8.61 (t, 1H), 7.97 (t, 1H), 7.79 (d, 1H), 7.57 (d, 1H), 7.51 (t, 1H), 7.21 (t, 1H), 6.75 (t, 1H), 6.66 (s, 1H), 4.18 (t, 2H), 4.09 (t, 2H), 3.52-3.44 (m, 1H), 3.16 (q, 2H), 3.12 (t, 2H), 2.74 (t, 2H), 2.34-2.25 (m, 2H), 2.13 (s, 6H), 1.80-1.71 (m, 2H), 1.70-1.63 (m, 2H), 1.50-1.41 (m, 1H), 1.34 (s, 9H), 1.31-1.23 (m, 1H), 0.96-0.77 (m, 4H); m/z (M+H)⁺ 567.6.

### Example 218

### tert-Butyl ({trans-4-[({[2-(4-aminophenyl)quinolin-4-yl]carbonyl}amino)-methyl]cyclohexyl}methyl)carbamate

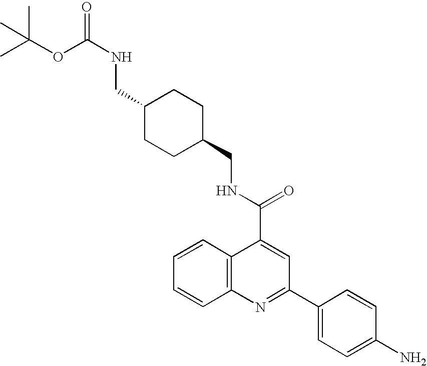

An aq. solution of K₂CO₃ (0.64 g, 4.6 mmol) was added to a suspension of tert-butyl {[trans-4-({[(2-chloroquinolin-4-yl)carbonyl]amino}methyl)cyclohexyl]methyl}-carbamate (Example 35) (0.50 g, 1.16 mmol), (4-aminophenyl)boronic acid hydrochloride (0.24 g, 1.39 mmol) and Pd(PPh₃)₄ (67 mg, 0.058 mmol) in degassed dioxane (15 mL) and the reaction mixture was stirred for 16 h at 60° C. The reaction mixture was filtered and EtOAc and water were added to the solution. The layers were separated and the aqueous phase was extracted with EtOAc. The combined organic phases were dried (Na₂SO₄) and concentrated in vacuo to leave a residue, which was purified by flash column chromatography, using 40→100% EtOAc in heptane followed by 0→20% EtOH in toluene as eluent, to give the title compound (0.46 g, 81%). ¹H NMR (400 MHz, DMSO-d₆) δ 8.73 (t, 1H), 8.04-7.95 (m, 4H), 7.89 (s, 1H), 7.73-7.68 (m, 1H), 7.52-7.47 (m, 1H), 6.78 (t, 1H), 6.68 (d, 2H), 5.59 (s, 2H), 3.19 (t, 2H), 2.76 (t, 2H), 1.84-1.78 (m, 2H), 1.74-1.67 (m, 2H), 1.57-1.46 (m, 1H), 1.35 (s, 9H), 1.35-1.25 (m, 1H), 1.00-0.79 (m, 4H); m/z (M+H)⁺ 489.4.

### Example 219

### tert-Butyl {[trans-4-({[(2-{4-[2-(dimethylamino)ethyl]-3-oxopiperazin-1-yl}quinolin-4-yl)carbonyl]amino}methyl)cyclohexyl]methyl}carbamate

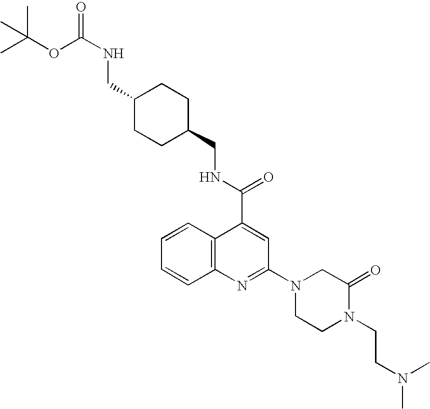

Example 219 was prepared by the method described in Example 185 (Method 22) using Intermediate IIII as starting material in place of 1-piperazineethanol. ¹H NMR (600 MHz, DMSO/DMSO-d₆*) δ 8.60 (t, 1H), 7.84-7.82 (m, 1H), 7.60 (d, 1H), 7.56-7.52 (m, 1H), 7.26-7.22 (m, 1H), 7.17 (s, 1H), 6.76 (t, 1H), 4.27 (s, 2H), 3.94-3.92 (m, 2H), 3.49-3.44 (m, 4H), 3.14 (t, 2H), 2.74 (t, 2H), 2.17 (s, 6H), 1.80-1.75 (m, 2H), 1.70-1.66 (m, 2H), 1.50-1.43 (m, 1H), 1.34 (s, 9H), 1.31-1.24 (m, 1H), 0.95-0.78 (m, 4H); m/z (M+H)⁺ 567.4.

### Example 220

### tert-Butyl [(trans-4-{[({2-[4-(hydroxyacetyl)piperazin-1-yl]quinolin-4-yl}carbonyl)amino]methyl}cyclohexyl)methyl]carbamate

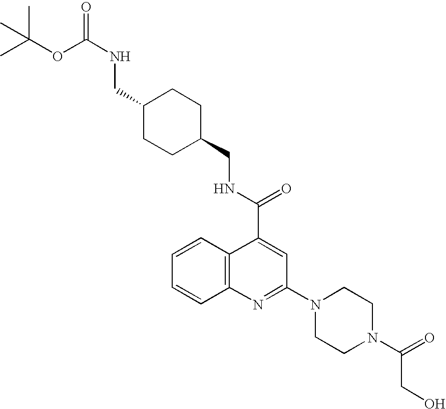

Example 220 was prepared by the method described in Example 185 (Method 22) using Intermediate JJJJ as starting material in place of 1-piperazineethanol. ¹H NMR (600 MHz, DMSO/DMSO-d₆*) δ 8.60 (t, 1H), 7.79 (d, 1H), 7.59-7.57 (m, 1H), 7.54-7.51 (m, 1H), 7.25-7.21 (m, 1H), 7.18 (s, 1H), 6.76 (t, 1H), 4.62 (t, 1H), 4.13 (d, 2H), 3.73-3.68 (m, 4H), 3.60-3.56 (m, 2H), 3.48-3.44 (m, 2H), 3.13 (t, 2H), 2.74 (t, 2H), 1.79-1.75 (m, 2H), 1.70-1.65 (m, 2H), 1.51-1.43 (m, 1H), 1.34 (s, 9H), 1.31-1.24 (m, 1H), 0.95-0.78 (m, 4H); m/z (M+H)⁺ 540.3.

### Example 221

### tert-Butyl {[trans-4-({[(2-{4-[(4-fluorobenzyl)oxy]piperidin-1-yl}quinolin-4-yl)carbonyl]amino}methyl)cyclohexyl]methyl}carbamate

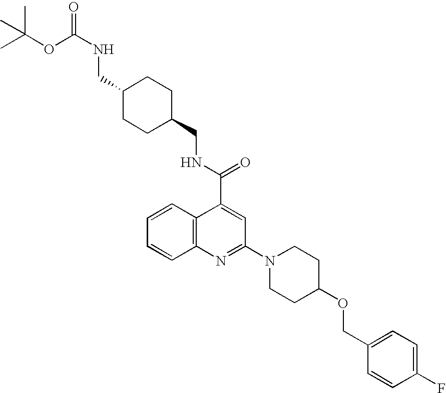

Example 221 was prepared by the method described in Example 185 (Method 22) using Intermediate KKKK as starting material in place of 1-piperazineethanol. ¹H NMR (600 MHz, DMSO/DMSO-d₆*) δ 8.58 (t, 1H), 7.76 (d, 1H), 7.57-7.48 (m, 2H), 7.38-7.35 (m, 2H), 7.21-7.12 (m, 4H), 6.76 (t, 1H), 4.52 (s, 2H), 4.16-4.11 (m, 2H), 3.69-3.64 (m, 1H), 3.13 (t, 2H), 2.74 (t, 2H), 1.97-1.92 (m, 2H), 1.79-1.74 (m, 2H), 1.69-1.65 (m, 2H), 1.55-1.43 (m, 3H), 1.34 (s, 9H), 1.31-1.24 (m, 1H), 0.94-0.78 (m, 4H); m/z (M+H)⁺ 605.5.

### Example 222

### tert-Butyl {[trans-4-({[(2-amino-1-benzothiophen-3-yl)carbonyl]amino}methyl)-cyclohexyl]methyl}carbamate

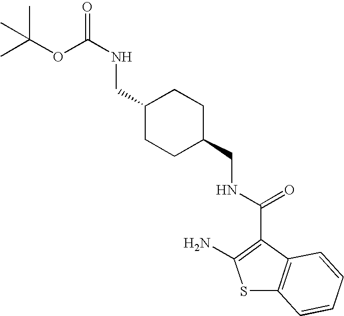

Water (3.4 mL), NMM (0.25 mL, 2.23 mmol) and tert-butyl {[trans-4-(aminomethyl)cyclohexyl]methyl}carbamate (0.278 g, 1.15 mmol) were added to a solution of 2-amino-1-benzothiophene-3-carboxylic acid (Intermediate LLLL, 0.22 g, 1.11 mmol) in methyl-THF (5 mL). An aq. solution of HOBT-NMM (20% HOBT-15% NMM, 0.38 mL) and EDC (0.28 g, 1.45 mmol) were added and the reaction mixture was stirred vigorously for 48 h. The reaction mixture was diluted with methyl-THF and water and the layers were separated. The aqueous layer was extracted with methyl-THF and the combined organic layers were dried (Na₂SO₄) and concentrated in vacuo to leave a residue. The residue was purified by flash column chromatography, using a gradient of 20→60% EtOAc in heptane as eluent, to give the title compound (97 mg, 21%). ¹H NMR (400 MHz, CDCl₃) δ 7.56 (d, 1H), 7.53 (d, 1H), 7.32 (t, 1H), 7.12 (t, 1H), 6.39 (s, 2H), 6.04-5.99 (m, 1H), 4.60-4.54 (m, 1H), 3.33 (t, 2H), 2.98 (t, 2H), 1.93-1.77 (m, 4H), 1.65-1.52 (m, 1H), 1.47-1.36 (m, 1H), 1.44 (s, 9H), 1.11-0.91 (m, 4H); m/z (M+H)⁺ 418.2.

### Example 223

### tert-Butyl [(trans-4-{[({2-[(ethylcarbamoyl)amino]-1-benzothiophen-3-yl}carbonyl)amino]methyl}cyclohexyl)methyl]carbamate

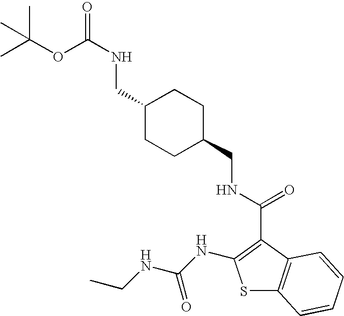

Ethyl isocyanate was added to a solution of tert-butyl {[trans-4-({[(2-amino-1-benzothiophen-3-yl)carbonyl]amino}methyl)cyclohexyl]methyl}carbamate (Example 222, 56 mg, 0.13 mmol) in pyridine (2 mL) and the reaction mixture was stirred at 60° C. for 16 h. The reaction mixture was concentrated in vacuo to leave a residue which was dissolved in pyridine (0.5 mL) and ethyl isocyanate was added (0.016 mL, 0.20 mmol) and the reaction mixture was stirred at 40° C. for 48 h and then an additional portion of ethyl isocyanate was added (0.016 mL, 0.20 mmol) and the reaction mixture was heated at 80° C. for 1.5 h in a microwave. The reaction mixture was concentrated in vacuo to leave a residue which was purified by flash column chromatography, using a gradient of 0→5% MeOH in DCM followed by 20%→80% tert-butyl methyl ether in toluene as eluent, to give the title compound (28 mg, 43%). ¹H NMR (400 MHz, CDCl₃) δ 11.46 (s, 1H), 7.76 (d, 1H), 7.66 (d, 1H), 7.39 (t, 1H), 7.24 (t, 1H), 6.28 (t, 1H), 4.98 (s, 1H), 4.57 (s, 1H), 3.41-3.32 (m, 4H), 3.01-2.94 (m, 2H), 1.94-1.78 (m, 4H), 1.67-1.55 (m, 1H), 1.46-1.37 (m, 1H), 1.44 (s, 9H), 1.21 (t, 3H), 1.12-0.91 (m, 4H); m/z (M+H)⁺ 489.3.

### Example 224

### Tetrahydro-2H-pyran-4-yl {[trans-4-({[(2-{4-[2-(dimethylamino)ethyl]piperidin-1-yl}quinolin-4-yl)carbonyl]amino}methyl)cyclohexyl]methyl}carbamate

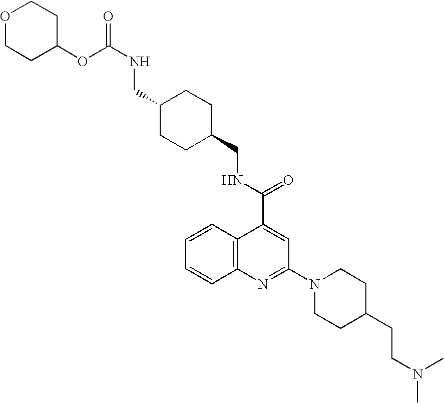

Example 224 was prepared by the method described in Example 202 using N-{[trans-4-(aminomethyl)cyclohexyl]methyl}-2-{4-[2-(dimethylamino)ethyl]-piperidin-1-yl}quinoline-4-carboxamide trihydrochloride (Intermediate GGGG) as starting material. ¹H NMR (600 MHz, DMSO/DMSO-d₆*) 68.57 (t, 1H), 7.75 (d, 1H), 7.54-7.46 (m, 2H), 7.19-7.15 (m, 1H), 7.12 (s, 1H), 7.08 (t, 1H), 4.65-4.60 (m, 1H), 4.52-4.47 (m, 2H), 3.78-3.74 (m, 2H), 3.40-3.34 (m, 2H), 3.12 (t, 2H), 2.89-2.83 (m, 2H), 2.80 (t, 2H), 2.25-2.21 (m, 2H), 2.09 (s, 6H), 1.82-1.65 (m, 8H), 1.62-1.54 (m, 1H), 1.50-1.40 (m, 3H), 1.35-1.27 (m, 3H), 1.14-1.06 (m, 2H), 0.94-0.79 (m, 4H); m/z (M+H)⁺ 580.4.

### Example 225

### tert-Butyl ({trans-4-[({[2-(1′-methyl-4,4′-bipiperidin-1-yl)quinolin-4-yl]carbonyl}amino)methyl]cyclohexyl}methyl)carbamate

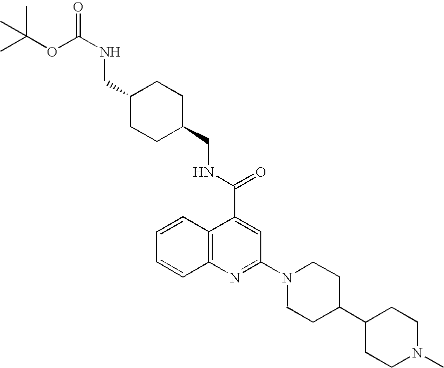

Example 225 was prepared by the method described in Example 185 (Method 22) using 1-methyl-4,4′-bipiperidine as starting material in place of 1-piperazineethanol. ¹H NMR (400 MHz, CDCl₃) δ 7.84 (dd, 1H), 7.69 (d, 1H), 7.56-7.51 (m, 1H), 7.25-7.20 (m, 1H), 7.04 (s, 1H), 5.98 (t, 1H), 4.60-4.53 (m, 3H), 3.38 (t, 2H), 2.98 (t, 2H), 2.93-2.84 (m, 4H), 2.25 (s, 3H), 1.91-1.78 (m, 8H), 1.74-1.67 (m, 2H), 1.63-1.54 (m, 1H), 1.44 (s, 9H), 1.42-1.22 (m, 6H), 1.11-0.91 (m, 5H); m/z (M+H)⁺ 578.2.

### Example 226

### Method 23

### tert-Butyl ({trans-4-[({[2-(4-{2-[(2-methoxyethyl)amino]ethyl}piperidin-1-yl)quinolin-4-yl]carbonyl}amino)methyl]cyclohexyl}methyl)carbamate

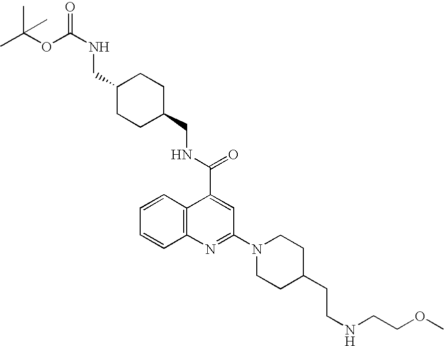

2-Methoxyethylamine (20 mg, 0.26 mmol, 1.5 eq.), sodium triacetoxyborohydride (66 mg, 0.31 mmol, 1.8 eq.) and one drop of AcOH were added to a solution of tert-butyl [(trans-4-{[({2-[4-(2-oxoethyl)piperidin-1-yl]quinolin-4-yl}carbonyl)amino]methyl}-cyclohexyl)methyl]carbamate (Intermediate DDDDi)), 89 mg, 0.17 mmol) in EtOH (3 mL) and the reaction mixture was heated at in a microwave at 120° C. for 10 min. The reaction mixture was concentrated in vacuo to leave a residue. DMSO (2 mL) was added to the residue and the mixture was filtered and the filtrate was purified by HPLC (Standard Method C) to give the title compound (50 mg, 51%). ¹H NMR (600 MHz, DMSO/DMSO-d₆*) δ 8.57 (t, 1H), 7.75 (d, 1H), 7.52 (d, 1H), 7.48 (dd, 1H), 7.17 (dd, 1H), 7.12 (s, 1H), 6.76 (t, 1H), 4.49 (d, 2H), 3.20 (s, 3H), 3.12 (t, 2H), 2.87 (t, 2H), 2.74 (t, 2H), 2.61 (t, 2H), 1.78-0.76 (m, 17H), 1.34 (s, 9H); m/z 582.8 (M+H)⁺.

### Examples 227-232

The following Examples 227-232 were prepared from Intermediate DDDDi) using essentially the same conditions as described for Example 226 with the appropriate amine in place of 2-methoxyethylamine.

| | | | MS |
|---|---|---|---|
| | | ¹H NMR (600 MHz, | m/z |
| Example | Name | DMSO/DMSO-d₆*) δ | (M + H)⁺ |
| 227 | tert-Butyl({trans-4- | 8.57 (t, 1H), 7.75 (d, 1H), | 596.8 |
| | [({[2-(4-{2-[(2- | 7.52 (d, 1H), 7.49 (dd, 1H), | |
| | methoxyethyl)(methyl)- | 7.17 (dd, 1H), 7.12 (s, 1H), | |
| | amino]ethyl}piperidin-1- | 6.76 (t, 1H), 4.49 (d, 2H), | |
| | yl)quinolin-4- | 3.20 (s, 3H), 3.12 (t, 2H), | |
| | yl]carbonyl}amino)- | 2.87 (t, 2H), 2.74 (t, 2H), | |
| | methyl]cyclohexyl}- | 2.13 (s, 3H), 1.77-1.66 (m, | |
| | methyl)carbamate | 6H), 1.57 (s, 1H), 1.46 (s, | |
| | | 1H), 1.34 (s, 9H), | |
| | | 1.33-1.25 (m, 3H), 1.14-1.06 (m, 2H), | |
| | | 0.93-0.78 (m, 4H) | |
| 228 | tert-Butyl{[trans-4- | 8.57 (t, 1H), 7.75 (d, 1H), | 608.7 |
| | ({[(2-{4-[2-(tetrahydro- | 7.52 (d, 1H), 7.48 (dd, 1H), | |
| | 2H-pyran-4- | 7.17 (dd, 1H), 7.12 (s, 1H), | |
| | ylamino)ethyl]piperidin- | 6.76 (t, 1H), 4.50 (d, 2H), | |
| | 1-yl}quinolin-4- | 3.78 (d, 2H), 3.24 (t, 2H), | |
| | yl)carbonyl]amino}- | 3.12 (t, 2H), 2.87 (t, 2H), | |
| | methyl)cyclohexyl]- | 2.77-2.71 (m, 2H), | |
| | methyl}carbamate | 1.77-1.06 (m, 17H), 1.34 (s, 9H), | |
| | | 0.93-0.78 (m, 4H) | |
| 229 | tert-Butyl{[trans-4- | 8.56 (t, 1H), 7.74 (d, 1H), | 594.7 |
| | ({[(2-{4-[2-(3- | 7.52 (d, 1H), 7.48 (dd, 1H), | |
| | methoxyazetidin-1- | 7.17 (dd, 1H), 7.11 (s, 1H), | |
| | yl)ethyl]piperidin-1- | 6.76 (t, 1H), 4.48 (d, 2H), | |
| | yl}quinolin-4- | 3.89 (m, 1H), 3.44 (t, 2H), | |
| | yl)carbonyl]amino}- | 3.12 (t, 2H), 3.10 (s, 3H), | |
| | methyl)cyclohexyl]- | 2.86 (t, 2H), 2.74 (t, 2H), | |
| | methyl}carbamate | 1.77-1.66 (m, 6H), 1.56 (s, | |
| | | 1H), 1.46 (s, 1H), 1.34 (s, | |
| | | 9H), 1.27 (s, 1H), | |
| | | 1.20-1.01 (m, 4H), 0.93-0.76 (m, 4H) | |
| 230 | tert-Butyl{[trans-4- | 8.57 (t, 1H), 7.75 (d, 1H), | 594.8 |
| | ({[(2-{4-[2-(3- | 7.52 (d, 1H), 7.48 (dd, 1H), | |
| | hydroxypyrrolidin-1- | 7.17 (dd, 1H), 7.12 (s, 1H), | |
| | yl)ethyl]piperidin-1- | 6.76 (t, 1H), 4.49 (d, 2H), | |
| | yl}quinolin-4- | 4.13 (s, 1H), 3.12 (t, 2H), | |
| | yl)carbonyl]amino}- | 2.87 (t, 2H), 2.74 (t, 2H), | |
| | methyl)cyclohexyl]- | 2.64 (dd, 2H), 2.40-2.34 (m, | |
| | methyl}carbamate | 1H), 2.24 (dd, 1H), | |
| | | 1.95-1.90 (m, 1H), 1.77-1.66 (m, 6H), | |
| | | 1.59 (s, 1H), 1.47 (s, 1H), | |
| | | 1.34 (s, 9H), 1.27 (s, 1H), | |
| | | 1.13-1.07 (m, 2H), | |
| | | 0.93-0.78 (m, 4H) | |
| 231 | tert-Butyl[(trans-4- | 8.57 (t, 1H), 7.75 (d, 1H), | 578.8 |
| | {[({2-[4-(2-pyrrolidin-1- | 7.52 (d, 1H), 7.48 (dd, 1H), | |
| | ylethyl)piperidin-1- | 7.17 (dd, 1H), 7.12 (s, 1H), | |
| | yl]quinolin-4- | 6.76 (t, 1H), 4.49 (d, 2H), | |
| | yl}carbonyl)amino]- | 3.12 (t, 2H), 2.87 (t, 2H), | |
| | methyl}cyclohexyl)- | 2.74 (t, 2H), 2.37 (m, 2H), | |
| | methyl]carbamate | 1.77-1.63 (m, 8H), 1.61 (s, | |
| | | 1H), 1.46 (s, 1H), | |
| | | 1.38-1.34 (m, 4H), 1.34 (s, 9H), | |
| | | 1.27 (s, 1H), 1.13-1.07 (m, 2H), | |
| | | 0.93-0.78 (m, 4H) | |
| 232 | tert-Butyl({trans-4- | 8.57 (t, 1H), 7.75 (d, 1H), | 582.7 |
| | [({[2-(4-{2-[(2- | 7.52 (d, 1H), 7.48 (t, 1H), | |
| | hydroxyethyl)(methyl)- | 7.17 (t, 1H), 7.12 (s, 1H), | |
| | amino]ethyl}piperidin-1- | 6.76 (t, 1H), 4.49 (d, 2H), | |
| | yl)quinolin-4- | 4.30 (s, 1H), 3.43 (dd, 2H), | |
| | yl]carbonyl}amino)- | 3.12 (t, 2H), 2.87 (t, 2H), | |
| | methyl]cyclohexyl}- | 2.74 (t, 2H), 2.35 (dd, 2H), | |
| | methyl)carbamate | 2.12 (s, 3H), 1.77-1.66 (m, | |
| | | 6H), 1.58 (s, 1H), 1.46 (s, | |
| | | 1H), 1.34 (s, 9H), | |
| | | 1.33-1.25 (m, 3H), 1.14-1.07 (m, 2H), | |
| | | 0.93-0.78 (m, 4H) | |

### Example 233

### tert-Butyl {[trans-4-({[(2-{4-[2-(3-hydroxyazetidin-1-yl)ethyl]piperidin-1-yl}quinolin-4-yl)carbonyl]amino}methyl)cyclohexyl]methyl}carbamate

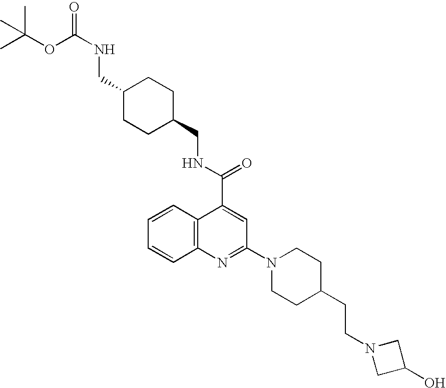

Example 233 was prepared by the method described in Example 226 (Method 23) using azetidin-3-ol (Intermediate 0000) as starting material in place of 2-methoxyethylamine. ¹H NMR (600 MHz, DMSO/DMSO-d₆*) δ 8.57 (t, 1H), 7.76-7.74 (m, 1H), 7.54-7.51 (m, 1H), 7.50-7.46 (m, 1H), 7.19-7.16 (m, 1H), 7.11 (s, 1H), 6.76 (t, 1H), 4.51-4.47 (m, 2H), 4.13-4.08 (m, 1H), 3.47-3.44 (m, 2H), 3.12 (t, 2H), 2.89-2.83 (m, 2H), 2.74 (t, 2H), 2.61-2.58 (m, 2H), 2.39-2.35 (m, 2H), 1.79-1.65 (m, 6H), 1.60-1.52 (m, 1H), 1.50-1.43 (m, 1H), 1.34 (s, 9H), 1.30-1.24 (m, 1H), 1.20-1.16 (m, 2H), 1.11-1.03 (m, 2H), 0.94-0.78 (m, 4H); m/z (M+H)⁺ 580.2.

### Example 234

### tert-Butyl [(trans-4-{[({2-[4-(2-azetidin-1-ylethyl)piperidin-1-yl]quinolin-4-yl}carbonyl)amino]methyl}cyclohexyl)methyl]carbamate

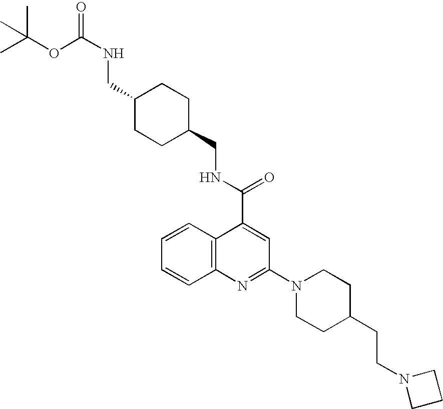

Example 234 was prepared by the method described in Example 226 (Method 23) using azetidine as starting material in place of 2-methoxyethylamine. ¹H NMR (600 MHz, DMSO/DMSO-d₆*) δ 8.56 (t, 1H), 7.75 (d, 1H), 7.53-7.51 (m, 1H), 7.50-7.46 (m, 1H), 7.19-7.16 (m, 1H), 7.11 (s, 1H), 6.76 (t, 1H), 4.51-4.47 (m, 2H), 3.14-3.11 (m, 2H), 3.08-3.03 (m, 2H), 2.89-2.83 (m, 2H), 2.74 (t, 2H), 2.46-2.44 (m, 2H), 2.37-2.34 (m, 2H), 1.94-1.88 (m, 2H), 1.79-1.65 (m, 6H), 1.60-1.53 (m, 1H), 1.50-1.43 (m, 1H), 1.34 (s, 9H), 1.30-1.24 (m, 1H), 1.19-1.14 (m, 2H), 1.11-1.03 (m, 2H), 0.94-0.78 (m, 4H); m/z (M+H)⁺ 564.2.

### Example 235

### 1-{2-[1-(4-{[(trans-4-{[(tert-Butoxycarbonyl)amino]methyl}cyclohexyl)methyl]-carbamoyl}quinolin-2-yl)piperidin-4-yl]ethyl}azetidine-3-carboxylic Acid

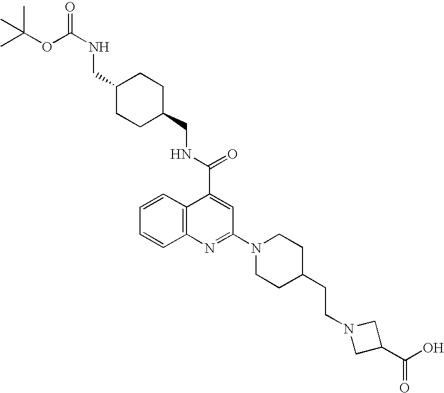

Example 235 was prepared by the method described in Example 226 (Method 23) using azetidine-3-carboxylic acid as starting material in place of 2-methoxyethylamine. ¹H NMR (600 MHz, DMSO/DMSO-d₆*) δ 8.39 (t, 1H), 7.58 (d, 1H), 7.36-7.34 (m, 1H), 7.33-7.29 (m, 1H), 7.02-6.98 (m, 1H), 6.94 (s, 1H), 6.58 (t, 1H), 4.34-4.30 (m, 2H), 2.99-2.93 (m, 4H), 2.72-2.66 (m, 2H), 2.57 (t, 2H), 2.37-2.34 (m, 2H), 2.30-2.27 (m, 2H), 1.87 (s, 1H), 1.62-1.48 (m, 6H), 1.43-1.35 (m, 1H), 1.33-1.26 (m, 1H), 1.17 (s, 9H), 1.13-1.07 (m, 1H), 1.05-1.00 (m, 2H), 0.95-0.87 (m, 2H), 0.77-0.61 (m, 4H); m/z (M+H)⁺ 608.2.

### Example 236

### tert-Butyl {[trans-4-({[(2-{4-[2-(3-aminoazetidin-1-yl)ethyl]piperidin-1-yl}quinolin-4-yl)carbonyl]amino}methyl)cyclohexyl]methyl}carbamate

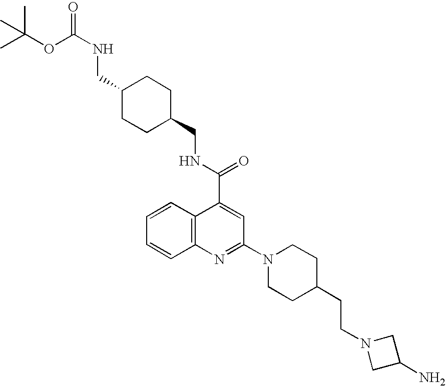

A mixture of benzyl (1-{2-[1-(4-{[(trans-4-{[(tert-butoxycarbonyl)amino]methyl}-cyclohexyl)methyl]carbamoyl}quinolin-2-yl)piperidin-4-yl]ethyl}azetidin-3-yl)-carbamate (Intermediate MMMM, 0.095 g, 0.13 mmol) and 5% palladium on carbon (95 mg) in MeOH (10 mL) was stirred under a hydrogen atmosphere at rt for 2 h. The reaction mixture was filtered through a pad of celite and the filtrate was concentrated in vacuo to leave a residue which was purified by preparative HPLC (Standard Method C) to give the title compound (46 mg, 60%). ¹H NMR (600 MHz, DMSO/DMSO-d₆*) δ 8.56 (t, 1H), 7.75 (d, 1H), 7.54-7.51 (m, 1H), 7.50-7.46 (m, 1H), 7.19-7.15 (m, 1H), 7.11 (s, 1H), 6.76 (t, 1H), 4.52-4.46 (m, 2H), 3.43-3.40 (m, 2H), 3.12 (t, 2H), 2.89-2.83 (m, 2H), 2.74 (t, 2H), 2.44-2.41 (m, 2H), 2.32 (t, 2H), 1.80-1.65 (m, 6H), 1.59-1.52 (m, 1H), 1.50-1.43 (m, 1H), 1.34 (s, 9H), 1.31-1.24 (m, 1H), 1.19-1.14 (m, 2H), 1.11-1.03 (m, 2H), 0.94-0.78 (m, 4H); m/z (M+H)⁺ 579.2

### Example 237

### tert-Butyl {[trans-4-({[(2-{4-[3-(dimethylamino)-2-fluoropropyl]piperidin-1-yl}quinolin-4-yl)carbonyl]amino}methyl)cyclohexyl]methyl}carbamate

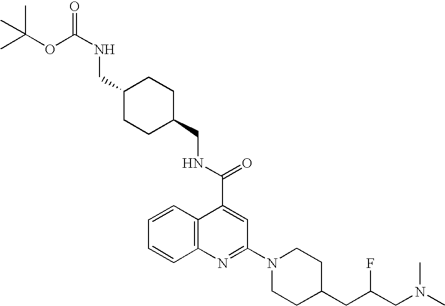

(Diethylamino)sulfur trifluoride (0.039 mL, 0.30 mmol) was added slowly to a solution of tert-butyl [(trans-4-{[({2-[4-(2-hydroxy-3-pyrrolidin-1-ylpropyl)piperidin-1-yl]quinolin-4-yl}carbonyl)amino]methyl}cyclohexyl)methyl]carbamate (Example 206), 0.058 g, 0.10 mmol) in DCM (1 mL) at −78° C. and the reaction mixture was allowed to reach room temperature and stirred for 16 h. EtOAc and a saturated aq. solution of NaHCO₃ were added and the layers were separated. The aqueous layer was extracted with EtOAc and the combined organic layers were dried (Na₂SO₄) and concentrated in vacuo to leave a residue which was purified by HPLC (Standard Method C) to give the title compound (0.013 g, 22%). ¹H NMR (600 MHz, DMSO/DMSO-d₆*) δ 8.57 (t, 1H), 7.75 (d, 1H), 7.54-7.52 (m, 1H), 7.50-7.47 (m, 1H), 7.19-7.16 (m, 1H), 7.13 (s, 1H), 6.76 (t, 1H), 4.82-4.68 (m, 1H), 4.54-4.47 (m, 2H), 3.13 (t, 2H), 2.93-2.85 (m, 2H), 2.74 (t, 2H), 2.16 (s, 6H), 1.86-1.65 (m, 6H), 1.60-1.37 (m, 2H), 1.34 (s, 9H), 1.31-1.24 (m, 1H), 1.21-1.07 (m, 3H), 0.94-0.78 (m, 4H), 2.24 (s, 2H); m/z (M+H)⁺ 584.2.

### Example 238

### tert-Butyl {[trans-4-({[(2-{3-[2-(dimethylamino)ethoxy]azetidin-1-yl}quinolin-4-yl)carbonyl]amino}methyl)cyclohexyl]methyl}carbamate

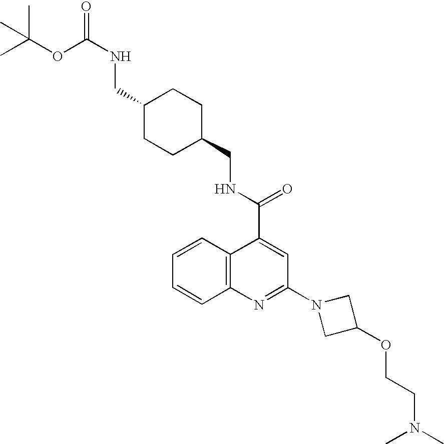

Sodium hydride (60% in mineral oil, 21 mg, 0.53 mmol) was added to a stirred solution of tert-butyl ({trans-4-[({[2-(3-hydroxyazetidin-1-yl)quinolin-4-yl]-carbonyl}amino)methyl]cyclohexyl}methyl)carbamate (Intermediate NNNN, 0.10 g, 0.21 mmol) in DMF (2 mL) at 0° C. After 30 min 2-chloro-N,N-dimethylethanamine hydrochloride (34 mg, 0.23 mmol) was added and the reaction mixture was stirred at 50° C. for 16 h. The reaction mixture was filtered and purified by preparative HPLC (Standard Method C) to give the title compound (10 mg, 9%). ¹H NMR (600 MHz, CDCl₃) δ 7.87 (d, 1H), 7.72 (d, 1H), 7.57-7.53 (m, 1H), 7.26-7.23 (m, 1H), 6.61 (s, 1H), 5.97 (t, 1H), 4.59-4.55 (m, 1H), 4.50-4.46 (m, 1H), 4.37-4.34 (m, 2H), 4.09 (dd, 2H), 3.54 (t, 2H), 3.38 (t, 2H), 2.98 (t, 2H), 2.53 (t, 2H), 2.27 (s, 6H), 1.89-1.79 (m, 4H), 1.61-1.52 (m, 1H), 1.45-1.38 (m, 1H), 1.43 (s, 9H), 1.09-0.93 (m, 4H); m/z (M+H)⁺ 540.2.

### Example 239

### Method 24

### tert-Butyl [(trans-4-{[({2-[3-(aminomethyl)phenyl]-6-(1H-pyrazol-4-yl)pyridin-4-yl}carbonyl)amino]methyl}cyclohexyl)methyl]carbamate

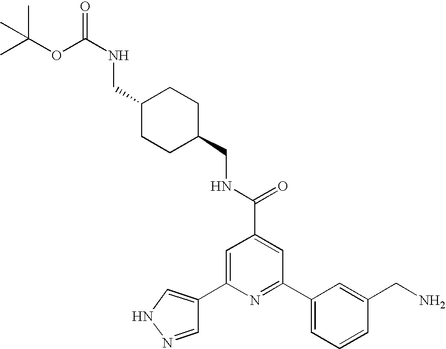

4-(4,4,5,5-Tetramethyl-1,3,2-dioxaborolan-2-yl)-1H-pyrazole (47 mg, 0.24 mmol, 1.0 eq.), a solution of cesium carbonate (130 mg, 0.4 mmol, 1.7 eq.) in water (1 mL) and PEPPSI (8 mg, 0.012 mmol, 0.05 eq.) were added to a stirred solution of tert-butyl [(trans-4-{[({2-[3-(aminomethyl)phenyl]-6-chloropyridin-4-yl}carbonyl)amino]methyl}cyclohexyl)methyl]-carbamate (Intermediate PPPP, 115 mg, 0.24 mmol) in dioxane (3 mL) and the reaction mixture was stirred in a microwave at 140° C. for 15 min. The reaction mixture was concentrated in vacuo and a saturated aq. solution of NaHCO₃ was added. The mixture was extracted with EtOAc and methyl-THF. The combined organic layers were concentrated in vacuo to leave a residue which was purified by HPLC (Standard method C) to give the title compound (21 mg, 17%). ¹H NMR (600 MHz, DMSO/DMSO-d₆*) δ 8.76 (t, 1H), 8.32 (s, 1H), 8.22 (s, 1H), 8.09 (d, 1H), 8.04 (s, 1H), 7.94 (s, 1H), 7.48 (dd, 1H), 7.44 (d, 1H), 6.76 (t, 1H), 3.92 (s, 2H), 3.15 (t, 2H), 2.72 (t, 2H), 1.76 (d, 2H), 1.67 (d, 2H), 1.50 (s, 1H), 1.34 (s, 9H), 1.28 (s, 1H), 0.93-0.78 (m, 4H); m/z 519.3 (M+H)⁺.

### Examples 240-241

The following Examples 240-241 were prepared using essentially the same conditions as described for Example 239 from the Intermediate PPPP and the appropriate boronic ester or acid in place of 4-(4,4,5,5-tetramethyl-1,3,2-dioxaborolan-2-yl)-1H-pyrazole.

### Example 240

### tert-Butyl [(trans-4-{[({6-[3-(aminomethyl)phenyl]-2,3′-bipyridin-4-yl}carbonyl)amino]methyl}cyclohexyl)methyl]carbamate

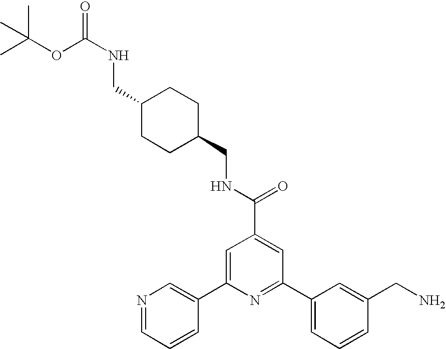

¹H NMR (600 MHz, DMSO/DMSO-d₆*) δ 9.41 (s, 1H), 8.88 (t, 1H), 8.67 (d, 1H), 8.58 (d, 1H), 8.31 (s, 1H), 8.28 (s, 1H), 8.21 (s, 1H), 8.09 (d, 1H), 7.57 (dd, 1H), 7.49-7.44 (m, 2H), 6.76 (t, 1H), 3.85 (s, 2H), 3.18 (t, 2H), 2.74 (t, 2H), 1.77 (d, 2H), 1.68 (d, 2H), 1.53-1.48 (m, 1H), 1.34 (s, 9H), 1.30-1.27 (m, 1H), 0.94-0.78 (m, 4H); m/z 530.4 (M+H)⁺.

### Example 241

### tert-Butyl [(trans-4-{[({6′-amino-6-[3-(aminomethyl)phenyl]-2,3′-bipyridin-4-yl}carbonyl)amino]methyl}cyclohexyl)methyl]carbamate

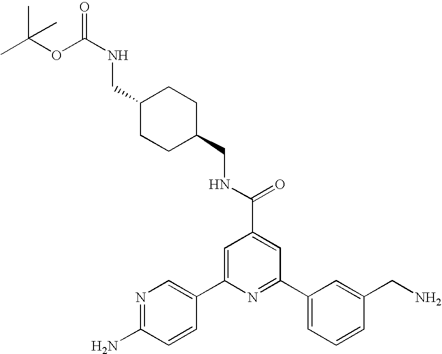

¹H NMR (600 MHz, DMSO/DMSO-d₆*) δ 8.82-8.80 (m, 2H), 8.30 (s, 1H), 8.25-8.22 (m, 2H), 8.11-8.09 (m, 2H), 7.51-7.45 (m, 2H), 6.76 (t, 1H), 6.56 (d, 1H), 6.34 (s, 2H), 3.96 (s, 2H), 3.16 (t, 2H), 2.74 (t, 2H), 1.76 (d, 2H), 1.68 (d, 2H), 1.54-1.47 (m, 1H), 1.33 (s, 9H), 1.31-1.26 (m, 1H), 0.94-0.78 (m, 4H); m/z 545.4 (M+H)⁺.

### Example 242

### tert-Butyl {[trans-4-({[(2-{1-[2-(dimethylamino)ethyl]-1H-pyrazol-4-yl}quinolin-4-yl)carbonyl]amino}methyl)cyclohexyl]methyl}carbamate

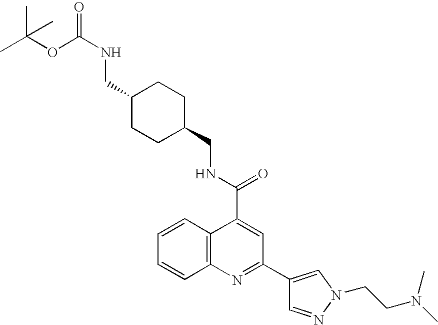

Cesium carbonate (90 mg, 0.28 mmol) and 2-dimethylaminochloride hydrochloride (24 mg, 0.17 mmol) were added to a solution of tert-butyl {[trans-4-({[(2-{1H-pyrazol-4-yl}quinolin-4-yl)carbonyl]amino}methyl)cyclohexyl]methyl}carbamate (Intermediate QQQQ, 64 mg, 0.14 mmol) in EtOH (1 mL) and the reaction mixture was heated in a microwave at 140° C. for 20 min. The reaction mixture was concentrated in vacuo to leave a residue which was purified by preparative HPLC (Standard Method G) to give the title compound (37 mg, 50%). ¹H NMR (400 MHz, DMSO-d₆) δ 8.73 (t, 1H), 8.53 (s, 1H), 8.18 (s, 1H), 7.99-7.94 (m, 2H), 7.78 (s, 1H), 7.72 (t, 1H), 7.52 (t, 1H), 6.77 (t, 1H), 4.25 (t, 2H), 3.19 (t, 2H), 2.76 (t, 2H), 2.69 (t, 2H), 2.17 (s, 6H), 1.81 (d, 2H), 1.70 (d, 2H), 1.54-1.47 (m, 1H), 1.35-1.25 (m, 10H), 0.98-0.79 (m, 4H); m/z 535.3 (M+H)⁺.

### Example 243

### tert-Butyl [(trans-4-{[({2-[1-(2-hydroxyethyl)-1H-pyrazol-4-yl]quinolin-4-yl}carbonyl)amino]methyl}cyclohexyl)methyl]carbamate

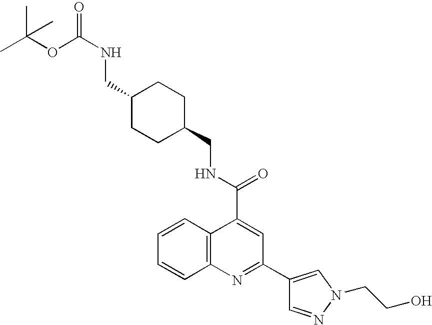

Example 243 was prepared using essentially the same conditions as described for Example 242 using 2-bromoethanol in place of 2-dimethylaminochloride hydrochloride. ¹H NMR (400 MHz, DMSO-d₆) δ 8.64 (t, 1H), 8.50 (s, 1H), 8.20 (s, 1H), 7.99-7.94 (m, 2H), 7.80 (s, 1H), 7.72 (t, 1H), 7.52 (t, 1H), 6.77 (t, 1H), 4.95 (t, 1H), 4.21 (t, 2H), 3.78 (q, 2H), 3.19 (t, 2H), 2.76 (t, 2H), 1.81 (d, 2H), 1.70 (d, 2H), 1.51 (s, 1H), 1.35 (s, 9H), 1.30 (m, 1H), 0.99-0.79 (m, 4H); m/z 508.3 (M+H)⁺.

### Example 244

### tert-Butyl ({trans-4-[({[2-(4-{2-[(2-fluoroethylamino]ethyl}piperidin-1-yl)quinolin-4-yl]carbonyl}amino)methyl]cyclohexyl}methyl)carbamate

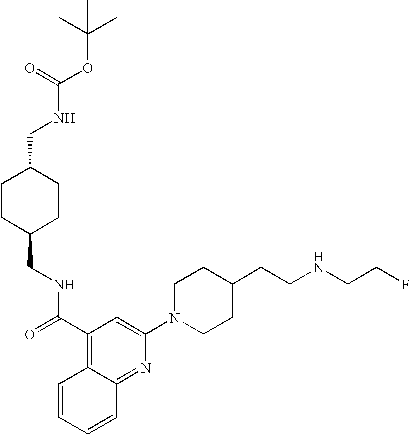

TEA (0.318 mL, 2.28 mmol) was added in one portion to a mixture of tert-butyl [(trans-4-{[({2-[4-(2-oxoethyl)piperidin-1-yl]quinolin-4-yl}carbonyl)amino]methyl}-cyclohexyl)methyl]carbamate (Intermediate DDDDi, 397 mg, 0.76 mmol) in EtOH (18 mL) and DCM (4.5 mL). 2-Fluoroethanamine hydrochloride (121 mg, 1.22 mmol) was added to the solution and the reaction mixture was stirred for 30 min and then sodium triacetoxyborohydride (322 mg, 1.52 mmol) was added in one portion. The reaction mixture was stirred at rt for 16 h and then water and a saturated aq. solution of NaHCO₃ were added. DCM was added and the layers were separated. The second portion of DCM was added and the layers were separated. The combined organic layers were dried using a phase separator and concentrated in vacuo to leave a solid. A portion of the solid (50 mg) was dissolved in a mixture of THF/DMSO/MeOH and purified using preparative HPLC (XBridge C18 column (10 μm 250×19 ID mm), flow rate 19 mL/min) using a gradient of 40-95% ACN in a water/ACN/ammonia 95/5/0.2 buffer over 20 mins to give the product as a solid (27 mg obtained from 50 mg of the crude material). The remaining unpurified solid (380 mg) was used without further purification for the synthesis of Example 245. ¹H NMR (400 MHz, THF-d₈) δ 7.93 (d, 1H), 7.68 (t, 1H), 7.58 (d, 1H), 7.44 (m, 1H), 7.13 (m, 1H), 7.11 (s, 1H), 5.99 (t, 1H), 4.60 (d, 2H), 4.49 (t, 1H), 4.37 (t, 1H), 3.58 (s, 3H), 3.25 (t, 2H), 2.89 (m, 5H), 2.80 (t, 1H), 2.68 (t, 2H), 1.93-1.50 (m, 12H), 1.45-1.32 (m, 6H), 1.23 (m, 2H), 0.98 (m, 4H); m/z 570.3 (M+H)⁺.

### Example 245

### tert-Butyl ({trans-4-[({[2-(4-(2-((2-fluoroethyl)(methyl)amino)ethyl}piperidin-1-yl)quinolin-4-yl]carbonyl}amino)methyl]cyclohexyl}methyl)carbamate

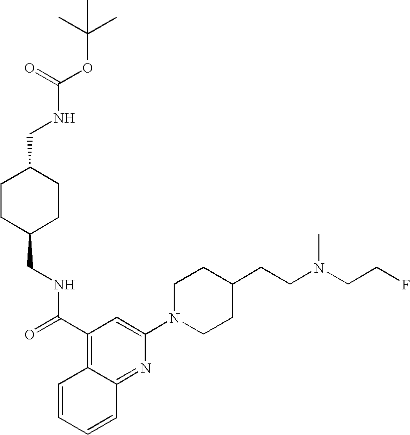

A solution of formaldehyde in water (110 μl, 1.54 mmol) was added to a solution of crude tert-butyl ({trans-4-[({[2-(4-{2-[(2-fluoroethylamino]ethyl}piperidin-1-yl)quinolin-4-yl]carbonyl}amino)methyl]cyclohexyl}methyl)carbamate (Example 244, 350 mg, 0.61 mmol) in THF and the mixture was stirred at rt for 30 min. Sodium cyanoborohydride (1M in THF, 1.84 mL, 1.84 mmol) was added in one portion and the mixture was stirred at rt for 16 h. Water and a saturated aq. solution of NaHCO₃ were added and the layers were separated. DCM was added to the aq. layer, the layers were separated and the combined organic layers were dried using a phase separator and concentrated in vacuo to leave an oil. The oil was dissolved in DMSO/MeOH and purified using preparative HPLC (XBridge C18 column (10 μm 250×50 ID mm), flow rate 100 mL/min) using a gradient of 35-95% ACN in water/ACN/ammonia 95/5/0.2 buffer over 25 mins, to give the product (72 mg, 20%). ¹H NMR (400 MHz, CDCl₃) δ 7.76 (d, 1H), 7.60 (d, 1H), 7.47 (m, 1H), 7.16 (m, 1H), 6.85 (s, 1H), 6.51 (t, 1H), 4.65 (t, 1H), 4.56 (t, 1H), 4.44 (t, 1H), 4.38 (d, 2H), 3.30 (t, 2H), 2.95-2.77 (m, 4H), 2.69 (t, 1H), 2.63 (t, 1H), 2.44 (t, 2H), 2.27 (s, 2H), 1.95 (s, 1H), 1.87-1.68 (m, 6H), 1.57 (m, 2H), 1.41 (m, 12H), 1.20 (m, 2H), 0.95 (m, 4H); m/z 584.3 (M+H)⁺.

### Preparation of Intermediates and Starting Materials Intermediate A

### 2-[6-(4-Methylpiperazin-1-yl)pyridin-3-yl]quinoline-4-carboxylic Acid

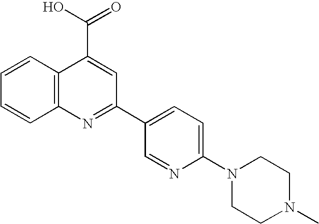

2-Chloroquinoline-4-carboxylic acid (0.15 g, 0.72 mmol), 6-(4-methylpiperazin-1-yl)pyridine-5-boronic acid pinacol ester (0.26 g, 0.87 mmol) and Pd(PPh₃)₄ (42 mg, 0.036 mmol) were added to a mixture of dioxane (2 mL) and a 1M aq. solution of K₂CO₃ (2 mL). The reaction mixture was degassed, sealed, and heated in the microwave at 140° C. for 15 min. The reaction mixture was concentrated in vacuo to leave a residue which was purified by HPLC (Standard method D) to give the title compound (188 mg, 75%). ¹H NMR (400 MHz, DMSO-d₆) δ 8.99 (d, 1H), 8.53 (d, 1H), 8.41 (dd, 1H), 8.26 (s, 1H), 8.02 (d, 1H), 7.77-7.71 (m, 1H), 7.59-7.53 (m, 1H), 6.98 (d, 1H), 3.65 (t, 4H), 2.55 (t, 4H), 2.31 (s, 3H); m/z 349.2 (M+H)⁺.

### Intermediate B

### 2-(2-Methyl-6-oxo-1,6-dihydropyridin-3-yl)quinoline-4-carboxylic acid

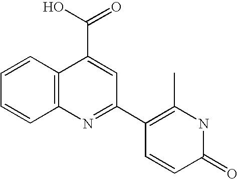

Potassium hydroxide (0.57 g, 10 mmol) was added to a mixture of 2,3-dihydro-indole-2,3-dione (isatin) (0.50 g, 3.4 mmol) and 5-acetyl-6-methyl-2(1H)-pyridinone (0.62 g, 4.1 mmol) in EtOH (4 mL) and the reaction mixture was heated at 80° C. for 20 h. The reaction mixture was diluted with water and purified by HPLC (standard method D) to give the title compound (0.20 g, 17%). ¹H NMR (400 MHz, DMSO-d₆) δ 8.60 (d, 1H), 7.86 (d, 1H), 7.70 (d, 1H), 7.64-7.58 (m, 1H), 7.50 (s, 1H), 7.47-7.40 (m, 1H), 6.26 (d, 1H), 1.86 (s, 3H); m/z 281.1 (M+H)⁺.

### Intermediate C

### 2-Pyrazin-2-ylquinoline-4-carboxylic Acid

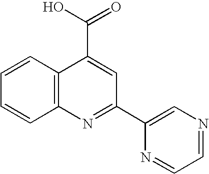

The title compound (0.64 g, 38%) was prepared using 2,3-dihydroindole-2,3-dione (isatin) and 2-acetylpyrazine, in place of 5-acetyl-6-methyl-2(1H)-pyridinone, in a manner essentially similar to that described for Intermediate B. ¹H NMR (400 MHz, DMSO-d₆) δ 9.72 (d, 1H), 8.81-8.75 (m, 2H), 8.73 (d, 1H), 8.18 (d, 1H), 7.87-7.82 (m, 1H), 7.73-7.68 (m, 1H), 7.33-6.97 (m, 1H); m/z 252.0 (M+H)⁺.

### Intermediate D

### 2-(4-Ethoxyphenyl)quinoline-4-carboxylic Acid

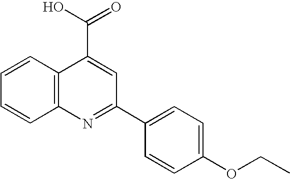

Potassium hydroxide (0.57 g, 10 mmol) was added to a mixture of 2,3-dihydro-indole-2,3-dione (isatin) (0.50 g, 3.4 mmol) and 4-ethoxy-acetophenone (0.67 g, 4.1 mmol) in EtOH (4 mL) and the reaction mixture was heated at 80° C. for 24 h. The reaction mixture was concentrated in vacuo to leave a residue which was dissolved in water and then EtOAc was added. The layers were separated and the water phase was acidified to pH 1 with a 37% aq. solution of HCl to give a precipitate. The mixture was filtered to leave a solid which was washed with water, and dried to give the title compound (0.51 g, 52%). ¹H NMR (400 MHz, DMSO-d₆) δ 13.87 (s, 1H), 8.57 (d, 1H), 8.37 (s, 1H), 8.22 (d, 2H), 8.08 (d, 1H), 7.81-7.76 (m, 1H), 7.65-7.60 (m, 1H), 7.06 (d, 2H), 4.09 (q, 2H), 1.33 (t, 3H); m/z 294.0 (M+H)⁺.

### Intermediate E

### 2-(6-Carbamoylpyridin-3-yl)quinoline-4-carboxylic Acid

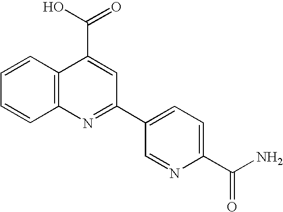

2-Chloroquinoline-4-carboxylic acid (0.15 g, 0.72 mmol), 2-cyanopyridine-5-boronic acid pinacol ester (0.20 g, 0.87 mmol) and Pd(PPh₃)₄ (42 mg, 0.036 mmol) were added to a mixture of dioxane (2 mL) and a 1M aq. solution of K₂CO₃ (2 mL). The reaction mixture was degassed, sealed, and heated in a microwave at 140° C. for 15 min. The reaction mixture was concentrated in vacuo to remove the dioxane and the residual water phase was lyophilized to give the title compound (0.12 g, 57%), which was used in the next step with no further purification; m/z 294.2 (M+H)⁺.

### Intermediate F

### 2-Pyridin-4-ylquinoline-4-carboxylic Acid

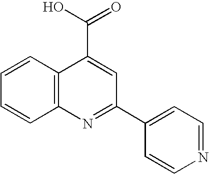

The title compound (0.61 g, 36%) was prepared from 2,3-dihydroindole-2,3-dione (isatin) and 4-acetylpyridine, in place of 5-acetyl-6-methyl-2(1H)-pyridinone, in a manner essentially similar to that described for Intermediate B. ¹H NMR (500 MHz, DMSO-d₆) δ 8.79 (d, 2H), 8.68 (d, 1H), 8.54 (s, 1H), 8.27 (dd, 2H), 8.23 (d, 1H), 7.91 (t, 1H), 7.77 (t, 1H); m/z 251.1 (M+H)⁺.

### Intermediate G

### 2-(3-Carbamoylphenyl)quinoline-4-carboxylic Acid

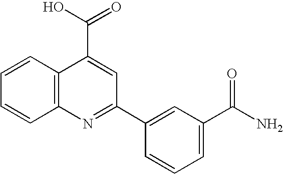

The title compound was prepared from 2-chloroquinoline-4-carboxylic acid and 3-carbamoyl-phenyl boronic acid, in place of 6-(4-methylpiperazin-1-yl)pyridine-5-boronic acid pinacol ester, using essentially the same method as described for Intermediate A. The title compound precipitated from the reaction mixture. It was collected by filtration and used in the next step without further purification. m/z 293.6 (M+H)⁺.

### Intermediate H

### 2-(4-Carbamoylphenyl)quinoline-4-carboxylic Acid

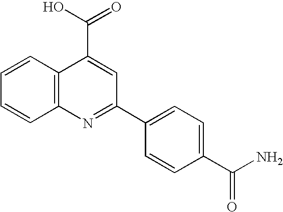

The title compound was prepared from 2-chloroquinoline-4-carboxylic acid and 4-carbamoyl-phenyl boronic acid, in place of 6-(4-methylpiperazin-1-yl)pyridine-5-boronic acid pinacol ester, using essentially the same method as described for Intermediate A. The title compound precipitated from the reaction mixture. It was collected by filtration and used in the next step without further purification. m/z 293.6 (M+H)⁺.

### Intermediate I

### 2-{6-[3-(Dimethylamino)propoxy]pyridin-3-yl}quinoline-4-carboxylic Acid

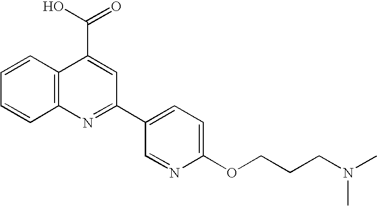

The title compound was prepared from 2-chloroquinoline-4-carboxylic acid and 6-(3-dimethylaminopropoxy)-pyridine-3-boronic acid pinacol ester, in place of 6-(4-methylpiperazin-1-yl)pyridine-5-boronic acid pinacol ester, using essentially the same method as described for Intermediate A. The title compound precipitated from the reaction mixture. It was collected by filtration and used in the next step without further purification. m/z 352.2 (M+H)⁺.

### Intermediate J

### 2-[6-(Dimethylamino)pyridin-3-yl]quinoline-4-carboxylic Acid

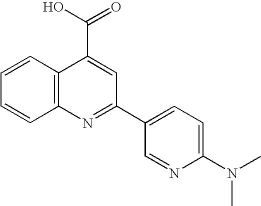

The title compound was prepared from 2-chloroquinoline-4-carboxylic acid and 6-(dimethylamino)-pyridine-3-yl boronic acid dihydrochloride, in place of 6-(4-methylpiperazin-1-yl)pyridine-5-boronic acid pinacol ester, using essentially the same method as described for Intermediate A. The title compound precipitated from the reaction mixture, and was collected by filtration. The dioxane filtrate was concentrated in vacuo to leave a residue which was lyophilized to leave a solid. The filtered and lyophilized solid were combined and used in the next step without further purification. m/z 294.2 (M+H)⁺.

### Intermediate K

### 2-[4-(Trifluoromethyl)phenyl]quinoline-4-carboxylic Acid

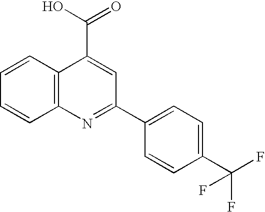

The title compound was prepared from 2,3-dihydroindole-2,3-dione (isatin) and 4-(trifluoromethyl)-acetophenone, in place of 5-acetyl-6-methyl-2(1H)-pyridinone, in a manner essentially similar to that described for Intermediate B. The reaction mixture was concentrated in vacuo and the residue was dissolved in water and washed with EtOAc. The layers were separated and the water phase was acidified to pH 1 with a 37% aq. solution of HCl to leave a precipitate. The precipitate was collected by filtration, washed with a 1M aq. solution of HCl, and recrystallized from EtOH to give the title compound. ¹H NMR (400 MHz, DMSO-d₆) δ 13.94 (s, 1H), 8.64 (d, 1H), 8.52-8.46 (m, 2H), 8.17 (d, 1H), 8.10 (d, 1H), 7.90 (d, 2H), 7.88-7.82 (m, 1H), 7.74-7.68 (m, 1H), m/z 318.0 (M+H)⁺.

### Intermediate L

### 2-(5-Acetyl-2-thienyl)quinoline-4-carboxylic Acid

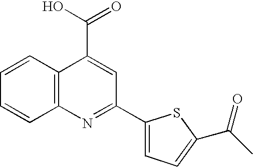

The title compound was prepared from 2-chloroquinoline-4-carboxylic acid and 5-acetyl-2-thiophene boronic acid, in place of 6-(4-methylpiperazin-1-yl)pyridine-5-boronic acid pinacol ester, using essentially the same method as described for Intermediate A. m/z 298.6 (M+H)⁺.

### Intermediate M

### 2-(4-Fluorophenyl)quinoline-4-carboxylic Acid

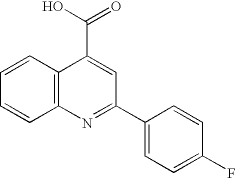

The title compound was prepared from 2-chloroquinoline-4-carboxylic acid and 4-fluorobenzene boronic acid, in place of 6-(4-methylpiperazin-1-yl)pyridine-5-boronic acid pinacol ester, using essentially the same method as described for Intermediate A. ¹H NMR (400 MHz, DMSO-d₆) δ 8.60 (d, 1H), 8.41 (s, 1H), 8.36-8.30 (m, 2H), 8.12 (d, 1H), 7.84-7.78 (m, 1H), 7.69-7.63 (m, 1H), 7.36 (t, 2H); m/z 268.5 (M+H)⁺.

### Intermediate N

### 2-(3,5-Dimethylisoxazol-4-yl)quinoline-4-carboxylic Acid

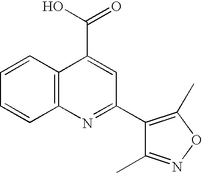

The title compound was prepared from 2-chloroquinoline-4-carboxylic acid and 3,5-dimethylisoxazole-4-boronic acid, in place of 6-(4-methylpiperazin-1-yl)pyridine-5-boronic acid pinacol ester, using essentially the same method as described for Intermediate A. m/z 269.6 (M+H)⁺

### Intermediate O

### 2-Pyridin-3-ylquinoline-4-carboxylic Acid

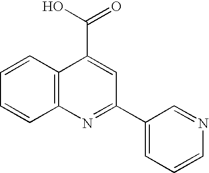

The title compound was prepared from 2-chloroquinoline-4-carboxylic acid and 3-pyridyl boronic acid, in place of 6-(4-methylpiperazin-1-yl)pyridine-5-boronic acid pinacol ester, using essentially the same method as described for Intermediate A. m/z 251.2 (M+H)⁺.

### Intermediate P

### Ethyl {[trans-4-(aminomethyl)cyclohexyl]methyl}carbamate hydrochloride

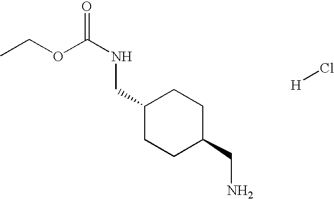

i) tert-Butyl ethyl [trans-cyclohexane-1,4-diylbis(methylene)]biscarbamate

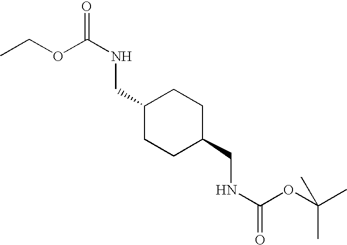

TEA (0.13 g, 1.2 mmol) was added to a solution of tert-butyl {[trans-4-(amino-methyl)cyclohexyl]methyl}carbamate (0.10 g, 0.41 mmol) in DCM (5 mL). The reaction mixture was cooled to 0° C. and a solution of ethyl chloroformate (49 mg, 0.45 mmol) dissolved in DCM (1 mL) was added dropwise. The reaction mixture was stirred at 0° C. to rt for 3 h and then concentrated in vacuo to leave a residue. The residue was purified by flash column chromatography using increasingly polar mixtures of heptane and EtOAc as eluent to give the title compound (81 mg, 62%). ¹H NMR (400 MHz, DMSO-d₆) δ 6.99 (t, 1H), 6.72 (t, 1H), 3.91 (q, 2H), 2.76 (t, 2H), 2.70 (t, 2H), 1.69-1.56 (m, 1H), 1.62 (d, 4H), 1.32 (s, 9H), 1.28-1.17 (m, 1H), 1.10 (t, 3H), 0.83-0.68 (m, 4H); m/z 215.21 (M-tert-butyl and ethyloxy+H)⁺.

ii) A 4M solution of HCl in EtOAc (10 mL) was added to a solution of tert-butyl ethyl [trans-cyclohexane-1,4-diylbis(methylene)]biscarbamate (26 mg, 0.083 mmol) in DCM (10 mL) and the reaction mixture was stirred at rt for 2 h. The reaction mixture was concentrated in vacuo to give the crude title compound (Intermediate P) that was used in the next step with no further purification. ¹H NMR (400 MHz, DMSO-d₆) δ 7.80 (s, 2H), 7.03 (t, 1H), 3.92 (q, 2H), 2.78 (t, 2H), 2.59 (t, 2H), 1.77-1.61 (m, 4H), 1.50-1.36 (m, 1H), 1.32-1.20 (m, 1H), 1.10 (t, 3H), 0.93-0.72 (m, 4H); m/z 215.2 (M+H)⁺.

### Intermediate Q

### 2-[4-(Dimethylcarbamoyl)phenyl]quinoline-4-carboxylic acid

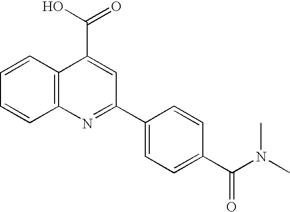

The title compound was prepared from 2-chloroquinoline-4-carboxylic acid and 4-dimethylaminocarbonylphenyl boronic acid, in place of 6-(4-methylpiperazin-1-yl)pyridine-5-boronic acid pinacol ester, using essentially the same method as described for Intermediate A. The title compound precipitated from the reaction mixture and was collected by filtration and used in the next step with no further purification, m/z 321.2 (M+H)⁺.

### Intermediate R

### 2-(4-Methylphenyl)quinoline-4-carboxylic Acid

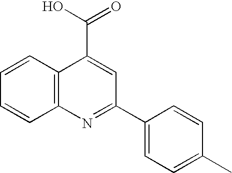

The title compound was prepared from 2,3-dihydroindole-2,3-dione (isatin) and 1-p-tolyl-ethanone, in place of 5-acetyl-6-methyl-2(1H)-pyridinone, using essentially the same method as described for Intermediate B. The reaction mixture was concentrated in vacuo to leave a residue which was diluted with water and EtOAc was added. The layers were separated and the water phase was acidified to pH 1 with a 37% aq. solution of HCl to leave a precipitate. The precipitate was collected by filtration, washed with water, and recrystallized from EtOH to give the title compound. ¹H NMR (400 MHz, DMSO-d₆) δ 13.93 (bs, 1H), 8.60 (d, 1H), 8.39 (s, 1H), 8.19-8.08 (m, 3H), 7.83-7.77 (m, 1H), 7.68-7.61 (m, 1H), 7.34 (d, 2H), 2.36 (s, 3H); m/z 264.0 (M+H)⁺.

### Intermediate S

### 2-(4-Chlorophenyl)quinoline-4-carboxylic Acid

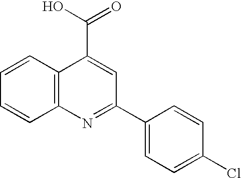

The title compound was prepared from 2,3-dihydroindole-2,3-dione (isatin) and 1-(4-chloro-phenyl)-ethanone, in place of 5-acetyl-6-methyl-2(1H)-pyridinone, using essentially the same reaction method as described for Intermediate B and the same isolation method as described for Intermediate R. ¹H NMR (400 MHz, DMSO-d₆) δ 13.97 (bs, 1H), 8.61 (d, 1H), 8.43 (s, 1H), 8.33-8.27 (m, 2H), 8.13 (d, 1H), 7.86-7.79 (m, 1H), 7.71-7.65 (m, 1H), 7.62-7.57 (m, 2H); m/z 284.0 (M+H)⁺.

### Intermediate T

### 2-(3-Methoxyphenyl)quinoline-4-carboxylic Acid

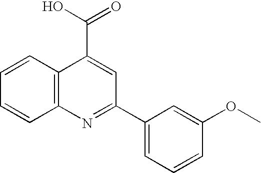

The title compound was prepared from 2,3-dihydroindole-2,3-dione (isatin) and 1-(3-methoxy-phenyl)-ethanone, in place of 5-acetyl-6-methyl-2(1H)-pyridinone, using essentially the same reaction method as described for Intermediate B and the same isolation method as described for Intermediate R. ¹H NMR (400 MHz, DMSO-d₆) δ 13.94 (s, 1H), 8.60 (d, 1H), 8.41 (s, 1H), 8.13 (d, 1H), 7.85-7.78 (m, 3H), 7.67 (t, 1H), 7.45 (t, 1H), 7.07 (dd, 1H), 3.85 (s, 3H); m/z 280.0 (M+H)⁺.

### Intermediate U

### 2-(2-Methoxyphenyl)quinoline-4-carboxylic Acid

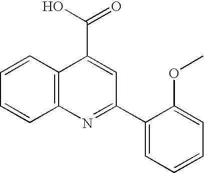

The title compound was prepared from 2,3-dihydroindole-2,3-dione (isatin) and 2-methoxy-acetophenone, in place of 5-acetyl-6-methyl-2(1H)-pyridinone, using essentially the same method as described for Intermediate B. The reaction mixture was concentrated in vacuo to leave a residue which was diluted with water and washed with DCM. The layers were separated and the aqueous phase was acidified to pH 1 with a 37% aq. solution of HCl. The aqueous layer was lyophilized to leave a residue which was purified by HPLC (Standard Method D) to give the title compound. ¹H NMR (400 MHz, DMSO-d₆) δ 8.66 (d, 1H), 8.02-7.95 (m, 2H), 7.78-7.65 (m, 2H), 7.57-7.42 (m, 2H), 7.19 (d, 1H), 7.13-7.07 (m, 1H), 3.85 (s, 3H); m/z 280.0 (M+H)⁺.

### Intermediate V

### 2-[4-(Methylthio)phenyl]quinoline-4-carboxylic Acid

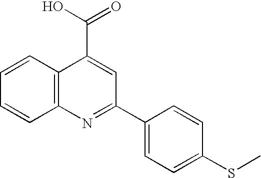

The title compound was prepared from 2,3-dihydroindole-2,3-dione (isatin) and 4-methylthio-acetophenone, in place of 5-acetyl-6-methyl-2(1H)-pyridinone, using essentially the same reaction method as described for Intermediate B and the same isolation method as described for Intermediate R. ¹H NMR (400 MHz, DMSO-d₆) δ 13.92 (bs, 1H), 8.59 (d, 1H), 8.40 (s, 1H), 8.25-8.19 (m, 2H), 8.10 (d, 1H), 7.83-7.76 (m, 1H), 7.68-7.61 (m, 1H), 7.42-7.37 (m, 2H), 2.52 (s, 3H); m/z 296.0 (M+H)⁺.

### Intermediate AA

### 2-(2,4-Dimethyl-1,3-thiazol-5-yl)quinoline-4-carboxylic Acid

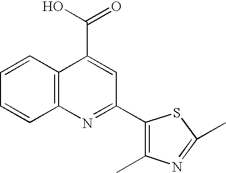

The title compound was prepared from 2,3-dihydroindole-2,3-dione (isatin) and 5-acetyl-2,4-dimethylthiazole, in place of 5-acetyl-6-methyl-2(1H)-pyridinone, using essentially the same reaction method as described for Intermediate B and the same isolation method as described for Intermediate U. ¹H NMR (400 MHz, DMSO-d₆) δ 8.67 (d, 1H), 7.89 (d, 1H), 7.79 (s, 1H), 7.71-7.65 (m, 1H), 7.52-7.30 (m, 2H), 2.69 (s, 3H), 2.65 (s, 3H); m/z 285.0 (M+H)⁺.

### Intermediate BB

### 2-Pyrimidin-5-ylquinoline-4-carboxylic acid

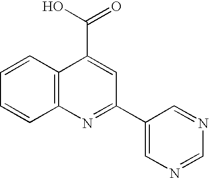

The title compound was prepared from 2-chloroquinoline-4-carboxylic acid and pyrimidine-5-boronic acid, in place of 6-(4-methylpiperazin-1-yl)pyridine-5-boronic acid pinacol ester, using essentially the same method as described for Intermediate A. m/z 252.07 (M+H)⁺.

### Intermediate CC

### 2-(4-Cyanophenyl)quinoline-4-carboxylic Acid

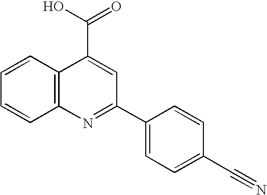

The title compound was prepared from 2-chloroquinoline-4-carboxylic acid and 4-cyanophenyl-boronic acid, in place of 6-(4-methylpiperazin-1-yl)pyridine-5-boronic acid pinacol ester, using essentially the same method as described for Intermediate A. m/z 275.08 (M+H)⁺.

### Intermediate DD

### N-{[trans-4-(Aminomethyl)cyclohexyl]methyl}naphthalene-2-sulfonamide

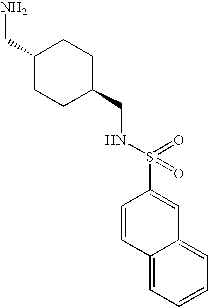

i) tert-Butyl [(trans-4-{[(2-naphthylsulfonyl)amino]methyl}cyclohexyl)methyl]-carbamate

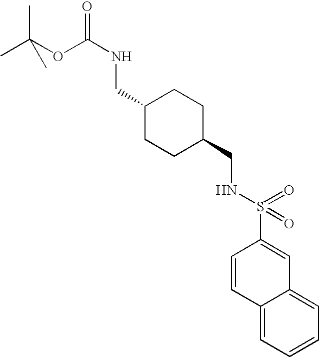

The title compound was prepared from tert-butyl {[trans-4-(aminomethyl)cyclohexyl]-methyl}carbamate (1.3 g, 5.4 mmol) and 2-naphthalenesulfonyl chloride (1.3 g, 5.5 mmol) using essentially the same method as described for Example 63 (Method 6). The crude title compound (2.2 g, 86%) was used directly in the next step with no further purification. ¹H NMR (500 MHz, DMSO-d₆) δ 8.40 (s, 1H), 8.15 (d, 1H), 8.11 (d, 1H), 8.03 (d, 1H), 7.80 (dd, 1H), 7.71-7.62 (m, 3H), 6.73 (t, 1H), 2.70 (t, 2H), 2.61-2.55 (m, 2H), 1.69-1.57 (m, 4H), 1.34 (s, 9H), 1.30-1.16 (m, 2H), 0.79-0.67 (m, 4H).

ii) TFA (15 mL) was added to a solution of tert-butyl [(trans-4-{[(2-naphthylsulfonyl)-amino]methyl}cyclohexyl)methyl]carbamate (1.0 g, 2.3 mmol) in DCM (24 mL) and the reaction mixture was stirred at rt for 30 min. The reaction mixture was concentrated in vacuo to leave the crude title compound that was used directly in the next step with no further purification. ¹H NMR (500 MHz, DMSO-d₆) δ 8.40 (s, 1H), 8.15 (d, 1H), 8.12 (d, 1H), 8.03 (d, 1H), 7.80 (dd, 1H), 7.72-7.61 (m, 3H), 7.26-7.22 (m, 1H), 7.18-7.11 (m, 1H), 2.63-2.58 (m, 4H), 1.70 (d, 4H), 1.45-1.35 (m, 1H), 1.34-1.25 (m, 1H), 0.88-0.73 (m, 4H).

### Intermediate EE

### tert-Butyl [(trans-4-{[(2-chloro-6-methoxyisonicotinoyl)amino]methyl}cyclohexyl)-methyl]carbamate

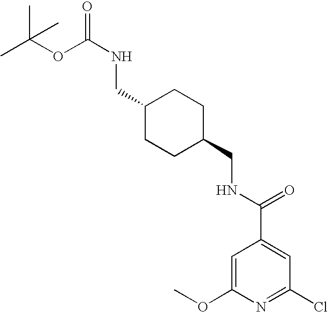

DIPEA (533 mg, 4.1 mmol) and a solution of tert-butyl {[trans-4-(aminomethyl)-cyclohexyl]methyl}carbamate (500 mg, 2.1 mmol) in DMF (1 mL) were added to a solution of 2-chloro-6-methoxyisonicotinic acid (425 mg, 2.3 mmol) and TBTU (795 mg, 2.5 mmol) in DMF (4 mL) and the reaction mixture was stirred for 16 h at rt. Water was added to give a precipitate that was collected by filtration and washed with a mixture of water/MeOH (5:1) to give the title compound (800 mg, 94%). ¹H NMR (400 MHz, CDCl₃) δ 7.18 (d, 1H), 6.93 (d, 1H), 3.91 (s, 3H), 3.18 (d, 2H), 2.99 (s, 2H), 2.89 (m, 2H), 1.81-1.67 (m, 4H), 1.54-1.43 (m, 1H), 1.38 (s, 10H), 1.00-0.80 (m, 4H); m/z 410.0 (M−H)⁻.

### Intermediate FF

### tert-Butyl [(trans-4-{[(2,5-dichloroisonicotinoyl)amino]methyl}cyclohexyl)-methyl]carbamate

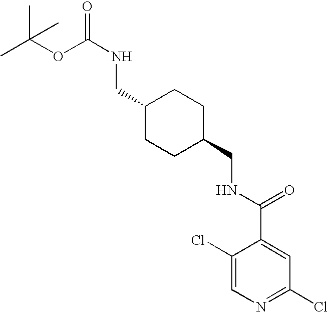

DIPEA (533 mg, 4.1 mmol) and a solution of tert-butyl {[trans-4-(aminomethyl)cyclo-hexyl]methyl}carbamate (500 mg, 2.1 mmol) in DMF (1 mL) were added to a solution of 2,5-dichloroisonicotinic acid (435 mg, 2.3 mmol) and TBTU (795 mg, 2.5 mmol) in DMF (4 mL) and the reaction mixture was stirred for 16 h at rt. Water was added to give a precipitate which was collected by filtration and washed with a mixture of water/MeOH (5:1) to give the title compound (638 mg, 74%). ¹H NMR (400 MHz, CDCl₃) δ 8.37 (s, 1H), 7.46 (s, 1H), 3.24 (d, 2H), 2.95-2.86 (m, 2H), 2.71-2.64 (m, 2H), 1.85-1.69 (m, 4H), 1.57-1.44 (m, 1H), 1.38 (s, 10H), 1.04-0.83 (m, 4H); m/z 414.0 (M−H)⁻.

### Intermediate GG

### tert-Butyl [(trans-4-{[(2-chloro-6-methylisonicotinoyl)amino]methyl}cyclohexyl)-methyl]carbamate

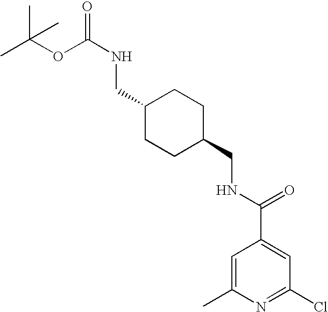

DIPEA (533 mg, 4.1 mmol) and a solution of tert-butyl {[trans-4-(aminomethyl)-cyclohexyl]methyl}carbamate (500 mg, 2.1 mmol) dissolved in DMF (1 mL) were added to a solution of 2-chloro-6-methylisonicotinic acid (389 mg, 2.3 mmol) and TBTU (795 mg, 2.5 mmol) in DMF (4 mL) and the reaction mixture was stirred for 16 h at rt. Water was added to give a precipitate which was collected by filtration and washed with a mixture of water/MeOH (5:1) to give the title compound (764 mg, 93%). ¹H NMR (400 MHz, CDCl₃) δ 7.40 (s, 1H), 7.38 (s, 1H), 6.12 (s, 1H), 4.57 (s, 1H), 3.31 (t, 2H), 3.01-2.93 (m, 2H), 2.60 (s, 3H), 1.87-1.76 (m, 4H), 1.61-1.49 (m, 1H), 1.44 (s, 10H), 1.09-0.88 (m, 4H); m/z 396.1 (M+H)⁺.

### Intermediate HH

### tert-Butyl [(trans-4-{[(2-bromoisonicotinoyl)amino]methyl}cyclohexyl)methyl]-carbamate

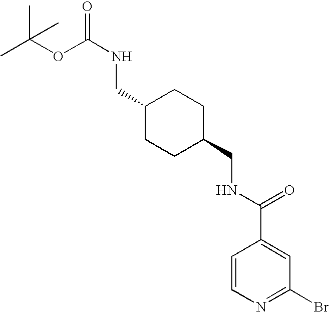

2-Bromo-isonicotinic acid (0.92 g, 4.5 mmol), TBTU (1.6 g, 5.0 mmol) and DIPEA (1.1 g, 8.3 mmol) were added to a solution of tert-butyl {[trans-4-(aminomethyl)cyclohexyl]methyl}-carbamate (1.0 g, 4.1 mmol) in DMF (10 mL) and the reaction mixture was stirred at rt for 22 h. Water was added to give a precipitate which was filtered to give the title compound (1.7 g, 96%). ¹H NMR (400 MHz, CDCl₃) δ 8.48 (d, 1H), 7.78 (s, 1H), 7.55 (dd, 1H), 6.21 (s, 1H), 4.58 (s, 1H), 3.31 (t, 2H), 2.97 (t, 2H), 1.87-1.76 (m, 4H), 1.62-1.49 (m, 2H), 1.44 (s, 9H), 1.08-0.88 (m, 4H); m/z 426.14 (M+H)⁺.

### Intermediate II

### tert-Butyl {[trans-4-({[(5-bromopyridin-3-yl)carbonyl]amino}methyl)cyclohexyl]-methyl}carbamate

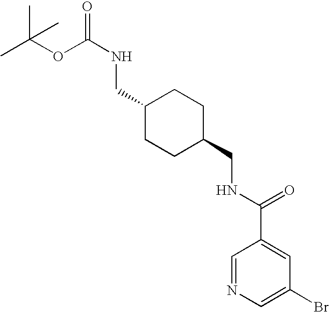

The title compound was prepared from tert-butyl {[trans-4-(aminomethyl)cyclohexyl]-methyl}carbamate and 5-bromo-nicotinic acid, in place of 2-bromo-isonicotinic acid, using essentially the same method as described for Intermediate HH. ¹H NMR (600 MHz, DMSO-d₆) δ 8.91 (d, 1H), 8.80 (d, 1H), 8.67 (t, 1H), 8.36 (t, 1H), 6.74 (t, 1H), 3.07 (t, 2H), 2.72 (t, 2H), 1.71 (d, 2H), 1.64 (d, 2H), 1.47-1.38 (m, 1H), 1.32 (s, 9H), 1.29-1.20 (m, 1H), 0.89-0.73 (m, 4H); m/z 426.14 (M−H)⁻.

### Intermediate JJ

### N-{[trans-4-(Aminomethyl)cyclohexyl]methyl}-2-pyridin-4-ylquinoline-4-carboxamide Hydrochloride

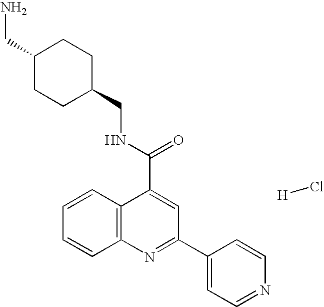

A solution of EtOAc (5 mL) saturated with HCl (gas) was added to a mixture of tert-butyl {[trans-4-({[(2-pyridin-4-ylquinolin-4-yl)carbonyl]amino}methyl)cyclohexyl]methyl}-carbamate [Example 7] (0.15 mg, 0.32 mmol) in DCM (5 mL) and the reaction mixture was stirred at rt for 1 h. The reaction mixture was concentrated in vacuo to give the crude title compound (0.13 g, 98%), which was used directly in the next step with no further purification. ¹H NMR (400 MHz, DMSO-d₆) δ 9.01 (d, 2H), 8.96 (t, 1H), 8.74 (d, 2H), 8.40 (s, 1H), 8.20 (t, 2H), 7.94 (s, 2H), 7.89 (t, 1H), 7.75 (t, 1H), 3.23 (t, 2H), 2.61 (t, 2H), 1.88-1.75 (m, 4H), 1.60-1.47 (m, 2H), 1.05-0.85 (m, 4H); m/z 375.3 (M+H)⁺.

### Intermediate KK

### N-{[trans-4-(Aminomethyl)cyclohexyl]methyl}-2-(4-carbamoylphenyl)quinoline-4-carboxamide Hydrochloride

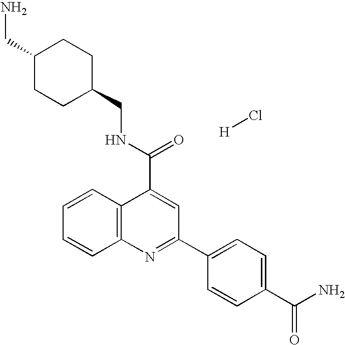

4M HCl in dioxane (20 mL) was added to a solution of tert-butyl ({trans-4-[({[2-(4-carbamoylphenyl)quinolin-4-yl]carbonyl}amino)methyl]cyclohexyl}methyl)carbamate [Example 9] (0.45 g, 0.87 mmol) in DCM (5 mL) and the reaction mixture was stirred at rt for 1 h. The reaction mixture was concentrated in vacuo to give the crude title compound (0.39 g, 99%) which was used directly in the next step with no further purification. ¹H NMR (400 MHz, DMSO-d₆) δ 8.90 (t, 1H), 8.36 (d, 2H), 8.18-8.11 (m, 4H), 8.05 (d, 2H), 8.01 (s, 2H), 7.83 (t, 1H), 7.65 (t, 1H), 7.45 (s, 1H), 3.22 (t, 2H), 2.65-2.56 (m, 2H), 1.81 (t, 4H), 1.59-1.47 (m, 2H), 1.03-0.86 (m, 4H); m/z (M+H)⁺ 417.2.

### Intermediate LL

### N-{[trans-4-(Aminomethyl)cyclohexyl]methyl}-2-phenylquinoline-4-carboxamide

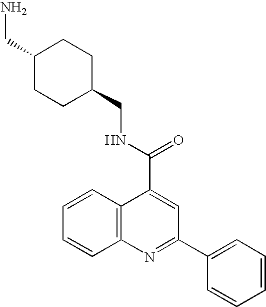

TFA (1.5 g, 13 mmol) was added to a solution of tert-butyl {[trans-4-({[(2-phenylquinolin-4-yl)carbonyl]amino}methyl)cyclohexyl]methyl}carbamate [Example 6] (0.20 g, 0.41 mmol) in DCM (8.0 mL) and the reaction mixture was stirred at rt for 1 h. The reaction mixture was concentrated in vacuo to leave a residue, which was dissolved in DCM and a saturated aq. solution of NaHCO₃ was added. The layers were separated and the organic layer was dried (phase separator) and concentrated in vacuo to give the crude title compound (0.80 g, 52%) which was used directly in the next step with no further purification. ¹H NMR (400 MHz, MeOH-d₄) δ 8.48 (d, 1H), 8.39-8.34 (m, 2H), 8.23-8.14 (m, 3H), 7.96 (t, 1H), 7.80-7.69 (m, 3H), 3.41 (d, 2H), 2.81 (d, 2H), 2.03-1.85 (m, 4H), 1.78-1.61 (m, 2H), 1.22-1.03 (m, 4H); m/z 274.18 (M+H)⁺.

### Intermediate NN

### Methyl 4-(4-{[(trans-4-{[(tert-butoxycarbonyl)amino]methyl}cyclohexyl)methyl]-carbamoyl}quinolin-2-yl)benzoate

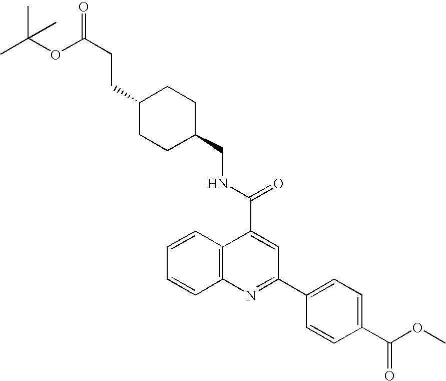

i) 2-[4-(Methoxycarbonyl)phenyl]quinoline-4-carboxylic acid

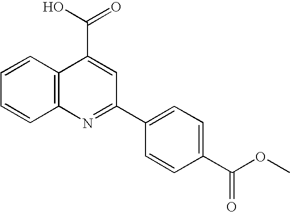

2-Chloro-quinoline-4-carboxylic acid (0.23 g, 1.4 mmol) was dissolved in dioxane (5 mL) and (4-methoxycarbonylphenyl)boronic acid (0.39 g, 2.2 mmol), Pd(PPh₃)₄ (0.20 g, 0.17 mmol) and K₂CO₃ (0.73 g, 5.3 mmol) were added. The reaction mixture was degassed, sealed, and heated in the microwave at 150° C. for 30 min. The reaction mixture was filtered and concentrated in vacuo to leave a residue. The residue was purified by flash column chromatography, using a 1:2 mixture of EtOAc/heptane with 1% acetic acid as eluent, to give the title compound (0.23 g, 53%). m/z 308.06 (M+H)⁺.

ii) TBTU (86 mg, 0.27 mmol) and NMM (39 mg, 0.38 mmol) were added to a solution of 2-[4-(methoxycarbonyl)phenyl]quinoline-4-carboxylic acid (29 mg, 0.10 mmol) in DMF (2 mL) and the reaction mixture was stirred at rt for 10 min. tert-Butyl {[trans-4-(amino-methyl)cyclohexyl]methyl}carbamate (35 mg, 0.15 mmol) was then added and the reaction mixture was stirred at rt for 2 h. The reaction mixture was concentrated in vacuo to leave a residue, which was dissolved in DCM and washed with a saturated aq. solution of NaHCO₃ and dried (phase separator). The mixture was concentrated in vacuo to leave a residue which was dissolved in DMSO and purified by HPLC (Standard method A) to give the title compound (Intermediate NN, 12 mg, 24%). m/z 530.32 (M+H)⁺.

### Intermediate OO

### 2-Phenylquinoline-4-thiol

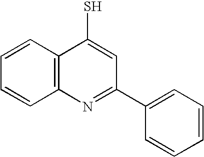

4-Chloro-2-phenyl-quinoline (0.5 g, 2.1 mmol) and sodium ethanethiolate (0.88 g, 10 mmol) were dissolved in DMF (7.5 mL) and heated at 153° C. under a nitrogen atmosphere for 1.5 h. The reaction mixture was concentrated in vacuo to half of its initial volume and then a saturated aq. solution of NH₄Cl was added and the pH was adjusted to 5 by the addition of a 2M aq. solution of HCl. The mixture was filtered and the solid was washed with a weakly acidic aq. solution to give the title compound (0.45 g, 91%). ¹H NMR (400 MHz, DMSO-d₆) δ 12.72 (s, 1H), 8.65 (dd, 1H), 7.90-7.83 (m, 3H), 7.76-7.70 (m, 1H), 7.61-7.57 (m, 3H), 7.54 (s, 1H), 7.48-7.42 (m, 1H); m/z 238.1 (M+H)⁺.

### Intermediate PP

### N-{[trans-4-(Aminomethyl)cyclohexyl]methyl}-2-[6-(4-methylpiperazin-1-yl)pyridin-3-yl]quinoline-4-carboxamide

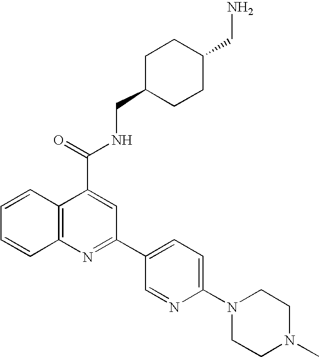

TFA (15 mL) was added to a solution of tert-butyl [(trans-4-{[({2-[6-(4-methylpiperazin-1-yl)pyridin-3-yl]quinolin-4-yl}carbonyl)amino]methyl}cyclohexyl)methyl]carbamate (Example 1) (1.5 g, 2.6 mmol) in DCM (30 mL) and the reaction mixture was stirred at rt for 1 h. The reaction mixture was concentrated in vacuo to give the title compound (1.2 g, 99%), which was used with no further purification; m/z (M+H)⁺ 473.3.

### Intermediate QQ

### 4-Nitrophenyl tetrahydro-2H-pyran-4-yl carbonate

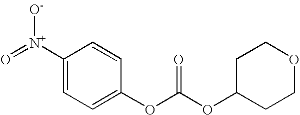

4-Hydroxy tetrahydropyran (0.56 g, 5.5 mmol) and TEA (0.90 mL, 6.4 mmol) were added to a solution of 4-nitrophenyl chloroformate (1.0 g, 5.0 mmol) in DCM (15 mL) and the reaction mixture was stirred at rt for 2 h. The reaction mixture was purified directly by flash column chromatography, using a 20→80% gradient of EtOAc in heptane as eluent, to give the title compound (1.1 g, 84%). ¹H NMR (400 MHz, CDCl₃) δ 8.23 (d, 2H), 7.34 (d, 2H), 4.94-4.88 (m, 1H), 3.99-3.89 (m, 2H), 3.60-3.49 (m, 2H), 2.08-1.97 (m, 2H), 1.87-1.73 (m, 2H).

### Intermediate RR-OOO

The Intermediates RR-OOO were prepared by the general procedure of Intermediate QQ using the appropriate alcohol in place of 4-hydroxy tetrahydropyran.

| Inter- | | ¹H NMR (400 MHz, |
|---|---|---|
| mediate | Name | CDCl₃) δ |
| RR | 1-Methylpiperidin-4-yl 4- | 8.26 (d, 2H), 7.37 (d, 2H), |
| | nitrophenyl carbonate | 4.82-4.71 (m, 1H), |
| | | 2.74-2.62 (m, 2H), |
| | | 2.34-2.20 (m, 2H), 2.28 (s, 3H), |
| | | 2.08-1.98 (m, 2H), |
| | | 1.93-1.81 (m, 2H) |
| SS | 4-Nitrophenyl oxetan-2- | 8.27 (d, 2H), 7.38 (d, 2H), |
| | ylmethyl carbonate | 5.10-5.03 (m, 1H), |
| | | 4.74-4.66 (m, 1H), |
| | | 4.64-4.57 (m, 1H), 4.48-4.36 (m, |
| | | 2H), 2.83-2.72 (m, 1H), |
| | | 2.66-2.56 (m, 1H) |
| TT | (1S)-2-Methoxy-1- | 8.25 (d, 2H), 7.37 (d, 2H), |
| | methylethyl 4-nitrophenyl | 5.10-5.00 (m, 1H), |
| | carbonate | 3.57-3.43 (m, 2H), 3.40 (s, 3H), |
| | | 1.36 (d, 3H) |
| UU | (1R)-2-Methoxy-1- | 8.25 (d, 2H), 7.37 (d, 2H), |
| | methylethyl 4-nitrophenyl | 5.10-5.00 (m, 1H), |
| | carbonate | 3.56-3.45 (m, 2H), 3.40 (s, 3H), |
| | | 1.36 (d, 3H) |
| VV | 4-Nitrophenyl | 8.30 (d, 2H), 7.40 (d, 2H), |
| | tetrahydrofuran-3-yl | 5.40-5.34 (m, 1H), |
| | carbonate | 4.08-3.89 (m, 4H), |
| | | 2.36-2.15 (m, 2H) |
| WW | 4-Nitrophenyl 2-(2- | 8.26 (d, 2H), 7.37 (d, 2H), |
| | oxopyrrolidin-1-yl)ethyl | 4.39 (t, 2H), 3.65 (t, 2H), |
| | carbonate | 3.53-3.47 (m, 2H), |
| | | 2.10-2.01 (m, 2H), |
| | | 2.43-2.35 (m, 2H) |
| XX | 4-Nitrophenyl(3S)- | 8.26 (d, 2H), 7.36 (d, 2H), |
| | tetrahydrofuran-3-yl | 5.36-5.31 (m, 1H), |
| | carbonate | 4.03-3.86 (m, 4H), |
| | | 2.32-2.13 (m, 2H) |
| YY | 2,2-Difluoroethyl 4- | 8.28 (d, 2H), 7.39 (d, 2H), |
| | nitrophenyl carbonate | 6.19-5.89 (m, 1H), |
| | | 4.50-4.41 (m, 2H) |
| ZZ | 2-Fluoroethyl 4- | 8.27 (d, 2H), |
| | | 7.38 (d, 2H), |
| | nitrophenyl carbonate | 4.77-4.73 (m, 1H), |
| | | 4.65-4.61 (m, 1H), |
| | | 4.57-4.53 (m, 1H), 4.50-4.47 |
| | | (m, 1H) |
| AAA | Ethyl 4-nitrophenyl | 8.26 (d, 2H), 7.36 (d, 2H), |
| | carbonate | 4.34 (q, 2H), 1.39 (t, 3H) |
| BBB | 2-Methoxyethyl 4- | 8.26 (d, 2H), 7.37 (d, 2H), |
| | nitrophenyl carbonate | 4.44-4.39 (m, 2H), |
| | | 3.71-3.66 (m, 2H), 3.42 (s, 3H) |
| CCC | (1S)-2-tert-Butoxy-1- | 8.25 (d, 2H), 7.37 (d, 2H), |
| | methylethyl 4-nitrophenyl | 5.01-4.91 (m, 1H), |
| | carbonate | 3.52-3.41 (m, 2H), 1.36 (d, 3H), |
| | | 1.19 (s, 9H) |
| DDD | 1-Cyanoethyl 4- | 8.29 (d, 2H), 7.40 (d, 2H), |
| | nitrophenyl carbonate | 5.37 (q, 1H), 1.78 (d, 3H) |
| EEE | 2-Acetamidoethyl 4- | 8.26 (d, 2H), 7.37 (d, 2H), |
| | nitrophenyl carbonate | 5.86 (s, 1H), 4.36 (t, 2H), |
| | | 3.64 (q, 2H), 2.03 (s, 3H) |
| FFF | (3-Methyloxetan-3- | 8.27 (d, 2H), 7.38 (d, 2H), |
| | yl)methyl 4-nitrophenyl | 4.55 (d, 2H), 4.44 (d, 2H), |
| | carbonate | 4.38 (s, 2H), 1.40 (s, 3H) |
| GGG | 4-Nitrophenyl(3R)-5- | 8.27 (d, 2H), 7.36 (d, 2H), |
| | oxopyrrolidin-3-yl | 5.54 (s, 1H), 5.45-5.40 (m, |
| | carbonate | 1H), 3.87-3.81 (m, 1H), |
| | | 3.61-3.57 (m, 1H), |
| | | 2.83-2.75 (m, 1H), |
| | | 2.59-2.53 (m, 1H) |
| HHH | 4-Nitrophenyl(3S)-5- | 8.27 (d, 2H), 7.36 (d, 2H), |
| | oxopyrrolidin-3-yl | 5.54 (s, 1H), 5.45-5.40 (m, |
| | carbonate | 1H), 3.87-3.81 (m, 1H), |
| | | 3.61-3.57 (m, 1H), |
| | | 2.83-2.75 (m, 1H), |
| | | 2.59-2.53 (m, 1H) |
| III | 4-Nitrophenyl | 8.26 (d, 2H), 7.36 (d, 2H), |
| | tetrahydrofuran-3-ylmethyl | 4.29-4.14 (m, 2H), |
| | carbonate | 3.88 (m, 2H), 3.79-3.72 (m, |
| | | 1H), 3.67-3.62 (m, 1H), |
| | | 2.74-2.62 (m, 1H), |
| | | 2.15-2.03 (m, 1H), |
| | | 1.73-1.62 (m, 1H) |
| JJJ | 4-Nitrophenyl | 8.25 (d, 2H), 7.36 (d, 2H), |
| | tetrahydrofuran-2-ylmethyl | 4.34-4.17 (m, 3H), |
| | carbonate | 3.94-3.78 (m, 2H), |
| | | 2.11-1.99 (m, 1H), 1.99-1.87 (m, |
| | | 2H), 1.72-1.60 (m, 1H) |
| KKK | (5-Methylisoxazol-3- | 8.26 (d, 2H), 7.37 (d, 2H), |
| | yl)methyl 4-nitrophenyl | 6.10 (s, 1H), 5.30 (s, 2H), |
| | carbonate | 2.44 (s, 3H) |
| LLL | 4-Nitrophenyl 2-(1H- | 8.25 (d, 2H), 7.58-7.56 (m, |
| | pyrazol-1-yl)ethyl | 1H), 7.49-7.47 (m, 1H), |
| | carbonate | 7.31 (d, 2H), 6.32-6.29 (m, |
| | | 1H), 4.64 (t, 2H), 4.48 (t, |
| | | 2H) |
| MMM | 4-Nitrophenyl 1,3-thiazol- | 8.27 (d, 2H), 7.85 (d, 1H), |
| | 2-ylmethyl carbonate | 7.45 (d, 1H), 7.39 (d, 2H), |
| | | 5.57 (s, 2H) |
| NNN | 4-Nitrophenyl pyrazin-2- | 8.77 (s, 1H), 8.70-8.58 (m, |
| | ylmethyl carbonate | 2H), 8.28 (d, 2H), 7.56 (d, |
| | | 2H), 5.42 (s, 2H) |
| OOO | 2-Cyanoethyl 4- | 8.28 (d, 2H), 7.38 (d, 2H), |
| | nitrophenyl carbonate | 4.48 (t, 2H), 2.83 (t, 2H) |

### Intermediate PPP

### (1S)-2-tert-Butoxy-1-methylethyl [(trans-4-{[({2-[6-(4-methylpiperazin-1-yl)-pyridin-3-yl]quinolin-4-yl}carbonyl)amino]methyl}cyclohexyl)methyl]carbamate

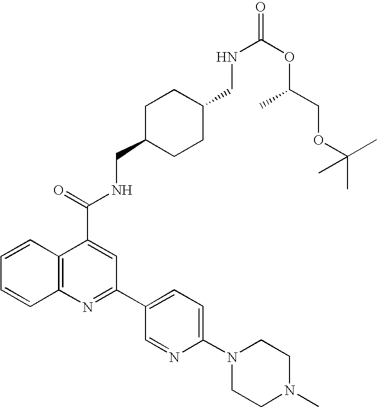

Intermediate PPP was synthesised according to the general method described for Example 109 (Method 19) by using (1S)-2-tert-butoxy-1-methylethyl 4-nitrophenyl carbonate (Intermediate CCC) in place of 4-nitrophenyl tetrahydro-2H-pyran-4-yl carbonate. ¹H NMR (400 MHz, CDCl₃) δ 9.00 (s, 1H), 8.75-8.70 (m, 1H), 8.42-8.38 (m, 1H), 8.06-7.96 (m, 3H), 7.76-7.69 (m, 1H), 7.56-7.49 (m, 1H), 7.06-7.00 (m, 1H), 6.98-6.93 (m, 1H), 4.67-4.56 (m, 1H), 3.63-3.54 (m, 4H), 3.26-3.24 (m, 2H), 3.21-3.15 (m, 2H), 2.82-2.74 (m, 2H), 2.65-2.61 (m, 4H), 2.39-2.35 (m, 3H), 1.84-1.76 (m, 2H), 1.73-1.66 (m, 2H), 1.55-1.46 (m, 1H), 1.36-1.26 (m, 1H), 1.10-1.06 (m, 12H), 0.99-0.78 (m, 4H); m/z (M+H)⁺ 631.3.

### Intermediate QQQ

### 2-{4-[(Methylamino)sulfonyl]phenyl}quinoline-4-carboxylic Acid

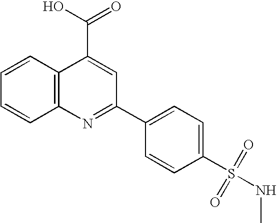

A solution of K₂CO₃ (0.37 g, 2.65 mmol) in water (2.5 mL) was added to a suspension of 2-chloroquinoline-4-carboxylic acid (0.20 g, 0.963 mmol), {4-[(methylamino) sulfonyl]phenyl}boronic acid (0.25 g, 1.16 mmol) and PEPPSI (20 mg, 29 μmol) in dioxane (2.5 mL) and the reaction mixture was heated at 140° C. in a microwave for 15 min. The reaction mixture was filtered and the filtrate was partitioned between EtOAc and a 1% aq. solution of citric acid. The layers were separated and the aqueous phase was washed with EtOAc (×2) and the combined organic phases were dried (Na₂SO₄) and concentrated in vacuo to leave the crude title compound (399 mg), which was used in the next step with no further purification. ¹H NMR (500 MHz, DMSO-d₆) δ 14.07 (s, 1H), 8.68-8.66 (m, 1H), 8.53-8.50 (m, 3H), 8.20 (d, 1H), 7.97-7.94 (m, 2H), 7.90-7.86 (m, 1H), 7.76-7.72 (m, 1H), 7.58 (m, 1H), 2.47 (d, 3H); m/z (M+H)⁺ 343.0.

### Intermediate RRR

### 2-[4-(Aminosulfonyl)phenyl]-quinoline-4-carboxylic Acid

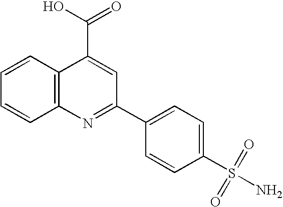

The title compound was prepared by the general procedure of Intermediate QQQ using {4-[(amino)sulfonyl]-phenyl}boronic acid in place of {4-[(methylamino)sulfonyl]-phenyl}boronic acid. ¹H NMR (400 MHz, DMSO-d₆) δ 14.03 (s, 1H), 8.63 (d, 1H), 8.50-8.43 (m, 3H), 8.16 (d, 1H), 7.99-7.95 (m, 2H), 7.87-7.82 (m, 1H), 7.73-7.68 (m, 1H), 7.45 (s, 2H); m/z (M+H)⁺ 328.9.

### Intermediate SSS-UUU

The following Intermediates SSS-UUU were prepared by the general procedure of Intermediate PP using the appropriate starting material selected from Example 134 or Example 135 in place of tert-butyl N—[[trans-4-[[[2-[6-(4-methylpiperazin-1-yl)pyridin-3-yl]quinoline-4-carbonyl]amino]methyl]cyclohexyl]methyl]carbamate.

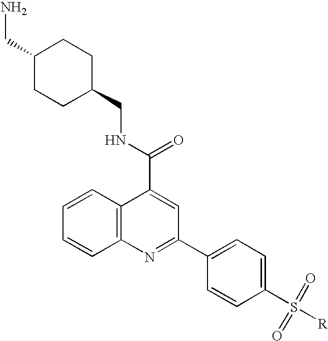

| | | | MS |
|---|---|---|---|
| Inter- | | | m/z |
| mediate | Name | R | (M + H)⁺ |
| SSS | N-{[trans-4-(Aminomethyl)cyclohexyl]- | NHMe | 467.2 |
| | methyl}-2-{4- | | |
| | [(methylamino)sulfonyl]phenyl}- | | |
| | quinoline-4-carboxamide | | |
| TTT | N-{[trans-4-(Aminomethyl)cyclohexyl]- | NH₂ | 453.1 |
| | methyl}-2-[4-(aminosulfonyl)phenyl]- | | |
| | quinoline-4-carboxamide | | |
| UUU | N-{[trans-4-(Aminomethyl)cyclohexyl]- | NMe₂ | 481.1 |
| | methyl}-2-{4-[(dimethylamino)sulfonyl]- | | |
| | phenyl}quinoline-4-carboxamide | | |

### Intermediate VVV

### tert-Butyl ({trans-4-[({[2-(6-fluoropyridin-3-yl)quinolin-4-yl]carbonyl}amino)-methyl]cyclohexyl}methyl)carbamate

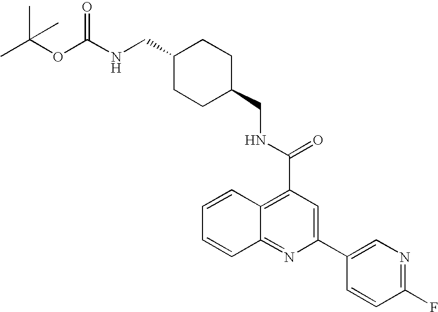

i) 2-(6-Fluoropyridin-3-yl)quinoline-4-carboxylic acid

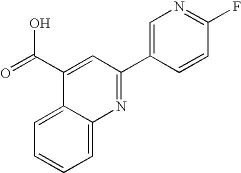

6-Fluoropyridine-3-boronic acid (1.6 g, 12 mmol), a 1M aq. solution of K₂CO₃ (25 mL) and PEPPSI (0.18 g, 0.26 mmol) were added sequentially to a solution of 2-chloro-quinoline-4-carboxylic acid (2.0 g, 9.6 mmol) in dioxane (25 mL). The reaction mixture was degassed and then heated at 100° C. under a nitrogen atmosphere for 2 h and then cooled to rt. The dioxane was removed by concentration in vacuo and the remaining residue was diluted with MeOH and citric acid to give a mixture of pH 4. The layers were separated and the aqueous phase was extracted with EtOAc. The combined organic layers were dried followed by concentration in vacuo to give the title compound (2.8 g, 94%). ¹H NMR (400 MHz, DMSO-d₆) δ 9.10 (s, 1H), 8.87-8.78 (m, 1H), 8.60 (d, 1H), 8.49 (s, 1H), 8.15 (d, 1H), 7.84 (t, 1H), 7.74-7.67 (m, 1H), 7.39-7.32 (m, 1H); m/z (M+H)⁺ 269.1.

ii) tert-Butyl {[trans-4-(aminomethyl)cyclohexyl]methyl}carbamate (1.1 g, 4.5 mmol), DIPEA (1.4 mL, 8.2 mmol) and TBTU (1.4 g, 4.5 mmol) were added sequentially to a solution of 2-(6-fluoropyridin-3-yl)quinoline-4-carboxylic acid (1.0 g, 3.7 mmol) in DMF (10 mL) at 0° C. and the reaction mixture was stirred at rt for 2 h. Water (10 mL) was added to give a precipitate and the mixture was filtered. The solid obtained was washed with a 1:1 mixture of water/MeOH to give the title compound (Intermediate VVV, 1.3 g, 70%). ¹H NMR (400 MHz, DMSO-d₆) δ 9.12 (d, 1H), 8.87-8.80 (m, 1H), 8.79-8.74 (m, 1H), 8.17 (s, 1H), 8.14-8.09 (m, 2H), 7.84-7.78 (m, 1H), 7.67-7.61 (m, 1H), 7.37 (dd, 1H), 6.80-6.67 (m, 1H), 3.23-3.16 (m, 2H), 2.77-2.72 (m, 2H), 1.86-1.76 (m, 2H), 1.73-1.65 (m, 2H), 1.55-1.45 (m, 1H), 1.34 (s, 9H), 1.33-1.28 (m, 1H), 1.00-0.78 (m, 4H); m/z (M+H)⁺ 493.1.

### Intermediate WWW

### 3-(4-Acetylpiperazin-1-yl)propan-1-ol

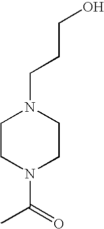

3-Bromo-1-propanol (1.1 g, 7.8 mmol) and NaHCO₃ (0.66 g, 7.8 mmol) were added sequentially to a mixture of 1-acetylpiperazine (1.0 g, 7.8 mmol) in DCM (10 mL) at 40° C. The reaction mixture was stirred at 40° C. for 4 h and then stirred at rt for 24 h. DCM, a saturated aq. solution of NaHCO₃ and brine were added and the layers were separated. The organic layer was dried (phase separator) and concentrated in vacuo to give the title compound (0.67 g, 46%). ¹H NMR (400 MHz, CDCl₃) δ 3.82-3.74 (m, 2H), 3.64-3.56 (m, 2H), 3.48-3.42 (m, 2H), 2.64-2.58 (m, 2H), 2.52-2.44 (m, 4H), 2.06 (s, 3H), 1.76-1.68 (m, 2H).

### Intermediate XXX

### tert-Butyl [(trans-4-{[({2-[4-(2-azidoethyl)piperidin-1-yl]quinolin-4-yl}carbonyl)amino]methyl}cyclohexyl)methyl]carbamate

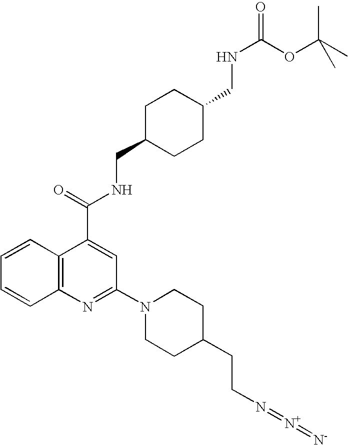

i) 2-[1-(4-{[(trans-4-{[(tert-Butoxycarbonyl)amino]methyl}cyclohexyl)methyl]-carbamoyl}quinolin-2-yl)piperidin-4-yl]ethyl methanesulfonate

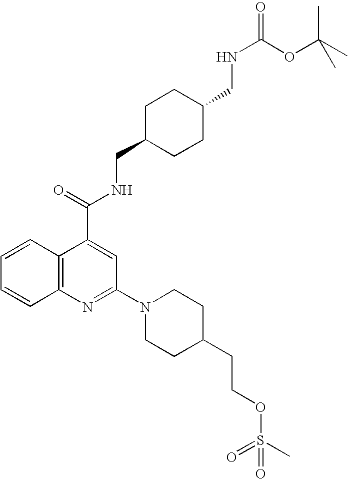

DIPEA (0.3 mL, 1.73 mmol) and methanesulfonyl chloride (158 mg, 1.40 mmol) were added to a solution of tert-butyl [(trans-4-{[({2-[4-(2-hydroxyethyl)piperidin-1-yl]quinolin-4-yl}carbonyl)amino]methyl}cyclohexyl)methyl]carbamate (Example 189) (363 mg, 0.69 mmol) in DCM (10 mL) at 0° C. and the reaction mixture was stirred for 1 h at rt. The reaction mixture was concentrated in vacuo to leave a residue which was used in the next step with no further purification. m/z (M+H)⁺ 603.3.

ii) Sodium azide (97 mg, 1.5 mmol) was added to a solution of 2-[1-(4-{[(trans-4-{[(tert-butoxycarbonyl)amino]methyl}cyclohexyl)methyl]carbamoyl}quinolin-2-yl)piperidin-4-yl]ethyl methanesulfonate (300 mg, 0.5 mmol) in DMF (3 ml) and the reaction mixture was stirred for 20 h at rt. The reaction mixture was purified by flash column chromatography, using a gradient of 50-100% EtOAc in heptane as eluent, to give the title compound (197 mg, 72%). ¹H NMR (300 MHz, CDCl₃) δ 7.86-7.84 (m, 1H), 7.71-7.69 (m, 1H), 7.57-7.52 (m, 1H), 7.26-7.22 (m, 2H), 7.05 (s, 1H), 5.98-5.93 (m, 1H), 4.60-4.51 (m, 2H), 3.41-3.35 (m, 4H), 3.01-2.92 (m, 4H), 1.90-1.79 (m, 6H), 1.77-1.68 (m, 1H), 1.62-1.56 (m, 4H), 1.55 (s, 9H), 1.34-1.25 (m, 2H), 1.11-0.92 (m, 4H); m/z (M+H)⁺ 550.3.

### Intermediate YYY

### 4-(Acetamidoethyl)piperidinium trifluoroacetate

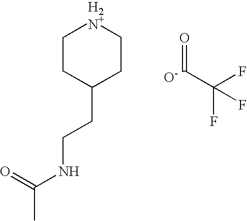

i) tert-Butyl 4-(2-acetamidoethyl)piperidine-1-carboxylate

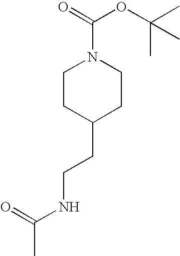

Acetic anhydride (0.45 g, 4.38 mmol) was added to a solution of tert-butyl 4-(2-aminoethyl)piperidine-1-carboxylate (1.0 g, 4.38 mmol) in DCM (10 mL) and the reaction mixture was stirred for 1 h at rt. A saturated aq. solution of NaHCO₃ was added and the layers were separated. The organic layer was dried (phase separator) and concentrated in vacuo to give the title compound (1.2 g, 100%), which was used with no further purification. ¹H NMR (300 MHz, CDCl₃) δ 5.46-5.36 (m, 1H), 4.12-3.98 (m, 2H), 3.29-3.22 (m, 2H), 2.70-2.60 (m, 2H), 1.94 (s, 3H), 1.68-1.60 (m, 3H), 1.43 (s, 9H), 1.45-1.38 (m, 2H), 1.15-1.03 (m, 2H); m/z (M+H)⁺ 271.3.

ii) TFA (4.0 mL, 53 mmol) was added to a solution of tert-butyl 4-(2-acetamido-ethyl)piperidine-1-carboxylate (420 mg, 1.54 mmol) in DCM (10 mL) and the reaction mixture was stirred for 1 h at rt. The reaction mixture was concentrated in vacuo to leave a residue, which was used directly in the next step with no further purification. m/z (M+H)⁺ 171.3 (minus TFA salt).

### Intermediate ZZZ

### 4-(Acetamidomethyl)piperidinium trifluoroacetate

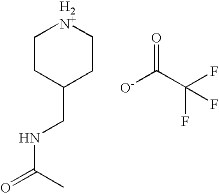

Intermediate ZZZ was prepared using the same procedure described for Intermediate YYY using tert-butyl 4-(2-aminomethyl)piperidine-1-carboxylate as starting material in place of tert-butyl 4-(2-aminoethyl)piperidine-1-carboxylate.

### Intermediate AAAA

### Tetrahydro-2H-pyran-4-yl ({trans-4-[({[2-(6-fluoropyridin-3-yl)quinolin-4-yl]carbonyl}amino)methyl]cyclohexyl}methyl)carbamate

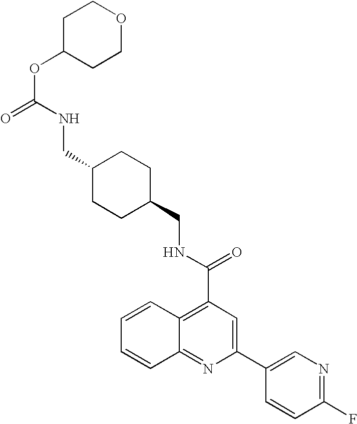

i) N-{[trans-4-(Aminomethyl)cyclohexyl]methyl}-2-(6-fluoropyridin-3-yl)quinoline-4-carboxamide

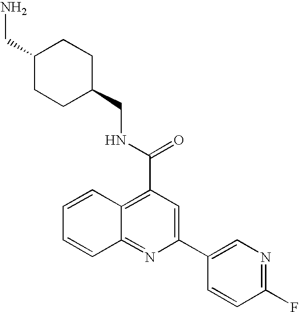

TFA (2.5 mL, 33 mmol) was added to a solution of tert-butyl({trans-4-[({[2-(6-fluoropyridin-3-yl)quinolin-4-yl]carbonyl}amino)methyl]cyclohexyl}methyl)-carbamate (Intermediate VVV) (650 mg, 1.32 mmol) in DCM (8 mL) and the reaction mixture was stirred at rt for 1 h. The reaction mixture was concentrated in vacuo to leave the crude product that was used in the next step with no further purification. m/z (M+H)⁺ 393.1.

ii) 4-Nitrophenyl tetrahydro-2H-pyran-4-yl carbonate (Intermediate QQ) (410 mg, 1.5 mmol) and TEA (1.6 mL, 11.5 mmol) were added sequentially to a stirred solution of N-{[trans-4-(aminomethyl)cyclohexyl]methyl}-2-(6-fluoropyridin-3-yl)quinoline-4-carboxamide (500 mg, 1.3 mmol) in THF (15 mL) and the reaction mixture was stirred at rt for 3 h and then at 40° C. for 1 h. The reaction mixture was concentrated in vacuo and DCM was added to give a precipitate. The mixture was filtered to leave a solid, which was washed with DCM to give the title compound (380 mg, 57%). ¹H NMR (400 MHz, DMSO-d₆) δ 9.51-9.43 (m, 1H), 9.24-9.08 (m, 2H), 8.55-8.41 (m, 3H), 8.22-8.11 (m, 1H), 8.05-7.96 (m, 1H), 7.76-7.68 (m, 1H), 7.48-7.41 (m, 1H), 5.04-4.92 (m, 1H), 4.18-4.04 (m, 2H), 3.81-3.67 (m, 2H), 3.58-3.48 (m, 2H), 3.21-3.10 (m, 2H), 2.22-1.98 (m, 5H), 1.91-1.72 (m, 3H), 1.70-1.58 (m, 1H), 1.53-1.41 (m, 1H), 1.34-1.14 (m, 4H); m/z (M+H)⁺ 521.

### Intermediate BBBB

### (3S)-Tetrahydrofuran-3-yl ({trans-4-[({[2-(6-fluoropyridin-3-yl)quinolin-4-yl]carbonyl}amino)methyl]cyclohexyl}methyl)carbamate

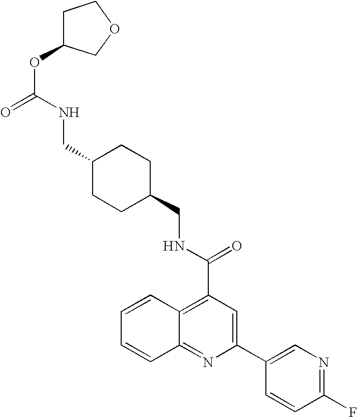

Intermediate BBBB was prepared using the same procedure as Intermediate AAAA using 4-nitrophenyl (3S)-tetrahydrofuran-3-yl carbonate (Intermediate XX) in place of 4-nitrophenyl tetrahydro-2H-pyran-4-yl carbonate in Step ii) to give the title compound (477 mg, 72%). ¹H NMR (400 MHz, DMSO-d₆) δ 9.13-9.10 (m, 1H), 8.86-8.74 (m, 2H), 8.14-8.08 (m, 3H), 7.83-7.77 (m, 1H), 7.66-7.61 (m, 1H), 7.39-7.34 (m, 1H), 7.18-7.13 (m, 1H), 5.07-5.02 (m, 1H), 3.75-3.56 (m, 4H), 3.21-3.16 (m, 2H), 2.81-2.76 (m, 2H), 2.09-1.99 (m, 1H), 1.83-1.76 (m, 3H), 1.70-1.65 (m, 2H), 1.54-1.45 (m, 1H), 1.34-1.25 (m, 1H), 0.98-0.77 (m, 4H); m/z (M+H)⁺ 507.1.

### Intermediate CCCC

### tert-Butyl ({trans-4-[({[2-(4-hydroxyphenyl)quinolin-4-yl]carbonyl}amino)-methyl]cyclohexyl}methyl)carbamate

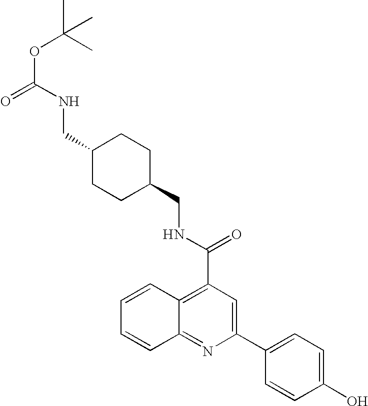

A solution of K₂CO₃ (86 mg, 0.63 mmol) in degassed water (1 mL) was added to a stirred solution of tert-butyl {[trans-4-({[(2-chloroquinolin-4-yl)carbonyl]amino}methyl)cyclohexyl]-methyl}carbamate (Example 35) (90 mg, 0.21 mmol), (4-hydroxyphenyl)boronic acid (35 mg, 0.25 mmol) and Pd(PPh₃)₄ (12 mg, 0.01 mmol) in degassed dioxane (3 mL) and the reaction mixture was stirred for 16 h at 60° C. The mixture was cooled to rt and filtered and the filtrate was partitioned between EtOAc and water. The aqueous phase was extracted with EtOAc and the combined organic layers were dried (Na₂SO₄) and concentrated in vacuo to leave a residue. The residue was purified by flash column chromatography, using a gradient of 40-100% EtOAc in heptane as eluent, to give the title compound (88 mg, 86%). ¹H NMR (400 MHz, DMSO-d₆) δ 9.90-9.84 (m, 1H), 8.76-8.72 (m, 1H), 8.14-8.10 (m, 2H), 8.07-7.99 (m, 2H), 7.95 (s, 1H), 7.76-7.70 (m, 1H), 7.56-7.51 (m, 1H), 6.91-6.87 (m, 2H), 6.78-6.73 (m, 1H), 3.20-3.15 (m, 2H), 2.77-2.71 (m, 2H), 1.82-1.76 (m, 2H), 1.71-1.65 (m, 2H), 1.54-1.46 (m, 1H), 1.33 (s, 9H), 1.29-1.23 (m, 1H), 0.99-0.76 (m, 4H); m/z (M+H)⁺ 490.2.

### Intermediate DDDD

### tert-Butyl [(trans-4-{[({2-[4-(oxiran-2-ylmethyl)piperidin-1-yl]quinolin-4-yl}carbonyl)amino]methyl}cyclohexyl)methyl]carbamate

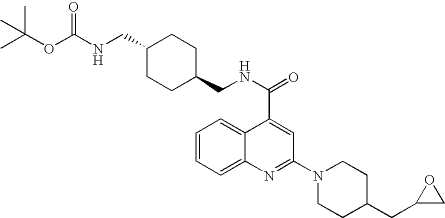

i) tert-Butyl [(trans-4-{[({2-[4-(2-oxoethyl)piperidin-1-yl]quinolin-4-yl}carbonyl)-amino]methyl}cyclohexyl)methyl]carbamate

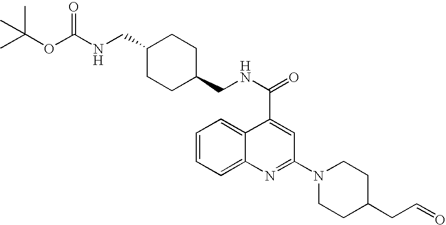

Oxalyl chloride (0.26 mL, 3.1 mmol, 3 eq.) was added dropwise to a solution of DMSO (0.44 mL, 6.2 mmol, 6 eq.) in DCM (15 mL) at −78° C. and the temperature was monitored to not exceed -60° C. A solution of tert-butyl [(trans-4-{[({2-[4-(2-hydroxyethyl)piperidin-1-yl]quinolin-4-yl}carbonyl)amino]methyl}cyclohexyl)-methyl]carbamate (Example 189) (0.54 g, 1.0 mmol) in DCM (10 mL) was added in portions, maintaining the temperature below −60° C. The mixture was stirred for 30 min and then TEA (2 mL, 14.5 mmol, 14 eq.) was added. The reaction mixture was warmed to rt and stirred for 30 min. The reaction mixture was diluted with DCM (100 mL) and water was added. The layers were separated and the aq. layer was extracted with DCM (50 mL). The combined organic layers were dried (Na₂SO₄), filtered and concentrated in vacuo to leave a crude residue. The crude residue was purified with HPLC chromatography, using a Kromasil C8 column, 10 μm, 250×50 ID mm, 10-95% mobilephase B over 30 min (Mobile phase A=water:ACN:formic acid 95:5:0.2, mobilephase B=ACN). The required fractions were concentrated in vacuo to leave the water phase, which was diluted further with water and pH of the solution was raised to 11 by adding saturated aq. sodium carbonate solution and extracted with EtOAc (2×150 mL). The combined organic layers were dried (Na₂SO₄), filtered and concentrated in vacuo to leave the title compound (0.52 g, 96%). ¹H NMR (500 MHz, CDCl₃) δ 9.8 (s, 1H), 7.86-7.80 (m, 1H), 7.70-7.64 (m, 1H), 7.56-7.50 (m, 1H), 7.26-7.20 (m, 1H), 6.98 (s, 1H), 6.18-6.1 (m, 1H), 4.64-4.55 (m, 1H), 4.53-4.44 (m, 2H), 3.40-3.33 (m, 2H), 3.02-2.92 (m, 4H), 2.43-2.38 (m, 2H), 2.25-2.14 (m, 1H), 1.90-1.77 (m, 6H), 1.63-1.52 (m, 1H), 1.43 (s, 9H), 1.37-1.26 (m, 3H), 1.1-0.9 (m, 4H); m/z (M+H)⁺ 523.4.

ii) Sodium hydride (60% in mineral oil, 79 mg, 1.98 mmol, 3 eq.) was added to a solution of trimethylsulfonium iodide (404 mg, 1.98 mmol, 3 eq.) in DMSO (5 mL) and the reaction mixture was stirred at rt for 15 min. A solution of tert-butyl [(trans-4-{[({2-[4-(2-oxoethyl)piperidin-1-yl]quinolin-4-yl}carbonyl)amino]methyl}-cyclohexyl)-methyl]carbamate (345 mg, 0.66 mmol) in DMSO (16 mL) was added and the reaction mixture was stirred at rt for 2 h. Water and a saturated aq. solution of sodium carbonate was added so the pH was 11. The mixture was extracted with DCM (2×150 mL) and the combined organic layers were dried (Na₂SO₄), filtered and concentrated in vacuo to give a residue. The residue was purified with HPLC chromatography, using a Kromasil C8 column, 10 μm, 250×50 ID mm, 20-90% mobilephase B over 25 min (Mobilephase A=water:ACN:formic acid 95:5:0.2, mobilephase B=ACN). The required fractions were concentrated in vacuo to leave the water phase, which was diluted with water and a saturated aq. solution of sodium carbonate was added so that the solution was pH 11. The mixture was extracted with EtOAc (2×150 mL) and the combined organic layers was dried (Na₂SO₄), filtered and concentrated in vacuo to leave the title compound (Intermediate DDDD), 0.43 g, 65%). ¹H NMR (500 MHz, CDCl₃) δ 7.82-7.76 (m, 1H), 7.66-7.60 (m, 1H), 7.53-7.47 (m, 1H), 7.22-7.15 (m, 1H), 6.91 (s, 1H), 6.42-6.36 (m, 1H), 4.69-4.60 (m, 1H), 4.51-4.39 (m, 2H), 3.36-3.29 (m, 2H), 2.99-2.85 (m, 5H), 2.77-2.73 (m, 1H), 2.45-2.41 (m, 1H), 1.91-1.73 (m, 7H), 1.60-1.47 (m, 2H), 1.47-1.24 (m, 4H), 1.42 (s, 9H), 1.07-0.86 (m, 4H); m/z (M+H)⁺ 537.4.

### Intermediate EEEE

### N,N-Dimethyl-2-(piperidin-4-yloxy)acetamide trifluoroacetate

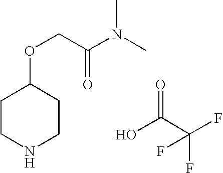

i) tert-Butyl 4-[2-(dimethylamino)-2-oxoethoxy]piperidine-1-carboxylate

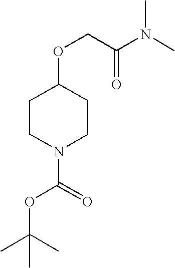

Sodium hydride (60% in mineral oil) (219 mg, 5.47 mmol, 1.1 eq.) was added to a solution of tert-butyl 4-hydroxypiperidine-1-carboxylate (1.0 g, 4.97 mmol), 2-chloro-N,N-dimethylacetamide (0.77 mL, 7.45 mmol, 1.5 eq.) and sodium iodide (149 mg, 0.99 mmol, 0.2 eq.) in DMF (10 mL) and the reaction mixture was stirred at rt for 18 h. The reaction mixture was diluted with water and a 1M aq. solution of citric acid was added to give a solution of pH 5. The layers were separated and the aq. phase was extracted with EtOAc (2×150 mL). The combined organic layers were dried (Na₂SO₄), filtered and concentrated in vacuo to give a residue. The residue was purified using flash column chromatography, using a mixture of EtOAc:MeOH (98:2) as eluent, to give the title compound (0.45 g, 32%). ¹H NMR (500 MHz, CDCl₃) δ 4.08 (s, 2H), 3.71-3.61 (m, 2H), 3.51-3.45 (m, 1H), 3.02-2.95 (m, 2H), 2.94 (s, 3H), 2.85 (s, 3H), 1.81-1.73 (m, 2H), 1.50-1.40 (m, 2H), 1.35 (s, 9H).

ii) TFA (2 mL, 26 mmol, 50 eq.) was added to a solution of tert-butyl 4-[2-(dimethylamino)-2-oxoethoxy]piperidine-1-carboxylate (150 mg, 0.52 mmol) in DCM (2 mL) and the reaction mixture was stirred at rt for 30 min. The reaction mixture was concentrated in vacuo to leave a residue. DCM (3×2 mL) was added and the solution was concentrated in vacuo each time to give the title compound (Intermediate EEEE, 155 mg, 98%), which was used in the next step without purification.

### Intermediate FFFF

### N,N-Dimethyl-2-(piperidin-4-yloxy)ethanamine bis(trifluoroacetate)

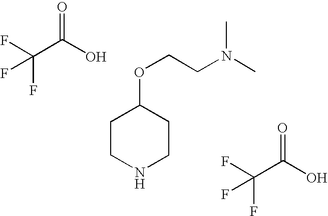

i) tert-Butyl 4-[2-(dimethylamino)ethoxy]piperidine-1-carboxylate

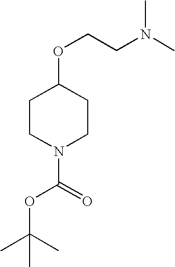

Borane-dimethylsulfide complex (2M in THF, 0.79 mL, 1.57 mmol, 1.5 eq.) was added to a solution of tert-butyl 4-[2-(dimethylamino)-2-oxoethoxy]piperidine-1-carboxylate (Intermediate 123) (0.3 g, 1.05 mmol) in THF (5 mL) and the reaction mixture was stirred at rt for 16 h. The reaction mixture was concentrated in vacuo to leave a residue. Water and a saturated aq. solution of sodium carbonate was added so the pH was 11. The mixture was extracted with EtOAc (2×100 mL) and the combined organic layers were dried (Na₂SO₄), filtered and concentrated in vacuo to leave a residue, which was purified with flash column chromatography, using a mixture of heptane:EtOAc (60:40) as eluent, to give the title compound (0.27 g, 95%). ¹H NMR (500 MHz, CDCl₃) δ 3.83-3.78 (m, 2H), 3.70-3.61 (m, 2H), 3.47-3.41 (m, 1H), 3.14-3.06 (m, 2H), 2.96-2.90 (m, 2H), 2.61 (s, 6H), 1.82-1.73 (m, 2H), 1.52-1.42 (m, 2H), 1.41 (s, 9H).

ii) TFA (3 mL, 38.9 mmol, 39 eq.) was added to a solution of tert-butyl 4-[2-(dimethylamino)ethoxy]piperidine-1-carboxylate (0.27 g, 0.99 mmol) in DCM (3 mL) and the reaction mixture was stirred at rt for 30 min. The reaction mixture was concentrated in vacuo to leave a residue. DCM (3×3 mL) was added and the mixture was concentrated in vacuo each time to give the title compound (Intermediate FFFF, 395 mg, 99%), which was used in the next step without purification.

### Intermediate GGGG

### N-{[trans-4-(Aminomethyl)cyclohexyl]methyl}-2-{4-[2-(dimethylamino)ethyl]-piperidin-1-yl}quinoline-4-carboxamide trihydrochloride

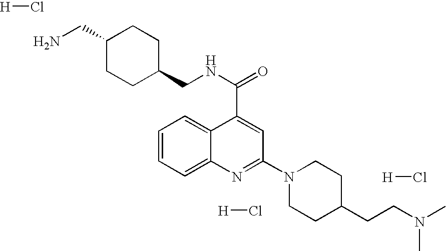

Hydrogen chloride (4M in 1,4-dioxane, 6 mL, 24 mmol) was added to a solution of tert-butyl {[trans-4-({[(2-{4-[2-(dimethylamino)ethyl]piperidin-1-yl}quinolin-4-yl)carbonyl]amino}methyl)cyclohexyl]methyl}carbamate (Example 193) (0.2 g, 0.36 mmol) in a mixture of 1,4-dioxane and water 1:1 (6 mL) and the reaction mixture was stirred at rt for 15 min. The reaction mixture was concentrated in vacuo to leave a residue. 1,4-Dioxane (3×5 mL) was added and the mixture was concentrated in vacuo each time to give the title compound (0.356 g, 98%) that was used with no further purification; m/z (M+H)⁺ 452.4.

### Intermediate HHHH

### 1-(4-{[(trans-4-{[(tert-Butoxycarbonyl)amino]methyl}cyclohexyl)methyl]-carbamoyl}quinolin-2-yl)azetidine-3-carboxylic Acid

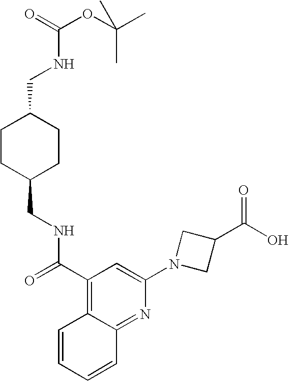

tert-Butyl {[trans-4-({[(2-chloroquinolin-4-yl)carbonyl]amino}methyl)cyclohexyl]-methyl}carbamate (Example 35) (0.40 g, 0.93 mmol) was added to a microwave vial (2-5 mL) followed by addition of pyridine (3 mL) and azetidine-3-carboxylic acid (0.37 g, 3.7 mmol). The reaction mixture was heated at 145° C. for 45 min using a microwave and then a second portion of azetidine-3-carboxylic acid (370 mg, 3.70 mmol) and K₂CO₃ (0.51 g, 3.7 mmol) was added. The reaction mixture was then heated at 180° C. for 90 min in a microwave. The reaction mixture was concentrated in vacuo to leave a residue, which was purified by flash column chromatography, using a gradient of MeOH in DCM (5 to 100%) as eluent, to give the title compound (0.07 g, 15%). ¹H NMR (400 MHz, CD₃OD) δ 7.80 (d, 1H), 7.59 (d, 1H), 7.46 (t, 1H), 7.17 (t, 1H), 6.53 (s, 1H), 4.36-4.19 (m, 2H), 3.45-3.18 (m, 5H), 2.94-2.84 (m, 2H), 1.90-1.71 (m, 4H), 1.62-1.47 (m, 1H), 1.39 (s, 9H), 1.36-1.28 (m, 1H), 1.08-0.79 (m, 4H); m/z (M+H)⁺ 497.2.

### Intermediate IIII

### 1-[2-(Dimethylamino)ethyl]piperazin-2-one

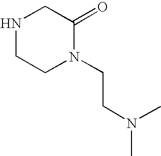

i) tert-Butyl 4-[2-(dimethylamino)ethyl]-3-oxopiperazine-1-carboxylate

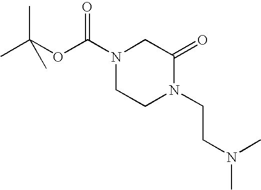

Sodium hydride (60% in mineral oil, 0.33 g, 8.12 mmol) was added to a solution of tert-butyl 3-oxopiperazine-1-carboxylate (0.50 g, 2.50 mmol) in DMF (8 mL) at 0° C. and the reaction mixture was stirred at 0° C. for 30 min. 2-(Dimethylamino)ethyl chloride hydrochloride (0.40 g, 2.75 mmol) was then added and the reaction mixture was stirred at 70° C. for 16 h. The reaction mixture was poured into an ice-water mixture and extracted with diethylether (×4). The combined organic layers were dried (Na₂SO₄) and concentrated in vacuo to leave a residue, which was purified by preparative HPLC (Standard Method C) to give the title compound (0.18 g, 26%). ¹H NMR (600 MHz, DMSO/DMSO-d₆*) δ 3.84 (s, 2H), 3.48 (s, 2H), 3.36 (t, 2H), 2.31 (t, 2H), 2.11 (s, 6H), 1.38 (s, 9H); m/z (M+H)⁺ 272.1.

ii) TFA (1 mL) was added to a solution of tert-butyl 4-[2-(dimethylamino)ethyl]-3-oxopiperazine-1-carboxylate (0.176 g, 0.649 mmol) in DCM (1 mL) and the reaction mixture was stirred at rt for 2 h. The reaction mixture was then concentrated in vacuo to leave the crude title compound (Intermediate IIII) as a TFA salt that was used with no further purification; m/z (M+H)⁺ 172.0.

### Intermediate JJJJ

### 2-Oxo-2-piperazin-1-ylethanol

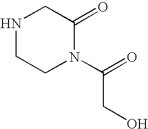

i) tert-Butyl 4-(hydroxyacetyl)piperazine-1-carboxylate

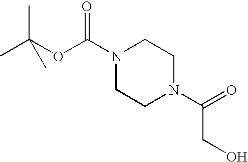

DIPEA (1.41 mL, 8.10 mmol), hydroxyacetic acid (0.23 g, 2.97 mmol) and finally TBTU (1.04 g, 3.24 mmol) were added to a solution of tert-butyl piperazine-1-carboxylate (0.50 g, 2.70 mmol) in DCM (10 mL). The reaction mixture was stirred for 2 h and then purified by flash column chromatography, using a gradient of 40→100% EtOAc in heptane followed by 0→10% MeOH in DCM as eluent, to give the title compound (0.33 g, 50%). ¹H NMR (400 MHz, CDCl₃) δ 4.17 (s, 2H), 3.66-3.62 (m, 2H), 3.48-3.43 (m, 4H), 3.26-3.22 (m, 2H), 2.80 (s, 1H), 1.47 (s, 9H).

ii) TFA (1 mL) was added to a solution of tert-butyl 4-(hydroxyacetyl)piperazine-1-carboxylate (0.176 g, 1.19 mmol) in DCM (1 mL) and the reaction mixture was stirred at room temperature for 2 h. The reaction mixture was then concentrated in vacuo to leave the crude title compound (Intermediate JJJJ) as a TFA salt that was used with no further purification; m/z (M+H)⁺ 144.9.

### Intermediate KKKK

### 4-[(4-Fluorobenzyl)oxy]piperidine

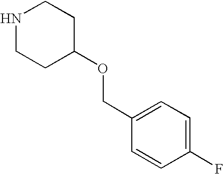

i) tert-Butyl 4-[(4-fluorobenzyl)oxy]piperidine-1-carboxylate

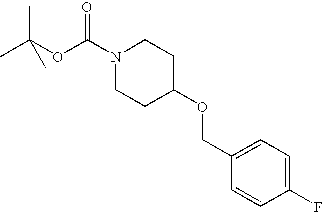

An aq. solution of NaOH (50%, 7 mL), tetrabutylammonium hydrogensulphate (0.17 g, 0.50 mmol) and 4-fluorobenzyl bromide (0.66 g, 3.48 mmol) were added to a solution of tert-butyl 4-hydroxypiperidine-1-carboxylate (0.50 g, 2.48 mmol) in toluene (15 mL). The reaction mixture was stirred vigorously for 16 h and then diethylether and water were added. The layers were separated and the aqueous phase was extracted with DCM. The combined organic layers were dried (phase separator) and concentrated in vacuo to leave a residue which was purified by flash column chromatography, using a gradient of 0→55% EtOAc in heptane as eluent, to give the title compound (0.69 g, 90%). ¹H NMR (400 MHz, CDCl₃) δ 7.33-7.28 (m, 2H), 7.05-6.99 (m, 2H), 4.51 (s, 2H), 3.81-3.73 (m, 2H), 3.58-3.51 (m, 1H), 3.13-3.05 (m, 2H), 1.89-1.81 (m, 2H), 1.62-1.52 (m, 2H), 1.45 (s, 9H).

ii) TFA (1.5 mL) was added to tert-butyl 4-(hydroxyacetyl)piperazine-1-carboxylate (0.69 g, 2.23 mmol) in DCM (1.5 mL) and the reaction mixture was stirred at rt for 3 h. Toluene was added and the reaction mixture was concentrated in vacuo to leave the crude title compound (Intermediate KKKK) as a TFA salt that was used with no further purification. m/z (M+H)⁺ 209.9.

### Intermediate LLLL

### 2-Amino-1-benzothiophene-3-carboxylic Acid

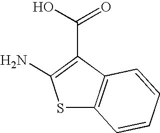

An aq. solution of NaOH (1M, 20 mL) was added to a solution of ethyl 2-amino-1-benzothiophene-3-carboxylate (0.66 g, 3.00 mmol) in dioxane (10 mL) and the reaction mixture was heated at 100° C. for 2 h. The reaction mixture was concentrated in vacuo to leave an aqueous residue and then a 2M aq. solution of HCl was added such that the pH=4 to give a precipitate. The mixture was filtered and the collected solid was washed with water to give the crude title compound (0.42 g, 72%). ¹H NMR (400 MHz, DMSO/DMSO-d₆)*) δ 12.25 (s, 1H), 7.95 (d, 1H), 7.89 (s, 2H), 7.56 (d, 1H), 7.22-7.17 (m, 1H), 7.04-6.99 (m, 1H); m/z (M−H)⁻ 192.2.

### Intermediate MMMM

### Benzyl (1-{2-[1-(4-{[(trans-4-{[(tert-butoxycarbonyl)amino]methyl}cyclohexyl)-methyl]carbamoyl}quinolin-2-yl)piperidin-4-yl]ethyl}azetidin-3-yl)carbamate

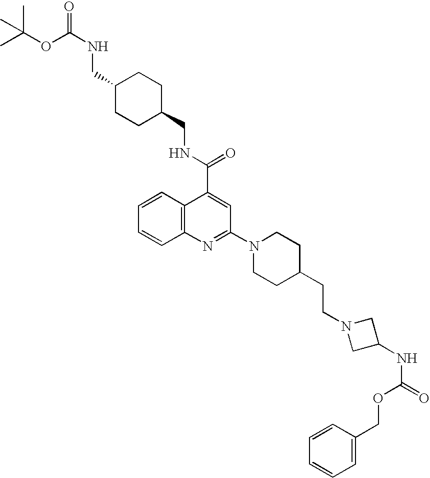

Intermediate MMMM was prepared by the method described in Example 226 (Method 23) using benzyl azetidin-3-ylcarbamate as starting material in place of 2-methoxyethylamine. ¹H NMR (400 MHz, CDCl₃) δ 7.84 (d, 1H), 7.68 (d, 1H), 7.56-7.51 (m, 1H), 7.39-7.29 (m, 5H), 7.25-7.20 (m, 1H), 7.03 (s, 1H), 5.97 (t, 1H), 5.09 (s, 3H), 4.62-4.54 (m, 1H), 4.54-4.47 (m, 2H), 4.39-4.30 (m, 1H), 3.61 (t, 2H), 3.38 (t, 2H), 3.02-2.89 (m, 4H), 2.86-2.79 (m, 2H), 2.47-2.44 (m, 2H), 1.90-1.74 (m, 6H), 1.64-1.53 (m, 2H), 1.47-1.37 (m, 1H), 1.44 (s, 9H), 1.33-1.18 (m, 4H), 1.11-0.91 (m, 4H); m/z (M+H)⁺ 713.2.

### Intermediate NNNN

### tert-Butyl ({trans-4-[({[2-(3-hydroxyazetidin-1-yl)quinolin-4-yl]carbonyl}amino)methyl]cyclohexyl}methyl)carbamate

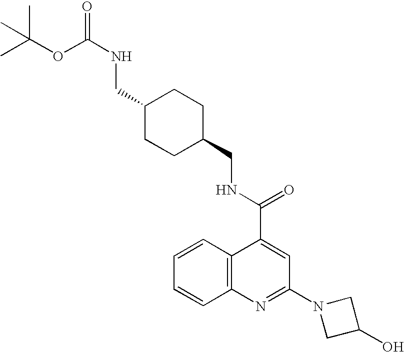

Intermediate NNNN was prepared by the method described in Example 185 (Method 22) using azetidin-3-ol (Intermediate OOOO) as starting material in place of 1-piperazineethanol. ¹H NMR (600 MHz, CD₃OD) δ 7.84-7.82 (m, 1H), 7.68 (d, 1H), 7.57-7.54 (m, 1H), 7.27-7.23 (m, 1H), 6.69 (s, 1H), 4.75-4.71 (m, 1H), 4.45-4.41 (m, 2H), 3.98 (dd, 2H), 3.31-3.27 (m, 2H), 2.89 (d, 2H), 1.92-1.87 (m, 2H), 1.84-1.79 (m, 2H), 1.64-1.56 (m, 1H), 1.46-1.38 (m, 1H), 1.43 (s, 9H), 1.09-0.93 (m, 4H); m/z (M+H)⁺ 469.1.

### Intermediate OOOO

### Azetidin-3-ol

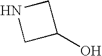

A mixture of benzyl 3-hydroxyazetidine-1-carboxylate (2.05 g, 9.89 mmol) and 5% palladium on carbon (2.05 g, 0.96 mmol) in MeOH (40 mL) was stirred under a hydrogen atmosphere at rt for 3 h. The reaction mixture was filtered and the filtrate was concentrated in vacuo to leave the title compound which was used with no further purification. m/z (M+H)⁺ 73.9.

### Intermediate PPPP

### tert-Butyl [(trans-4-{[({2-[3-(aminomethyl)phenyl]-6-chloropyridin-4-yl}carbonyl)amino]methyl}cyclohexyl)methyl]carbamate

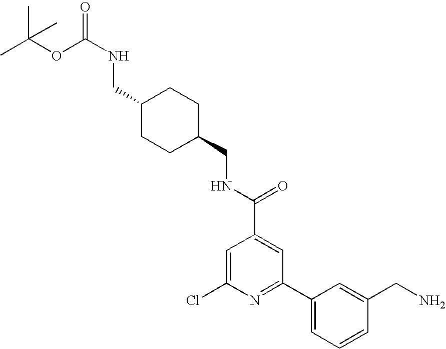

i) tert-Butyl {[trans-4-({[(2,6-dichloropyridin-4-yl)carbonyl]amino}methyl)-cyclohexyl]methyl}carbamate

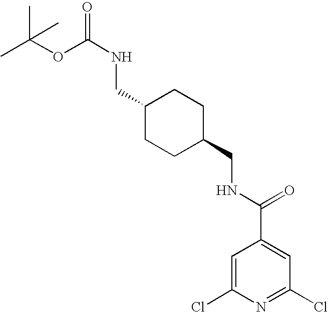

2,6-Dichloroisonicotinic acid (3.00 g, 15.0 mmol) was dissolved in DMF and tert-butyl {[trans-4-(aminomethyl)cyclohexyl]methyl}carbamate (4.17 g, 17.2 mmol), TBTU (6.02 g, 18.8 mmol) and NMM (2.37 g, 2.58 mL, 23.4 mmol) were added. The reaction mixture was stirred at rt for 16 h. Water was added and the reaction mixture was filtered. The solid that was collected was re-crystallized from a mixture of MeOH and water to give the title compound (4.29 g, 66%). ¹H NMR (400 MHz, CDCl₃) δ 7.56 (s, 2H), 6.29 (s, 1H), 4.59 (s, 1H), 3.30 (t, 2H), 2.97 (t, 2H), 1.84-1.76 (m, 4H), 1.55 (s, 1H), 1.44-1.37 (m, 10H), 1.05-0.90 (m, 4H).

ii) (3-Aminomethylphenyl)boronic acid hydrochloride (0.17 g, 0.91 mmol), bis(triphenylphosphine)palladium(II) chloride (30 mg, 0.04 mmol) and a solution of cesium carbonate (0.70 g, 2.16 mmol) in water (3 mL) were added to a solution of tert-butyl {[trans-4-({[(2,6-dichloropyridin-4-yl)carbonyl]amino}methyl)cyclohexyl]methyl}carbamate (0.36 g, 0.86 mmol) in dioxane (9 mL). The reaction mixture was heated in a microwave at 130° C. for 60 min. The reaction mixture was concentrated in vacuo and a saturated aq. solution of NaHCO₃ was added. The layers were separated and the aq. layer was extracted with methyl-THF. The combined organic layers were dried (Na₂SO₄) and concentrated in vacuo to leave a residue, which was purified by flash column chromatography, using a gradient of EtOAc to MeOH as eluent, to give the title compound (Intermediate PPPP, 0.12 g, 29%). ¹H NMR (400 MHz, CDCl₃) δ 8.01 (s, 1H), 7.98 (d, 1H), 7.91 (d, 1H), 7.50 (s, 1H), 7.48-7.41 (m, 2H), 6.27 (t, 1H), 4.58 (s, 1H), 3.97 (s, 2H), 3.34 (t, 2H), 2.98 (t, 2H), 1.83 (t, 4H), 1.58 (s, 1H), 1.44 (s, 10H), 1.08-0.91 (m, 4H).

### Intermediate QQQQ

### tert-Butyl ({trans-4-[({[2-(1H-pyrazol-4-yl)quinolin-4-yl]carbonyl}amino) methyl]cyclohexyl}methyl)carbamate

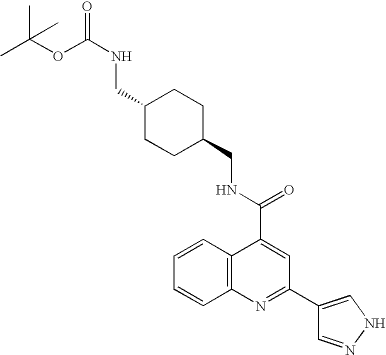

tert-Butyl {[trans-4-({[(2-chloroquinolin-4-yl)carbonyl]amino}methyl)cyclohexyl]-methyl}carbamate (Example 35, 275 mg, 0.64 mmol) was dissolved in dioxane (6 mL) and PEPPSI (4 mg, 1 mol %), 4-(4,4,5,5-tetramethyl-1,3,2-dioxaborolan-2-yl)-1H-pyrazole (200 mg, 1.03 mmol) and a solution of K₂CO₃ (176 mg, 1.27 mmol) in water (3 mL) were added. The reaction mixture was heated in a microwave at 140° C. for 30 min and then a saturated aq. solution of NaHCO₃ was added. The layers were separated and the aq. layer was extracted with EtOAc. The combined organic layers were concentrated in vacuo to give the crude title compound (286 mg, 97%), which was used with no further purification. ¹H NMR (400 MHz, DMSO-d₆) δ 8.73 (t, 1H), 8.56 (s, 1H), 8.24 (s, 1H), 7.98-7.95 (m, 2H), 7.84 (s, 1H), 7.72 (t, 1H), 7.52 (t, 1H), 6.78 (t, 1H), 6.23 (s, 1H), 3.19 (t, 2H), 2.76 (t, 2H), 1.81 (d, 2H), 1.69 (d, 2H), 1.51 (s, 1H), 1.35 (s, 9H), 1.32 (m, 1H), 0.99-0.79 (m, 4H).

Various modifications of the invention, in addition to those described herein, will be apparent to those skilled in the art from the foregoing description. Such modifications are also intended to fall within the scope of the appended claims. It is also understood that any embodiment described herein can be combined with any other embodiment described herein.

What is claimed is:

1. A compound of formula (I) 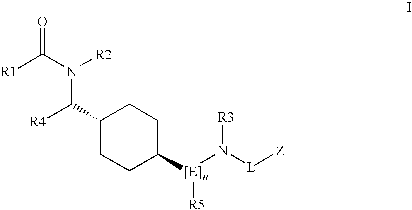
or a pharmaceutically acceptable salt thereof, in which:
R₁ represents —OR⁶ or —NR^{7a}R^{7b};
R₂ and R₃ independently represent hydrogen, C₁-C₆ alkyl, C₂-C₆ alkenyl, C₂-C₆ alkynyl, C₁-C₆ alkoxy, C₃-C₈ cycloalkyl, C₃-C₈ cycloalkenyl (which alkyl, alkenyl, alkynyl, alkoxy, cycloalkyl, cycloalkenyl groups are optionally substituted by one or more halo, OH, cyano, —C(O)NR^{8a}R^{8b}, —C(O)OR⁹, aryl, heteroaryl, heterocyclyl);
R₄ represents hydrogen or C₁-C₆ alkyl which alkyl group is optionally substituted by one or more halo, OH, —C(O)NR^{10a}R^{10b}, C₁-C₄ alkoxy optionally substituted by one or more halo;
R₅ represents hydrogen or C₁-C₆ alkyl which alkyl group is optionally substituted by one or more halo, OH, —C(O)NR^{11a}R^{11b}, C₁-C₄ alkoxy optionally substituted by one or more halo;
E represents C₁-alkylene;
n is an integer of 0 or 1;
L represents C(O) or SO₂;
Z represents aryl, heteroaryl, heterocyclyl, which groups are optionally substituted by one or more groups independently selected from any of A), B) and C) as defined:
A) halo, —NR¹²C(O)R¹³, —NR¹⁴C(O)N(R^{15a})(R^{15b}), —N(R^{15a})(R^{15b});
B) C₁-C₆ alkyl, C₁-C₆ alkoxy, (which alkyl and alkoxy groups are optionally substituted by halo, OH, —N(R^{16a})(R^{16b}), heterocyclyl, heteroaryl (which heterocyclyl, heteroaryl groups are optionally substituted by OH, oxo, C₁-C₆ alkyl, C(O)R¹⁷));
C) C₃-C₈ cycloalkyl, C₃-C₈ cycloalkenyl, heterocyclyl, —(O)_{0 or 1} aryl, —(O)_{0 or 1}-heteroaryl, which cycloalkyl, cycloalkenyl, heterocyclyl, aryl, heteroaryl groups (group C) are optionally substituted by one or more groups independently selected from any of a), b) and c) as defined:
a) halo, —OH, oxo, cyano, —C(O)N(R^{18a})(R^{18b}), —N(R^{19a})(R^{19b}), —SR²⁰, —OR^{20a}, —N(R²¹)C(O)R²², —SO₂(R²³), —N(R²⁴)(CHR²⁵)ₘN(R^{26a})(R^{26b}) or C(O)R³⁸;
b) C₁-C₆ alkyl, C₁-C₆ alkoxy, C₃-C₈ cycloalkyl, C₃-C₈ cycloalkenyl (which alkyl, alkoxy, cycloalkyl, cycloalkenyl groups are optionally substituted by one or more halo, —OH, cyano, N(R^{27a})(R^{27b}), —N(R²⁸)SO(R²), —N(R³⁰)C(O)R³¹, —N(R³²)C(O)N(R^{33a})(R^{33b}), —N(R³⁴)C(O)OR³⁵, —SO₍₁₋₂₎(R³⁶), —C(O)OR³⁷, —C(O)R³⁸, —C(O)N(R^{39a})(R^{39b}), heterocyclyl (optionally substituted by C₁-C₄ alkyl, C₁-C₄ alkoxy, oxo, hydroxy, carboxy, amino, C₁-C₆alkylamino, di(C₁-C₆alkyl)amino in which the alkyl groups may be the same or different));
c) aryl, heteroaryl, heterocyclyl (which aryl, heteroaryl, heterocyclyl are optionally substituted by —OH, —C(O)R⁴⁰, —C(O)N(R^{41a})(R^{41b}), oxo, —N(R^{42a})(R^{42b}), —O(R⁴³), C₁-C₆ alkyl, C₁-C₆ alkoxy (which alkyl and alkoxy groups are optionally substituted by halo, —OH));
R⁶ represents C₁-C₆ alkyl, C₃-C₈ cycloalkyl, C₃-C₈ cycloalkenyl, aryl, heteroaryl, heterocyclyl (which groups are optionally substituted by one or more of —OH, halo, cyano, C₁-C₆ alkyl, C₁-C₆ alkoxy, oxo, —NR⁴⁴C(O)N(R^{45a})(R^{45b}), —NR⁴⁴C(O)(R⁴⁰), heterocyclyl or heteroaryl (which heterocyclyl or heteroaryl groups are optionally substituted by C₁-C₄ alkyl, oxo));
R^{7a}, R^{7b}, R^{8a}, R^{8b}, R^{10a}, R^{10b}, R^{11a}, R^{11b}, R^{15a}, R^{15b}, R^{16a}, R^{16b}, R^{18a}, R^{18b}, R^{19a}, R^{19b}, R^{26a}, R^{26b}, R^{27a}, R^{27b}, R^{33a}, R^{33b}, R^{39a}, R^{39b}, R^{41a}, R^{41b}, R^{42a}, R^{45a}, R^{45b}, R^{46a}, R^{46b} independently represent hydrogen, heterocyclyl or a C₁-C₆ alkyl optionally substituted by OH, halo, C₁-C₄ alkoxy, cyano, amino, C₁-C₆alkylamino, di(C₁-C₆alkyl)amino in which the alkyl groups may be the same or different, heterocyclyl or heteroaryl (which heterocyclyl and heteroaryl groups are optionally substituted by OH, halo, C₁-C₄ alkyl, C₁-C₄ alkoxy);
R⁹, R¹³, R¹⁷, R²⁰, R²¹, R²², R²⁴, R²⁵, R³¹, R³⁴, R³⁵, R³⁷, R³⁸, R⁴⁰ independently represent hydrogen, C₁-C₆ alkyl, or C₁-C₆ alkoxy (which alkyl and alkyloxy are optionally substituted by halo, OH, cyano, C(O)NH₂);
R¹², R¹⁴, R²⁸, R³⁰, R³², R³⁴, R⁴⁴ represents hydrogen, C₁-C₆ alkyl (which alkyl is optionally substituted by halo, OH, cyano, C(O)NH₂);
R^{20a} represents phenyl(CH₂)₀₋₄—O— in which the phenyl is optionally substituted by halo, C₁-C₆ alkyl or C₁-C₆ alkoxy (which alkyl and alkoxy are optionally substituted by halo);
R²³, R²⁹, R³⁶ represent —N(R^{46a})(R^{46b}), C₁-C₆ alkyl, C₁-C₆ alkoxy (which alkyl and alkoxy are optionally substituted by halo, OH, cyano, C(O)NH₂);
R⁴³ represents heteroaryl, heterocyclyl;
m is an integer of 0, 1, 2, 3, 4, 5 or 6;
with the proviso that the compound is not:
a)
tert-Butyl [(4-{[(1-naphthylsulfonyl)amino]methyl}cyclohexyl)methyl]-carbamate;
tert-Butyl [(trans-4-{[(2-naphthylsulfonyl)amino]methyl}cyclohexyl)methyl] carbamate;
tert-Butyl({trans-4-[({[2-trifluoromethyl)phenyl]sulfonyl}amino)methyl]cyclohexyl)methyl)carbamate; or
b)
tert-Butyl [(trans-4-{[(1-naphthylsulfonyl)amino]methyl}cyclohexyl)methyl] carbamate;
N-{[4-({[(Cyclopropylmethyl)carbamoyl]amino}methyl)cyclohexyl]methyl}-4-fluorobenzamide;
2-Fluoro-N-{[4-({[(3-methoxypropyl)carbamoyl]amino}methyl)cyclohexyl]methyl}benzamide;
2-Fluoro-N-{[4-({[(2-methoxypropyl)carbamoyl]amino}methyl)cyclohexyl]methyl}benzamide;
3,4-Dimethoxy-N-{[4-({[(4-methylbenzyl)carbamoyl]amino}methyl)cyclohexyl]methyl}benzenesulfonamide;
3-Methyl-N-({4-[({[2-(trifluoromethyl)benzyl]carbamoyl}amino)methyl]cyclohexyl}methyl)benzamide;
6-Chloro-N-{[4-({[(4-fluorobenzyl)carbamoyl]amino}methyl)cyclohexyl]methyl}nicotinamide;
6-Chloro-N-{[4-({[(2-methoxyethyl)carbamoyl]amino}methyl)cyclohexyl]methyl}nicotinamide;
2,3-Dichloro-N-{[4-({[(2-phenylethyl)carbamoyl]amino}methyl)cyclohexyl]methyl}benzamide;
4-Methyl-N-[(4-{[(methylcarbamoyl)amino]methyl}cyclohexyl)methyl] benzamide;
3-Chloro-4-fluoro-N-{[4-({[(2-phenylethyl)carbamoyl]amino}methyl)cyclohexyl]methyl}benzenesulfonamide;
N-[(4-{[(Benzylcarbamoyl)amino]methyl}cyclohexyl)methyl]-4-methylbenzenesulfonamide;
N-{[4-({[(2-Chlorobenzyl)carbamoyl]amino}methyl)cyclohexyl]methyl}-4-methylbenzenesulfonamide;
4-Methyl-N-{[4-({[(2-phenylethyl)carbamoyl]amino}methyl)cyclohexyl]methyl}benzenesulfonamide;
4-Methyl-N-{[4-({[(4-methylbenzyl)carbamoyl]amino}methyl)cyclohexyl]methyl}benzenesulfonamide;
N-{[4-({[(2-Chlorobenzyl)carbamoyl]amino}methyl)cyclohexyl]methyl}-3,4-dimethoxybenzenesulfonamide;
3,4-Dimethoxy-N-{[4-({[(2-phenylethyl)carbamoyl]amino}methyl)cyclohexyl]methyl}benzenesulfonamide;
4-Methyl-N-{[4-({[(4-methylbenzyl)carbamoyl]amino}methyl)cyclohexyl]methyl}benzamide;
N-{[4-({[(2-Chlorobenzyl)carbamoyl]amino}methyl)cyclohexyl]methyl}-4-fluorobenzamide;
4-Chloro-N-{[4-({[(4-fluorobenzyl)carbamoyl]amino}methyl)cyclohexyl]methyl}benzamide;
2-Fluoro-N-({4-[({[2-(trifluoromethyl)benzyl]carbamoyl}amino)methyl]cyclohexyl}methyl)benzamide;
4-Nitro-N-({4-[({[2-(trifluoromethyl)benzyl]carbamoyl}amino)methyl]cyclohexyl}methyl)benzamide;
N-{[4-({[(4-Fluorobenzyl)carbamoyl]amino}methyl)cyclohexyl]methyl}-3-methylbenzamide;
N-[(4-{[(Benzylcarbamoyl)amino]methyl}cyclohexyl)methyl]-4-methylbenzamide;
2,3-Dichloro-N-({4-[({[2-(trifluoromethyl)benzyl]carbamoyl}amino)methyl]cyclohexyl}methyl)benzamide;
4-Chloro-N-{[4-({[(3-chlorobenzyl)carbamoyl]amino}methyl)cyclohexyl]methyl}benzamide;
6-Chloro-N-{[4-({[(2-chlorobenzyl)carbamoyl]amino}methyl)cyclohexyl]methyl}nicotinamide;
2,3-Dichloro-N-{[4-({[(cyclopropylmethyl)carbamoyl]amino}methyl)cyclohexyl]methyl}benzamide;
6-Chloro-N-[(4-{[(propylcarbamoyl)amino]methyl}cyclohexyl)methyl]nicotinamide;
6-Chloro-N-[(4-{[(isobutylcarbamoyl)amino]methyl}cyclohexyl)methyl]nicotinamide;
2-Fluoro-N-{[4-({[(4-fluorobenzyl)carbamoyl]amino}methyl)cyclohexyl]methyl} benzamide;
3-Cyano-N-({4-[({[2(trifluoromethyl)benzyl]carbamoyl}amino)methyl]cyclohexyl} methyl)benzamide;
4-Fluoro-N-{[4-({[(4-fluorobenzyl)carbamoyl]amino}methyl)cyclohexyl]methyl}benzamide;
N-{[4-({[(Cyclopropylmethyl)carbamoyl]amino}methyl)cyclohexyl]methyl}-2-fluorobenzamide;
N-[(4-{[(Isobutylcarbamoyl)amino]methyl}cyclohexyl)methyl]-4-methylbenzamide;
2,3-Dichloro-N-{[4-({[(4-methylbenzyl)carbamoyl]amino}methyl)cyclohexyl]methyl}benzamide;
N-({4-[({[2-(Trifluoromethyl)benzyl]carbamoyl}amino)methyl]cyclohexyl} methyl)nicotinamide;
N-{[4-({[(2-Methoxyethyl)carbamoyl]amino}methyl)cyclohexyl]methyl}-3-methylbenzamide;
3-Methyl-N-{[4-({[(2-phenylethyl)carbamoyl]amino}methyl)cyclohexyl]methyl}benzamide;
2,3-Dichloro-N-{[4-({[(2-methoxyethyl)carbamoyl]amino}methyl)cyclohexyl]methyl}benzamide;
4-Fluoro-N-[(4-{[(propylcarbamoyl)amino]methyl}cyclohexyl)methyl]benzamide;
N-{[4-({[(3-Methoxypropyl)carbamoyl]amino}methyl)cyclohexyl]methyl}-4-methylbenzamide;
6-Chloro-N-({4-[({[2-(trifluoromethyl)benzyl]carbamoyl}amino)methyl]cyclohexyl}methyl)nicotinamide;
3-Cyano-N-{[4-({[(cyclopropylmethyl)carbamoyl]amino}methyl)cyclohexyl]methyl}benzamide;
3-Methyl-N-[(4-{[(propylcarbamoyl)amino]methyl}cyclohexyl)methyl] benzamide;
4-Methyl-N-{[4-({[(2-phenylethyl)carbamoyl]amino}methyl)cyclohexyl]methyl}benzamide;
3-Cyano-N-{[4-({[(2-methoxyethyl)carbamoyl]amino}methyl)cyclohexyl]methyl}benzamide;
N-{[4-({[(Cyclopropylmethyl)carbamoyl]amino}methyl)cyclohexyl]methyl}-4-fluorobenzamide;
4-Fluoro-N-{[4-({[(2-methoxyethyl)carbamoyl]amino}methyl)cyclohexyl]methyl}benzamide;
4-Methoxy-N-[(4-{[(propylcarbamoyl)amino]methyl}cyclohexyl)methyl] benzamide;
N-[(4-{[(Isobutylcarbamoyl)amino]methyl}cyclohexyl)methyl]-4-methoxybenzamide;
2,3-Dichloro-N-[(4-{[(isobutylcarbamoyl)amino]methyl}cyclohexyl)methyl] benzamide;
2-Fluoro-N-[(4-{[(isobutylcarbamoyl)amino]methyl}cyclohexyl)methyl] benzamide;
6-Chloro-N-{[4-({[(2-phenylethyl)carbamoyl]amino}methyl)cyclohexyl]methyl}nicotinamide;
N-[(4-{[(Benzylcarbamoyl)amino]methyl}cyclohexyl)methyl]-4-chlorobenzamide;
2,3-Dichloro-N-{[4-({[(2-chlorobenzyl)carbamoyl]amino}methyl)cyclohexyl]methyl}benzamide;
4-Fluoro-N-({4-[({[2-(trifluoromethyl)benzyl]carbamoyl}amino)methyl]cyclohexyl}methyl)benzamide;
6-Chloro-N-{[4-({[(cyclopropylmethyl)carbamoyl]amino}methyl)cyclohexyl]methyl}nicotinamide;
N-{[4-({[(Cyclopropylmethyl)carbamoyl]amino}methyl)cyclohexyl]methyl}-4-methoxybenzamide;
3-Cyano-N-[(4-{[(propylcarbamoyl)amino]methyl}cyclohexyl)methyl]benzamide;
N-{[4-({[(2-Chlorobenzyl)carbamoyl]amino}methyl)cyclohexyl]methyl}-3-methylbenzamide;
2,3-Dichloro-N-{[4-({[(3-chlorobenzyl)carbamoyl]amino}methyl)cyclohexyl]methyl}benzamide;
tert-Butyl ({4-[({[3′-({[2-(4-hydroxyphenyl)ethyl]amino}methyl)biphenyl-3-yl]carbonyl}amino)methyl]cyclohexyl}methyl)carbamate;
tert-Butyl [(trans-4-{[(1-naphthylsulfonyl)amino]methyl}cyclohexyl)methyl] carbamate;
3-Chloro-N-{[4-({[(2-chlorobenzyl)carbamoyl]amino}methyl)cyclohexyl]methyl}-4-fluorobenzenesulfonamide;
2,3-Dichloro-N-{[4-({[(2-chlorobenzyl)carbamoyl]amino}methyl)cyclohexyl]methyl}benzamide;
2,3-Dichloro-N-[(4-{[(propylcarbamoyl)amino]methyl}cyclohexyl)methyl] benzamide;
4-Chloro-N-{[4-({[(3-methoxypropyl)carbamoyl]amino}methyl)cyclohexyl]methyl}benzamide;
3-Chloro-4-fluoro-N-{[4-({[(4-methylbenzyl)carbamoyl]amino}methyl)cyclohexyl]methyl}benzenesulfonamide;
N-[(trans-4-{[(Benzylcarbamoyl)(isopropyl)amino]methyl}cyclohexyl)methyl]benzenesulfonamide;
tert-Butyl [(4-{[(1-Naphthylsulfonyl)amino]methyl}cyclohexyl)methyl]carbamate;
tert-Butyl [(trans-4-{[(2-naphthylsulfonyl)amino]methyl}cyclohexyl)methyl]carbamate;
tert-Butyl [(trans-4-{[(1-naphthylsulfonyl)amino]methyl}cyclohexyl)methyl]carbamate;
N-{[4-({[(Cyclopropylmethyl)carbamoyl]amino}methyl)cyclohexyl]methyl}-3-fluorobenzamide;
2-Fluoro-N-{[4-({[(3-methoxypropyl)carbamoyl]amino}methyl)cyclohexyl]methyl}benzamide;
2-Fluoro-N-[[4-[[[[(2-methoxyethyl)amino]carbonyl]amino]methyl]cyclohexyl]methyl] benzamide;
3,4-Dimethoxy-N-{[4-({[(4-methylbenzyl)carbamoyl]amino}methyl)cyclohexyl]methyl}benzenesulfonamide;
3-Methyl-N-({4-[({[2-(trifluoromethyl)benzyl]carbamoyl}amino)methyl]cyclohexyl}methyl)benzamide;
6-Chloro-N-{[4-({[(4-fluorobenzyl)carbamoyl]amino}methyl)cyclohexyl]methyl}nicotinamide;
6-Chloro-N-{[4-({[(2-methoxyethyl)carbamoyl]amino}methyl)cyclohexyl]methyl}nicotinamide;
2,3-Dichloro-N-{[4-({[(2-phenylethyl)carbamoyl]amino}methyl)cyclohexyl]methyl}benzamide;
4-Methyl-N-[(4-{[(methylcarbamoyl)amino]methyl}cyclohexyl)methyl]benzamide;
3-Chloro-4-fluoro-N-{[4-({[(2-phenylethyl)carbamoyl]amino}methyl)cyclohexyl]methyl}benzenesulfonamide;
N-[(4-{[(Benzylcarbamoyl)amino]methyl}cyclohexyl)methyl]-4-methylbenzenesulfonamide;
N-{[4-({[(2-Chlorobenzyl)carbamoyl]amino}methyl)cyclohexyl]methyl}-4-methylbenzenesulfonamide;
4-Methyl-N-{[4-({[(2-phenylethyl)carbamoyl]amino}methyl)cyclohexyl]methyl}benzenesulfonamide;
4-Methyl-N-{[4-({[(4-methylbenzyl)carbamoyl]amino}methyl)cyclohexyl]methyl}benzenesulfonamide;
N-{[4-({[(2-Chlorobenzyl)carbamoyl]amino}methyl)cyclohexyl]methyl}-3,4-dimethoxybenzenesulfonamide; and
3,4-Dimethoxy-N-{[4-({[(2-phenylethyl)carbamoyl]amino}methyl)cyclo-hexyl]methyl}benzenesulfonamide.

2. A compound according to claim 1 wherein L represents C(O).

3. A compound according to claim 1 wherein n is 1.

4. A compound according to claim 1 wherein Z represents 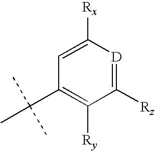
wherein:
D represents N or C, optionally substituted by hydrogen, halo, C₁-C₄ alkyl, aryl, NR⁴⁷C(O)R⁴⁸;
Rₓ and R_{z} independently represent hydrogen, halo, —OH, —OR⁴⁹, —SR⁵⁰, —N(R^{51a})(R^{51b}), aryl, C₃-C₈ cycloalkyl, C₃-C₈ cycloalkenyl, heterocyclyl, heteroaryl, (which aryl, cycloalkyl, cycloalkenyl, heterocyclyl and heteroaryl groups are optionally substituted by one or more groups of the following halo, OH, oxo, —C(O)N(R^{52a})(R^{52b}), —OR⁵³, —SR⁵⁴, —C(O)OR⁵⁵, cyano, —N(R⁵⁶)C(O)OR⁵⁷, —N(R⁵⁶)C(O)N(R^{52a})(R^{52b}), —N(R⁵⁶)S(O)₂R⁶⁰—S(O)₂ N(R^{52a})(R^{52b}), —N(R⁵⁶)(CHR⁵⁸)ₘN(R^{52a})(R^{52b}), —N(R^{52a})(R^{52b}), —C(O)R⁵⁹, —N(R⁵⁶)C(O)R⁵⁹, —N(R⁵⁶)(CHR⁵⁸)ₘOR⁵⁵, —N(R⁵⁶)(CHR⁵⁸)ₘC(O)N(R^{52a})(R^{52b}), —N(R⁵⁶)(CHR⁵⁸)ₘC(O)O(R⁵⁷), —S(O)₍₁₋₂₎R⁶⁰, heteroaryl, heterocyclyl (which heteroaryl, heterocyclyl groups are optionally substituted by one or more of the following —OH, oxo, —C(O)R⁶¹, —C(O)N(R^{62a})(R^{62b}), C₁-C₄ alkyl (which alkyl is optionally substituted by OH)), C₁-C₆ alkyl, C₁-C₆ alkoxy (which alkyl and alkoxy groups are optionally substituted by one or more of the following —OH, halo, —N(R^{63a})(R^{63b}), —C(O)OR⁶⁴, N(R⁶⁵)C(O)OR⁶⁴, —N(R⁶⁵)C(O)R⁶⁶, —N(R⁶⁵)C(O)N(R^{63a})(R^{63b}), —N(R⁶⁵)S(O)₍₁₋₂₎(R⁶⁶), —C(O)N(R^{63a})(R^{63b}), heterocyclyl, heteroaryl (which heteroaryl, heterocyclyl groups are optionally substituted by one or more C₁-C₄ alkyl, oxo, —C(O)R⁶⁷));
R_{y} represents hydrogen, halo, C₁-C₆ alkyl, C₁-C₆ alkoxy;
R_{y} and R_{z} may together with the carbon atoms to which they are attached represent a benzene ring;
wherein:
R^{51a}, R^{52a}, R^{52b}, R^{62a}, R^{62b}, R^{63a}, R^{63b} independently represent hydrogen, C₁-C₆ alkyl (optionally substituted by one or more OH, halo, heteroaryl (which heteroaryl optionally is substituted by one or more C₁-C₄ alkyl or C₁-C₄ alkoxy));
R⁴⁶, R⁴⁸, R⁵⁰, R⁵⁴, R⁵⁹, R⁶¹, R⁶⁶ independently represent C₁-C₆ alkyl, optionally substituted by one or more OH, halo, heteroaryl, heterocyclyl, which groups optionally are substituted by one or more C₁-C₆ alkyl, OH;
R⁴⁵, R⁴⁷, R⁵⁵, R⁵⁶, R⁵⁸, R⁶⁰, R⁶⁴, R⁶⁵ independently represent hydrogen, C₁-C₆ alkyl, optionally substituted by one or more OH, halo, heteroaryl, heterocyclyl (which heteroaryl, heterocyclyl groups optionally are substituted by one or more C₁-C₆ alkyl, OH;
R⁵⁰, R⁵⁷, R⁶⁷ independently represent hydrogen, C₁-C₆ alkyl (optionally substituted by one or more OH, halo);
R⁴⁹, R⁵³ independently represent C₃-C₈ cycloalkyl, C₃-C₈ cycloalkenyl, heteroaryl, heterocyclic, C₁-C₆ alkyl (which alkyl is optionally substituted by one or more OH, halo, N(R^{63a})(R^{63b}), heteroaryl, heterocyclyl (which heteroaryl and heterocyclyl groups optionally are substituted by one or more OH, oxo, C₁-C₆ alkyl, C₁-C₆ alkoxy, —C(O)R⁶⁶)); and
m is an integer of 0, 1, 2, 3, 4, 5, 6.

5. A compound according to claim 1 wherein R_{z} represents hydrogen, halo, C₁-C₆ alkyl, C₁-C₆ alkoxy, aryl or heteroaryl (which aryl and heteroaryl groups are optionally substituted by one or more of —OH, halo, cyano, amino, C₁-C₆ alkyl, C₁-C₆ alkoxy, oxo, —NR⁵⁶C(O)R⁵⁹).

6. A compound according to claim 1 wherein R₁ represents —OR⁶.

7. A compound according to claim 1 wherein Z represents 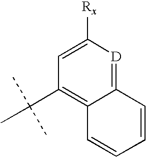
wherein:
D represents N or C, optionally substituted by hydrogen, halo, C₁-C₄ alkyl, aryl;
R^{x} represents hydrogen, halo, —OR⁶⁸, —SR⁶⁹, —N(R⁷⁰)C(O)R⁷¹, —N(R^{72a})(R^{72b}) aryl, C₃-C₈ cycloalkyl, C₃-C₈ cycloalkenyl, heterocyclyl, heteroaryl, (which aryl, cycloalkyl, cycloalkenyl, heterocyclyl, heteroaryl groups are optionally substituted by one or more groups selected from halo, OH, oxo, —C(O)N(R^{73a})(R^{73b}), OR⁷⁴, —SR⁷⁵, —C(O)OR⁷⁶, cyano, —N(R⁷⁷)C(O)OR⁷⁸, —N(R⁷⁷)C(O)N(R^{73a})(R^{73b}), —N(R⁷⁷)S(O)₍₁₋₂₎R⁷⁸, —N(R⁷⁷)(CHR⁷⁹)ₘN(R^{73a})(R^{73b}), —N(R^{73a})(R^{73b}), —C(O)R⁸⁰, —N(R⁷⁷)C(O)R⁸⁰, —N(R⁷⁷)(CHR⁷⁹)ₘOR⁷⁹, —N(R⁷⁷)(CHR⁷⁹)ₘOH, —N(R⁷⁷)(CHR⁷⁹)ₘC(O)N(R^{73a})(R^{73b}), —N(R⁷⁷)(CHR⁷⁹)ₘC(O)O(R⁷⁶), —N(R⁷⁷)C(O)R⁸⁰, —S(O)₍₁₋₂₎R⁷⁸, —S(O)₍₁₋₂₎N(R^{73a})(R^{73b}), heteroaryl, heterocyclyl (which heteroaryl, heterocyclyl groups are optionally substituted by one or more of the following OH, oxo, C(O)R⁸¹, C(O)N(R^{82a})(R^{82b}), —N(R^{82a})(R^{82b}), C₁-C₄ alkyl, C₁-C₄ alkoxy (which alkyl or alkoxy is optionally substituted by one or more OH, —N(R^{82a})(R^{82b}))), C₁-C₆ alkyl, C₁-C₆ alkoxy (which alkyl and alkoxy groups are optionally substituted by one or more of the following —OH, halo, —C(O)OR⁸³, —N(R^{84a})(R^{84b}), —N(R⁸⁵)C(O)OR⁸³, —N(R⁸⁵)C(O)R⁸⁶, —N(R⁸⁵)C(O)N(R^{84a})(R^{84b}), —N(R⁸⁵)S(O)₍₁₋₂₎R⁸⁷, —C(O)N(R^{84a})(R^{84b}), heteroaryl, heterocyclyl (which heteroaryl, heterocyclyl groups are optionally substituted by one or more C₁-C₄ alkyl, oxo, —C(O)R⁸⁸));
R^{72a}, R^{72b}, R^{73a}, R^{73b}, R^{82a}, R^{82b}, R^{84a}, R^{84b} independently represent hydrogen, C₁-C₆ alkyl (optionally substituted by one or more OH, halo, heteroaryl (which heteroaryl optionally is substituted by one or more C₁-C₄ alkyl));
R⁶⁸, R⁶⁹, R⁷⁴, R⁷⁵, R⁷⁸, R⁸⁷ independently represent C₃-C₈ cycloalkyl, C₃-C₈ cycloalkenyl, heteroaryl, heterocyclyl, C₁-C₆ alkyl (which groups are optionally substituted by one or more OH, halo, —N(R^{84a})(R^{84b}), heteroaryl, heterocyclyl (which heteroaryl, heterocyclyl groups optionally are substituted by one or more OH, oxo, C₁-C₆ alkyl, C₁-C₆ alkoxy, C(O)R⁸⁹));
R⁷¹, R⁷⁶, R⁷⁸, R⁷⁹, R⁸⁰, R⁸¹, R⁸³, R⁸⁶, R⁸⁷, R⁸⁸, R⁸⁹ independently represent hydrogen, C₁-C₆ alkyl, optionally substituted by one or more OH, halo, heteroaryl, heterocyclyl (which groups optionally are substituted by one or more OH, C₁-C₆ alkyl, C(O)R⁸⁹);
R⁷⁰, R⁷⁷, R⁸⁵ independently represent hydrogen, C₁-C₆ alkyl (optionally substituted by one or more OH, halo); and
m is an integer of 0, 1, 2, 3, 4, 5 or 6.

8. A compound according to claim 4 or 7 wherein D represents N.

9. A compound according to claim 1 as represented by formula II 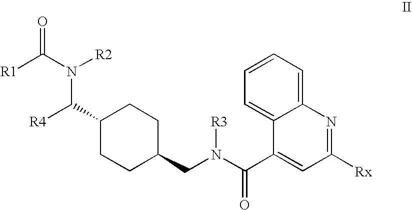
or a pharmaceutically acceptable salt thereof in which R₁, R₂, R₃ and R₄ are as defined in claim 1 and Rx represents a group a), b) or c):
a) 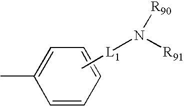
in which L₁ represents a bond or a C₁-C₄ alkylene chain and R⁹⁰ and R⁹¹ independently represent H or C₁-C₆ alkyl optionally substituted by hydroxy or C₁-C₄ alkoxy;
b) 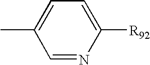
in which R₉₂ represents a group -L₂-L₃-NR⁹³R⁹⁴ in which L₂ is O or N, L₃ is a C₂-C₄ alkylene chain and R⁹³ and R⁹⁴ independently represent H or C₁-C₆ alkyl optionally substituted by hydroxy or C₁-C₄ alkoxy or R₉₂ represents azetidino, pyrrolidino, piperidino, morpholino or piperazino each of which is optionally substituted by one or more of the following: hydroxy, a C₁-C₄ alkyl, C₁-C₄ alkoxy or a C₁-C₄ alkanoyl;
c) 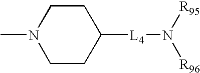
in which L₄ is a C₁-C₄ alkylene chain optionally substituted by hydroxy or fluoro provided that no carbon atom in the chain is attached to two hetero atoms (that is O or N) and R⁹⁵ and R⁹⁶ independently represent H or C₁-C₆ alkyl optionally substituted by hydroxy or C₁-C₄ alkoxy or R⁹⁵ and R⁹⁶ together with the nitrogen atom to which they are attached represent azetidino, pyrrolidino, piperidino, morpholino or piperazino each of which is optionally substituted by one or more of the following: hydroxy, a C₁-C₄ alkyl or C₁-C₄ alkoxy.

10. A compound according to claim 9, or a pharmaceutically acceptable salt thereof, wherein R^{x} represents group a) in which L₁ represents a C₁ alkylene chain, and both R⁹⁰ and R⁹¹ represent H, or wherein R^{x} represents group b), in which R₉₂ represents a group -L₂-L₃-NR⁹³R⁹⁴ in which L₂ is O or N, L₃ is a C₂-C₃ alkylene chain, and R⁹³ and R⁹⁴ both represent H, or wherein or R₉₂ represents piperidino, morpholino or piperazino each of which is optionally substituted by one or more of the following: hydroxy, a C₁ alkyl, C₁ alkoxy or a C₁ alkanoyl, or wherein R^{x} represents group c) in which L₄ is a C₂-C₃ alkylene chain, which chain is optionally substituted by hydroxy provided that no carbon atom in the chain is attached to two hetero atom (that is O or N) and R⁹⁵ and R⁹⁶ independently C₁-C₂ alkyl optionally substituted by hydroxy, or R⁹⁵ and R⁹⁶ together with the nitrogen atom to which they are attached represent azetidino which is optionally substituted by a hydroxy.

11. A compound according to claim 1, or a pharmaceutically acceptable salt thereof, wherein:
R₁ represents —OR⁶;
R₂ and R₃ independently represent hydrogen or C₁-C₆ alkyl;
R₄ represents hydrogen or C₁-C₆ alkyl;
R₅ represents hydrogen or C₁-C₆ alkyl; and
R⁶ represents C₃-C₅ alkyl.

12. A compound selected from one or more of the following compounds:
tert-Butyl [(trans-4-{[({2-[6-(4-methylpiperazin-1-yl)pyridin-3-yl]quinolin-4-yl}carbonyl)amino]methyl}cyclohexyl)methyl]carbamate;
tert-Butyl ({trans-4-[({[2-(2-methyl-6-oxo-1,6-dihydropyridin-3-yl)quinolin-4-yl]carbonyl}amino)methyl]cyclohexyl}methyl)carbamate;
tert-Butyl {[trans-4-({[(2-pyrazin-2-ylquinolin-4-yl)carbonyl]amino}methyl)cyclo-hexyl]methyl}carbamate;
tert-Butyl ({trans-4-[({[2-(4-ethoxyphenyl)quinolin-4-yl]carbonyl}amino)methyl]-cyclohexyl}methyl)carbamate;
tert-Butyl ({trans-4-[({[2-(6-carbamoylpyridin-3-yl)quinolin-4-yl]carbonyl}amino)methyl]cyclohexyl}methyl)carbamate;
tert-Butyl {[trans-4-({[(2-phenylquinolin-4-yl)carbonyl]amino}methyl)cyclohexyl]-methyl}carbamate;
tert-Butyl {[trans-4-({[(2-pyridin-4-ylquinolin-4-yl)carbonyl]amino}methyl)cyclohexyl]methyl}carbamate;
tert-Butyl ({trans-4-[({[2-(3-carbamoylphenyl)quinolin-4-yl]carbonyl}amino)methyl]-cyclohexyl}methyl)carbamate;
tert-Butyl ({trans-4-[({[2-(4-carbamoylphenyl)quinolin-4-yl]carbonyl}amino)methyl]-cyclohexyl}methyl)carbamate;
tert-Butyl {[trans-4-({[(2-{6-[3-(dimethylamino)propoxy]pyridin-3-yl}quinolin-4-yl)carbonyl]amino}methyl)cyclohexyl]methyl}carbamate;
tert-Butyl [(trans-4-{[({2-[6-(dimethylamino)pyridin-3-yl]quinolin-4-yl}carbonyl)amino]-methyl}cyclohexyl)methyl]carbamate;
tert-Butyl [(trans-4-{[({2-[4-(trifluoromethyl)phenyl]quinolin-4-yl}carbonyl)amino]-methyl}cyclohexyl)methyl]carbamate;
tert-Butyl ({trans-4-[({[2-(5-acetyl-2-thienyl)quinolin-4-yl]carbonyl}amino)methyl]-cyclohexyl}methyl)carbamate;
tert-Butyl ({trans-4-[({[2-(4-fluorophenyl)quinolin-4-yl]carbonyl}amino)methyl]-cyclohexyl}methyl)carbamate;
tert-Butyl ({trans-4-[({[2-(3,5-dimethylisoxazol-4-yl)quinolin-4-yl]carbonyl}amino)-methyl]cyclohexyl}methyl)carbamate;
tert-Butyl {[trans-4-({[(2-pyridin-3-ylquinolin-4-yl)carbonyl]amino}methyl)cyclo-hexyl]methyl}carbamate;
Ethyl ({trans-4-[({[2-(4-fluorophenyl)quinolin-4-yl]carbonyl}amino)methyl]cyclo-hexyl}methyl)carbamate;
Ethyl [(trans-4-{[({2-[4-(dimethylcarbamoyl)phenyl]quinolin-4-yl}carbonyl)amino]-methyl}cyclohexyl)methyl]carbamate;
Ethyl ({trans-4-[({[2-(3-carbamoylphenyl)quinolin-4-yl]carbonyl}amino)methyl]-cyclohexyl}methyl)carbamate;
tert-Butyl ({trans-4-[({[2-(4-methylphenyl)quinolin-4-yl]carbonyl}amino)methyl]-cyclohexyl}methyl)carbamate;
tert-Butyl ({trans-4-[({[2-(4-chlorophenyl)quinolin-4-yl]carbonyl}amino)methyl]-cyclohexyl}methyl)carbamate;
tert-Butyl ({trans-4-[({[2-(3-methoxyphenyl)quinolin-4-yl]carbonyl}amino)methyl]cyclohexyl}methyl)carbamate;
tert-Butyl ({trans-4-[({[2-(2-methoxyphenyl)quinolin-4-yl]carbonyl}amino)methyl]-cyclohexyl}methyl)carbamate;
tert-Butyl [(trans-4-{[({2-[4-(methylthio)phenyl]quinolin-4-yl}carbonyl)amino]-methyl}cyclohexyl)methyl]carbamate;
tert-Butyl ({trans-4-[({[2-(2,4-dimethyl-1,3-thiazol-5-yl)quinolin-4-yl]carbonyl}amino)-methyl]cyclohexyl}methyl)carbamate;
Ethyl {[trans-4-({[(2-pyrazin-2-ylquinolin-4-yl)carbonyl]amino}methyl)cyclohexyl]-methyl}carbamate;
Ethyl ({trans-4-[({[2-(3-methoxyphenyl)quinolin-4-yl]carbonyl}amino)methyl]-cyclohexyl}methyl)carbamate;
Ethyl ({trans-4-[({[2-(4-methylphenyl)quinolin-4-yl]carbonyl}amino)methyl]cyclo-hexyl}methyl)carbamate;
Ethyl [(trans-4-{[({2-[4-(methylthio)phenyl]quinolin-4-yl}carbonyl)amino]methyl}-cyclohexyl)methyl]carbamate;
Ethyl ({trans-4-[({[2-(4-ethoxyphenyl)quinolin-4-yl]carbonyl}amino)methyl]-cyclohexyl}methyl)carbamate;
Ethyl ({trans-4-[({[2-(2-methoxyphenyl)quinolin-4-yl]carbonyl}amino)methyl]-cyclohexyl}methyl)carbamate;
Ethyl ({trans-4-[({[2-(4-chlorophenyl)quinolin-4-yl]carbonyl}amino)methyl]cyclo-hexyl}methyl)carbamate;
tert-Butyl {[trans-4-({[(2-pyrimidin-5-ylquinolin-4-yl)carbonyl]amino}methyl)cyclohexyl]methyl}carbamate;
tert-Butyl ({trans-4-[({[2-(4-cyanophenyl)quinolin-4-yl]carbonyl}amino)methyl]-cyclohexyl}methyl)carbamate;
tert-Butyl {[trans-4-({[(2-chloroquinolin-4-yl)carbonyl]amino}methyl)cyclohexyl]-methyl}carbamate;
tert-Butyl ({trans-4-[({2-[4-(aminomethyl)phenyl]-6-methoxyisonicotinoyl}-amino)methyl]cyclohexyl}methyl)carbamate;
tert-Butyl ({trans-4-[({2-[4-(aminomethyl)phenyl]-5-chloroisonicotinoyl}amino)methyl]cyclohexyl}methyl)carbamate;
tert-Butyl ({trans-4-[({2-[4-(aminomethyl)phenyl]-6-methylisonicotinoyl}amino)methyl]cyclohexyl}methyl)carbamate;
tert-Butyl [(trans-4-{[({2-[3-(aminomethyl)phenyl]quinolin-4-yl}carbonyl)amino]-methyl}cyclohexyl)methyl]carbamate;
[3-(4-{[(trans-4-{[(tert-Butoxycarbonyl)amino]methyl}cyclohexyl)methyl]-carbamoyl}quinolin-2-yl)phenyl]acetic acid;
tert-Butyl [(trans-4-{[(2-phenylisonicotinoyl)amino]methyl}cyclohexyl)methyl]-carbamate;
tert-Butyl {[trans-4-({[2-(4-methoxyphenyl)isonicotinoyl]amino}methyl)cyclo-hexyl]methyl}carbamate;
tert-Butyl [(trans-4-{[(2-methyl-6-phenylisonicotinoyl)amino]methyl}cyclohexyl)methyl]carbamate;
tert-Butyl {[trans-4-({[(5-phenylpyridin-3-yl)carbonyl]amino}methyl)cyclohexyl]-methyl}carbamate;
tert-Butyl {[trans-4-({[2-(1-benzothiophen-2-yl)isonicotinoyl]amino}methyl)cyclo-hexyl]methyl}carbamate;
tert-Butyl {[trans-4-({[(6′-ethoxy-2,3′-bipyridin-4-yl)carbonyl]amino}methyl)cyclo-hexyl]methyl}carbamate;
tert-Butyl {[trans-4-({[2-(2,4-dimethoxyphenyl)isonicotinoyl]amino}methyl)cyclo-hexyl]methyl}carbamate;
tert-Butyl {[trans-4-({[(2′,6′-dimethoxy-2,3′-bipyridin-4-yl)carbonyl]amino}methyl)-cyclohexyl]methyl}carbamate;
tert-Butyl {[trans-4-({[(6′-methoxy-2,3′-bipyridin-4-yl)carbonyl]amino}methyl)cyclo-hexyl]methyl}carbamate;
tert-Butyl {[trans-4-({[2-(4-carbamoylphenyl)isonicotinoyl]amino}methyl)cyclohexyl]methyl}carbamate;
tert-Butyl {[trans-4-({[2-(3-chlorophenyl)isonicotinoyl]amino}methyl)cyclohexyl]-methyl}carbamate;
tert-Butyl ({trans-4-[({2-[4-(aminomethyl)phenyl]isonicotinoyl}amino)methyl]cyclo-hexyl}methyl)carbamate;
tert-Butyl {[trans-4-({[2-(3,4-difluorophenyl)isonicotinoyl]amino}methyl)cyclohexyl]-methyl}carbamate;
Ethyl [(trans-4-{[(2-naphthylsulfonyl)amino]methyl}cyclohexyl)methyl]carbamate;
2,2-Dimethylpropyl {[trans-4-({[(2-pyridin-4-ylquinolin-4-yl)carbonyl]amino}methyl)-cyclohexyl]methyl}carbamate;
Cyclopentyl {[trans-4-({[(2-pyridin-4-ylquinolin-4-yl)carbonyl]amino}methyl)cyclo-hexyl]methyl}carbamate;
Isopropyl {[trans-4-({[(2-pyridin-4-ylquinolin-4-yl)carbonyl]amino}methyl)cyclo-hexyl]methyl}carbamate;
Propyl {[trans-4-({[(2-pyridin-4-ylquinolin-4-yl)carbonyl]amino}methyl)cyclohexyl]-methyl}carbamate;
2-Methoxyethyl {[trans-4-({[(2-pyridin-4-ylquinolin-4-yl)carbonyl]amino}-methyl)cyclohexyl]methyl}carbamate;
Ethyl {[trans-4-({[(2-pyridin-4-ylquinolin-4-yl)carbonyl]amino}methyl)cyclo-hexyl]methyl}carbamate;
Ethyl ({trans-4-[({[2-(4-carbamoylphenyl)quinolin-4-yl]carbonyl}amino)methyl]-cyclohexyl}methyl)carbamate;
Ethyl {[trans-4-({[(2-phenylquinolin-4-yl)carbonyl]amino}methyl)cyclohexyl]methyl}-carbamate;
tert-Butyl [(trans-4-{[(biphenyl-3-ylsulfonyl)amino]methyl}cyclohexyl)methyl]-carbamate;
tert-Butyl {[trans-4-({[(3,4-dibromophenyl)sulfonyl]amino}methyl)cyclohexyl]-methyl}carbamate;
tert-Butyl {[trans-4-({[(3,4-dichlorophenyl)sulfonyl]amino}methyl)cyclohexyl]methyl}-carbamate;
tert-Butyl [(trans-4-{[(quinolin-8-ylsulfonyl)amino]methyl}cyclohexyl)methyl]-carbamate;
tert-Butyl [(trans-4-{[(biphenyl-4-ylsulfonyl)amino]methyl}cyclohexyl)methyl]-carbamate;
tert-Butyl {[trans-4-({[(4-isopropylphenyl)sulfonyl]amino}methyl)cyclohexyl]-methyl}carbamate;
tert-Butyl [(trans-4-{[(2,3-dihydro-1,4-benzodioxin-6-ylsulfonyl)amino]methyl}cyclo-hexyl)methyl]carbamate;
tert-Butyl ({trans-4-[({[5-(2-methyl-1,3-thiazol-4-yl)-2-thienyl]sulfonyl}amino)methyl]cyclohexyl}methyl)carbamate;
tert-Butyl {[trans-4-({[(5-acetamido-1-naphthyl)sulfonyl]amino}methyl)cyclohexyl]-methyl}carbamate;
tert-Butyl {[trans-4-({[(5-isoxazol-5-yl-2-thienyl)sulfonyl]amino}methyl)cyclo-hexyl]methyl}carbamate;
tert-Butyl {[trans-4-({[(4-acetamidophenyl)sulfonyl]amino}methyl)cyclohexyl]-methyl}carbamate;
tert-Butyl [(trans-4-{[(3-thienylsulfonyl)amino]methyl}cyclohexyl)methyl]-carbamate;
tert-Butyl ({trans-4-[({[3-(5-methyl-1,2,4-oxadiazol-3-yl)phenyl]sulfonyl}amino)methyl]cyclohexyl}methyl)carbamate;
tert-Butyl [(trans-4-{[(isoquinolin-5-ylsulfonyl)amino]methyl}cyclohexyl)methyl]carbamate;
tert-Butyl {[trans-4-({[(4-acetamido-2-methyl-1,3-thiazol-5-yl)sulfonyl]amino}-methyl)cyclohexyl]methyl}carbamate;
tert-Butyl [(trans-4-{[(1,3-benzothiazol-6-ylsulfonyl)amino]methyl}cyclohexyl)methyl]carbamate;
tert-Butyl [(trans-4-{[ethyl(2-naphthylsulfonyl)amino]methyl}cyclohexyl)methyl]-carbamate;
tert-Butyl [(trans-4-{[(2-naphthylsulfonyl)(2-phenylethyl)amino]methyl}cyclohexyl)-methyl]carbamate;
Ethyl N-[(trans-4-{[(tert-butoxycarbonyl)amino]methyl}cyclohexyl)methyl]-N-(2-naphthylsulfonyl)glycinate;
tert-Butyl [(trans-4-{[(2-naphthylsulfonyl)(tetrahydro-2H-pyran-4-ylmethyl)amino]-methyl}cyclohexyl)methyl]carbamate;
tert-Butyl [(trans-4-{[(cyanomethyl)(2-naphthylsulfonyl)amino]methyl}cyclohexyl)-methyl]carbamate;
tert-Butyl [(trans-4-{[allyl(2-naphthylsulfonyl)amino]methyl}cyclohexyl)methyl]-carbamate;
tert-Butyl [(trans-4-{[butyl(2-naphthylsulfonyl)amino]methyl}cyclohexyl)methyl]-carbamate;
tert-Butyl [(trans-4-{[(2-naphthylsulfonyl)(2,2,2-trifluoroethyl)amino]methyl}cyclo-hexyl)methyl]carbamate;
tert-Butyl [(trans-4-{[(2-naphthylsulfonyl)(propyl)amino]methyl}cyclohexyl)methyl]carbamate;
tert-Butyl [(trans-4-{[methyl(2-naphthylsulfonyl)amino]methyl}cyclohexyl)methyl]carbamate;
tert-Butyl ({trans-4-[(methyl {[5-(2-methyl-1,3-thiazol-4-yl)-2-thienyl]sulfonyl}-amino)methyl]cyclohexyl}methyl)carbamate;
tert-Butyl ({trans-4-[(ethyl {[5-(2-methyl-1,3-thiazol-4-yl)-2-thienyl]sulfonyl} amino)-methyl]cyclohexyl}methyl)carbamate;
tert-Butyl {[trans-4-({methyl[(2-phenylquinolin-4-yl)carbonyl]amino}methyl)cyclo-hexyl]methyl}carbamate;
tert-Butyl [(trans-4-{[({2-[4-(aminomethyl)phenyl]quinolin-4-yl}carbonyl)amino]-methyl}cyclohexyl)methyl]carbamate acetate;
4-(4-{[(trans-4-{[(tert-Butoxycarbonyl)amino]methyl}cyclohexyl)methyl]carbamoyl}-quinolin-2-yl)benzoic acid;
tert-Butyl ({trans-4-[({[2-(1-oxidopyridin-4-yl)quinolin-4-yl]carbonyl}amino) methyl]-cyclohexyl}methyl)carbamate;
tert-Butyl {[trans-4-({[(2-phenylquinolin-4-yl)sulfonyl]amino}methyl)cyclohexyl]-methyl}carbamate;
tert-Butyl ({trans-4-[(1-naphthoylamino)methyl]cyclohexyl}methyl)carbamate;
N-[(trans-4-{[(tert-Butylcarbamoyl)amino]methyl}cyclohexyl)methyl]-2-pyridin-4-ylquinoline-4-carboxamide;
tert-Butyl ({trans-4-[({[2-(3-{[(methylsulfonyl)amino]methyl}phenyl)quinolin-4-yl]carbonyl}amino)methyl]cyclohexyl}methyl)carbamate;
tert-Butyl {[trans-4-({[(2-{3-[(carbamoylamino)methyl]phenyl}quinolin-4-yl)carbonyl]amino}methyl)cyclohexyl]methyl}carbamate;
tert-Butyl [(trans-4-{[({2-[3-(acetamidomethyl)phenyl]quinolin-4-yl}carbonyl)amino]-methyl}cyclohexyl)methyl]carbamate;
Methyl [3-(4-{[(trans-4-{[(tert-butoxycarbonyl)amino]methyl}cyclohexyl)methyl]-carbamoyl}quinolin-2-yl)benzyl]carbamate;
tert-Butyl [(trans-4-{[({2-[4-(2-aminoethyl)phenyl]quinolin-4-yl}carbonyl)amino]-methyl}cyclohexyl)methyl]carbamate;
tert-Butyl [(trans-4-{[({2-[4-(acetamidomethyl)phenyl]quinolin-4-yl}carbonyl)amino]methyl}cyclohexyl)methyl]carbamate;
Methyl [4-(4-{[(trans-4-{[(tert-butoxycarbonyl)amino]methyl}cyclohexyl)methyl]-carbamoyl}quinolin-2-yl)benzyl]carbamate;
tert-Butyl {[trans-4-({[(2-{4-[(carbamoylamino)methyl]phenyl}quinolin-4-yl)carbonyl]amino}methyl)cyclohexyl]methyl}carbamate;
tert-Butyl ({trans-4-[({[2-(4-{[(methylsulfonyl)amino]methyl}phenyl)quinolin-4-yl]carbonyl}amino)methyl]cyclohexyl}methyl)carbamate;
[4-(4-{[(trans-4-{[(tert-Butoxycarbonyl)amino]methyl}cyclohexyl)methyl]-carbamoyl}quinolin-2-yl)phenyl]acetic acid;
3-(4-{[(trans-4-{[(tert-Butoxycarbonyl)amino]methyl}cyclohexyl)methyl]-carbamoyl}quinolin-2-yl)benzoic acid;
Tetrahydro-2H-pyran-4-yl [(trans-4-{[({2-[6-(4-methylpiperazin-1-yl)pyridin-3-yl]quinolin-4-yl}carbonyl)amino]methyl}cyclohexyl)methyl]carbamate;
1-Methylpiperidin-4-yl [(trans-4-{[({2-[6-(4-methylpiperazin-1-yl)pyridin-3-yl]quinolin-4-yl}carbonyl)amino]methyl}cyclohexyl)methyl]carbamate;
Oxetan-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
(1S)-2-Methoxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
(1R)-2-Methoxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
Tetrahydrofuran-3-yl [(trans-4-{[({2-[6-(4-methylpiperazin-1-yl)pyridin-3-yl]quinolin-4-yl}carbonyl)amino]methyl}cyclohexyl)methyl]carbamate;
2-(2-Oxopyrrolidin-1-yl)ethyl [(trans-4-{[({2-[6-(4-methylpiperazin-1-yl)pyridin-3-yl]quinolin-4-yl}carbonyl)amino]methyl}cyclohexyl)methyl]carbamate;
(3S)-Tetrahydrofuran-3-yl [(trans-4-{[({2-[6-(4-methylpiperazin-1-yl)pyridin-3-yl]quinolin-4-yl}carbonyl)amino]methyl}cyclohexyl)methyl]carbamate;
2,2-Difluoroethyl [(trans-4-{[({2-[6-(4-methylpiperazin-1-yl)pyridin-3-yl]quinolin-4-yl}carbonyl)amino]methyl}cyclohexyl)methyl]carbamate;
2-Fluoroethyl [(trans-4-{[({2-[6-(4-methylpiperazin-1-yl)pyridin-3-yl]quinolin-4-yl}carbonyl)amino]methyl}cyclohexyl)methyl]carbamate;
Ethyl [(trans-4-{[({2-[6-(4-methylpiperazin-1-yl)pyridin-3-yl]quinolin-4-yl}carbonyl)amino]methyl}cyclohexyl)methyl]carbamate;
2-Methoxyethyl [(trans-4-{[({2-[6-(4-methylpiperazin-1-yl)pyridin-3-yl]quinolin-4-yl}carbonyl)amino]methyl}cyclohexyl)methyl]carbamate;
1-Cyanoethyl [(trans-4-{[({2-[6-(4-methylpiperazin-1-yl)pyridin-3-yl]quinolin-4-yl}carbonyl)amino]methyl}cyclohexyl)methyl]carbamate;
2-Acetamidoethyl [(trans-4-{[({2-[6-(4-methylpiperazin-1-yl)pyridin-3-yl]quinolin-4-yl}carbonyl)amino]methyl}cyclohexyl)methyl]carbamate;
(3-Methyloxetan-3-yl)methyl [(trans-4-{[({2-[6-(4-methylpiperazin-1-yl)pyridin-3-yl]quinolin-4-yl}carbonyl)amino]methyl}cyclohexyl)methyl]carbamate;
(3R)-5-Oxopyrrolidin-3-yl [(trans-4-{[({2-[6-(4-methylpiperazin-1-yl)pyridin-3-yl]quinolin-4-yl}carbonyl)amino]methyl}cyclohexyl)methyl]carbamate;
(3S)-5-Oxopyrrolidin-3-yl [(trans-4-{[({2-[6-(4-methylpiperazin-1-yl)pyridin-3-yl]quinolin-4-yl}carbonyl)amino]methyl}cyclohexyl)methyl]carbamate;
Tetrahydrofuran-3-ylmethyl [(trans-4-{[({2-[6-(4-methylpiperazin-1-yl)pyridin-3-yl]quinolin-4-yl}carbonyl)amino]methyl}cyclohexyl)methyl]carbamate;
Tetrahydrofuran-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
(5-Methylisoxazol-3-yl)methyl [(trans-4-{[({2-[6-(4-methylpiperazin-1-yl)pyridin-3-yl]quinolin-4-yl}carbonyl)amino]methyl}cyclohexyl)methyl]carbamate;
2-(1H-Pyrazol-1-yl)ethyl [(trans-4-{[({2-[6-(4-methylpiperazin-1-yl)pyridin-3-yl]quinolin-4-yl}carbonyl)amino]methyl}cyclohexyl)methyl]carbamate;
1,3-Thiazol-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
Pyrazin-2-ylmethyl [(trans-4-{[({2-[6-(4-methylpiperazin-1-yl)pyridin-3-yl]quinolin-4-yl}carbonyl)amino]methyl}cyclohexyl)methyl]carbamate;
2-Cyanoethyl [(trans-4-{[({2-[6-(4-methylpiperazin-1-yl)pyridin-3-yl]quinolin-4-yl}carbonyl)amino]methyl}cyclohexyl)methyl]carbamate;
(1S)-2-Hydroxy-1-methylethyl [(trans-4-{[({2-[6-(4-methylpiperazin-1-yl)pyridin-3-yl]quinolin-4-yl}carbonyl)amino]methyl}cyclohexyl)methyl]carbamate;
tert-Butyl {[trans-4-({[(2-{4-[(methylamino)sulfonyl]-phenyl}quinolin-4-yl)carbonyl]amino}methyl)cyclohexyl]methyl}-carbamate;
tert-Butyl [(trans-4-{[({2-[4-(aminosulfonyl)phenyl]-quinolin-4-yl}carbonyl)amino]-methyl}-cyclohexyl)methyl]-carbamate;
Tetrahydro-2H-pyran-4-yl {[trans-4-({[(2-{4-[(methylamino)sulfonyl]phenyl}-quinolin-4-yl)carbonyl]amino}methyl)cyclohexyl]methyl}carbamate;
(3S)-Tetrahydrofuran-3-yl {[trans-4-({[(2-{4-[(methylamino)sulfonyl]phenyl}-quinolin-4-yl)carbonyl]amino}methyl)cyclohexyl]methyl}-carbamate;
Tetrahydro-2H-pyran-4-yl [(trans-4-{[({2-[4-(aminosulfonyl)phenyl]-quinolin-4-yl}carbonyl)amino]-methyl}cyclohexyl)-methyl]carbamate;
(3S)-Tetrahydrofuran-3-yl [(trans-4-{[({2-[4-(aminosulfonyl)phenyl]-quinolin-4-yl}carbonyl)amino]-methyl}cyclohexyl)-methyl]carbamate;
Tetrahydro-2H-pyran-4-yl {[trans-4-({[(2-{4-[(dimethylamino)sulfonyl]phenyl}-quinolin-4-yl)carbonyl]amino}methyl)cyclohexyl]-methyl}carbamate;
(3S)-Tetrahydrofuran-3-yl {[trans-4-({[(2-{4-[(dimethylamino)sulfonyl]phenyl}-quinolin-4-yl)carbonyl]amino}methyl)cyclohexyl]-methyl}carbamate;
tert-Butyl [(trans-4-{[({2-[6-(4-acetylpiperazin-1-yl)pyridin-3-yl]quinolin-4-yl}carbonyl)amino]methyl}cyclohexyl)methyl]carbamate;
tert-Butyl {[trans-4-({[(2-{6-[(2-hydroxyethyl)amino]pyridin-3-yl}quinolin-4-yl)carbonyl]amino}methyl)cyclohexyl]methyl}carbamate;
tert-Butyl ({trans-4-[({[2-(6-pyrrolidin-1-ylpyridin-3-yl)quinolin-4-yl]carbonyl}amino)methyl]cyclohexyl}methyl)carbamate;
tert-Butyl ({trans-4-[({[2-(6-morpholin-4-ylpyridin-3-yl)quinolin-4-yl]carbonyl}amino)methyl]cyclohexyl}methyl)carbamate;
tert-Butyl ({trans-4-[({[2-(6-{[2-(dimethylamino)ethyl]amino}pyridin-3-yl)quinolin-4-yl]carbonyl}amino)methyl]cyclohexyl}methyl)carbamate;
tert-Butyl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
tert-Butyl ({trans-4-[({[2-(6-{[(1S)-2-hydroxy-1-methylethyl]amino}pyridin-3-yl)quinolin-4-yl]carbonyl}amino)methyl]cyclohexyl}methyl)carbamate;
tert-Butyl ({trans-4-[({[2-(6-{[(1R)-2-hydroxy-1-methylethyl]amino}pyridin-3-yl)quinolin-4-yl]carbonyl}amino)methyl]cyclohexyl}methyl)carbamate;
tert-Butyl {[trans-4-({[(2-{6-[(3-hydroxypropyl)amino]pyridin-3-yl}quinolin-4-yl)carbonyl]amino}methyl)cyclohexyl]methyl}carbamate;
tert-Butyl [(trans-4-{[({2-[6-(4-hydroxypiperidin-1-yl)pyridin-3-yl]quinolin-4-yl}carbonyl)amino]methyl}cyclohexyl)methyl]carbamate;
tert-Butyl {[trans-4-({[(2-{6-[bis(2-hydroxyethyl)amino]pyridin-3-yl}quinolin-4-yl)carbonyl]amino}methyl)cyclohexyl]methyl}carbamate;
tert-Butyl [(trans-4-{[({2-[6-(4-carbamoylpiperidin-1-yl)pyridin-3-yl]quinolin-4-yl}carbonyl)amino]methyl}cyclohexyl)methyl]carbamate;
tert-Butyl ({trans-4-[({[2-(6-piperazin-1-ylpyridin-3-yl)quinolin-4-yl]carbonyl}amino)methyl]cyclohexyl}methyl)carbamate;
tert-Butyl ({trans-4-[({[2-(6-{[2-(2-hydroxyethoxy)ethyl]amino}pyridin-3-yl)quinolin-4-yl]carbonyl}amino)methyl]cyclohexyl}methyl)carbamate;
tert-Butyl {[trans-4-({[(2-{6-[(2-methoxyethyl)amino]pyridin-3-yl}quinolin-4-yl)carbonyl]amino}methyl)cyclohexyl]methyl}carbamate;
tert-Butyl [(trans-4-{[({2-[6-(3-hydroxypiperidin-1-yl)pyridin-3-yl]quinolin-4-yl}carbonyl)amino]methyl}cyclohexyl)methyl]carbamate;
3-{[5-(4-{[(trans-4-{[(tert-Butoxycarbonyl)amino]methyl}cyclohexyl)methyl]-carbamoyl}quinolin-2-yl)pyridin-2-yl]amino}-2-methylpropanoic acid;
tert-Butyl ({trans-4-[({[2-(6-{[(5-methylpyrazin-2-yl)methyl]amino}pyridin-3-yl)quinolin-4-yl]carbonyl}amino)methyl]cyclohexyl}methyl)carbamate;
tert-Butyl {[trans-4-({[(2-{6-[(3S)-3-(dimethylamino)pyrrolidin-1-yl]pyridin-3-yl}quinolin-4-yl)carbonyl]amino}methyl)cyclohexyl]methyl}carbamate;
tert-Butyl {[trans-4-({[(2-{6-[4-(hydroxymethyl)piperidin-1-yl]pyridin-3-yl}quinolin-4-yl)carbonyl]amino}methyl)cyclohexyl]methyl}carbamate;
tert-Butyl {[trans-4-({[(2-{6-[(2,3-dihydroxypropyl)amino]pyridin-3-yl}quinolin-4-yl)carbonyl]amino}methyl)cyclohexyl]methyl}carbamate;
tert-Butyl {[trans-4-({[(2-{6-[(2S)-2-carbamoylpyrrolidin-1-yl]pyridin-3-yl}quinolin-4-yl)carbonyl]amino}methyl)cyclohexyl]methyl}carbamate;
tert-Butyl ({trans-4-[({[2-(6-{[2-hydroxy-1-(hydroxymethyl)ethyl]amino}pyridin-3-yl)quinolin-4-yl]carbonyl}amino)methyl]cyclohexyl}methyl)carbamate;
tert-Butyl [(trans-4-{[({2-[6-(3-oxopiperazin-1-yl)pyridin-3-yl]quinolin-4-yl}carbonyl)amino]methyl}cyclohexyl)methyl]carbamate;
tert-Butyl ({trans-4-[({[2-(6-{[(1-methyl-1H-pyrazol-4-yl)methyl]amino}pyridin-3-yl)quinolin-4-yl]carbonyl}amino)methyl]cyclohexyl}methyl)carbamate;
tert-Butyl {[trans-4-({[(2-{6-[(3-amino-3-oxopropyl)amino]pyridin-3-yl}quinolin-4-yl)carbonyl]amino}methyl)cyclohexyl]methyl}carbamate;
tert-Butyl [(trans-4-{[({2-[6-(3-hydroxyazetidin-1-yl)pyridin-3-yl]quinolin-4-yl}carbonyl)amino]methyl}cyclohexyl)methyl]carbamate;
tert-Butyl ({trans-4-[({[2-(6-{[(1S)-1-carbamoylpropyl]amino}pyridin-3-yl)quinolin-4-yl]carbonyl}amino)methyl]cyclohexyl}methyl)carbamate;
tert-Butyl ({trans-4-[({[2-(6-{[(5-methyl-1,2,4-oxadiazol-3-yl)methyl]amino}pyridin-3-yl)quinolin-4-yl]carbonyl}amino)methyl]cyclohexyl}methyl)carbamate;
tert-Butyl {[trans-4-({[(2-{6-[2-(hydroxymethyl)morpholin-4-yl]pyridin-3-yl}quinolin-4-yl)carbonyl]amino}methyl)cyclohexyl]methyl}carbamate;
tert-Butyl [(trans-4-{[({2-[6-(4-oxoimidazolidin-1-yl)pyridin-3-yl]quinolin-4-yl}carbonyl)amino]methyl}cyclohexyl)methyl]carbamate;
tert-Butyl [(trans-4-{[({2-[6-(tetrahydrofuran-3-ylmethoxy)pyridin-3-yl]quinolin-4-yl}carbonyl)amino]methyl}cyclohexyl)methyl]carbamate;
tert-Butyl [(trans-4-{[({2-[6-(2-hydroxyethoxy)pyridin-3-yl]quinolin-4-yl}carbonyl)amino]methyl}cyclohexyl)methyl]carbamate;
tert-Butyl [(trans-4-{[({2-[6-(tetrahydro-2H-pyran-4-yloxy)pyridin-3-yl]quinolin-4-yl}carbonyl)amino]methyl}cyclohexyl)methyl]carbamate;
tert-Butyl [(trans-4-{[({2-[6-(2-hydroxy-1-methylpropoxy)pyridin-3-yl]quinolin-4-yl}carbonyl)amino]methyl}cyclohexyl)methyl]carbamate;
tert-Butyl {[trans-4-({[(2-{6-[(2-oxopyrrolidin-1-yl)methoxy]pyridin-3-yl}quinolin-4-yl)carbonyl]amino}methyl)cyclohexyl]methyl}carbamate;
tert-Butyl [(trans-4-{[({2-[6-(2-amino-2-oxoethoxy)pyridin-3-yl]quinolin-4-yl}carbonyl)amino]methyl}cyclohexyl)methyl]carbamate;
tert-Butyl {[trans-4-({[(2-{6-[2-(4-methylpiperazin-1-yl)ethoxy]pyridin-3-yl}quinolin-4-yl)carbonyl]amino}methyl)cyclohexyl]methyl}carbamate;
tert-Butyl [(trans-4-{[({2-[3-(4-methylpiperazin-1-yl)propoxy]quinolin-4-yl}carbonyl)amino]methyl}cyclohexyl)methyl]carbamate;
tert-Butyl [(trans-4-{[({2-[3-(dimethylamino)propoxy]quinolin-4-yl}carbonyl)amino]methyl}cyclohexyl)methyl]carbamate;
tert-Butyl [(trans-4-{[({2-[2-(4-methylpiperazin-1-yl)ethoxy]quinolin-4-yl}carbonyl)amino]methyl}cyclohexyl)methyl]carbamate;
tert-Butyl [(trans-4-{[({2-[3-(4-acetylpiperazin-1-yl)propoxy]quinolin-4-yl}carbonyl)amino]methyl}cyclohexyl)methyl]carbamate;
tert-Butyl {[trans-4-({[(2-{6-[2-(dimethylamino)ethoxy]pyridin-3-yl}quinolin-4-yl)carbonyl]amino}methyl)cyclohexyl]methyl}carbamate;
tert-Butyl [(trans-4-{[({2-[4-(2-hydroxyethyl)piperazin-1-yl]quinolin-4-yl}carbonyl)amino]methyl}cyclohexyl)methyl]carbamate;
tert-Butyl {[trans-4-({[(2-{4-[2-(dimethylamino)ethyl]-piperazin-1-yl}quinolin-4-yl)carbonyl]amino}-methyl)cyclohexyl]methyl}carbamate;
tert-Butyl ({trans-4-[({[2-(4-hydroxypiperidin-1-yl)quinolin-4-yl]carbonyl}amino)methyl]cyclohexyl}methyl)carbamate;
tert-Butyl [(trans-4-{[({2-[4-(hydroxymethyl)-piperidin-1-yl]quinolin-4-yl}carbonyl)amino]-methyl}cyclohexyl)-methyl]carbamate;
tert-Butyl [(trans-4-{[({2-[4-(2-hydroxyethyl)piperidin-1-yl]quinolin-4-yl}carbonyl)amino]-methyl}cyclohexyl)-methyl]carbamate;
tert-Butyl {[trans-4-({[(2-piperazin-1-ylquinolin-4-yl)carbonyl]amino}methyl)cyclohexyl]methyl}carbamate;
tert-Butyl [(trans-4-{[({2-[4-(acetamidomethyl)piperidin-1-yl]quinolin-4-yl}carbonyl)amino]methyl}cyclohexyl)methyl]carbamate;
tert-Butyl [(trans-4-{[({2-[4-(2-acetamidoethyl)piperidin-1-yl]quinolin-4-yl}carbonyl)amino]methyl}cyclohexyl)methyl]carbamate;
tert-Butyl {[trans-4-({[(2-{4-[2-(dimethylamino)ethyl]piperidin-1-yl}quinolin-4-yl)carbonyl]amino}methyl)cyclohexyl]methyl}carbamate;
tert-Butyl [(trans-4-{[({2-[4-(2-aminoethyl)piperidin-1-yl]quinolin-4-yl}carbonyl)amino]methyl}cyclohexyl)methyl]carbamate;
Tetrahydro-2H-pyran-4-yl {[trans-4-({[(2-{6-[2-(dimethylamino)ethoxy]pyridin-3-yl}quinolin-4-yl)carbonyl]amino}methyl)cyclohexyl]methyl}carbamate;
(3S)-Tetrahydrofuran-3-yl {[trans-4-({[(2-{6-[2-(dimethylamino)ethoxy]pyridin-3-yl}quinolin-4-yl)carbonyl]amino}methyl)cyclohexyl]methyl}carbamate;
Tetrahydro-2H-pyran-4-yl {[trans-4-({[(2-{6-[(2-hydroxyethyl)amino]pyridin-3-yl}quinolin-4-yl)carbonyl]amino}methyl)cyclohexyl]methyl}carbamate;
Tetrahydro-2H-pyran-4-yl ({trans-4-[({[2-(6-{[2-(dimethylamino)ethyl]amino}-pyridin-3-yl)quinolin-4-yl]carbonyl}amino)methyl]cyclohexyl}methyl)carbamate;
(3S)-Tetrahydrofuran-3-yl ({trans-4-[({[2-(6-{[2-(dimethylamino)ethyl]amino}-pyridin-3-yl)quinolin-4-yl]carbonyl}amino)methyl]cyclohexyl}methyl)carbamate;
(3S)-Tetrahydrofuran-3-yl {[trans-4-({[(2-{6-[(2-hydroxyethyl)amino]pyridin-3-yl}quinolin-4-yl)carbonyl]amino}methyl)cyclohexyl]methyl}carbamate;
(3S)-Tetrahydrofuran-3-yl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
Tetrahydro-2H-pyran-4-yl {[trans-4-({[(2-{6-[(3R)-3-hydroxypyrrolidin-1-yl]pyridin-3-yl}quinolin-4-yl)carbonyl]amino}methyl)cyclohexyl]methyl}carbamate;
tert-Butyl {[trans-4-({[(2-{4-[2-(dimethylamino)ethoxy]phenyl}quinolin-4-yl)carbonyl]amino}methyl)cyclohexyl]methyl}carbamate;
tert-Butyl ({trans-4-[({2-[6-(4-methylpiperazin-1-yl)pyridin-3-yl]quinolin-4-yl}carbonyl)amino]cyclohexyl}methyl)carbamate;
tert-Butyl {[trans-4-({[(2-{4-[3-(dimethylamino)-2-hydroxypropyl]piperidin-1-yl}quinolin-4-yl)carbonyl]amino}methyl)cyclohexyl]methyl}carbamate;
tert-Butyl [(trans-4-{[({2-[4-(2-hydroxy-3-pyrrolidin-1-ylpropyl)-piperidin-1-yl]quinolin-4-yl}carbonyl)amino]-methyl}cyclohexyl)methyl]carbamate;
tert-Butyl [(trans-4-{[({2-[4-(2-hydroxy-3-morpholin-4-ylpropyl)piperidin-1-yl]quinolin-4-yl}-carbonyl)amino]methyl}cyclohexyl)methyl]-carbamate;
(R) or (S) tert-Butyl [(trans-4-{[({2-[4-(2-hydroxy-3-morpholin-4-ylpropyl) piperidin-1-yl]quinolin-4-yl}-carbonyl)amino]methyl}cyclohexyl)methyl]-carbamate;
(S) or (R) tert-Butyl [(trans-4-{[({2-[4-(2-hydroxy-3-pyrrolidin-1-ylpropyl)piperidin-1-yl]quinolin-4-yl}carbonyl)amino]-methyl}cyclohexyl)methyl]carbamate;
tert-Butyl {[trans-4-({[(2-{4-[2-(dimethylamino)-2-oxoethoxy]piperidin-1-yl}quinolin-4-yl)carbonyl]amino}methyl)cyclohexyl]methyl}carbamate;
tert-Butyl {[trans-4-({[(2-{4-[2-(dimethylamino)ethoxy]piperidin-1-yl}quinolin-4-yl)carbonyl]amino}methyl)cyclohexyl]methyl}carbamate;
2,2-Dimethylpropyl {[trans-4-({[(2-{4-[2-(dimethylamino)ethyl]piperidin-1-yl}quinolin-4-yl)carbonyl]amino}methyl)cyclohexyl]methyl}carbamate;
tert-Butyl [(trans-4-{[({2-[4-(morpholin-4-ylmethyl)piperidin-1-yl]quinolin-4-yl}carbonyl)amino]methyl}cyclohexyl)methyl]-carbamate;
tert-Butyl [(trans-4-{[({2-[4-(4-methylpiperazin-1-yl)piperidin-1-yl]quinolin-4-yl}carbonyl)amino]methyl}cyclohexyl)methyl]-carbamate;
tert-Butyl ({trans-4-[({[2-(pyridin-3-yloxy)quinolin-4-yl]carbonyl}amino)methyl]cyclohexyl}methyl)carbamate;
tert-Butyl [(trans-4-{[({2-[(1-methylpiperidin-4-yl)methoxy]quinolin-4-yl}carbonyl)amino]methyl}cyclohexyl)methyl]-carbamate;
tert-Butyl ({trans-4-[({[2-(3-{[2-(dimethylamino)ethyl]carbamoyl}azetidin-1-yl)quinolin-4-yl]carbonyl}amino)methyl]cyclohexyl}methyl)carbamate;
tert-Butyl ({trans-4-[({[2-(4-aminophenyl)quinolin-4-yl]carbonyl}amino)methyl]cyclohexyl}methyl)carbamate;
tert-Butyl {[trans-4-({[(2-{4-[2-(dimethylamino)ethyl]-3-oxopiperazin-1-yl}quinolin-4-yl)carbonyl]amino}methyl)cyclohexyl]methyl}carbamate;
tert-Butyl [(trans-4-{[({2-[4-(hydroxyacetyl)piperazin-1-yl]quinolin-4-yl}carbonyl)amino]methyl}cyclohexyl)methyl]carbamate;
tert-Butyl {[trans-4-({[(2-{4-[(4-fluorobenzyl)oxy]piperidin-1-yl}quinolin-4-yl)carbonyl]amino}methyl)cyclohexyl]methyl}carbamate;
tert-Butyl {[trans-4-({[(2-amino-1-benzothiophen-3-yl)carbonyl]amino}methyl)cyclohexyl]methyl}carbamate;
tert-Butyl [(trans-4-{[({2-[(ethylcarbamoyl)amino]-1-benzothiophen-3-yl}carbonyl)amino]methyl}cyclohexyl)methyl]carbamate;
Tetrahydro-2H-pyran-4-yl {[trans-4-({[(2-{4-[2-(dimethylamino)ethyl]piperidin-1-yl}quinolin-4-yl)carbonyl]amino}methyl)cyclohexyl]methyl}carbamate;
tert-Butyl ({trans-4-[({[2-(1′-methyl-4,4′-bipiperidin-1-yl)quinolin-4-yl]carbonyl}amino)methyl]cyclohexyl}methyl)carbamate;
tert-Butyl ({trans-4-[({[2-(4-{2-[(2-methoxyethyl)amino]ethyl}piperidin-1-yl)quinolin-4-yl]carbonyl}amino)methyl]cyclohexyl}methyl)carbamate;
tert-Butyl ({trans-4-[({[2-(4-{2-[(2-methoxyethyl)(methyl)-amino]ethyl}piperidin-1-yl)quinolin-4-yl]carbonyl}amino)-methyl]cyclohexyl}-methyl)carbamate;
tert-Butyl {[trans-4-({[(2-{4-[2-(tetrahydro-2H-pyran-4-ylamino)ethyl]piperidin-1-yl}quinolin-4-yl)carbonyl]amino}-methyl)cyclohexyl]-methyl}carbamate;
tert-Butyl {[trans-4-({[(2-{4-[2-(3-methoxyazetidin-1-yl)ethyl]piperidin-1-yl}quinolin-4-yl)carbonyl]amino}-methyl)cyclohexyl]-methyl}carbamate;
tert-Butyl {[trans-4-({[(2-{4-[2-(3-hydroxypyrrolidin-1-yl)ethyl]piperidin-1-yl}quinolin-4-yl)carbonyl]amino}-methyl)cyclohexyl]-methyl}carbamate;
tert-Butyl [(trans-4-{[({2-[4-(2-pyrrolidin-1-ylethyl)piperidin-1-yl]quinolin-4-yl}carbonyl)amino]-methyl}cyclohexyl)-methyl]carbamate;
tert-Butyl ({trans-4-[({[2-(4-{2-[(2-hydroxyethyl)(methyl)-amino]ethyl}piperidin-1-yl)quinolin-4-yl]carbonyl}amino)-methyl]cyclohexyl}-methyl)carbamate;
tert-Butyl {[trans-4-({[(2-{4-[2-(3-hydroxyazetidin-1-yl)ethyl]piperidin-1-yl}quinolin-4-yl)carbonyl]amino}methyl)cyclohexyl]methyl}carbamate;
tert-Butyl [(trans-4-{[({2-[4-(2-azetidin-1-ylethyl)piperidin-1-yl]quinolin-4-yl}carbonyl)amino]methyl}cyclohexyl)methyl]carbamate;
1-{2-[1-(4-{[(trans-4-{[(tert-Butoxycarbonyl)amino]methyl}cyclohexyl)methyl]-carbamoyl}quinolin-2-yl)piperidin-4-yl]ethyl}azetidine-3-carboxylic acid;
tert-Butyl {[trans-4-({[(2-{4-[2-(3-aminoazetidin-1-yl)ethyl]piperidin-1-yl}quinolin-4-yl)carbonyl]amino}methyl)cyclohexyl]methyl}carbamate;
tert-Butyl {[trans-4-({[(2-{4-[3-(dimethylamino)-2-fluoropropyl]piperidin-1-yl}quinolin-4-yl)carbonyl]amino}methyl)cyclohexyl]methyl}carbamate;
tert-Butyl {[trans-4-({[(2-{3-[2-(dimethylamino)ethoxy]azetidin-1-yl}quinolin-4-yl)carbonyl]amino}methyl)cyclohexyl]methyl}carbamate;
tert-Butyl [(trans-4-{[({2-[3-(aminomethyl)phenyl]-6-(1H-pyrazol-4-yl)pyridin-4-yl}carbonyl)amino]methyl}cyclohexyl)methyl]carbamate;
tert-Butyl [(trans-4-{[({6-[3-(aminomethyl)phenyl]-2,3′-bipyridin-4-yl}carbonyl)amino]methyl}cyclohexyl)methyl]carbamate;
tert-Butyl [(trans-4-{[({6′-amino-6-[3-(aminomethyl)phenyl]-2,3′-bipyridin-4-yl}carbonyl)amino]methyl}cyclohexyl)methyl]carbamate;
tert-Butyl {[trans-4-({[(2-{1-[2-(dimethylamino)ethyl]-1H-pyrazol-4-yl}quinolin-4-yl)carbonyl]amino}methyl)cyclohexyl]methyl}carbamate;
tert-Butyl [(trans-4-{[({2-[1-(2-hydroxyethyl)-1H-pyrazol-4-yl]quinolin-4-yl}carbonyl)amino]methyl}cyclohexyl)methyl]carbamate;
tert-Butyl ({trans-4-[({[2-(4-{2-[(2-fluoroethylamino]ethyl}piperidin-1-yl)quinolin-4-yl]carbonyl}amino)methyl]cyclohexyl}methyl)carbamate;
tert-Butyl ({trans-4-[({[2-(4-(2-((2-fluoroethyl)(methyl)amino)ethyl}piperidin-1-yl)quinolin-4-yl]carbonyl}amino)methyl]cyclohexyl}methyl)carbamate;
or a pharmaceutically acceptable salt thereof,
or an enantiomer thereof.

13. A method of treating obesity or being overweight, an eating disorder, dyslipidemia, atherosclerosis, heart failure, stroke, type 2 diabetes mellitus or preventing type 2 diabetes comprising administering a pharmacologically effective amount of a compound of formula (I) or formula (II) according to claim 1, including proviso b), to a patient in need thereof.

14. A process for preparing a compound of formula (I) or formula (II) as described herein.
